SEQUENCE LISTING (347 sequences)

SEQ ID NO: 1 - protein, 157 aa, Homo sapiens
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 2 - protein, 157 aa, synthetic construct
YFPKLRSKLSVIRNLNDQVLFIDQGNRPLFEDMTDSDCRDNAPRTIFIISMYKHSDKRGMAVTISVKCEKISTLSCENKIISFKEMNPPDVIKDTKSDIIFFQYSVPGHDNKMQFESSSYEGYFLACEKERDLFKLILKKEDELGDRSIMFTVQNED

SEQ ID NO: 3 - protein, 157 aa, synthetic construct
YFPKLKSKLSVIRNLNDQVLFIDQGNRPLFEDMTDSDCRDNAPRTIFIISMYKQSIWRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 4 - protein, 157 aa, synthetic construct
YFEKLTSKLSVIRNLNDQVLFIDQGNRPLFEDMTDSDCRDNAPRTIFIISMYKWSPWRGMAVTISVKCEKISTLSCENKIISFKEMNPPDVIKDTKSDIIFFQTSVPGHDNKMQFESSSYEGYFLACEKERDLFKLILKKEDELGDRSIMFTVQKED

SEQ ID NO: 5 - protein, 157 aa, synthetic construct
YFPKLRSKLSVIRNLNDQVLFIDQGNRPLFEDMTDSDCRDNAPRTIFIISMYKHSTVRGMAVTISVKCEKISTLSCENKIISFKEMNPPDVIKDTKSDIIFFQRSVPGHDNKMQFESSSYEGYFLACEKERDLFKLILKKEDELGDRSIMFTVQNED

SEQ ID NO: 6 - protein, 157 aa, synthetic construct
YFPKLSSKLSVIRNLNDQVLFIDQGNRPLFEDMTDSDCRDNAPRTIFIISMYKWSPWRGMAVTISVKCEKISTLSCENKIISFKEMNPPDDIKDTKSDIIFFQRSVPGHDNKMQFESSSYEGYFLACEKERDLFKLILKKEDELGDRSIMFTVQNED

SEQ ID NO: 7 - protein, 157 aa, synthetic construct
YFPKLKSKLSVIRNLNDQVLFIDQGNRPLFEDMTDSDCRDNAPRTIFIISMYKHSTVRGMAVTISVKCEKISTLSCENKIISFKEMNPPDVIKDTKSDIIFFQRSVPGHDNKMQFESSSYEGYFLACEKERDLFKLILKKEDELGDRSIMFTVQNED

SEQ ID NO: 8 - protein, 157 aa, synthetic construct
YFPKLGSKLSVIRNLNDQVLFIDQGNRPLFEDMTDSDCRDNAPRTIFIISMYKQSVWRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 9 - protein, 157 aa, synthetic construct
YFPKLRSKLSVIRNLNDQVLFIDQGNRPLFEDMTDSDCRDNAPRTIFIISMYKSSYTRGMAVTISVKCEKISTLSCENKIISFKEMNPPDGIKDTKSDIIFFQRSVPGHDNKMQFESSSYEGYFLACEKERDLFKLILKKEDELGDRSIMFTVQNED

SEQ ID NO: 10 - protein, 157 aa, synthetic construct
YFTKLKSKLSVIRNLNDQVLFIDQGNRPLFEDMTDSDCRDNAPRTIFIISMYKWSSDRGMAVTISVKCEKISTLSCENKIISFKEMNPPDAIKDTKSDIIFFQVSVPGHDNKMQFESSSYEGYFLACEKERDLFKLILKKEDELGDRSIMFTVQNED

SEQ ID NO: 11 - protein, 157 aa, synthetic construct
YFPKLRSKLSVIRNLNDQVLFIDQGNRPLFEDMTDSDCRDNAPRTIFIISMYKHSTARGMAVTISVKCEKISTLSCENKIISFKEMNPPDAIKDTKSDIIFFQRSVPGHDNKMQFESSSYEGYFLACEKERDLFKLILKKEDELGDRSIMFTVQNED

SEQ ID NO: 12 - protein, 157 aa, synthetic construct
YFPKLKSKLSVIRNLNDQVLFIDQGNRPLFEDMTDSDCRDNAPRTIFIISMYKWSHVRGMAVTISVKCEKISTLSCENKIISFKEMNPPDAIKDTKSDIIFFQYSVPGHDNKMQFESSSYEGYFLACEKERDLFKLILKKEDELGDRSIMFTVQNED

SEQ ID NO: 13 - protein, 157 aa, synthetic construct
YFPKLRSKLSVIRNLNDQVLFIDQGNRPLFEDMTDSDCRDNAPRTIFIISMYKWSKTRGMAVTISVKCEKISTLSCENKIISFKEMNPPDVIKDTKSDIIFFQYSVPGHDNKMQFESSSYEGYFLACEKERDLFKLILKKEDELGDRSIMFTVQNED

SEQ ID NO: 14 - protein, 157 aa, synthetic construct
YFPKLRSKLSVIRNLNDQVLFIDQGNRPLFEDMTDSDCRDNAPRTIFIISMYKYSTARGMAVTISVKCEKISTLSCENKIISFKEMNPPDVIKDTKSDIIFFQHSVPGHDNKMQFESSSYEGYFLACEKERDLFKLILKKEDELGDRSIMFTVQNED

SEQ ID NO: 15 - protein, 157 aa, synthetic construct
YFPKLRSKLSVIRNLNDQVLFIDQGNRPLFEDMTDSDCRDNAPRTIFIISMYKSSRGRGMAVTISVKCEKISTLSCENKIISFKEMNPPDVIKDTKSDIIFFQTSVPGHDNKMQFESSSYEGYFLACEKERDLFKLILKKEDELGDRSIMFTVQNED

SEQ ID NO: 16 - protein, 157 aa, synthetic construct
YFPKLTSKLSVIRNLNDQVLFIDQGNRPLFEDMTDSDCRDNAPRTIFIISMYKHSTYRGMAVTISVKCEKISTLSCENKIISFKEMNPPDAIKDTKSDIIFFQRSVPGHDNKMQFESSSYEGYFLACEKERDLFKLILKKEDELGDRSIMFTVQKED

SEQ ID NO: 17 - protein, 157 aa, synthetic construct
YFPKLRSKLSVIRNLNDQVLFIDQGNRPLFEDMTDSDCRDNAPRTIFIISMYKGSGARGMAVTISVKCEKISTLSCENKIISFKEMNPPDGIKDTKSDIIFFQRSVPGHDNKMQFESSSYEGYFLACEKERDLFKLILKKEDELGDRSIMFTVQNED

SEQ ID NO: 18 - protein, 157 aa, synthetic construct
YLPKLRSKLSVIRNLNDQVLFIDQGNRPLFEDMTDSDCRDNAPRTIFIISMYKWSTSRGMAVTISVKCEKISTLSCENKIISFKEMNPPDSIKDTKSDIIFFQSSVPGHDNKMQFESSSYEGYFLACEKERDLFKLILKKEDELGDRSIMFTVQKED

SEQ ID NO: 19 - protein, 157 aa, synthetic construct
YFPKLRSKLSVIRNLNDQVLFIDQGNRPLFEDMTDSDCRDNAPRTIFIISMYKWSYPRGMAVTISVKCEKISTLSCENKIISFKEMNPPDSIKDTKSDIIFFQSSVPGHDNKMQFESSSYEGYFLACEKERDLFKLILKKEDELGDRSIMFTVQNED

SEQ ID NO: 20 - protein, 157 aa, synthetic construct
YFPKLKSKLSVIRNLNDQVLFIDQGNRPLFEDMTDSDCRDNAPRTIFIISMYKFSTVRGMAVTISVKCEKISTLSCENKIISFKEMNPPDGIKDTKSDIIFFQHSVPGHDNKMQFESSSYEGYFLACEKERDLFKLILKKEDELGDRSIMFTVQNED

SEQ ID NO: 21 - protein, 157 aa, synthetic construct
YFSKLKSKLSVIRNLNDQVLFIDQGNRPLFEDMTDSDCRDNAPRTIFIISMYKWSPDRGMAVTISVKCEKISTLSCENKIISFKEMNPPDGIKDTKSDIIFFQTSVPGHDNKMQFESSSYEGYFLACEKERDLFKLILKKEDELGDRSIMFTVQNED

SEQ ID NO: 22 - protein, 157 aa, synthetic construct
YFPKLKSKLSVIRNLNDQVLFIDQGNRPLFEDMTDSDCRDNAPRTIFIISMYKLSTQRGMAVTISVKCEKISTLSCENKIISFKEMNPPDVIKDTKSDIIFFQRSVPGHDNKMQFESSSYEGYFLACEKERDLFKLILKKEDELGDRSIMFTVQNED

SEQ ID NO: 23 - protein, 157 aa, synthetic construct
YFPKLKSKLSVIRNLNDQVLFIDQGNRPLFEDMTDSDCRDNAPRTIFIISMYKGSGTRGMAVTISVKCEKISTLSCENKIISFKEMNPPDAIKDTKSDIIFFQRSVPGHDNKMQFESSSYEGYFLACEKERDLFKLILKKEDELGDRSIMFTVQNED

SEQ ID NO: 24 - protein, 157 aa, synthetic construct
YFDKLGSKLSVIRNLNDQVLFIDQGNRPLFEDMTDSDCRDNAPRTIFIISMYKWSPWRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 25 - protein, 157 aa, synthetic construct
YFPKLKSKLSVIRNLNDQVLFIDQGNRPLFEDMTDSDCRDNAPRTIFIISMYKLSGVRGMAVTISVKCEKISTLSCENKIISFKEMNPPDTIKDTKSDIIFFQRSVPGHDNKMQFESSSYEGYFLACEKERDLFKLILKKEDELGDRSIMFTVQNED

SEQ ID NO: 26 - protein, 157 aa, synthetic construct
YFPKLKSKLSVIRNLNDQVLFIDQGNRPLFEDMTDSDCRDNAPRTIFIISMYKHSEQRGMAVTISVKCEKISTLSCENKIISFKEMNPPDAIKDTKSDIIFFQRSVPGHDNKMQFESSSYEGYFLACEKERDLFKLILKKEDELGDRSIMFTVQNED

SEQ ID NO: 27 - protein, 157 aa, synthetic construct
YFPKLRSKLSVIRNLNDQVLFIDQGNRPLFEDMTDSDCRDNAPRTIFIISMYKSSSTRGMAVTISVKCEKISTLSCENKIISFKEMNPPDGIKDTKSDIIFFQSSVPGHDNKMQFESSSYEGYFLACEKERDLFKLILKKEDELGDRSIMFTVQNED

SEQ ID NO: 28 - protein, 157 aa, synthetic construct
YFPKLKSKLSVIRNLNDQVLFIDQGNRPLFEDMTDSDCRDNAPRTIFIISMYKHSDRRGMAVTISVKCEKISTLSCENKIISFKEMNPPDVIKDTKSDIIFFQVSVPGHDNKMQFESSSYEGYFLACEKERDLFKLILKKEDELGDRSIMFTVQNED

SEQ ID NO: 29 - protein, 157 aa, synthetic construct
YFPKLTSKLSVIRNLNDQVLFIDQGNRPLFEDMTDSDCRDNAPRTIFIISMYKHSYARGMAVTISVKCEKISTLSCENKIISFKEMNPPDVIKDTKSDIIFFQRSVPGHDNKMQFESSSYEGYFLACEKERDLFKLILKKEDELGDRSIMFTVQNED

SEQ ID NO: 30 - protein, 157 aa, synthetic construct
YFPKLKSKLSVIRNLNDQVLFIDQGNRPLFEDMTDSDCRDNAPRTIFIISMYKHSIHRGMAVTISVKCEKISTLSCENKIISFKEMNPPDVIKDTKSDIIFFQVSVPGHDNKMQFESSSYEGYFLACEKERDLFKLILKKEDELGDRSIMFTVQNED

SEQ ID NO: 31 - protein, 157 aa, synthetic construct
YFPKLKSKLSVIRNLNDQVLFIDQGNRPLFEDMTDSDCRDNAPRTIFIISMYKWSARRGMAVTISVKCEKISTLSCENKIISFKEMNPPDPIKDTKSDIIFFQSSVPGHDNKMQFESSSYEGYFLACEKERDLFKLILKKEDELGDRSIMFTVQNED

SEQ ID NO: 32 - protein, 157 aa, synthetic construct
YFPKLRSKLSVIRNLNDQVLFIDQGNRPLFEDMTDSDCRDNAPRTIFIISMYKHSSRRGMAVTISVKCEKISTLSCENKIISFKEMNPPDIIKDTKSDIIFFQYSVPGHDNKMQFESSSYEGYFLACEKERDLFKLILKKEDELGDRSIMFTVQNED

SEQ ID NO: 33 - protein, 157 aa, synthetic construct
YFGKLKSKLSVIRNLNDQVLFIDQGNRPLFEDMTDSDCRDNAPRTIFIISMYKLSTRRGMAVTISVKCEKISTLSCENKIISFKEMNPPDPIKDTKSDIIFFQRSVPGHDNKMQFESSSYEGYFLACEKERDLFKLILKKEDELGDRSIMFTVQNED

SEQ ID NO: 34 - protein, 157 aa, synthetic construct
YFPKLKSKLSVIRNLNDQVLFIDQGNRPLFEDMTDSDCRDNAPRTIFIISMYKQSTWRGMAVTISVKCEKISTLSCENKIISFKEMNPPDCIKDTKSDIIFFQRSVPGHDNKMQFESSSYEGYFLACEKERDLFKLILKKEDELGDRSIMFTVQNED

SEQ ID NO: 35 - protein, 157 aa, synthetic construct
YFPKLRSKLSVIRNLNDQVLFIDQGNRPLFEDMTDSDCRDNAPRTIFIISMYKSSRNRGMAVTISVKCEKISTLSCENKIISFKEMNPPDGIKDTKSDIIFFQSSVPGHDNKMQFESSSYEGYFLACEKERDLFKLILKKEDELGDRSIMFTVQNED

SEQ ID NO: 36 - protein, 157 aa, synthetic construct
YFPKLRSKLSVIRNLNDQVLFIDQGNRPLFEDMTDSDCRDNAPRTIFIISMYKWSRPRGMAVTISVKCEKISTLSCENKIISFKEMNPPDSIKDTKSDIIFFQYSVPGHDNKMQFESSSYEGYFLACEKERDLFKLILKKEDELGDRSIMFTVQNED

SEQ ID NO: 37 - protein, 157 aa, synthetic construct
YFPKLRSKLSVIRNLNDQVLFIDQGNRPLFEDMTDSDCRDNAPRTIFIISMYKGSGIRGMAVTISVKCEKISTLSCENKIISFKEMNPPDGIKDTKSDIIFFQRSVPGHDNKMQFESSSYEGYFLACEKERDLFKLILKKEDELGDRSIMFTVQNED

SEQ ID NO: 38 - protein, 157 aa, synthetic construct
YFNKLKSKLSVIRNLNDQVLFIDQGNRPLFEDMTDSDCRDNAPRTIFIISMYKPSSSRGMAVTISVKCEKISTLSCENKIISFKEMNPPDRIKDTKSDIIFFQASVPGHDNKMQFESSSYEGYFLACEKERDLFKLILKKEDELGDRSIMFTVQNED

SEQ ID NO: 39 - protein, 157 aa, synthetic construct
YFPKLKSKLSVIRNLNDQVLFIDQGNRPLFEDMTDSDCRDNAPRTIFIISMYKGSGARGMAVTISVKCEKISTLSCENKIISFKEMNPPDTIKDTKSDIIFFQRSVPGHDNKMQFESSSYEGYFLACEKERDLFKLILKKEDELGDRSIMFTVQNED

SEQ ID NO: 40 - protein, 157 aa, synthetic construct
YFPKLKSKLSVIRNLNDQVLFIDQGNRPLFEDMTDSDCRDNAPRTIFIISMYKLSGARGMAVTISVKCEKISTLSCENKIISFKEMNPPDVIKDTKSDIIFFQRSVPGHDNKMQFESSSYEGYFLACEKERDLFKLILKKEDELGDRSIMFTVQNED

SEQ ID NO: 41 - protein, 157 aa, synthetic construct
YFPKLRSKLSVIRNLNDQVLFIDQGNRPLFEDMTDSDCRDNAPRTIFIISMYKHSRNRGMAVTISVKCEKISTLSCENKIISFKEMNPPDVIKDTKSDIIFFQESVPGHDNKMQFESSSYEGYFLACEKERDLFKLILKKEDELGDRSIMFTVQNED

SEQ ID NO: 42 - protein, 157 aa, synthetic construct
YFEKLRSKLSVIRNLNDQVLFIDQGNRPLFEDMTDSDCRDNAPRTIFIISMYKWSPDRGMAVTISVKCEKISTLSCENKIISFKEMNPPDVIKDTKSDIIFFQVSVPGHDNKMQFESSSYEGYFLACEKERDLFKLILKKEDELGDRSIMFTVQNED

SEQ ID NO: 43 - protein, 157 aa, synthetic construct
YFPKLRSKLSVIRNLNDQVLFIDQGNRPLFEDMTDSDCRDNAPRTIFIISMYKLSTARGMAVTISVKCEKISTLSCENKIISFKEMNPPDGIKDTKSDIIFFQRSVPGHDNKMQFESSSYEGYFLACEKERDLFKLILKKEDELGDRSIMFTVQNED

SEQ ID NO: 44 - protein, 157 aa, synthetic construct
YFPKLRSKLSVIRNLNDQVLFIDQGNRPLFEDMTDSDCRDNAPRTIFIISMYKSSRSRGMAVTISVKCEKISTLSCENKIISFKEMNPPDGIKDTKSDIIFFQSSVPGHDNKMQFESSSYEGYFLACEKERDLFKLILKKEDELGDRSIMFTVQNED

SEQ ID NO: 45 - protein, 157 aa, synthetic construct
YFPKLRSKLSVIRNLNDQVLFIDQGNRPLFEDMTDSDCRDNAPRTIFIISMYKHSYGRGMAVTISVKCEKISTLSCENKIISFKEMNPPDAIKDTKSDIIFFQTSVPGHDNKMQFESSSYEGYFLACEKERDLFKLILKKEDELGDRSIMFTVQNED

SEQ ID NO: 46 - protein, 157 aa, synthetic construct
YFPKLGSKLSVIRNLNDQVLFIDQGNRPLFEDMTDSDCRDNAPRTIFIISMYKHSDPRGMAVTISVKCEKISTLSCENKIISFKEMNPPDVIKDTKSDIIFFQMSVPGHDNKMQFESSSYEGYFLACEKERDLFKLILKKEDELGDRSIMFTVQNED

SEQ ID NO: 47 - protein, 157 aa, synthetic construct
YFPKLRSKLSVIRNLNDQVLFIDQGNRPLFEDMTDSDCRDNAPRTIFIISMYKLSTVRGMAVTISVKCEKISTLSCENKIISFKEMNPPDGIKDTKSDIIFFQHSVPGHDNKMQFESSSYEGYFLACEKERDLFKLILKKEDELGDRSIMFTVQNED

SEQ ID NO: 48 - protein, 157 aa, synthetic construct
YFFKLKSKLSVIRNLNDQVLFIDQGNRPLFEDMTDSDCRDNAPRTIFIISMYKPSPPRGMAVTISVKCEKISTLSCENKIISFKEMNPPDTIKDTKSDIIFFQYSVPGHDNKMQFESSSYEGYFLACEKERDLFKLILKKEDELGDRSIMFTVQNED

SEQ ID NO: 49 - protein, 157 aa, synthetic construct
YFPKLRSKLSVIRNLNDQVLFIDQGNRPLFEDMTDSDCRDNAPRTIFIISMYKSSRNRGMAVTISVKCEKISTLSCENKIISFKEMNPPDGIKDTKSDIIFFQTSVPGHDNKMQFESSSYEGYFLACEKERDLFKLILKKEDELGDRSIMFTVQNED

SEQ ID NO: 50 - protein, 157 aa, synthetic construct
YFPKLASKLSVIRNLNDQVLFIDQGNRPLFEDMTDSDCRDNAPRTIFIISMYKWSGGRGMAVTISVKCEKISTLSCENKIISFKEMNPPDVIKDTKSDIIFFQYSVPGHDNKMQFESSSYEGYFLACEKERDLFKLILKKEDELGDRSIMFTVQNED

SEQ ID NO: 51 - protein, 157 aa, synthetic construct
YFPKLRSKLSVIRNLNDQVLFIDQGNRPLFEDMTDSDCRDNAPRTIFIISMYKWSAVRGMAVTISVKCEKISTLSCENKIISFKEMNPPDSIKDTKSDIIFFQSSVPGHDNKMQFESSSYEGYFLACEKERDLFKLILKKEDELGDRSIMFTVQNED

SEQ ID NO: 52 - protein, 157 aa, synthetic construct
YFWKLKSKLSVIRNLNDQVLFIDQGNRPLFEDMTDSDCRDNAPRTIFIISMYKPSSHRGMAVTISVKCEKISTLSCENKIISFKEMNPPDVIKDTKSDIIFFQLSVPGHDNKMQFESSSYEGYFLACEKERDLFKLILKKEDELGDRSIMFTVQNED

SEQ ID NO: 53 - protein, 157 aa, synthetic construct
YFPKLRSKLSVIRNLNDQVLFIDQGNRPLFEDMTDSDCRDNAPRTIFIISMYKLSGWRGMAVTISVKCEKISTLSCENKIISFKEMNPPDVIKDTKSDIIFFQRSVPGHDNKMQFESSSYEGYFLACEKERDLFKLILKKEDELGDRSIMFTVQNED

SEQ ID NO: 54 - protein, 157 aa, synthetic construct
YFPKLRSKLSVIRNLNDQVLFIDQGNRPLFEDMTDSDCRDNAPRTIFIISMYKSSRRRGMAVTISVKCEKISTLSCENKIISFKEMNPPDGIKDTKSDIIFFQSSVPGHDNKMQFESSSYEGYFLACEKERDLFKLILKKEDELGDRSIMFTVQNED

SEQ ID NO: 55 - protein, 157 aa, synthetic construct
YFPKLSSKLSVIRNLNDQVLFIDQGNRPLFEDMTDSDCRDNAPRTIFIISMYKWSGARGMAVTISVKCEKISTLSCENKIISFKEMNPPDVIKDTKSDIIFFQISVPGHDNKMQFESSSYEGYFLACEKERDLFKLILKKEDELGDRSIMFTVQNED

SEQ ID NO: 56 - protein, 157 aa, synthetic construct
YFPKLRSKLSVIRNLNDQVLFIDQGNRPLFEDMTDSDCRDNAPRTIFIISMYKWSTPRGMAVTISVKCEKISTLSCENKIISFKEMNPPDVIKDTKSDIIFFQTSVPGHDNKMQFESSSYEGYFLACEKERDLFKLILKKEDELGDRSIMFTVQNED

SEQ ID NO: 57 - protein, 157 aa, synthetic construct
YFPKLKSKLSVIRNLNDQVLFIDQGNRPLFEDMTDSDCRDNAPRTIFIISMYKHSRARGMAVTISVKCEKISTLSCENKIISFKEMNPPDSIKDTKSDIIFFQTSVPGHDNKMQFESSSYEGYFLACEKERDLFKLILKKEDELGDRSIMFTVQNED

SEQ ID NO: 58 - protein, 157 aa, synthetic construct
YFPKLRSKLSVIRNLNDQVLFIDQGNRPLFEDMTDSDCRDNAPRTIFIISMYKHSYTRGMAVTISVKCEKISTLSCENKIISFKEMNPPDVIKDTKSDIIFFQYSVPGHDNKMQFESSSYEGYFLACEKERDLFKLILKKEDELGDRSIMFTVQNED

SEQ ID NO: 59 - protein, 157 aa, synthetic construct
YFPKLRSKLSVIRNLNDQVLFIDQGNRPLFEDMTDSDCRDNAPRTIFIISMYKWSPGRGMAVTISVKCEKISTLSCENKIISFKEMNPPDVIKDTKSDIIFFQLSVPGHDNKMQFESSSYEGYFLACEKERDLFKLILKKEDELGDRSIMFTVQNED

SEQ ID NO: 60 - protein, 157 aa, synthetic construct
YFPKLKSKLSVIRNLNDQVLFIDQGNRPLFEDMTDSDCRDNAPRTIFIISMYKHSSTRGMAVTISVKCEKISTLSCENKIISFKEMNPPDVIKDTKSDIIFFQCSVPGHDNKMQFESSSYEGYFLACEKERDLFKLILKKEDELGDRSIMFTVQNED

SEQ ID NO: 61 - protein, 157 aa, synthetic construct
YFPKLKSKLSVIRNLNDQVLFIDQGNRPLFEDMTDSDCRDNAPRTIFIISMYKFSTERGMAVTISVKCEKISTLSCENKIISFKEMNPPDPIKDTKSDIIFFQRSVPGHDNKMQFESSSYEGYFLACEKERDLFKLILKKEDELGDRSIMFTVQNED

SEQ ID NO: 62 - protein, 157 aa, synthetic construct
YFPKLRSKLSVIRNLNDQVLFIDQGNRPLFEDMTDSDCRDNAPRTIFIISMYKWSPARGMAVTISVKCEKISTLSCENKIISFKEMNPPDAIKDTKSDIIFFQLSVPGHDNKMQFESSSYEGYFLACEKERDLFKLILKKEDELGDRSIMFTVQNED

SEQ ID NO: 63 - protein, 157 aa, synthetic construct
YFPKLQSKLSVIRNLNDQVLFIDQGNRPLFEDMTDSDCRDNAPRTIFIISMYKLSTPRGMAVTISVKCEKISTLSCENKIISFKEMNPPDAIKDTKSDIIFFQRSVPGHDNKMQFESSSYEGYFLACEKERDLFKLILKKEDELGDRSIMFTVQNED

SEQ ID NO: 64 - protein, 157 aa, synthetic construct
YFPKLNSKLSVIRNLNDQVLFIDQGNRPLFEDMTDSDCRDNAPRTIFIISMYKVPTARGMAVTISVKCEKISTLSCENKIISFKEMNPPDAIKDTKSDIIFFQRSVPGHDNKMQFESSSYEGYFLACEKERDLFKLILKKEDELGDRSIMFTVQNED

SEQ ID NO: 65 - protein, 157 aa, synthetic construct
YFPKLRSKLSVIRNLNDQVLFIDQGNRPLFEDMTDSDCRDNAPRTIFIISMYKHSIHRGMAVTISVKCEKISTLSCENKIISFKEMNPPDIIKDTKSDIIFFQYSVPGHDNKMQFESSSYEGYFLACEKERDLFKLILKKEDELGDRSIMFTVQNED

SEQ ID NO: 66 - protein, 157 aa, synthetic construct
YFPKLRSKLSVIRNLNDQVLFIDQGNRPLFEDMTDSDCRDNAPRTIFIISMYKQSLWRGMAVTISVKCEKISTLSCENKIISFKEMNPPDSIKDTKSDIIFFQRSVPGHDNKMQFESSSYEGYFLACEKERDLFKLILKKEDELGDRSIMFTVQNED

SEQ ID NO: 67 - protein, 157 aa, synthetic construct
YFTKLRSKLSVIRNLNDQVLFIDQGNRPLFEDMTDSDCRDNAPRTIFIISMYKLSHLRGIAVTISVKCEKISTLSCENKIISFKEMNPPDAIKDTKSDIIFFQSSVPGHDNKMQFESSSYEGYFLACEKERDLFKLILKKEDELGDRSIMFTVQNED

SEQ ID NO: 68 - protein, 157 aa, synthetic construct
YFPKLKSKLSVIRNLNDQVLFIDQGNRPLFEDMTDSDCRDNAPRTIFIISMYKWSPWRGMAVTISVKCEKISTLSCENKIISFKEMNPPDNIKDTKSDIIFFQTSVPGHDNKMQFESSSYEGYFLACEKERDLFKLILKKEDELGDRSIMFTVQNED

SEQ ID NO: 69 - protein, 157 aa, synthetic construct
YFPKLVSKLSVIRNLNDQVLFIDQGNRPLFEDMTDSDCRDNAPRTIFIISMYKWSAIRGMAVTISVKCEKISTLSCENKIISFKEMNPPDVIKDTKSDIIFFQTSVPGHDNKMQFESSSYEGYFLACEKERDLFKLILKKEDELGDRSIMFTVQNED

SEQ ID NO: 70 - protein, 157 aa, synthetic construct
YFPKLRSKLSVIRNLNDQVLFIDQGNRPLFEDMTDSDCRDNAPRTIFIISMYKWSGGRGMAVTISVKCEKISTLSCENKIISFKEMNPPDAIKDTKSDIIFFQLSVPGHDNKMQFESSSYEGYFLACEKERDLFKLILKKEDELGDRSIMFTVQNED

SEQ ID NO: 71 - protein, 157 aa, synthetic construct
YFPKLYSKLSVIRNLNDQVLFIDQGNRPLFEDMTDSDCRDNAPRTIFIISMYKHSTNRGMAVTISVKCEKISTLSCENKIISFKEMNPPDGIKDTKSDIIFFQRSVPGHDNKMQFESSSYEGYFLACEKERDLFKLILKKEDELGDRSIMFTVQNED

SEQ ID NO: 72 - protein, 157 aa, synthetic construct
YFPKLKSKLSVIRNLNDQVLFIDQGNRPLFEDMTDSDCRDNAPRTIFIISMYKWSGVRGMAVTISVKCEKISTLSCENKIISFKEMNPPDVIKDTKSDIIFFQFSVPGHDNKMQFESSSYEGYFLACEKERDLFKLILKKEDELGDRSIMFTVQNED

SEQ ID NO: 73 - protein, 157 aa, synthetic construct
YFDKLKSKLSVIRNLNDQVLFIDQGNRPLFEDMTDSDCRDNAPRTIFIISMYKPSSWRGMAVTISVKCEKISTLSCENKIISFKEMNPPDWIKDTKSDIIFFQRSVPGHDNKMQFESSSYEGYFLACEKERDLFKLILKKEDELGDRSIMFTVQNED

SEQ ID NO: 74 - protein, 157 aa, synthetic construct
YFPKLRSKLSVIRNLNDQVLFIDQGNRPLFEDMTDSDCRDNAPRTIFIISMYKWSRARGMAVTISVKCEKISTLSCENKIISFKEMNPPDSIKDTKSDIIFFQSSVPGHDNKMQFESSSYEGYFLACEKERDLFKLILKKEDELGDRSIMFTVQNED

SEQ ID NO: 75 - protein, 157 aa, synthetic construct
YFPKLGSKLSVIRNLNDQVLFIDQGNRPLFEDMTDSDCRDNAPRTIFIISMYKHSSRRGMAVTISVKCEKISTLSCENKIISFKEMNPPDVIKDTKSDIIFFQNSVPGHDNKMQFESSSYEGYFLACEKERDLFKLILKKEDELGDRSIMFTVQNED

SEQ ID NO: 76 - protein, 157 aa, synthetic construct
YFPKLKSKLSVIRNLNDQVLFIDQGNRPLFEDMTDSDCRDNAPRTIFIISMYKWSWARGMAVTISVKCEKISTLSCENKIISFKEMNPPDLIKDTKSDIIFFQYSVPGHDNKMQFESSSYEGYFLACEKERDLFKLILKKEDELGDRSIMFTVQNED

SEQ ID NO: 77 - protein, 157 aa, synthetic construct
YFPKLRSKLSVIRNLNDQVLFIDQGNRPLFEDMTDSDCRDNAPRTIFIISMYKHSYTRGMAVTISVKCEKISTLSCENKIISFKEMNPPDIIKDTKSDIIFFQYSVPGHDNKMQFESSSYEGYFLACEKERDLFKLILKKEDELGDRSIMFTVQNED

SEQ ID NO: 78 - protein, 157 aa, synthetic construct
YFKKLPSKLSVIHNLNDQVLFIDQGNRPLFEDMTDSDCRDNAPRTIFIISMYKLSSERGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 79 - protein, 157 aa, synthetic construct
YFPKLGSKLSVIRNLNDQVLFIDQGNRPLFEDMTDSDCRDNAPRTIFIISMYKWSQPRGMAVTISVKCEKISTLSCENKIISFKEMNPPDVIKDTKSDIIFFQYSVPGHDNKMQFESSSYEGYFLACEKERDLFKLILKKEDELGDRSIMFTVQNED

SEQ ID NO: 80 - protein, 157 aa, synthetic construct
YFPKLRSKLSVIRNLNDQVLFIDQGNRPLFEDMTDSDCRDNAPRTIFIISMYKWSYPRGMAVTISVKCEKISTLSCENKIISFKEMNPPDGIKDTKSDIIFFQFSVPGHDNKMQFESSSYEGYFLACEKERDLFKLILKKEDELGDRSIMFTVQNED

SEQ ID NO: 81 - protein, 157 aa, synthetic construct
YFPKLSSKLSVIRNLNDQVLFIDQGNRPLFEDMTDSDCRDNAPRTIFIISMYKASDQRGMAVTISVKCEKISTLSCENKIISFKEMNPPDGIKDTKSDIIFFQRSVPGHDNKMQFESSSYEGYFLACEKERDLFKLILKKEDELGDRSIMFTVQNED

SEQ ID NO: 82 - protein, 157 aa, synthetic construct
YFPKLRSKLSVIRNLNDQVLFIDQGNRPLFEDMTDSDCRDNAPRTIFIISMYKLSTIRGMAVTISVKCEKISTLSCENKIISFKEMNPPDYIKDTKSDIIFFQRSVPGHDNKMQFESSSYEGYFLACEKERDLFKLILKKEDELGDRSIMFTVQNED

SEQ ID NO: 83 - protein, 157 aa, synthetic construct
YFWKLWSKLSVIRNLNDQVLFIDQGNRPLFEDMTDSDCRDNAPRTIFIISMYKPSNFRGMAVTISVKCEKISTLSCENKIISFKEMNPPDVIKDTKSDIIFFQGSVPGHDNKMQFESSSYEGYFLACEKERDLFKLILKKEDELGDRSIMFTVQNED

SEQ ID NO: 84 - protein, 157 aa, synthetic construct
YFPKLKSKLSVIRNLNDQVLFIDQGNRPLFEDMTDSDCRDNAPRTIFIISMYKMSSARGMAVTISVKCEKISTLSCENKIISFKEMNPPDGIKDTKSDIIFFQSSVPGHDNKMQFESSSYEGYFLACEKERDLFKLILKKEDELGDRSIMFTVQNED

SEQ ID NO: 85 - protein, 157 aa, synthetic construct
YFPKLKSKLSVIRNLNDQVLFIDQGNRPLFEDMTDSDCRDNAPRTIFIISMYKWSGGRGMAVTISVKCEKISTLSCENKIISFKEMNPPDSIKDTKSDIIFFQFSVPGHDNKMQFESSSYEGYFLACEKERDLFKLILKKEDELGDRSIMFTVQNED

SEQ ID NO: 86 - protein, 157 aa, synthetic construct
YFPKLNSKLSVIRNLNDQVLFIDQGNRPLFEDMTDSDCRDNAPRTIFIISMYKLSTVRGMAVTISVKCEKISTLSCENKIISFKEMNPPDPIKDTKSDIIFFQHSVPGHDNKMQFESSSYEGYFLACEKERDLFKLILKKEDELGDRSIMFTVQNED

SEQ ID NO: 87 - protein, 157 aa, synthetic construct
YFPKLGSKLSVIRNLNDQVLFIDQGNRPLFEDMTDSDCRDNAPRTIFIISMYKGSHFRGMAVTISVKCEKISTLSCENKIISFKEMNPPDGIKDTKSDIIFFQSSVPGHDNKMQFESSSYEGYFLACEKERDLFKLILKKEDELGDRSIMFTVQNED

SEQ ID NO: 88 - protein, 157 aa, synthetic construct
YFPKLSSKLSVIRNLNDQVLFIDQGNRPLFEDMTDSDCRDNAPRTIFIISMYKHSSRRGMAVTISVKCEKISTLSCENKIISFKEMNPPDVIKDTKSDIIFFQASVPGHDNKMQFESSSYEGYFLACEKERDLFKLILKKEDELGDRSIMFTVQNED

SEQ ID NO: 89 - protein, 157 aa, synthetic construct
YFPKLASKLSVIRNLNDQVLFIDQGNRPLFEDMTDSDCRDNAPRTIFIISMYKHSSKRGMAVTISVKCEKISTLSCENKIISFKEMNPPDVIKDTKSDIIFFQVSVPGHDNKMQFESSSYEGYFLACEKERDLFKLILKKEDELGDRSIMFTVQNED

SEQ ID NO: 90 - protein, 157 aa, synthetic construct
YFPKLRSKLSVIRNLNDQVLFIDQGNRPLFEDMTDSDCRDNAPRTIFIISMYKSSLKRGMAVTISVKCEKISTLSCENKIISFKEMNPPDTIKDTKSDIIFFQSSVPGHDNKMQFESSSYEGYFLACEKERDLFKLILKKEDELGDRSIMFTVQNED

SEQ ID NO: 91 - protein, 157 aa, synthetic construct
YFPKLRSKLSVIRNLNDQVLFIDQGNRPLFEDMTDSDCRDNAPRTIFIISMYKYSRGRGMAVTISVKCEKISTLSCENKIISFKEMNPPDKIKDTKSDIIFFQSSVPGHDNKMQFESSSYEGYFLACEKERDLFKLILKKEDELGDRSIMFTVQNED

SEQ ID NO: 92 - protein, 157 aa, synthetic construct
YFWKLSSKLSVIRNLNDQVLFIDQGNRPLFEDMTDSDCRDNAPRTIFIISMYKQSLRRGMAVTISVKCEKISTLSCENKIISFKEMNPPDRIKDTKSDIIFFQASVPGHDNKMQFESSSYEGYFLACEKERDLFKLILKKEDELGDRSIMFTVQNED

SEQ ID NO: 93 - protein, 157 aa, synthetic construct
YFGKLPSKLSVIRNLNDQVLFIDQGNRPLFEDMTDSDCRDNAPRTIFIISMYKLSNRRGMAVTISVKCEKISTLSCENKIISFKEMNPPDGIKDTKSDIIFFQRSVPGHDNKMQFESSSYEGYFLACEKERDLFKLILKKEDELGDRSIMFTVQNED

SEQ ID NO: 94 - protein, 157 aa, synthetic construct
YFPKLGSKLSVIRNLNDQVLFIDQGNRPLFEDMTDSDCRDNAPRTIFIISMYKSSSRRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 95 - protein, 157 aa, synthetic construct
YFWKLSSKLSVIRNLNDQVLFIDQGNRPLFEDMTDSDCRDNAPRTIFIISMYKLSTWRGMAVTISVKCEKISTLSCENKIISFKEMNPPDYIKDTKSDIIFFQRSVPGHDNKMQFESSSYEGYFLACEKERDLFKLILKKEDELGDRSIMFTVQNED

SEQ ID NO: 96 - protein, 157 aa, synthetic construct
YFPKLHSKLSVIRNLNDQVLFIDQGNRPLFEDMTDSDCRDNAPRTIFIISMYKRSTLRGMAVTISVKCEKISTLSCENKIISFKEMNPPDGIKDTKSDIIFFQRSVPGHDNKMQFESSSYEGYFLACEKERDLFKLILKKEDELGDRSIMFTVQNED

SEQ ID NO: 97 - protein, 157 aa, synthetic construct
YFPKLKSKLSVIRNLNDQVLFIDQGNRPLFEDMTDSDCRDNAPRTIFIISMYKTSFNRGMAVTISVKCEKISTLSCENKIISFKEMNPPDVIKDTKSDIIFFQRSVPGHDNKMQFESSSYEGYFLACEKERDLFKLILKKEDELGDRSIMFTVQNED

SEQ ID NO: 98 - protein, 157 aa, synthetic construct
YFPKLTSKLSVIRNLNDQVLFIDQGNRPLFEDMTDSDCRDNAPRTIFIISMYKYSTRRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 99 - protein, 157 aa, synthetic construct
YFPKLRSKLSVIRNLNDQVLFIDQGNRPLFEDMTDSDCRDNAPRTIFIISMYKYSTRRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 100 - protein, 157 aa, synthetic construct
YFSKLPSKLSVIRNLNDQVLFIDQGNRPLFEDMTDSDCRDNAPRTIFIISMYKYSTWRGMAVTISVKCEKISTLSCENKIISFKEMNPPDFIKDTKSDIIFFQSSVPGHDNKMQFESSSYEGYFLACEKERDLFKLILKKEDELGDRSIMFTVQNED

SEQ ID NO: 101 - protein, 157 aa, synthetic construct
YFPKLKSKLSVIRNLNDQVLFIDQGNRPLFEDMTDSDCRDNAPRTIFIISMYKLSRTRGMAVTISVKCEKISTLSCENKIISFKEMNPPDFIKDTKSDIIFFQSSVPGHDNKMQFESSSYEGYFLACEKERDLFKLILKKEDELGDRSIMFTVQNED

SEQ ID NO: 102 - protein, 157 aa, synthetic construct
YFPKLRSKLSVIRNLNDQVLFIDQGNRPLFEDMTDSDCRDNAPRTIFIISMYKFSNFRGMAVTISVKCEKISTLSCENKIISFKEMNPPDAIKDTKSDIIFFQSSVPGHDNKMQFESSSYEGYFLACEKERDLFKLILKKEDELGDRSIMFTVQNED

SEQ ID NO: 103 - protein, 157 aa, synthetic construct
YFSKLPSKLSVIRNLNDQVLFIDQGNRPLFEDMTDSDCRDNAPRTIFIISMYKWSPRRGMAVTISVKCEKISTLSCENKIISFKEMNPPDIIKDTKSDIIFFQSSVPGHDNKMQFESSSYEGYFLACEKERDLFKLILKKEDELGDRSIMFTVQNED

SEQ ID NO: 104 - protein, 157 aa, synthetic construct
YFPKLRSKLSVIRNLNDQVLFIDQGNRPLFEDMTDSDCRDNAPRTIFIISMYKLSTTRGMAVTISVKCEKISTLSCENKIISFKEMNPPDRIKDTKSDIIFFQRSVPGHDNKMQFESSSYEGYFLACEKERDLFKLILKKEDELGDRSIMFTVQNED

SEQ ID NO: 105 - protein, 157 aa, synthetic construct
YFPKLRSKLSVIRNLNDQVLFIDQGNRPLFEDMTDSDCRDNAPRTIFIISMYKYSTSRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 106 - protein, 157 aa, synthetic construct
YFPKLSSKLSVIRNLNDQVLFIDQGNRPLFEDMTDSDCRDNAPRTIFIISMYKASSLRGMAVTISVKCEKISTLSCENKIISFKEMNPPDSIKDTKSDIIFFQRSVPGHDNKMQFESSSYEGYFLACEKERDLFKLILKKEDELGDRSIMFTVQNED

SEQ ID NO: 107 - protein, 157 aa, synthetic construct
YFPKLTSKLSVIRNLNDQVLFIDQGNRPLFEDMTDSDCRDNAPRTIFIISMYKTSYERGMAVTISVKCEKISTLSCENKIISFKEMNPPDVIKDTKSDIIFFQRSVPGHDNKMQFESSSYEGYFLACEKERDLFKLILKKEDELGDRSIMFTVQNED

SEQ ID NO: 108 - protein, 157 aa, synthetic construct
YFPKLGSKLSVIRNLNDQVLFIDQGNRPLFEDMTDSDCRDNAPRTIFIISMYKQSHYRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 109 - protein, 157 aa, synthetic construct
YFKKLPSKLSVIRNLNDQVLFIDQGNRPLFEDMTDSDCRDNAPRTIFIISMYKLSSERGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 110 - protein, 157 aa, synthetic construct
YFPKLNSKLSVIRNLNDQVLFIDQGNRPLFEDMTDSDCRDNAPRTIFIISMYKLSPRRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 111 - protein, 157 aa, synthetic construct
YFPKLESKLSVIRNLNDQVLFIDQGNRPLFEDMTDSDCRDNAPRTIFIISMYKYSSKRGMAVTISVKCEKISTLSCENKIISFKEMNPPDGIKDTKSDIIFFQRSVPGHDNKMQFESSSYEGYFLACEKERDLFKLILKKEDELGDRSIMFTVQNED

SEQ ID NO: 112 - protein, 157 aa, synthetic construct
YFPKLRSKLSVIRNLNDQVLFIDQGNRPLFEDMTDSDCRDNAPRTIFIISMYKHSTNRGMAVTISVKCEKISTLSCENKIISFKEMNPPDIIKDTKSDIIFFQISVPGHDNKMQFESSSYEGYFLACEKERDLFKLILKKEDELGDRSIMFTVQNED

SEQ ID NO: 113 - protein, 157 aa, synthetic construct
YFPKLRSKLSVIRNLNDQVLFIDQGNRPLFEDMTDSDCRDNAPRTIFIISMYKISISRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 114 - protein, 157 aa, synthetic construct
YFPKLLSKLSVIRNLNDQVLFIDQGNRPLFEDMTDSDCRDNAPRTIFIISMYKHSTVRGMAVTISVKCEKISTLSCENKIISFKEMNPPDSIKDTKSDIIFFQRSVPGHDNKMQFESSSYEGYFLACEKERDLFKLILKKEDELGDRSIMFTVQNED

SEQ ID NO: 115 - protein, 157 aa, synthetic construct
YFPKLGSKLSVIRNLNDQVLFIDQGNRPLFEDMTDSDCRDNAPRTIFIISMYKISHDRGMAVTISVKCEKISTLSCENKIISFKEMNPPDTIKDTKSDIIFFQSSVPGHDNKMQFESSSYEGYFLACEKERDLFKLILKKEDELGDRSIMFTVQNED

SEQ ID NO: 116 - protein, 157 aa, synthetic construct
YFPKLMSKLSVIRNLNDQVLFIDQGNRPLFEDMTDSDCRDNAPRTIFIISMYKFSDRRGMAVTISVKCEKISTLSCENKIISFKEMNPPDPIKDTKSDIIFFQRSVPGHDNKMQFESSSYEGYFLACEKERDLFKLILKKEDELGDRSIMFTVQNED

SEQ ID NO: 117 - protein, 157 aa, synthetic construct
YFEKLHSKLSVIRNLNDQVLFIDQGNRPLFEDMTDSDCRDNAPRTIFIISMYKRSTHRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 118 - protein, 157 aa, synthetic construct
YFPKLASKLSVIRNLNDQVLFIDQGNRPLFEDMTDSDCRDNAPRTIFIISMYKWSNARGMAVTISVKCEKISTLSCENKIISFKEMNPPDSIKDTKSDIIFFQRSVPGHDNKMQFESSSYEGYFLACEKERDLFKLILKKEDELGDRSIMFTVQNED

SEQ ID NO: 119 - protein, 157 aa, synthetic construct
YFPKLKSKLSVIRNLNDQVLFIDQGNRPLFEDMTDSDCRDNAPRTIFIISMYKYSCARGMAVTISVKCEKISTLSCENKIISFKEMNPPDAIKDTKSDIIFFQRSVPGHDNKMQFESSSYEGYFLACEKERDLFKLILKKEDELGDRSIMFTVQNED

SEQ ID NO: 120 - protein, 157 aa, synthetic construct
YFPKLVSKLSVIRNLNDQVLFIDQGNRPLFEDMTDSDCRDNAPRTIFIISMYKWSPWRGMAVTISVKCEKISTLSCENKIISFKEMNPPDSIKDTKSDIIFFQRSVPGHDNKMQFESSSYEGYFLACEKERDLFKLILKKEDELGDRSIMFTVQNED

SEQ ID NO: 121 - protein, 157 aa, synthetic construct
YFPKLKSKLSVIRNLNDQVLFIDQGNRPLFEDMTDSDCRDNAPRTIFIISMYKGSGARGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 122 - protein, 157 aa, synthetic construct
YFPKLRSKLSVIRNLNDQVLFIDQGNRPLFEDMTDSDCRDNAPRTIFIISMYKLSGSRGMAVTISVKCEKISTLSCENKIISFKEMNPPDVIKDTKSDIIFFQRSVPGHDNKMQFESSSYEGYFLACEKERDLFKLILKKEDELGDRSIMFTVQNED

SEQ ID NO: 123 - protein, 157 aa, synthetic construct
YFPKLRSKLSIIRNLNDQVLFIDQGNRPLFEDMTDSDCRDNAPRTIFIISMYKGSGARGMAVTISVKCEKISTLSCENKIISFKEMNPPDGIKDTKSDIIFFQRSVPGHDNKMQFESSSYEGYFLACEKERDLFKLILKKEDELGDRSIMFTVQNED

SEQ ID NO: 124 - protein, 157 aa, synthetic construct
YFPKLKSKLSVIRNLNDQVLFIDQGNRPLFEDMTDSDCRDNAPRTIFIISMYKHSYSRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 125 - protein, 157 aa, synthetic construct
YFGKLRSKLSVIRNLNDQVLFIDQGNRPLFEDMTDSDCRDNAPRTIFIISMYKWSSQRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 126 - protein, 157 aa, synthetic construct
YFPKLVSKLSVIRNLNDQVLFIDQGNRPLFEDMTDSDCRDNAPRTIFIISMYKRSSLRGMAVTISVKCEKISTLSCENKIISFKEMNPPDDIKDTKSDIIFFQFSVPGHDNKMQFESSSYEGYFLACEKERDLFKLILKKEDELGDRSIMFTVQNED

SEQ ID NO: 127 - protein, 157 aa, synthetic construct
YFAKLYSKLSVIRNLNDQVLFIDQGNRPLFEDMTDSDCRDNAPRTIFIISMYKYSTARGMAVTISVKCEKISTLSCENKIISFKEMNPPDVIKDTKSDIIFFQHSVPGHDNKMQFESSSYEGYFLACEKERDLFKLILKKEDELGDRSIMFTVQNED

SEQ ID NO: 128 - protein, 157 aa, synthetic construct
YFPKLRSKLSVIRNLNDQVLFIDQGNRPLFEDMTDSDCRDNAPRTIFIISMYKGSGARGMAVTISVKCEKISTLSCENKIISFKEMNPPDVIKDTKSDIIFFQRSVPGHDNKMQFESSSYEGYFLACEKERDLFKLILKKEDELGDRSIMFTVQNED

SEQ ID NO: 129 - protein, 157 aa, synthetic construct
YFPKLRSKLSVIRNLNDQVLFIDQGNRPLFEDMTDSDCRDNAPRTIFIISMYKFSDVRGMAVTISVKCEKISTLSCENKIISFKEMNPPDAIKDTKSDIIFFQRSVPGHDNKMQFESSSYEGYFLACEKERDLFKLILKKEDELGDRSIMFTVQNED

SEQ ID NO: 130 - protein, 157 aa, synthetic construct
YFPKLRSKLSVIRNLNDQVLFIDQGNRPLFEDMTDSDCRDNAPRTIFIISMYKLSPWRGMAVTISVKCEKISTLSCENKIISFKEMNPPDVIKDTKSDIIFFQRSVPGHDNKMQFESSSYEGYFLACEKERDLFKLILKKEDELGDRSIMFTVQNED

SEQ ID NO: 131 - protein, 157 aa, synthetic construct
YFAKLYSKLSVIRNLNDQVLFIDQGNRPLFEDMTDSDCRDNAPRTIFIISMYKRSSLRGMAVTISVKCEKISTLSCENKIISFKEMNPPDGIKDTKSDIIFFQRSVPGHDNKMQFESSSYEGYFLACEKERDLFKLILKKEDELGDRSIMFTVQNED

SEQ ID NO: 132 - protein, 157 aa, synthetic construct
YFPKLRSKLSVIRNLNDQVLFIDQGNRPLFEDMTDSDCRDNAPRTIFIISMYKLSTIRGMAVTISVKCEKISTLSCENKIISFKEMNPPDGIKDTKSDIIFFQRSVPGHDNKMQFESSSYEGYFLACEKERDLFKLILKKEDELGDRSIMFTVQNED

SEQ ID NO: 133 - protein, 157 aa, synthetic construct
YFGKLGSKLSVIRNLNDQVLFIDQGNRPLFEDMTDSDCRDNAPRTIFIISMYKLSTERGMAVTISVKCEKISTLSCENKIISFKEMNPPDGIKDTKSDIIFFQSSVPGHDNKMQFESSSYEGYFLACEKERDLFKLILKKEDELGDRSIMFTVQNED

SEQ ID NO: 134 - protein, 157 aa, synthetic construct
YFAKLYSKLSVIRNLNDQVLFIDQGNRPLFEDMTDSDCRDNAPRTIFIISMYKRSSLRGMAVTISVKCEKISTLSCENKIISFKEMNPPDAIKDTKSDIIFFQRSVPGHDNKMQFESSSYEGYFLACEKERDLFKLILKKEDELGDRSIMFTVQNED

SEQ ID NO: 135 - protein, 157 aa, synthetic construct
YFPKLRSKLSVIRNLNDQVLFIDQGNRPLFEDMTDSDCRDNAPRTIFIISMYKHSWARGMAVTISVKCEKISTLSCENKIISFKEMNPPDGIKDTKSDIIFFQRSVPGHDNKMQFESSSYEGYFLACEKERDLFKLILKKEDELGDRSIMFTVQNED

SEQ ID NO: 136 - protein, 157 aa, synthetic construct
YFPKLSSKLSVIRNLNDQVLFIDQGNRPLFEDMTDSDCRDNAPRTIFIISMYKRSSLRGMAVTISVKCEKISTLSCENKIISFKEMNPPDDIKDTKSDIIFFQFSVPGHDNKMQFESSSYEGYFLACEKERDLFKLILKKEDELGDRSIMFTVQNED

SEQ ID NO: 137 - protein, 157 aa, synthetic construct
YFPKLRSKLSVIRNLNDQVLFIDQGNRPLFEDMTDSDCRDNAPRTIFIISMYKHSTSRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 138 - protein, 157 aa, synthetic construct
YFAKLYSKLSVIRNLNDQVLFIDQGNRPLFEDMTDSDCRDNAPRTIFIISMYKRSSLRGMAVTISVKCEKISTLSCENKIISFKEMNPPDYIKDTKSDIIFFQRSVPGHDNKMQFESSSYEGYFLACEKERDLFKLILKKEDELGDRSIMFTVQNED

SEQ ID NO: 139 - protein, 157 aa, synthetic construct
YFPKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 140 - protein, 157 aa, synthetic construct
YFGKLESKLSVIRNLNDQVLFIDQGNRPLFEDMTDSDCRDNAPRTIFIISMYKDSQPRGKAVTISVKCEKISTLSCENKIISFKEMNPPDNIKDTDSDIIFFQRSVPGHDNKMQFESSSYEGYFLACEKERDLFKLILKKEDELGDRSIMFTVQNED

SEQ ID NO: 141 - protein, 157 aa, synthetic construct
YFPKLRSKLSVIRNLNDQVLFIDQGNRPLFEDMTDSDCRDNAPRTIFIISMYKDSQPRGMAVTISVKCEKISTLSCENKIISFKEMNPPDNIKDTESDIIFFQRSVPGHDNKMQFESSSYEGYFLACEKERDLFKLILKKEDELGDRSIMFTVQNED

SEQ ID NO: 142 - protein, 157 aa, synthetic construct
YFGKLESKLSVIRNLNDQVLFIDQGNRPLFEDMTDSDCRDNAPRTIFIISMYKDSQPRGKAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 143 - protein, 157 aa, synthetic construct
YFPKLRSKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 144 - protein, 157 aa, synthetic construct
YFPKLKSKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 145 - protein, 157 aa, synthetic construct
YFDKLKSKLSVIRNLNDQVLFIDQGNRPLFEDMTDSDCRDNAPRTIFIISMYKDSQPRGMAVTISVKCEKISTLSCENKIISFKEMNPPDSIKDTKSDIIFFQRSVPGHDNKMQFESSSYEGYFLACEKERDLFKLILKKEDELGDRSIMFTVQNED

SEQ ID NO: 146 - protein, 157 aa, synthetic construct
YFSKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSMMFTVQNED

SEQ ID NO: 147 - protein, 157 aa, synthetic construct
YFPKLRSKLSVIRNLNDQVLFIDQGNRPLFEDMTDSDCRDNAPRTIFIISMYKDSQPRGMAVTISVKCEKISTLSCENKIISFKEMNPPDSIKDTKSDIIFFQRSVPGHDNKMQFESSSYEGYFLACEKERDLFKLILKKEDELGDRSIMFTVQNED

SEQ ID NO: 148 - protein, 157 aa, synthetic construct
YFGKLRSKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 149 - protein, 157 aa, synthetic construct
YFPKLKSKLSVIRNLNDQVLFIDQGNRPLFEDMTDSDCRDNAPRTIFIISMYKDSQPRGMAVTISVKCEKISTLSCENKIISFKEMNPPDSIKDTKSDIIFFQRSVPGHDNKMQFESSSYEGYFLACEKERDLFKLILKKEDELGDRSIMFTVQNED

SEQ ID NO: 150 - protein, 157 aa, synthetic construct
YFPKLNSKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 151 - protein, 157 aa, synthetic construct
YFGKLRSKLSVIRNLNDQVLFIDQGNRPLFEDMTDSDCRDNAPRTIFIISMYKDSQPRGMAVTISVKCEKISTLSCENKIISFKEMNPPDSIKDTKSDIIFFQRSVPGHDNKMQFESSSYEGYFLACEKERDLFKLILKKEDELGDRSIMFTVQNED

SEQ ID NO: 152 - protein, 157 aa, synthetic construct
YFGKLESKLSIIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 153 - protein, 157 aa, synthetic construct
YFSKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKREDELGDRSIMFTVQNED

SEQ ID NO: 154 - protein, 157 aa, synthetic construct
YFPKLGSKLSVIRNLNDQVLFIDQGNRPLFEDMTDSDCRDNAPRTIFIISMYKWSQPRGMAVTISVKCEKISTLSCENKIISFKEMNPPDVIKDTKSDIIFFQYSVPGHDNKMQFESSSYEGYFLACEKERDLFKLILKKEDELGDRSIMFTVQIED

SEQ ID NO: 155 - protein, 157 aa, synthetic construct
YFRKLLSKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 156 - protein, 157 aa, synthetic construct
YFSKLESKLSIIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 157 - protein, 157 aa, synthetic construct
YFPKLNSKLSVIRNLNDQVLFIDQGNRPLFEDMTDSDCRDNAPRTIFIISMYKDSQPRGMAVTISVKCEKISTLSCENKIISFKEMNPPDSIKDTKSDIIFFQRSVPGHDNKMQFESSSYEGYFLACEKERDLFKLILKKEDELGDRSIMFTVQNED

SEQ ID NO: 158 - protein, 157 aa, synthetic construct
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKVQFESSSYEGYFLACEKERDLFKLILKKEDELGDRSIMFTVQNED

SEQ ID NO: 159 - protein, 157 aa, synthetic construct
YFSKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 160 - protein, 157 aa, synthetic construct
YFGKLESKLSVIRNLNDQVLFIDQGNRPLFEDMTDSDCRDNAPRTIFIISMYKDSQPRGMAVTISVKCEKISTLSCENKIISFKEMNPPDGIKDTKSDIIFFQRSVPGHDNKMQFESSSYEGYFLACEKERDLFKLILKKEDELGDRSIMFTVQNED

SEQ ID NO: 161 - protein, 157 aa, synthetic construct
YFGKLESKLSVIRNLNDQVLFIDQGNRPFFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 162 - protein, 157 aa, synthetic construct
YFSKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKVQFESSSYEGYFLACEKERDLFKLILKKEDELGDRSIMFTVQNED

SEQ ID NO: 163 - protein, 157 aa, synthetic construct
YFGKLESKLSVIRNLNDQVLFIDQGNRPLFEDMTDSDCRDNAPRTIFIISMYKDSQPRGMAVTISVKCEKISTLSCENKIISFKEMNPPDSIKDTKSDIIFFQRSVPGHDNKMQFESSSYEGYFLACEKERDLFKLILKKEDELGDRSIMFTVQNED

SEQ ID NO: 164 - protein, 157 aa, synthetic construct
YFSKLESKLSVIRNLNDQVLFIDHGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 165 - protein, 157 aa, synthetic construct
YFSKLESKLSVIRNLNDQVLFIDQGNRPFFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 166 - protein, 157 aa, synthetic construct
YFTKLRSKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 167 - protein, 157 aa, synthetic construct
YFGKLESKLSVIRNLNDQVLFIDQGNRPLFEDMTDSDCRDNAPRTIFIISMYKDSQPRGMAVTISVKCEKISTLSCENKIISFKEMNPPDAIKDTKSDIIFFQRSVPGHDNKMQFESSSYEGYFLACEKERDLFKLILKKEDELGDRSIMFTVQNED

SEQ ID NO: 168 - protein, 157 aa, synthetic construct
YFSKLESKLSVIRNLNDQVLFIDQGNRPLFEDMTDSDCRDNAPRTIFIISMYKDSQPRGMAVTISVKCEKISTLSCENKIISFKEMNPPDAIKDTKSDIIFFQRSVPGHDNKMQFESSSYEGYFLACEKERDLFKLILKKEDELGDRSIMFTVQNED

SEQ ID NO: 169 - protein, 157 aa, synthetic construct
YFPKLLSKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 170 - protein, 157 aa, synthetic construct
YFAKLSSKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 171 - protein, 157 aa, synthetic construct
YFSKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKMEDELGDRSIMFTVQNED

SEQ ID NO: 172 - protein, 157 aa, synthetic construct
YFGKLESKLSVIRNLNDQVLFIDQGNRPLFEDMTDSDCRDNAPRTIFIISMYKDSQPRGMAVTISVKCEKISTLSCENKIISFKEMNPPDVIKDTKSDIIFFQRSVPGHDNKMQFESSSYEGYFLACEKERDLFKLILKKEDELGDRSIMFTVQNED

SEQ ID NO: 173 - protein, 157 aa, synthetic construct
YFGKLESKLSVIRNLNDQVLFIDQGNRPLFEDMTDSDCRDNAPRTIFIISMYKDSQPRGMAVTISVKCEKISTLSCENKIISFKEMNPPDFIKDTKSDIIFFQRSVPGHDNKMQFESSSYEGYFLACEKERDLFKLILKKEDELGDRSIMFTVQNED

SEQ ID NO: 174 - protein, 157 aa, synthetic construct
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQSSVPGHDNKMQFESSSYEGYFLACEKERDLFKLILKKEDELGDRSIMFTVQNED

SEQ ID NO: 175 - protein, 157 aa, synthetic construct
YFPKLVSKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 176 - protein, 157 aa, synthetic construct
YFPKLGSKLSVIRNLNDQVLFIDQGNRPLFEDMTDSDCRDNAPRTIFIISMYKWSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 177 - protein, 157 aa, synthetic construct
YFSKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQSSVPGHDNKMQFESSSYEGYFLACEKERDLFKLILKKEDELGDRSIMFTVQNED

SEQ ID NO: 178 - protein, 157 aa, synthetic construct
YFGKLESKLSVIRNLNDQVLFIDQGNRPLFEDMTDSDCRDNAPRTIFIISMYKDSQPRGMAVTISVKCEKISTLSCENKIISFKEMNPPDSIKDTKSDIIFFQSSVPGHDNKMQFESSSYEGYFLACEKERDLFKLILKKEDELGDRSIMFTVQNED

SEQ ID NO: 179 - protein, 157 aa, synthetic construct
YFPKLRSKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQTSVPGHDNKMQFESSSYEGYFLACEKERDLFKLILKKEDELGDRSIMFTVQNED

SEQ ID NO: 180 - protein, 157 aa, synthetic construct
YFPKLGSKLSVIRNLNDQVLFIDQGNRPLFEDMTDSDCRDNAPRTIFIISMYKWSQPRGMAVTISVKCEKISTLSCENKIISFKEMNPPDGIKDTKSDIIFFQRSVPGHDNKMQFESSSYEGYFLACEKERDLFKLILKKEDELGDRSIMFTVQNED

SEQ ID NO: 181 - protein, 157 aa, synthetic construct
YFPKLKSKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQTSVPGHDNKMQFESSSYEGYFLACEKERDLFKLILKKEDELGDRSIMFTVQNED

SEQ ID NO: 182 - protein, 157 aa, synthetic construct
YFPKLGSKLSVIRNLNDQVLFIDQGNRPLFEDMTDSDCRDNAPRTIFIISMYKWSQPRGMAVTISVKCEKISTLSCENKIISFKEMNPPDSIKDTKSDIIFFQRSVPGHDNKMQFESSSYEGYFLACEKERDLFKLILKKEDELGDRSIMFTVQNED

SEQ ID NO: 183 - protein, 157 aa, synthetic construct
YFGKLESKLSVIRNLNDQVLFIDQGNRPLFEDMTDSDCRDNAPRTIFIISMYKDSQPRGMAVTISVKCEKISTLSCENKIISFKEMNPPDPIKDTKSDIIFFQHSVPGHDNKMQFESSSYEGYFLACEKERDLFKLILKKEDELGDRSIMFTVQNED

SEQ ID NO: 184 - protein, 157 aa, synthetic construct
YFGKLESKLSVIRNLNDQVLFIDQGNRPLFEDMTDSDCRDNAPRTIFIISMYKDSQPRGMAVTISVKCEKISTLSCENKIISFKEMNPPDAIKDTKSDIIFFQSSVPGHDNKMQFESSSYEGYFLACEKERDLFKLILKKEDELGDRSIMFTVQNED

SEQ ID NO: 185 - protein, 157 aa, synthetic construct
YFTKLMSKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 186 - protein, 157 aa, synthetic construct
YFWKLKSKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 187 - protein, 157 aa, synthetic construct
YFPKLGSKLSVIRNLNDQVLFIDQGNRPLFEDMTDSDCRDNAPRTIFIISMYKWSQPRGMAVTISVKCEKISTLSCENKIISFKEMNPPDAIKDTKSDIIFFQRSVPGHDNKMQFESSSYEGYFLACEKERDLFKLILKKEDELGDRSIMFTVQNED

SEQ ID NO: 188 - protein, 157 aa, synthetic construct
YFAKLYSKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 189 - protein, 157 aa, synthetic construct
YFAKLYSKLSVIRNLNDQVLFIDQGNRPLFEDMTDSDCRDNAPRTIFIISMYKDSQPRGMAVTISVKCEKISTLSCENKIISFKEMNPPDSIKDTKSDIIFFQRSVPGHDNKMQFESSSYEGYFLACEKERDLFKLILKKEDELGDRSIMFTVQNED

SEQ ID NO: 190 - protein, 157 aa, synthetic construct
YFVKLPSKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 191 - protein, 157 aa, synthetic construct
YFTKLRSKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQTSVPGHDNKMQFESSSYEGYFLACEKERDLFKLILKREDELGDRSIMFTVQNED

SEQ ID NO: 192 - protein, 157 aa, synthetic construct
YFTKLRSKLSIIRNLNDQVLFIDQGNRPLFEDMTDSDCRDNAPRTIFIISMYKDSQPRGMAVTISVKCEKISTLSCENKIISFKEMNPPDNIKDTKSDIIFFQTSVPGHDNKMQFESSSYEGYFLACEKERDLFKLILKKEDELGDRSIMFTVQNED

SEQ ID NO: 193 - protein, 157 aa, synthetic construct
YFPKLGSKLSVIRNLNDQVLFIDQGNRPLFEDMTDSDCRDNAPRTIFIISMYKWSQPRGMAVTISVKCEKISTLSCENKIISFKEMNPPDVIKDTKSDIIFFQRSVPGHDNKMQFESSSYEGYFLACEKERDLFKLILKKEDELGDRSIMFTVQNED

SEQ ID NO: 194 - protein, 157 aa, synthetic construct
YFTKLRSKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQTSVPGHDNKMQFESSSYEGYFLACEKERDLFKLILKKEDELGDRSIMFTVQNED

SEQ ID NO: 195 - protein, 157 aa, synthetic construct
YFTKLRSKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQTSVPGHDNKVQFESSSYEGYFLACEKERDLFKLILKKEDELGDRSIMFTVQNED

SEQ ID NO: 196 - protein, 157 aa, synthetic construct
YFTKLRSKLSVIRNLNDQVLFIDHGNRPLFEDMTDSDCRDNAPRTIFIISMYKDSQPRGMAVTISVKCEKISTLSCENKIISFKEMNPPDNIKDTKSDIIFFQTSVPGHDNKMQFESSSYEGYFLACEKERDLFKLILKKEDELGDRSIMFTVQNED

SEQ ID NO: 197 - protein, 157 aa, synthetic construct
YFTKLRSKLSVIRNLNDQVLFIDQGNRPFFEDMTDSDCRDNAPRTIFIISMYKDSQPRGMAVTISVKCEKISTLSCENKIISFKEMNPPDNIKDTKSDIIFFQTSVPGHDNKMQFESSSYEGYFLACEKERDLFKLILKKEDELGDRSIMFTVQNED

SEQ ID NO: 198 - protein, 157 aa, synthetic construct
YFWKLSSKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 199 - protein, 157 aa, synthetic construct
YFPKLGSKLSVIRNLNDQVLFIDQGNRPLFEDMTDSDCRDNAPRTIFIISMYKWSQPRGMAVTISVKCEKISTLSCENKIISFKEMNPPDGIKDTKSDIIFFQSSVPGHDNKMQFESSSYEGYFLACEKERDLFKLILKKEDELGDRSIMFTVQNED

SEQ ID NO: 200 - protein, 157 aa, synthetic construct
YFPKLGSKLSVIRNLNDQVLFIDQGNRPLFEDMTDSDCRDNAPRTIFIISMYKWSQPRGMAVTISVKCEKISTLSCENKIISFKEMNPPDSIKDTKSDIIFFQSSVPGHDNKMQFESSSYEGYFLACEKERDLFKLILKKEDELGDRSIMFTVQNED

SEQ ID NO: 201 - protein, 157 aa, synthetic construct
YFWKLSSKLSVIRNLNDQVLFIDQGNRPLFEDMTDSDCRDNAPRTIFIISMYKDSQPRGMAVTISVKCEKISTLSCENKIISFKEMNPPDSIKDTKSDIIFFQRSVPGHDNKMQFESSSYEGYFLACEKERDLFKLILKKEDELGDRSIMFTVQNED

SEQ ID NO: 202 - protein, 157 aa, synthetic construct
YFPKLGSKLSVIRNLNDQVLFIDQGNRPLFEDMTDSDCRDNAPRTIFIISMYKWSQPRGMAVTISVKCEKISTLSCENKIISFKEMNPPDPIKDTKSDIIFFQHSVPGHDNKMQFESSSYEGYFLACEKERDLFKLILKKEDELGDRSIMFTVQNED

SEQ ID NO: 203 - protein, 157 aa, synthetic construct
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQCSVPGHDNKMQFESSSYEGYFLACEKERDLFKLILKKEDELGDRSIMFTVQNED

SEQ ID NO: 204 - protein, 157 aa, synthetic construct
YFPKLGSKLSVIRNLNDQVLFIDQGNRPLFEDMTDSDCRDNAPRTIFIISMYKWSQPRGMAVTISVKCEKISTLSCENKIISFKEMNPPDAIKDTKSDIIFFQSSVPGHDNKMQFESSSYEGYFLACEKERDLFKLILKKEDELGDRSIMFTVQNED

SEQ ID NO: 205 - protein, 157 aa, synthetic construct
YFPKLGSKLSVIRNLNDQVLFIDQGNRPLFEDMTDSDCRDNAPRTIFIISMYKWSQPRGMAVTISVKCEKISTLSCENKIISFKEMNPPDGIKDTKSDIIFFQTSVPGHDNKMQFESSSYEGYFLACEKERDLFKLILKKEDELGDRSIMFTVQNED

SEQ ID NO: 206 - protein, 157 aa, synthetic construct
YFPKLGSKLSVIRNLNDQVLFIDQGNRPLFEDMTDSDCRDNAPRTIFIISMYKWSQPRGMAVTISVKCEKISTLSCENKIISFKEMNPPDVIKDTKSDIIFFQGSVPGHDNKMQFESSSYEGYFLACEKERDLFKLILKKEDELGDRSIMFTVQNED

SEQ ID NO: 207 - protein, 157 aa, synthetic construct
YFPKLGSKLSVIRNLNDQVLFIDQGNRPLFEDMTDSDCRDNAPRTIFIISMYKWSQPRGMAVTISVKCEKISTLSCENKIISFKEMNPPDVIKDTKSDIIFFQSSVPGHDNKMQFESSSYEGYFLACEKERDLFKLILKKEDELGDRSIMFTVQNED

SEQ ID NO: 208 - protein, 157 aa, synthetic construct
YFPKLFSKLSVIRNLNDQVLFIDQGNRPLFEDMTDSDCRDNAPRTIFIISMYKHSQPRGMAVTISVKCEKISTLSCENKIISFKEMNPPDAIKDTKSDIIFFQSSVPGHDNKMQFESSSYEGYFLACEKERDLFKLILKKEDELGDRSIMFTVQNED

SEQ ID NO: 209 - protein, 157 aa, synthetic construct
YFPKLRSKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQISVPGHDNKMQFESSSYEGYFLACEKERDLFKLILKKEDELGDRSIMFTVQNED

SEQ ID NO: 210 - protein, 157 aa, synthetic construct
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQLSVPGHDNKMQFESSSYEGYFLACEKERDLFKLILKKEDELGDRSIMFTVQNED

SEQ ID NO: 211 - protein, 157 aa, synthetic construct
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQISVPGHDNKMQFESSSYEGYFLACEKERDLFKLILKKEDELGDRSMMFTVQNED

SEQ ID NO: 212 - protein, 157 aa, synthetic construct
YFPKLKSKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQISVPGHDNKMQFESSSYEGYFLACEKERDLFKLILKKEDELGDRSIMFTVQNED

SEQ ID NO: 213 - protein, 157 aa, synthetic construct
YFGKLESKLSVIRNLNDQVLFIDQGNRPLFEDMTDSDCRDNAPRTIFIISMYKDSQPRGMAVTISVKCEKISTLSCENKIISFKEMNPPDGIKDTKSDIIFFQLSVPGHDNKMQFESSSYEGYFLACEKERDLFKLILKKEDELGDRSIMFTVQNED

SEQ ID NO: 214 - protein, 157 aa, synthetic construct
YFPKLGSKLSVIRNLNDQVLFIDQGNRPLFEDMTDSDCRDNAPRTIFIISMYKWSQPRGMAVTISVKCEKISTLSCENKIISFKEMNPPDVIKDTKSDIIFFQHSVPGHDNKMQFESSSYEGYFLACEKERDLFKLILKKEDELGDRSIMFTVQNED

SEQ ID NO: 215 - protein, 157 aa, synthetic construct
YFGKLRSKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQISVPGHDNKMQFESSSYEGYFLACEKERDLFKLILKKEDELGDRSIMFTVQNED

SEQ ID NO: 216 - protein, 157 aa, synthetic construct
YFGKLESKLSVIRNLNDQVLFIDQGNRPLFEDMTDSDCRDNAPRTIFIISMYKDSQPRGMAVTISVKCEKISTLSCENKIISFKEMNPPDDIKDTKSDIIFFQFSVPGHDNKMQFESSSYEGYFLACEKERDLFKLILKKEDELGDRSIMFTVQNED

SEQ ID NO: 217 - protein, 157 aa, synthetic construct
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQISVPGHDNKMQFESSSYEGYFLACEKERDLFKLILKREDELGDRSIMFTVQNED

SEQ ID NO: 218 - protein, 157 aa, synthetic construct
YFPKLGSKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQISVPGHDNKMQFESSSYEGYFLACEKERDLFKLILKKEDELGDRSIMFTVQNED

SEQ ID NO: 219 - protein, 157 aa, synthetic construct
YFPKLSSKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQISVPGHDNKMQFESSSYEGYFLACEKERDLFKLILKKEDELGDRSIMFTVQNED

SEQ ID NO: 220 - protein, 157 aa, synthetic construct
YFTKLRSKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQLSVPGHDNKMQFESSSYEGYFLACEKERDLFKLILKKEDELGDRSIMFTVQNED

SEQ ID NO: 221 - protein, 157 aa, synthetic construct
YFGKLESKLSIIRNLNDQVLFIDQGNRPLFEDMTDSDCRDNAPRTIFIISMYKDSQPRGMAVTISVKCEKISTLSCENKIISFKEMNPPDNIKDTKSDIIFFQISVPGHDNKMQFESSSYEGYFLACEKERDLFKLILKKEDELGDRSIMFTVQNED

SEQ ID NO: 222 - protein, 157 aa, synthetic construct
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQYSVPGHDNKMQFESSSYEGYFLACEKERDLFKLILKKEDELGDRSIMFTVQNED

SEQ ID NO: 223 - protein, 157 aa, synthetic construct
YFSKLESKLSVIRNLNDQVLFIDQGNRPLFEDMTDSDCRDNAPRTIFIISMYKDSQPRGMAVTISVKCEKISTLSCENKIISFKEMNPPDDIKDTKSDIIFFQFSVPGHDNKMQFESSSYEGYFLACEKERDLFKLILKKEDELGDRSIMFTVQNED

SEQ ID NO: 224 - protein, 157 aa, synthetic construct
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQISVPGHDNKMQFESSSYEGYFLACEKERDLFKLILKKEDELGDRSIMFTVQNED

SEQ ID NO: 225 - protein, 157 aa, synthetic construct
YFPKLGSKLSVIRNLNDQVLFIDQGNRPLFEDMTDSDCRDNAPRTIFIISMYKWSQPRGMAVTISVKCEKISTLSCENKIISFKEMNPPDVIKDTKSDIIFFQTSVPGHDNKMQFESSSYEGYFLACEKERDLFKLILKKEDELGDRSIMFTVQNED

SEQ ID NO: 226 - protein, 157 aa, synthetic construct
YFSKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQYSVPGHDNKMQFESSSYEGYFLACEKERDLFKLILKKEDELGDRSIMFTVQNED

SEQ ID NO: 227 - protein, 157 aa, synthetic construct
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQISVPGHDNKVQFESSSYEGYFLACEKERDLFKLILKKEDELGDRSIMFTVQNED

SEQ ID NO: 228 - protein, 157 aa, synthetic construct
YFPKLKSKLSVIRNLNDQVLFIDQGNRPLFEDMTDSDCRDNAPRTIFIISMYKDSQPRGMAVTISVKCEKISTLSCENKIISFKEMNPPDVIKDTKSDIIFFQLSVPGHDNKMQFESSSYEGYFLACEKERDLFKLILKKEDELGDRSIMFTVQNED

SEQ ID NO: 229 - protein, 157 aa, synthetic construct
YFGKLESKLSVIRNLNDQVLFIDHGNRPLFEDMTDSDCRDNAPRTIFIISMYKDSQPRGMAVTISVKCEKISTLSCENKIISFKEMNPPDNIKDTKSDIIFFQISVPGHDNKMQFESSSYEGYFLACEKERDLFKLILKKEDELGDRSIMFTVQNED

SEQ ID NO: 230 - protein, 157 aa, synthetic construct
YFGKLESKLSVIRNLNDQVLFIDQGNRPFFEDMTDSDCRDNAPRTIFIISMYKDSQPRGMAVTISVKCEKISTLSCENKIISFKEMNPPDNIKDTKSDIIFFQISVPGHDNKMQFESSSYEGYFLACEKERDLFKLILKKEDELGDRSIMFTVQNED

SEQ ID NO: 231 - protein, 157 aa, synthetic construct
YFPKLTSKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQISVPGHDNKMQFESSSYEGYFLACEKERDLFKLILKKEDELGDRSIMFTVQNED

SEQ ID NO: 232 - protein, 157 aa, synthetic construct
YFPKLRSKLSVIRNLNDQVLFIDQGNRPLFEDMTDSDCRDNAPRTIFIISMYKDSQPRGMAVTISVKCEKISTLSCENKIISFKEMNPPDMIKDTKSDIIFFQISVPGHDNKMQFESSSYEGYFLACEKERDLFKLILKKEDELGDRSIMFTVQNED

SEQ ID NO: 233 - protein, 157 aa, synthetic construct
YFPKLGSKLSVIRNLNDQVLFIDQGNRPLFEDMTDSDCRDNAPRTIFIISMYKWSQPRGMAVTISVKCEKISTLSCENKIISFKEMNPPDVIKDTKSDIIFFPYSVPGHDNKMQFESSSYEGYFLACEKERDLFKLILKKEDELGDRSIMFTVQNED

SEQ ID NO: 234 - protein, 157 aa, synthetic construct
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQVSVPGHDNKMQFESSSYEGYFLACEKERDLFKLILKKEDELGDRSIMFTVQNED

SEQ ID NO: 235 - protein, 157 aa, synthetic construct
YFPKLRSKLSVIRNLNDQVLFIDQGNRPLFEDMTDSDCRDNAPRTIFIISMYKQSQPRGMAVTISVKCEKISTLSCENKIISFKEMNPPDAIKDTKSDIIFFQISVPGHDNKMQFESSSYEGYFLACEKERDLFKLILKKEDELGDRSIMFTVQNED

SEQ ID NO: 236 - protein, 157 aa, synthetic construct
YFPKLRSKLSVIRNLNDQVLFIDQGNRPLFEDMTDSDCRDNAPRTIFIISMYKHSQPRGMAVTISVKCEKISTLSCENKIISFKEMNPPDHIKDTKSDIIFFQYSVPGHDNKMQFESSSYEGYFLACEKERDLFKLILKKEDELGDRSIMFTVQNED

SEQ ID NO: 237 - protein, 157 aa, synthetic construct
YFPKLKSKLSVIRNLNDQVLFIDQGNRPLFEDMTDSDCRDNAPRTIFIISMYKQSQPRGMAVTISVKCEKISTLSCENKIISFKEMNPPDAIKDTKSDIIFFQISVPGHDNKMQFESSSYEGYFLACEKERDLFKLILKKEDELGDRSIMFTVQNED

SEQ ID NO: 238 - protein, 157 aa, synthetic construct
YFPKLGSKLSVIRNLNDQVLFIDQGNRPLFEDMTDSDCRDNAPRTIFIISMYKWSQPRGMAVTISVKCEKISTLSCENKIISFKEMNPPDVIKDTKSDIIFFKYSVPGHDNKMQFESSSYEGYFLACEKERDLFKLILKKEDELGDRSIMFTVQNED

SEQ ID NO: 239 - protein, 157 aa, synthetic construct
YFPKLGSKLSVIRNLNDQVLFIDQGNRPLFEDMTDSDCRDNAPRTIFIISMYKWSQPRGMAVTISVKCEKISTLSCENKIISFKEMNPPDAIKDTKSDIIFFQCSVPGHDNKMQFESSSYEGYFLACEKERDLFKLILKKEDELGDRSIMFTVQNED

SEQ ID NO: 240 - protein, 157 aa, synthetic construct
YFGKLESKLSVIRNLNDQVLFIDQGNRPLFEDMTDSDCRDNAPRTIFIISMYKDSQPRGMAVTISVKCEKISTLSCENKIISFKEMNPPDVIKDTKSDIIFFQLSVPGHDNKMQFESSSYEGYFLACEKERDLFKLILKKEDELGDRSIMFTVQNED

SEQ ID NO: 241 - protein, 157 aa, synthetic construct
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQFSVPGHDNKMQFESSSYEGYFLACEKERDLFKLILKKEDELGDRSIMFTVQNED

SEQ ID NO: 242 - protein, 157 aa, synthetic construct
YFPKLGSKLSVIRNLNDQVLFIDQGNRPLFEDMTDSDCRDNAPRTIFIISMYKRSQPRGMAVTISVKCEKISTLSCENKIISFKEMNPPDVIKDTKSDIIFFQYSVPGHDNKMQFESSSYEGYFLACEKERDLFKLILKKEDELGDRSIMFTVQNED

SEQ ID NO: 243 - protein, 157 aa, synthetic construct
YFPKLLSKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQISVPGHDNKMQFESSSYEGYFLACEKERDLFKLILKKEDELGDRSIMFTVQNED

SEQ ID NO: 244 - protein, 157 aa, synthetic construct
YFSKLESKLSVIRNLNDQVLFIDQGNRPLFEDMTDSDCRDNAPRTIFIISMYKDSQPRGMAVTISVKCEKISTLSCENKIISFKEMNPPDVIKDTKSDIIFFQLSVPGHDNKMQFESSSYEGYFLACEKERDLFKLILKKEDELGDRSIMFTVQNED

SEQ ID NO: 245 - protein, 157 aa, synthetic construct
YFTKLRSKLSVIRNLNDQVLFIDQGNRPLFEDMTDSDCRDNAPRTIFIISMYKDSQPRGMAVTISVKCEKISTLSCENKIISFKEMNPPDAIKDTKSDIIFFQYSVPGHDNKMQFESSSYEGYFLACEKERDLFKLILKKEDELGDRSIMFTVQNED

SEQ ID NO: 246 - protein, 157 aa, synthetic construct
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQISVPGHDNKMQFESSSYEGYFLACEKERDLFKLILKMEDELGDRSIMFTVQNED

SEQ ID NO: 247 - protein, 157 aa, synthetic construct
YFPKLGSKLSVIRNLNDQVLFIDQGNRPLFEDMTDSDCRDNAPRTIFIISMYKWSQPRGMAVTISVKCEKISTLSCENKIISFKEMNPPDGIKDTKSDIIFFQLSVPGHDNKMQFESSSYEGYFLACEKERDLFKLILKKEDELGDRSIMFTVQNED

SEQ ID NO: 248 - protein, 157 aa, synthetic construct
YFPKLTSKLSIIRNLNDQVLFIDQGNRPLFEDMTDSDCRDNAPRTIFIISMYKQSQPRGMAVTISVKCEKISTLSCENKIISFKEMNPPDAIKDTKSDIIFFQISVPGHDNKMQFESSSYEGYFLACEKERDLFKLILKKEDELGDRSIMFTVQNED

SEQ ID NO: 249 - protein, 157 aa, synthetic construct
YFTKLRSKLSVIRNLNDQVLFIDQGNRPLFEDMTDSDCRDNAPRTIFIISMYKDSQPRGMAVTISVKCEKISTLSCENKIISFKEMNPPDVIKDTKSDIIFFQLSVPGHDNKMQFESSSYEGYFLACEKERDLFKLILKKEDELGDRSIMFTVQNED

SEQ ID NO: 250 - protein, 157 aa, synthetic construct
YFGKLESKLSVIRNLNDQVLFIDQGNRPLFEDMTDSDCRDNAPRTIFIISMYKDSQPRGMAVTISVKCEKISTLSCENKIISFKEMNPPDVIKDTKSDIIFFQYSVPGHDNKMQFESSSYEGYFLACEKERDLFKLILKKEDELGDRSIMFTVQNED

SEQ ID NO: 251 - protein, 157 aa, synthetic construct
YFPKLGSKLSVIRNLNDQVLFIDQGNRPLFEDMTDSDCRDNAPRTIFIISMYKWSQPRGMAVTISVKCEKISTLSCENKIISFKEMNPPDVIKDTKSDIIFFQCSVPGHDNKMQFESSSYEGYFLACEKERDLFKLILKKEDELGDRSIMFTVQNED

SEQ ID NO: 252 - protein, 157 aa, synthetic construct
YFPKLTSKLSVIRNLNDQVLFIDQGNRPLFEDMTDSDCRDNAPRTIFIISMYKQSQPRGMAVTISVKCEKISTLSCENKIISFKEMNPPDAIKDTKSDIIFFQISVPGHDNKMQFESSSYEGYFLACEKERDLFKLILKKEDELGDRSIMFTVQNED

SEQ ID NO: 253 - protein, 157 aa, synthetic construct
YFSKLESKLSVIRNLNDQVLFIDQGNRPLFEDMTDSDCRDNAPRTIFIISMYKDSQPRGMAVTISVKCEKISTLSCENKIISFKEMNPPDVIKDTKSDIIFFQYSVPGHDNKMQFESSSYEGYFLACEKERDLFKLILKKEDELGDRSIMFTVQNED

SEQ ID NO: 254 - protein, 157 aa, synthetic construct
YFGKLESKLSVIRNLNDQVLFIDQGNRPLFEDMTDSDCRDNAPRTIFIISMYKYSQPRGMAVTISVKCEKISTLSCENKIISFKEMNPPDNIKDTKSDIIFFQISVPGHDNKMQFESSSYEGYFLACEKERDLFKLILKKEDELGDRSIMFTVQNED

SEQ ID NO: 255 - protein, 157 aa, synthetic construct
YFPKLGSKLSVIRNLNDQVLFIDQGNRPLFEDMTDSDCRDNAPRTIFIISMYKWSQPRGMAVTISVKCEKISTLSCENKIISFKEMNPPDAIKDTKSDIIFFQLSVPGHDNKMQFESSSYEGYFLACEKERDLFKLILKKEDELGDRSIMFTVQNED

SEQ ID NO: 256 - protein, 157 aa, synthetic construct
YFPKLGSKLSVIRNLNDQVLFIDQGNRPLFEDMTDSDCRDNAPRTIFIISMYKWSQPRGMAVTISVKCEKISTLSCENKIISFKEMNPPDGIKDTKSDIIFFQYSVPGHDNKMQFESSSYEGYFLACEKERDLFKLILKKEDELGDRSIMFTVQNED

SEQ ID NO: 257 - protein, 157 aa, synthetic construct
YFGKLESKLSVIRNLNDQVLFIDQGNRPLFEDMTDSDCRDNAPRTIFIISMYKDSQPRGMAVTISVKCEKISTLSCENKIISFKEMNPPDVIKDTKSDIIFFQVSVPGHDNKMQFESSSYEGYFLACEKERDLFKLILKKEDELGDRSIMFTVQNED

SEQ ID NO: 258 - protein, 157 aa, synthetic construct
YFTKLRSKLSVIRNLNDQVLFIDQGNRPLFEDMTDSDCRDNAPRTIFIISMYKDSQPRGMAVTISVKCEKISTLSCENKIISFKEMNPPDVIKDTKSDIIFFQYSVPGHDNKMQFESSSYEGYFLACEKERDLFKLILKKEDELGDRSIMFTVQNED

SEQ ID NO: 259 - protein, 157 aa, synthetic construct
YFPKLRSKLSVIRNLNDQVLFIDQGNRPLFEDMTDSDCRDNAPRTIFIISMYKWSQPRGMAVTISVKCEKISTLSCENKIISFKEMNPPDVIKDTKSDIIFFQLSVPGHDNKMQFESSSYEGYFLACEKERDLFKLILKKEDELGDRSIMFTVQNED

SEQ ID NO: 260 - protein, 157 aa, synthetic construct
YFPKLKSKLSVIRNLNDQVLFIDQGNRPLFEDMTDSDCRDNAPRTIFIISMYKWSQPRGMAVTISVKCEKISTLSCENKIISFKEMNPPDVIKDTKSDIIFFQLSVPGHDNKMQFESSSYEGYFLACEKERDLFKLILKKEDELGDRSIMFTVQNED

SEQ ID NO: 261 - protein, 157 aa, synthetic construct
YFSKLESKLSVIRNLNDQVLFIDQGNRPLFEDMTDSDCRDNAPRTIFIISMYKDSQPRGMAVTISVKCEKISTLSCENKIISFKEMNPPDVIKDTKSDIIFFQVSVPGHDNKMQFESSSYEGYFLACEKERDLFKLILKKEDELGDRSIMFTVQNED

SEQ ID NO: 262 - protein, 157 aa, synthetic construct
YLPKLRSKLSVIRNLNDQVLFIDQGNRPLFEDMTDSDCRDNAPRTIFIISMYKWSQPRGMAVTISVKCEKISTLSCENKIISFKEMNPPDVIKDTKSDIIFFQYSVPGHDNKMQFESSSYEGYFLACEKERDLFKLILKKEDELGDRSIMFTVQNED

SEQ ID NO: 263 - protein, 157 aa, synthetic construct
YFPKLGSKLSVIRNLNDQVLFIDQGNRPLFEDMTDSDCRDNAPRTIFIISMYKWSQPRGMAVTISVKCEKISTLSCENKIISFKEMNPPDAIKDTKSDIIFFQYSVPGHDNKMQFESSSYEGYFLACEKERDLFKLILKKEDELGDRSIMFTVQNED

SEQ ID NO: 264 - protein, 157 aa, synthetic construct
YFTKLRSKLSVIRNLNDQVLFIDQGNRPLFEDMTDSDCRDNAPRTIFIISMYKDSQPRGMAVTISVKCEKISTLSCENKIISFKEMNPPDVIKDTKSDIIFFQVSVPGHDNKMQFESSSYEGYFLACEKERDLFKLILKKEDELGDRSIMFTVQNED

SEQ ID NO: 265 - protein, 157 aa, synthetic construct
YFPKLGSKLSVIRNLNDQVLFIDQGNRPLFEDMTDSDCRDNAPRTIFIISMYKWSQPRGMAVTISVKCEKISTLSCENKIISFKEMNPPDVIKDTKSDIIFFQLSVPGHDNKMQFESSSYEGYFLACEKERDLFKLILKKEDELGDRSIMFTVQNED

SEQ ID NO: 266 - protein, 157 aa, synthetic construct
YFPKLRSKLSIIRNLNDQVLFIDQGNRPLFEDMTDSDCRDNAPRTIFIISMYKWSQPRGMAVTISVKCEKISTLSCENKIISFKEMNPPDVIKDTKSDIIFFQYSVPGHDNKMQFESSSYEGYFLACEKERDLFKLILKKEDELGDRSIMFTVQNED

SEQ ID NO: 267 - protein, 157 aa, synthetic construct
YFPKLGSKLSVIRNLNDQVLFIDQGNRPLFEDMTDSDCRDNAPRTIFIISMYKWSQPRGMAVTISVKCEKISTLSCENKIISFKEMNPPDVIKDTKSDIIFFQYSVPGHDNKMQFESSSYEGYFLACEKERDLFKLILKKEDELGDRSMMFTVQNED

SEQ ID NO: 268 - protein, 157 aa, synthetic construct
YFPKLRSKLSVIRNLNDQVLFIDQGNRPLFEDMTDSDCRDNAPRTIFIISMYKWSQPRGMAVTISVKCEKISTLSCENKIISFKEMNPPDVIKDTKSDIIFFQYSVPGHDNKMQFESSSYEGYFLACEKERDLFKLILKKEDELGDRSIMFTVQNED

SEQ ID NO: 269 - protein, 157 aa, synthetic construct
YFPKLGSKLSVIRNLNDQVLFIDQGNRPLFEDMTDSDCRDNAPRTIFIISMYKLSQPRGMAVTISVKCEKISTLSCENKIISFKEMNPPDVIKDTKSDIIFFQYSVPGHDNKMQFESSSYEGYFLACEKERDLFKLILKKEDELGDRSIMFTVQNED

SEQ ID NO: 270 - protein, 157 aa, synthetic construct
YFAKLYSKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQISVPGHDNKMQFESSSYEGYFLACEKERDLFKLILKKEDELGDRSIMFTVQNED

SEQ ID NO: 271 - protein, 157 aa, synthetic construct
YFPKLKSKLSVIRNLNDQVLFIDQGNRPLFEDMTDSDCRDNAPRTIFIISMYKWSQPRGMAVTISVKCEKISTLSCENKIISFKEMNPPDVIKDTKSDIIFFQYSVPGHDNKMQFESSSYEGYFLACEKERDLFKLILKKEDELGDRSIMFTVQNED

SEQ ID NO: 272 - protein, 157 aa, synthetic construct
YFPKLGSKLSVIRNLNDQVLFIDQGNRPLFEDMTDSDCRDNAPRTIFIISMYKWSQPRGMAVTISVKCEKISTLSCENKIISFKEMNPPDMIKDTKSDIIFFQYSVPGHDNKMQFESSSYEGYFLACEKERDLFKLILKKEDELGDRSIMFTVQNED

SEQ ID NO: 273 - protein, 157 aa, synthetic construct
YFPKLGSKLSVIRNLNDQVLFIDQGNRPLFEDMTDSDCRDNAPRTIFIISMYKWSQPRGMAVTISVKCEKISTLSCENKIISFKEMNPPDAIKDTKSDIIFFQVSVPGHDNKMQFESSSYEGYFLACEKERDLFKLILKKEDELGDRSIMFTVQNED

SEQ ID NO: 274 - protein, 157 aa, synthetic construct
YFGKLRSKLSVIRNLNDQVLFIDQGNRPLFEDMTDSDCRDNAPRTIFIISMYKWSQPRGMAVTISVKCEKISTLSCENKIISFKEMNPPDVIKDTKSDIIFFQYSVPGHDNKMQFESSSYEGYFLACEKERDLFKLILKKEDELGDRSIMFTVQNED

SEQ ID NO: 275 - protein, 157 aa, synthetic construct
YFPKLGSKLSVIRNLNDQVLFIDQGNRPLFEDMTDSDCRDNAPRTIFIISMYKCSQPRGMAVTISVKCEKISTLSCENKIISFKEMNPPDVIKDTKSDIIFFQYSVPGHDNKMQFESSSYEGYFLACEKERDLFKLILKKEDELGDRSIMFTVQNED

SEQ ID NO: 276 - protein, 157 aa, synthetic construct
YFPKLGSKLSVIRNLNDQVLFIDQGNRPLFEDMTDSDCRDNAPRTIFIISMYKWSQPRGMAVTISVKCEKISTLSCENKIISFKEMNPPDVIKDTKSDIIFFQYSVPGHDNKMQFESSSYEGYFLACEKERDLFKLILKREDELGDRSIMFTVQNED

SEQ ID NO: 277 - protein, 157 aa, synthetic construct
YFPKLGSKLSVIRNLNDQVLFIDQGNRPLFEDMTDSDCRDNAPRTIFIISMYKWSQPRGMAVTISVKCEKISTLSCENKIISFKEMNPPDIIKDTKSDIIFFQYSVPGHDNKMQFESSSYEGYFLACEKERDLFKLILKKEDELGDRSIMFTVQNED

SEQ ID NO: 278 - protein, 157 aa, synthetic construct
YFPKLGSKLSIIRNLNDQVLFIDQGNRPLFEDMTDSDCRDNAPRTIFIISMYKWSQPRGMAVTISVKCEKISTLSCENKIISFKEMNPPDVIKDTKSDIIFFQYSVPGHDNKMQFESSSYEGYFLACEKERDLFKLILKKEDELGDRSIMFTVQNED

SEQ ID NO: 279 - protein, 157 aa, synthetic construct
YFPKLNSKLSVIRNLNDQVLFIDQGNRPLFEDMTDSDCRDNAPRTIFIISMYKWSQPRGMAVTISVKCEKISTLSCENKIISFKEMNPPDVIKDTKSDIIFFQYSVPGHDNKMQFESSSYEGYFLACEKERDLFKLILKKEDELGDRSIMFTVQNED

SEQ ID NO: 280 - protein, 157 aa, synthetic construct
YFSKLKSKLSVIRNLNDQVLFIDQGNRPLFEDMTDSDCRDNAPRTIFIISMYKWSQPRGMAVTISVKCEKISTLSCENKIISFKEMNPPDVIKDTKSDIIFFQYSVPGHDNKMQFESSSYEGYFLACEKERDLFKLILKKEDELGDRSIMFTVQNED

SEQ ID NO: 281 - protein, 157 aa, synthetic construct
YFPKLSSKLSVIRNLNDQVLFIDQGNRPLFEDMTDSDCRDNAPRTIFIISMYKWSQPRGMAVTISVKCEKISTLSCENKIISFKEMNPPDVIKDTKSDIIFFQYSVPGHDNKMQFESSSYEGYFLACEKERDLFKLILKKEDELGDRSIMFTVQNED

SEQ ID NO: 282 - protein, 157 aa, synthetic construct
YFPKLGSKLSVIRNLNDQVLFIDQGNRPLFEDMTDSDCRDNAPRTIFIISMYKWSQPRGMAVTISVKCEKISTLSCENKIISFKEMNPPDVIKDTKSDIIFFQYSVPGHDNKVQFESSSYEGYFLACEKERDLFKLILKKEDELGDRSIMFTVQNED

SEQ ID NO: 283 - protein, 157 aa, synthetic construct
YFPKLGSKLSVIRNLNDQVLFIDHGNRPLFEDMTDSDCRDNAPRTIFIISMYKWSQPRGMAVTISVKCEKISTLSCENKIISFKEMNPPDVIKDTKSDIIFFQYSVPGHDNKMQFESSSYEGYFLACEKERDLFKLILKKEDELGDRSIMFTVQNED

SEQ ID NO: 284 - protein, 157 aa, synthetic construct
YFPKLGSKLSVIRNLNDQVLFIDQGNRPFFEDMTDSDCRDNAPRTIFIISMYKWSQPRGMAVTISVKCEKISTLSCENKIISFKEMNPPDVIKDTKSDIIFFQYSVPGHDNKMQFESSSYEGYFLACEKERDLFKLILKKEDELGDRSIMFTVQNED

SEQ ID NO: 285 - protein, 156 aa, synthetic construct
YFPKLASKLSVIRNLNDQVLFIDQGNRPLFEDMTDSDCRDNAPRTIFIISMYKWSQPRGMAVTISVKCEKISTLSCENKIISFKEMNPPDVIKDTKSDIFFQYSVPGHDNKMQFESSSYEGYFLACEKERDLFKLILKKEDELGDRSIMFTVQNED

SEQ ID NO: 286 - protein, 157 aa, synthetic construct
YFPKLTSKLSVIRNLNDQVLFIDQGNRPLFEDMTDSDCRDNAPRTIFIISMYKWSQPRGMAVTISVKCEKISTLSCENKIISFKEMNPPDVIKDTKSDIIFFQYSVPGHDNKMQFESSSYEGYFLACEKERDLFKLILKKEDELGDRSIMFTVQNED

SEQ ID NO: 287 - protein, 157 aa, synthetic construct
YFPKLGSKLSVIRNLNDQVLFIDQGNRPLFEDMTDSDCRDNAPRTIFIISMYKWSQPRGMAVTISVKCEKISTLSCENKIISFKEMNPPDVIKDTKSDIIFFQVSVPGHDNKMQFESSSYEGYFLACEKERDLFKLILKKEDELGDRSIMFTVQNED

SEQ ID NO: 288 - protein, 157 aa, synthetic construct
YFPKLGSKLSVIRNLNDQVLFIDQGNRPLFEDMTDSDCRDNAPRTIFIISMYKWSQPRGIAVTISVKCEKISTLSCENKIISFKEMNPPDVIKDTKSDIIFFQYSVPGHDNKMQFESSSYEGYFLACEKERDLFKLILKKEDELGDRSIMFTVQNED

SEQ ID NO: 289 - protein, 157 aa, synthetic construct
YFTKLRSKLSVIRNLNDQVLFIDQGNRPLFEDMTDSDCRDNAPRTIFIISMYKWSQPRGMAVTISVKCEKISTLSCENKIISFKEMNPPDVIKDTKSDIIFFQYSVPGHDNKMQFESSSYEGYFLACEKERDLFKLILKKEDELGDRSIMFTVQNED

SEQ ID NO: 290 - protein, 157 aa, synthetic construct
YFTKLKSKLSVIRNLNDQVLFIDQGNRPLFEDMTDSDCRDNAPRTIFIISMYKWSQPRGMAVTISVKCEKISTLSCENKIISFKEMNPPDVIKDTKSDIIFFQYSVPGHDNKMQFESSSYEGYFLACEKERDLFKLILKKEDELGDRSIMFTVQNED

SEQ ID NO: 291 - protein, 157 aa, synthetic construct
YFPKLGSKLSVIRNLNDQVLFIDQGNRPLFEDMTDSDCRDNAPRTIFIISMYKWSQPRGMAVTISVKCEKISTLSCENKIISFKEMNPPDVIKDTKSDIIFFQFSVPGHDNKMQFESSSYEGYFLACEKERDLFKLILKKEDELGDRSIMFTVQNED

SEQ ID NO: 292 - protein, 157 aa, synthetic construct
YFPKLLSKLSVIRNLNDQVLFIDQGNRPLFEDMTDSDCRDNAPRTIFIISMYKWSQPRGMAVTISVKCEKISTLSCENKIISFKEMNPPDVIKDTKSDIIFFQYSVPGHDNKMQFESSSYEGYFLACEKERDLFKLILKKEDELGDRSIMFTVQNED

SEQ ID NO: 293 - protein, 157 aa, synthetic construct
YFPKLGSKLSVIRNLNDQVLFIDQGNRPLFEDMTDSDCRDNAPRTIFIISMYKWSQPRGMAVTISVKCEKISTLSCENKIISFKEMNPPDVIKDTKSDIIFFQYSVPGHDNKMQFESSSYEGYFLACEKERDLFKLILKMEDELGDRSIMFTVQNED

SEQ ID NO: 294 - protein, 157 aa, synthetic construct
YFPKLGSKLSVIRNLNDQVLFIDQGNRPLFEDMTDSDCRDNAPRTIFIISMYKWSQPRGMAVTISVKCEKISTLSCENKIISFKEMNPPDVIKDTKSDIIFFQYSVTGHDNKMQFESSSYEGYFLACEKERDLFKLILKKEDELGDRSIMFTVQNED

SEQ ID NO: 295 - protein, 157 aa, synthetic construct
YFPKLCSKLSVIRNLNDQVLFIDQGNRPLFEDMTDSDCRDNAPRTIFIISMYKWSQPRGMAVTISVKCEKISTLSCENKIISFKEMNPPDVIKDTKSDIIFFQYSVPGHDNKMQFESSSYEGYFLACEKERDLFKLILKKEDELGDRSIMFTVQNED

SEQ ID NO: 296 - protein, 157 aa, synthetic construct
YFPKLGSKLSVIRNLNDQVLFIDQGNRPLFEDMTDSDCRDNAPRTIFIISMYKWSQPRGMVVTISVKCEKISTLSCENKIISFKEMNPPDVIKDTKSDIIFFQYSVPGHDNKMQFESSSYEGYFLACEKERDLFKLILKKEDELGDRSIMFTVQNED

SEQ ID NO: 297 - protein, 157 aa, synthetic construct
YFWKLRSKLSVIRNLNDQVLFIDQGNRPLFEDMTDSDCRDNAPRTIFIISMYKWSQPRGMAVTISVKCEKISTLSCENKIISFKEMNPPDVIKDTKSDIIFFQYSVPGHDNKMQFESSSYEGYFLACEKERDLFKLILKKEDELGDRSIMFTVQNED

SEQ ID NO: 298 - protein, 157 aa, synthetic construct
YFWKLKSKLSVIRNLNDQVLFIDQGNRPLFEDMTDSDCRDNAPRTIFIISMYKWSQPRGMAVTISVKCEKISTLSCENKIISFKEMNPPDVIKDTKSDIIFFQYSVPGHDNKMQFESSSYEGYFLACEKERDLFKLILKKEDELGDRSIMFTVQNED

SEQ ID NO: 299 - protein, 157 aa, synthetic construct
YFWKLSSKLSVIRNLNDQVLFIDQGNRPLFEDMTDSDCRDNAPRTIFIISMYKWSQPRGMAVTISVKCEKISTLSCENKIISFKEMNPPDVIKDTKSDIIFFQYSVPGHDNKMQFESSSYEGYFLACEKERDLFKLILKKEDELGDRSIMFTVQNED

SEQ ID NO: 300 - protein, 157 aa, synthetic construct
NFGRLHCTTAVIRNINDQVLFVDKRQPVFEDMTDIDQSASEPQTRLIIYMYKDSEVRGLAVTLSVKDSKMSTLSCKNKIISFEEMDPPENIDDIQSDLIFFQKRVPGHNKMEFESSLYEGHFLACQKEDDAFKLILKKKDENGDKSVMFTLTNLHQS

SEQ ID NO: 301 - DNA, 471 bp, synthetic construct
  source 1..471: mol_type = other DNA
tacttcccgaaactgaaaagtaaactgagtgtgattcgcaatctgaatgatcaggttctgtttattgatcagggtaatcgcccgctgtttgaagatatgaccgatagcgattgccgcgataatgcaccgcgtaccatttttattattagtatgtataagggtagcggtgcccgtggtatggcagtgaccattagtgttaaatgcgaaaaaattagcaccctgagctgtgaaaataagattattagtttcaaggagatgaacccgccggataccattaaggataccaaaagtgatattatcttcttccagcgtagtgttccgggtcatgataataagatgcagtttgaaagcagcagctatgaaggctattttctggcatgtgaaaaagaacgtgatctgtttaaactgattctgaaaaaagaggatgagctgggcgatcgcagcattatgtttaccgtgcagaatgaagat

SEQ ID NO: 302 - DNA, 471 bp, synthetic construct
  source 1..471: mol_type = other DNA
tacttcccgaaactgaaaagcaaactgagcgtgattcgcaatctgaatgatcaggttctgtttattgatcagggtaatcgtccgctgtttgaagatatgaccgatagtgattgtcgtgataatgcaccgcgcaccatttttattattagcatgtataagcagagcatctggcgcggtatggccgttaccattagcgttaaatgcgaaaaaattagtaccctgagttgtgaaaataagattattagcttcaaggagatgaacccgccggataatattaaggataccaaaagcgatatcatctttttccagcgtagtgttccgggccatgataataagatgcagtttgaaagtagcagttatgaaggctattttctggcatgtgaaaaagaacgcgatctgtttaaactgattctgaaaaaagaggatgagctgggcgatcgcagcattatgtttaccgttcagaatgaagat

SEQ ID NO: 303 - DNA, 471 bp, synthetic construct
  source 1..471: mol_type = other DNA
tatttcccgaaactgcgttctaaactgtccgttatccgtaacctgaacgatcaggtgctgttcatcgaccagggtaaccgtccgctgttcgaagatatgactgactctgactgtcgtgacaacgcaccgcgtaccatctttatcatttctatgtacaaactgtctactgcgcgtggcatggccgtgaccattagcgtgaaatgtgaaaaaatctccactctgtcttgcgaaaacaagattatcagcttcaaagaaatgaaccctcctgatggtatcaaagatactaaatccgacatcattttcttccaacgttctgttccgggtcatgataataaaatgcagttcgaaagcagcagctacgaaggctatttcctggcatgtgaaaaagagcgtgacctgttcaaactgatcctgaagaaagaagacgaactgggcgaccgctctatcatgttcaccgtacagaacgaagac

SEQ ID NO: 304 - DNA, 471 bp, synthetic construct
  source 1..471: mol_type = other DNA
tacttcccgaaactgcgcagcaaactgagtgtgattcgcaatctgaatgatcaggtgctgtttattgatcagggtaatcgcccgctgtttgaagatatgaccgatagtgattgtcgtgataatgccccgcgtaccatttttattattagtatgtataagtacagcaccagtcgtggtatggccgtgaccattagtgtgaaatgtgaaaaaattagcaccctgagttgtgaaaataagattattagcttcaaggagatgaacccgccggataatattaaggataccaaaagtgatatcatcttctttcagcgtagtgttccgggtcatgataataagatgcagtttgaaagcagcagctatgaaggctattttctggcatgtgaaaaagaacgtgatctgtttaaactgattctgaaaaaagaggatgagctgggcgatcgtagtattatgtttaccgtgcagaatgaagat

SEQ ID NO: 305 - DNA, 471 bp, synthetic construct
  source 1..471: mol_type = other DNA
tacttcggcaaactgggctctaagctgagcgtgatccgtaacctgaacgaccaggtactgttcattgaccagggtaaccgtccgctgttcgaggatatgaccgacagcgactgccgtgataacgctccgcgtaccattttcattatctctatgtacaaactgtccaccgagcgtggtatggcagttacgatctctgtcaaatgcgaaaaaatttctactctgtcttgcgaaaacaaaatcatctcctttaaagaaatgaacccgccagatggtattaaagatactaaatctgacatcattttcttccaatcttctgtgccgggtcacgacaacaaaatgcagtttgaatccagctcctatgaaggttacttcctggcttgtgaaaaagaacgtgatctgttcaaactgatcctgaaaaaagaggacgaactgggcgaccgtagcatcatgttcaccgttcaaaatgaagat

SEQ ID NO: 306 - DNA, 471 bp, synthetic construct
  source 1..471: mol_type = other DNA
tactttgataaactgaaatctaaactgagcgtaatccgtaacctgaacgaccaagtactgttcatcgaccaaggtaaccgtccgctgtttgaagacatgaccgactctgattgccgtgacaatgcaccgcgcactatctttattatcagcatgtataaagactctcagccgcgtggtatggctgttactatctccgttaaatgcgaaaaaatctccaccctgtcttgcgaaaacaagattatctccttcaaagaaatgaacccgccggactccatcaaagacactaaatccgacatcatcttcttccaacgctccgtaccgggtcacgataacaaaatgcagtttgaaagctcttcctacgaaggctacttcctggcgtgtgagaaagagcgtgatctgttcaaactgatcctgaaaaaggaagatgaactgggcgatcgttccatcatgttcaccgtacagaacgaggat

SEQ ID NO: 307 - protein, 157 aa, synthetic construct
YFGKLESKLSVIRNLNDQVLFIDQGNRPLFEDMTDSDSRDNAPRTIFIISMYKDSQPRGMAVTISVKSEKISTLSSENKIISFKEMNPPDNIKDTKSDIIFFQRSVPGHDNKMQFESSSYEGYFLACEKERDLFKLILKKEDELGDRSIMFTVQNED

SEQ ID NO: 308 - protein, 157 aa, synthetic construct
YFPKLKSKLSVIRNLNDQVLFIDQGNRPLFEDMTDSDSRDNAPRTIFIISMYKGSGARGMAVTISVKSEKISTLSSENKIISFKEMNPPDTIKDTKSDIIFFQRSVPGHDNKMQFESSSYEGYFLACEKERDLFKLILKKEDELGDRSIMFTVQNED

SEQ ID NO: 309 - protein, 157 aa, synthetic construct
YFGKLGSKLSVIRNLNDQVLFIDQGNRPLFEDMTDSDSRDNAPRTIFIISMYKLSTERGMAVTISVKSEKISTLSSENKIISFKEMNPPDGIKDTKSDIIFFQSSVPGHDNKMQFESSSYEGYFLACEKERDLFKLILKKEDELGDRSIMFTVQNED

SEQ ID NO: 310 - protein, 157 aa, synthetic construct
YFPKLKSKLSVIRNLNDQVLFIDQGNRPLFEDMTDSDSRDNAPRTIFIISMYKGSGARGMAVTISVKSEKISTLSSENKIISFKEMNPPDTIKDTKSDIIFFQRSVPGHDNKMQFESSSYEGYFLASEKERDLFKLILKKEDELGDRSIMFTVQNED

SEQ ID NO: 311 - protein, 157 aa, synthetic construct
YFPKLKSKLSVIRNLNDQVLFIDQGNRPLFEDMTDSDIRDNAPRTIFIISMYKGSGARGMAVTISVKSEKISTLSCENKIISFKEMNPPDTIKDTKSDIIFFQRSVPGHDNKMQFECSSYEGYFLACEKERDLFKLILKKEDELGDRSIMFTVQNED

SEQ ID NO: 312 - protein, 157 aa, synthetic construct
YFPKLKSKLSVIRNLNDQVLFIDQGNRPLFEDMTDSDVRDNAPRTIFIISMYKGSGARGMAVTISVKIEKISTLSCENKIISFKEMNPPDTIKDTKSDIIFFQRSVPGHDNKMQFECSSYEGYFLAAEKERDLFKLILKKEDELGDRSIMFTVQNED

SEQ ID NO: 313 - protein, 157 aa, synthetic construct
YFPKLKSKLSVIRNLNDQVLFIDQGNRPLFEDMTDSDSRDNAPRTIFIISMYKGSGARGMAVTISVKIEKISTLSCENKIISFKEMNPPDTIKDTKSDIIFFQRSVPGHDNKMQFECSSYEGYFLAIEKERDLFKLILKKEDELGDRSIMFTVQNED

SEQ ID NO: 314 - protein, 157 aa, synthetic construct
YFPKLKSKLSVIRNLNDQVLFIDQGNRPLFEDMTDSDIRDNAPRTIFIISMYKGSGARGMAVTISVKIEKISTLSVENKIISFKEMNPPDTIKDTKSDIIFFQRSVPGHDNKMQFESSSYEGYFLAIEKERDLFKLILKKEDELGDRSIMFTVQNED

SEQ ID NO: 315 - protein, 157 aa, synthetic construct
YFPKLKSKLSVIRNLNDQVLFIDQGNRPLFEDMTDSDIRDNAPRTIFIISMYKGSGARGMAVTISVKLEKISTLSYENKIISFKEMNPPDTIKDTKSDIIFFQRSVPGHDNKMQFESSSYEGYFLACEKERDLFKLILKKEDELGDRSIMFTVQNED

SEQ ID NO: 316 - protein, 157 aa, synthetic construct
YFGKLGSKLSVIRNLNDQVLFIDQGNRPLFEDMTDSDIRDNAPRTIFIISMYKLSTERGMAVTISVKSEKISTLSCENKIISFKEMNPPDGIKDTKSDIIFFQSSVPGHDNKMQFECSSYEGYFLACEKERDLFKLILKKEDELGDRSIMFTVQNED

SEQ ID NO: 317 - protein, 157 aa, synthetic construct
YFGKLESKLSVIRNLNDQVLFIDQGNRPLFEDMTDSDIRDNAPRTIFIISMYKDSQPRGMAVTISVKSEKISTLSCENKIISFKEMNPPDNIKDTKSDIIFFQRSVPGHDNKMQFECSSYEGYFLACEKERDLFKLILKKEDELGDRSIMFTVQNED

SEQ ID NO: 318 - protein, 157 aa, synthetic construct
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFECSSYEGYFLACEKERDLFKLILKKEDELGDRSIMFTVQNED

SEQ ID NO: 319 - protein, 389 aa, synthetic construct
YFGKLESKLSVIRNLNDQVLFIDQGNRPLFEDMTDSDSRDNAPRTIFIISMYKDSQPRGMAVTISVKSEKISTLSSENKIISFKEMNPPDNIKDTKSDIIFFQRSVPGHDNKMQFESSSYEGYFLACEKERDLFKLILKKEDELGDRSIMFTVQNEDEPKSADKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 320 - protein, 389 aa, synthetic construct
YFPKLKSKLSVIRNLNDQVLFIDQGNRPLFEDMTDSDSRDNAPRTIFIISMYKGSGARGMAVTISVKSEKISTLSSENKIISFKEMNPPDTIKDTKSDIIFFQRSVPGHDNKMQFESSSYEGYFLACEKERDLFKLILKKEDELGDRSIMFTVQNEDEPKSADKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 321 - protein, 389 aa, synthetic construct
YFGKLGSKLSVIRNLNDQVLFIDQGNRPLFEDMTDSDSRDNAPRTIFIISMYKLSTERGMAVTISVKSEKISTLSSENKIISFKEMNPPDGIKDTKSDIIFFQSSVPGHDNKMQFESSSYEGYFLACEKERDLFKLILKKEDELGDRSIMFTVQNEDEPKSADKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 322 - protein, 389 aa, synthetic construct
YFPKLKSKLSVIRNLNDQVLFIDQGNRPLFEDMTDSDSRDNAPRTIFIISMYKGSGARGMAVTISVKSEKISTLSSENKIISFKEMNPPDTIKDTKSDIIFFQRSVPGHDNKMQFESSSYEGYFLASEKERDLFKLILKKEDELGDRSIMFTVQNEDEPKSADKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 323 - protein, 389 aa, synthetic construct
YFPKLKSKLSVIRNLNDQVLFIDQGNRPLFEDMTDSDIRDNAPRTIFIISMYKGSGARGMAVTISVKSEKISTLSCENKIISFKEMNPPDTIKDTKSDIIFFQRSVPGHDNKMQFECSSYEGYFLACEKERDLFKLILKKEDELGDRSIMFTVQNEDEPKSADKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 324 - protein, 389 aa, synthetic construct
YFPKLKSKLSVIRNLNDQVLFIDQGNRPLFEDMTDSDVRDNAPRTIFIISMYKGSGARGMAVTISVKIEKISTLSCENKIISFKEMNPPDTIKDTKSDIIFFQRSVPGHDNKMQFECSSYEGYFLAAEKERDLFKLILKKEDELGDRSIMFTVQNEDEPKSADKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 325 - protein, 389 aa, synthetic construct
YFPKLKSKLSVIRNLNDQVLFIDQGNRPLFEDMTDSDSRDNAPRTIFIISMYKGSGARGMAVTISVKIEKISTLSCENKIISFKEMNPPDTIKDTKSDIIFFQRSVPGHDNKMQFECSSYEGYFLAIEKERDLFKLILKKEDELGDRSIMFTVQNEDEPKSADKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 326 - protein, 389 aa, synthetic construct
YFPKLKSKLSVIRNLNDQVLFIDQGNRPLFEDMTDSDIRDNAPRTIFIISMYKGSGARGMAVTISVKIEKISTLSVENKIISFKEMNPPDTIKDTKSDIIFFQRSVPGHDNKMQFESSSYEGYFLAIEKERDLFKLILKKEDELGDRSIMFTVQNEDEPKSADKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 327 - protein, 389 aa, synthetic construct
YFPKLKSKLSVIRNLNDQVLFIDQGNRPLFEDMTDSDIRDNAPRTIFIISMYKGSGARGMAVTISVKLEKISTLSYENKIISFKEMNPPDTIKDTKSDIIFFQRSVPGHDNKMQFESSSYEGYFLACEKERDLFKLILKKEDELGDRSIMFTVQNEDEPKSADKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 328 - protein, 389 aa, synthetic construct
YFGKLESKLSVIRNLNDQVLFIDQGNRPLFEDMTDSDIRDNAPRTIFIISMYKDSQPRGMAVTISVKSEKISTLSCENKIISFKEMNPPDNIKDTKSDIIFFQRSVPGHDNKMQFECSSYEGYFLACEKERDLFKLILKKEDELGDRSIMFTVQNEDEPKSADKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 329 - protein, 389 aa, synthetic construct
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFECSSYEGYFLACEKERDLFKLILKKEDELGDRSIMFTVQNEDEPKSADKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 330 - protein, 389 aa, synthetic construct
YFGKLGSKLSVIRNLNDQVLFIDQGNRPLFEDMTDSDIRDNAPRTIFIISMYKLSTERGMAVTISVKSEKISTLSCENKIISFKEMNPPDGIKDTKSDIIFFQSSVPGHDNKMQFECSSYEGYFLACEKERDLFKLILKKEDELGDRSIMFTVQNEDEPKSADKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 331 - protein, 404 aa, synthetic construct
EPKSADKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGSGGGYFPKLKSKLSVIRNLNDQVLFIDQGNRPLFEDMTDSDIRDNAPRTIFIISMYKGSGARGMAVTISVKSEKISTLSCENKIISFKEMNPPDTIKDTKSDIIFFQRSVPGHDNKMQFECSSYEGYFLACEKERDLFKLILKKEDELGDRSIMFTVQNED

SEQ ID NO: 332 - protein, 404 aa, synthetic construct
EPKSADKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGSGGGYFGKLGSKLSVIRNLNDQVLFIDQGNRPLFEDMTDSDIRDNAPRTIFIISMYKLSTERGMAVTISVKSEKISTLSCENKIISFKEMNPPDGIKDTKSDIIFFQSSVPGHDNKMQFECSSYEGYFLACEKERDLFKLILKKEDELGDRSIMFTVQNED

SEQ ID NO: 333 - DNA, 471 bp, synthetic construct
  source 1..471: mol_type = other DNA
tacttcggcaagctggagtccaagctgtccgtgatccggaacctgaacgaccaggtgctgttcatcgaccagggcaacaggcctctcttcgaggatatgaccgactccgacatccgcgacaacgctcctagaacaatcttcatcatctccatgtacaaggactcccagcccaggggaatggctgttacaatttccgtgaagtccgagaagatctccaccctgtcctgcgagaacaagatcatctccttcaaggagatgaacccccccgacaacatcaaagacaccaaatccgacatcatcttcttccagcgcagcgtgcccggacatgataacaaaatgcagttcgaatgctcctcctacgagggctacttcctcgcatgcgaaaaggaaagggacctgttcaagctgatcctgaagaaggaggacgagttgggggacaggagcatcatgttcaccgtccagaacgaggac

SEQ ID NO: 334 - DNA, 471 bp, synthetic construct
  source 1..471: mol_type = other DNA
tacttccccaagctgaaatccaaactgagtgtgattcggaacctgaatgaccaggtgctgttcatcgaccagggcaacaggcctctgtttgaagatatgactgacagtgacatccgggacaacgccccaagaaccatcttcatcatctctatgtacaagggctccggggctaggggaatggctgtgaccattagcgtgaagtctgagaagatttccaccctgagctgcgagaacaagatcatttccttcaaagagatgaacccccccgacaccatcaaggacaccaagtccgacatcatcttcttccagaggtccgtgcccggccacgacaacaagatgcagttcgagtgctcctcctacgagggctacttcctggcctgcgagaaggagagggacctgttcaagctgatcctgaagaaggaggacgagctgggcgacaggtccatcatgttcaccgtgcagaacgaggac

SEQ ID NO: 335 - DNA, 471 bp, synthetic construct
  source 1..471: mol_type = other DNA
tacttcggcaagctgggttccaagctgtccgtgatccggaacctgaacgaccaggtgctgttcatcgaccagggcaacaggcctctcttcgaggatatgaccgactccgacatccgcgacaacgctcctagaacaatcttcatcatctccatgtacaagctgtccaccgagagggggatggctgttacaatttccgtgaagtccgagaaaatcagcacactgtcctgcgagaacaaaatcatctccttcaaggagatgaacccccccgacggcatcaaggacaccaagtccgacatcatcttcttccagtcctccgtgcccggccacgacaacaagatgcagttcgagtgctcctcctacgagggctacttcctggcctgcgagaaggagagggacctgttcaagctgatcctgaagaaggaggacgagctgggcgacaggtccatcatgttcaccgtgcagaacgaggac

SEQ ID NO: 336 - protein, 388 aa, synthetic construct
YFGKLESKLSVIRNLNDQVLFIDQGNRPLFEDMTDSDSRDNAPRTIFIISMYKDSQPRGMAVTISVKSEKISTLSSENKIISFKEMNPPDNIKDTKSDIIFFQRSVPGHDNKMQFESSSYEGYFLACEKERDLFKLILKKEDELGDRSIMFTVQNEDEPKSADKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 337 - protein, 388 aa, synthetic construct
YFPKLKSKLSVIRNLNDQVLFIDQGNRPLFEDMTDSDSRDNAPRTIFIISMYKGSGARGMAVTISVKSEKISTLSSENKIISFKEMNPPDTIKDTKSDIIFFQRSVPGHDNKMQFESSSYEGYFLACEKERDLFKLILKKEDELGDRSIMFTVQNEDEPKSADKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 338 - protein, 388 aa, synthetic construct
YFGKLGSKLSVIRNLNDQVLFIDQGNRPLFEDMTDSDSRDNAPRTIFIISMYKLSTERGMAVTISVKSEKISTLSSENKIISFKEMNPPDGIKDTKSDIIFFQSSVPGHDNKMQFESSSYEGYFLACEKERDLFKLILKKEDELGDRSIMFTVQNEDEPKSADKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 339 - protein, 388 aa, synthetic construct
YFPKLKSKLSVIRNLNDQVLFIDQGNRPLFEDMTDSDSRDNAPRTIFIISMYKGSGARGMAVTISVKSEKISTLSSENKIISFKEMNPPDTIKDTKSDIIFFQRSVPGHDNKMQFESSSYEGYFLASEKERDLFKLILKKEDELGDRSIMFTVQNEDEPKSADKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 340 - protein, 388 aa, synthetic construct
YFPKLKSKLSVIRNLNDQVLFIDQGNRPLFEDMTDSDIRDNAPRTIFIISMYKGSGARGMAVTISVKSEKISTLSCENKIISFKEMNPPDTIKDTKSDIIFFQRSVPGHDNKMQFECSSYEGYFLACEKERDLFKLILKKEDELGDRSIMFTVQNEDEPKSADKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 341 - protein, 388 aa, synthetic construct
YFPKLKSKLSVIRNLNDQVLFIDQGNRPLFEDMTDSDVRDNAPRTIFIISMYKGSGARGMAVTISVKIEKISTLSCENKIISFKEMNPPDTIKDTKSDIIFFQRSVPGHDNKMQFECSSYEGYFLAAEKERDLFKLILKKEDELGDRSIMFTVQNEDEPKSADKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 342 - protein, 388 aa, synthetic construct
YFPKLKSKLSVIRNLNDQVLFIDQGNRPLFEDMTDSDSRDNAPRTIFIISMYKGSGARGMAVTISVKIEKISTLSCENKIISFKEMNPPDTIKDTKSDIIFFQRSVPGHDNKMQFECSSYEGYFLAIEKERDLFKLILKKEDELGDRSIMFTVQNEDEPKSADKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 343 - protein, 388 aa, synthetic construct
YFPKLKSKLSVIRNLNDQVLFIDQGNRPLFEDMTDSDIRDNAPRTIFIISMYKGSGARGMAVTISVKIEKISTLSVENKIISFKEMNPPDTIKDTKSDIIFFQRSVPGHDNKMQFESSSYEGYFLAIEKERDLFKLILKKEDELGDRSIMFTVQNEDEPKSADKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 344 - protein, 388 aa, synthetic construct
YFPKLKSKLSVIRNLNDQVLFIDQGNRPLFEDMTDSDIRDNAPRTIFIISMYKGSGARGMAVTISVKLEKISTLSYENKIISFKEMNPPDTIKDTKSDIIFFQRSVPGHDNKMQFESSSYEGYFLACEKERDLFKLILKKEDELGDRSIMFTVQNEDEPKSADKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 345 - protein, 388 aa, synthetic construct
YFGKLESKLSVIRNLNDQVLFIDQGNRPLFEDMTDSDIRDNAPRTIFIISMYKDSQPRGMAVTISVKSEKISTLSCENKIISFKEMNPPDNIKDTKSDIIFFQRSVPGHDNKMQFECSSYEGYFLACEKERDLFKLILKKEDELGDRSIMFTVQNEDEPKSADKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 346 - protein, 388 aa, synthetic construct
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFECSSYEGYFLACEKERDLFKLILKKEDELGDRSIMFTVQNEDEPKSADKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 347 - protein, 388 aa, synthetic construct
YFGKLGSKLSVIRNLNDQVLFIDQGNRPLFEDMTDSDIRDNAPRTIFIISMYKLSTERGMAVTISVKSEKISTLSCENKIISFKEMNPPDGIKDTKSDIIFFQSSVPGHDNKMQFECSSYEGYFLACEKERDLFKLILKKEDELGDRSIMFTVQNEDEPKSADKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

(19) United States
(12) Patent Application Publication
(10) Pub. No.: US 2026/0092093 A1
(43) Pub. Date: Apr. 2, 2026

(54) IL-18 VARIANT POLYPEPTIDES

(71) Applicant: TJ Biopharma (Shanghai) Co., Ltd., Shanghai (CN)

(72) Inventors: Min QIANG, Shanghai (CN); Xi CHEN, Shanghai (CN); Zhengyi WANG, Shanghai (CN); Hui ZENG, Shanghai (CN); Xin ZHENG, Beijing (CN); Fei ZHANG, Beijing (CN); Puwei YUAN, Beijing (CN); Fan LIU, Beijing (CN)

(21) Appl. No.: 19/108,967

(22) PCT Filed: Sep. 6, 2023
(86) PCT No.: PCT/CN2023/117197

§ 371 Date: Mar. 5, 2025

(30) Foreign Application Priority Data
Sep. 6, 2022 (WO) PCT/CN2022/117332
Jan. 18, 2023 (WO) PCT/CN2023/072886

(51) Int. Cl. C07K 14/54; A61K 38/00

(52) U.S. Cl. CPC C07K 14/54; A61K 38/00; C07K 2319/30

(57) ABSTRACT

Provided are interleukin 18 (IL-18) variant polypeptides that comprise mutation(s) at one or more of G3, E6, D54, and N91, wherein amino acid positions are relative to a wild-type human IL-18 set forth in SEQ ID NO: 1. Also provided are IL-18 variant polypeptide comprising (such as further comprising) at least one mutation at a residue selected from the group consisting of: C38, C68, C76, D98, S117, and C127, wherein the amino acid position is relative to a wild-type human IL-18 set forth in SEQ ID NO: 1. Also provided are IL-18 variant polypeptides bind IL-18Rα and exhibit substantially reduced binding to IL-18BP relative to the wild-type IL-18. Also provided are IL-18 variant polypeptides that exhibit increased binding IL-18Rα relative to the wild-type IL-18 and substantially reduced binding to IL-18BP relative to the wild-type IL-18. Also provided are related nucleic acids, vectors, host cells, methods of producing IL-18 variant polypeptides, pharmaceutical compositions comprising IL-18 variant polypeptides, and uses of such pharmaceutical compositions.

### CROSS-REFERENCE TO RELATED APPLICATION

This application claims priority to International Applications PCT/CN2022/117332, filed on Sep. 6, 2022, and PCT/CN2023/072886, filed on Jan. 18, 2023, the content of which are incorporated by reference in their entirety for all purposes.

### REFERENCE TO AN ELECTRONIC SEQUENCE LISTING

The contents of the electronic sequence listing (233002001542SEQLIST.xml; Size: 362,441 bytes; and Date of Creation: Sep. 1, 2023) is herein incorporated by reference in its entirety.

### FIELD OF THE APPLICATION

The present application relates to interleukin 18 (IL-18) variant polypeptides comprising at least one amino acid substitution relative to a wild-type IL-18 (e.g. a wild-type human IL-18), methods of making such variant polypeptides, and methods of using such variant polypeptides in therapeutic applications.

### BACKGROUND OF THE APPLICATION

IL-18 has been found to stimulate innate lymphocytes, myeloid cells, antigen-experienced, non-naïve T cells (see, e.g., Guo et al. (2012) Trends Immunol. 33, 598-606), and antigen-experienced natural killer (NK cells). Therapeutically, recombinant IL-18 has been reported to synergize with immune checkpoint inhibitors (ICIs) (Ma et al. (2016) Clin Cancer Res 22:2969-2980) and chimeric antigen receptor T (CAR-T) cells in pre-clinical models (Hu et al. (2017) Cell Rep 20, 3025-3033). IL-18 has been administered to patients in clinical trials and found to be safe and well-tolerated (Robertson et al. (2006) Clin Cancer Res 12, 4265-4273). However, clinical development of IL-18 has been curtailed by its limited efficacy. Thus, there is a need in the art for compositions and methods for activating IL-18 receptor-mediated signaling in an individual and/or stimulating antigen-experienced T cells or NK cells in an individual for treating cancer and other IL-18-mediated diseases and disorders.

### SUMMARY

In some embodiments, provided is an interleukin 18 (IL-18) variant polypeptide comprising at least one mutation at a residue selected from the group consisting of: F2, G3, E6, V11, Q24, L29, D54, A61, N91, K96, R107, K140, and I149, wherein the amino acid position is relative to a wild-type human IL-18 set forth in SEQ ID NO: 1. In some embodiments, the IL-18 variant polypeptide comprises at least one mutation at a residue selected from the group consisting of: G3, E6, D54, and N91, wherein the amino acid positions is relative to a wild-type human IL-18 set forth in SEQ ID NO: 1. In some embodiments, the IL-18 variant polypeptide further comprises at least one mutation at a residue selected from the group consisting of Q56, P57, M60, Q103, R104, M113, and N155, wherein the amino acid position is relative to a wild-type human IL-18 set forth in SEQ ID NO: 1.

In some embodiments, the IL-18 variant polypeptide further comprises at least one mutation at a residue selected from the group consisting of: C38, C68, C76, D98, S117, and C127, wherein the amino acid position is relative to a wild-type human IL-18 set forth in SEQ ID NO: 1. In some embodiments, the at least one mutation comprises a substitution at C38, a substitution at C68, and an S117C substitution. In some embodiments, the at least one mutation is selected from the group consisting of: C38S, C68S, C76S, and C127S. In some embodiments, the IL-18 variant polypeptide comprises (such as further comprises) C38S, C68S, and C76S mutations. In some embodiments, the IL-18 variant polypeptide further comprises a set of mutations selected from the group consisting of: (a) C38I, C68S, and S117C; (b) C38V, C68I, S117C, and C127A; (c) C38S, C68I, S117C, and C127I; (d) C38I, C68I, C76V, and C127I; and (e) C38I, C68L, and C76Y.

In some embodiments, provided is an interleukin 18 (IL-18) variant polypeptide comprising at least one mutation at a residue selected from the group consisting of: C38, C68, C76, D98, S117, and C127, wherein the amino acid position is relative to a wild-type human IL-18 set forth in SEQ ID NO: 1. In some embodiments, the at least one mutation comprises a substitution at C38, a substitution at C68, and an S117C substitution. In some embodiments, the at least one mutation is selected from the group consisting of: C38S, C68S, C76S, and C127S. In some embodiments, the IL-18 variant comprises C38S, C68S, and C76S mutations. In some embodiments, the IL-18 variant polypeptide comprises a set of mutations selected from the group consisting of: (a) C38I, C68S, and S117C; (b) C38V, C68I, S117C, and C127A; (c) C38S, C68I, S117C, and C127I; (d) C38I, C68I, C76V, and C127I; and (e) C38I, C68L, and C76Y.

In some embodiments the wild-type IL-18 is human IL-18 comprising the amino acid sequence of SEQ ID NO:1. In some embodiments, the IL-18 variant polypeptide specifically binds to IL-18 receptor α (IL-18Rα) and exhibits reduced binding to IL-18 binding protein (IL-18BP) relative to the wild-type IL-18. In some embodiments, the IL-18 variant polypeptide exhibits an increased binding to IL-18Rα relative to the wild-type IL-18. In some embodiments, the IL-18 variant polypeptide binds to IL-18Rα with a K_{D} of less than about 5×10⁻⁵ M. In some embodiments, the IL-18 variant polypeptide binds to IL-18Rα with a K_{D} of between about 5×10⁻⁵ and about 5×10⁻¹¹ M. In some embodiments, the IL-18 variant polypeptide binds to IL-18BP with a K_{D} of more than 5×10⁻⁹ M. In some embodiments, the IL-18 variant polypeptide exhibits no binding (such as no detectable binding, as measured, e.g., via surface plasmon resonance) to IL-18BP.

In some embodiments, the variant polypeptide comprises a mutation at residue G3, wherein the mutation is selected from the group consisting of: G3P, G3D, G3E, G3F, G3K, G3T, G3W, G3N, and G3S. In some embodiments, the mutation is G3P. In some embodiments, the variant polypeptide comprises a mutation at residue E6, wherein the mutation is selected from the group consisting of: E6R, E6K, E6G, E6T, E6A, E6S, E6H, E6L, E6M, E6N, E6P, E6Q, E6V, E6W, and E6Y. In some embodiments, the mutation is selected from the group consisting of: E6R, E6K, E6G, E6T, E6A, and E6S. In some embodiments, the mutation is selected from the group consisting of: E6R and E6K.

In some embodiments, the variant polypeptide comprises a mutation at residue D54, wherein the mutation is selected from the group consisting of: D54W, D54H, D54I, D54S, D54Q, D54L, D54M, D54Y, D54P, D54R, D54A, D54F, D54G, D54V, and D54T. In some embodiments, the mutation is selected from the group consisting of: D54W, D54H, D54S, D54Q, D54L, and D54Y.

In some embodiments, the variant polypeptide comprises a mutation at residue N91, wherein the mutation is selected from the group consisting of: N91V, N91A, N91D, N91F, N91G, N91S, N91I, N91P, N91R, N91L, N91T, N91C, N91K, N91Y and N91W. In some embodiments, the mutation is selected from the group consisting of: N91V, N91A, N91G, and N91S.

In some embodiments, the variant polypeptide further comprises a mutation at residue R104, wherein the mutation is selected from the group consisting of: R104S, R104Y, R104T, R104L, R104M, R104V, R104A, R104C, R104E, R104G, R104F, R104H, R104I, and R104N. In some embodiments, the mutation is selected from the group consisting of: R104S, R104Y, and R104T.

In some embodiments, the variant polypeptide comprises no mutation at residue R104.

In some embodiments, the variant polypeptide comprises a mutation at residue Q56, wherein the mutation is selected from the group consisting of: Q56A, Q56T, Q56G, Q56R, Q56S, Q56D, Q56P, Q56I, Q56Y, Q56H, Q56I, Q56K, Q56W, Q56L, Q56E, Q56F, Q56N, and Q56V. In some embodiments, the mutation is selected from the group consisting of: Q56T, Q56G, Q56R, Q56S, Q56D, Q56P, Q56I, and Q56Y.

In some embodiments, the variant polypeptide comprises no mutation at residue Q56.

In some embodiments, the variant polypeptide comprises a mutation at residue P57, wherein the mutation is selected from the group consisting of: P57A, P57E, P57F, P57G, P57R, P57W, P57S, P57T, P57V, P57Q, P57H, P57I, P57K, P57L, P57N, P57Y, and P57D. In some embodiments, the mutation is selected from the group consisting of: P57A, P57G, P57R, P57W, P57S, P57T, and P57V.

In some embodiments, the variant polypeptide comprises no mutation at residue P57.

In some embodiments, the variant polypeptide comprises mutations at G3, E6, D54, and N91. In some embodiments, the variant polypeptide comprises mutations: (i) G3P; (ii) E6R or E6K; (iii) D54W, D54H, D54S, or D54Q; and (iv) N91V, N91A, N91G, or N91S. In some embodiments, the variant polypeptide further comprises mutations at Q56 and P57.

In some embodiments, the variant polypeptide comprises the amino acid sequence set forth in any one of SEQ ID NOs: 2-299 and 307-318.

In some embodiments, provided is a fusion polypeptide comprising an IL-18 variant polypeptide described herein and a human IgG Fc domain or variant thereof. In some embodiments, the fusion polypeptide comprises a human IgG1 Fc domain variant that comprises an N297A mutation (EU numbering). In some embodiments, the C-terminus of the IL-18 variant polypeptide is fused to the N-terminus of the human IgG Fc domain or variant thereof. In some embodiments, the C-terminus of the human IgG Fc domain or variant thereof is fused to the N-terminus of the IL-18 variant polypeptide. In some embodiments, the fusion polypeptide comprises the amino acid sequence of any one of SEQ ID NOs: 319-332 and 336-347.

In some embodiments, provided is a dimer comprising two fusion polypeptides described herein. In some embodiments, the dimer is a homodimer. In some embodiments, the dimer is a heterodimer.

In some embodiments, provided is a nucleic acid encoding an IL-18 variant polypeptide or a fusion polypeptide described herein. In some embodiments, the nucleic acid comprises a polynucleotide sequence having at least 80% identity to any one of SEQ ID NOs: 301-306 and 333-335. In some embodiments, provided is a vector comprising a nucleic acid described herein. In some embodiments, provided is a host cell comprising a nucleic acid or a vector described herein. In some embodiments, provided is a method of producing an IL-18 variant polypeptide or fusion polypeptide, comprising: (a) culturing a host cell described herein under conditions where the IL-18 variant polypeptide or fusion polypeptide is expressed; and (b) recovering the IL-18 variant polypeptide or fusion polypeptide produced by the host cell. In some embodiments, the host cell is a mammalian host cell (e.g., a CHO cell or an HEK293 cell). In some embodiments, the method comprises (such as further comprises) purifying the IL-18 variant polypeptide or the fusion polypeptide.

In some embodiments, provided is a pharmaceutical composition comprising an IL-18 variant polypeptide described herein, a fusion polypeptide described herein, a nucleic acid described herein, or a vector described herein.

In some embodiments, provided is a method of treating a disease in an individual, comprising administering to the individual an effective amount of a pharmaceutical composition described herein. In some embodiments, the disease is cancer. In some embodiments, provided is a method of activating hIL-18 receptor mediated signal in an individual, comprising administering to the individual an effective amount of a pharmaceutical composition described herein. In some embodiments, provided is a method of stimulating antigen-experienced immune cells in an individual in need thereof, comprising administering to the individual an effective amount of a pharmaceutical composition described herein. In some embodiments, the stimulation comprises increasing activity and/or number of the antigen-experienced immune cells.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

The disclosures of all publications, patents, patent applications and published patent applications referred to herein are hereby incorporated herein by reference in their entirety.

### BRIEF DESCRIPTION OF THE DRAWINGS

FIG. 1A illustrates the screening process in which magnetic-activated cell sorting- (MACS-) or fluorescence-activated cell sorting- (FACS-) based methods were used to identify IL-18 variant polypeptides described herein.

FIG. 1B illustrates the abilities of the output library pools from Rounds 1, 2, 3, 4, and 5 to bind either 1 μM IL-18BP or 50 nM IL-18Rα.

FIG. 1C illustrates the abilities of representative single IL-18 variant clones M12 (SED ID NO: 39) and M17 (SEQ ID NO: 43) to bind IL-18Rα. SEQ ID NO: 1 is wild-type IL-18.

FIG. 2A provides an SDS-PAGE analysis and SEC-HPLC analysis of wild-type human IL-18 (SEQ ID NO: 1) after purification.

FIG. 2B provides an SDS-PAGE analysis and SEC-HPLC analysis of IL-18 variant M12 (SEQ ID NO: 39) after purification.

FIG. 2C provides an SDS-PAGE analysis and SEC-HPLC analysis of IL-18 variant MM5 (SEQ ID NO: 133) after purification.

FIG. 2D provides an SDS-PAGE analysis and SEC-HPLC analysis of IL-18 variant WM6 (SEQ ID NO: 145) after purification.

FIG. 2E provides an SDS-PAGE analysis and SEC-HPLC analysis of IL-18 variant M17 (SEQ ID NO: 303) after purification.

FIG. 2F provides an SDS-PAGE analysis and SEC-HPLC analysis of IL-18 variant M24 (SEQ ID NO: 105) after purification.

FIG. 3 illustrates activation of hIL-18 receptor mediated signaling by exemplary IL-18 variant polypeptides of the present application.

FIG. 4 illustrates hIFNγ release induced by exemplary IL-18 variant polypeptides of the present application.

FIG. 5A illustrates activation of hIL-18 receptor mediated signaling by wild-type IL-18, fusion polypeptide IL-18-SSS-Fc_N297A, and fusion polypeptide IL-18-SSS-Fc_N297A in the presence of IL-18 binding protein (“BP”).

FIG. 5B illustrates activation of hIL-18 receptor mediated signaling by IL-18 polypeptide variant M12, fusion polypeptide M12-SSS-Fc_N297A, and fusion polypeptide M12-SSS-Fc_N297A in the presence of IL-18 binding protein (“BP”).

FIG. 5C illustrates activation of hIL-18 receptor mediated signaling by IL-18 polypeptide variant MM5, fusion polypeptide MM5-SSS-Fc_N297A, and fusion polypeptide MM5-SSS-Fc_N297A in the presence of IL-18 binding protein (“BP”).

FIG. 6A illustrates activation of hIL-18 receptor mediated signaling by fusion polypeptide WT IL-18, WT IL-18 in the presence of IL-18 binding protein (“BP”), fusion polypeptide M12-DB6-Fc_N297A, and M12-DB6-Fc_N297A in the presence of IL-18 binding protein (“BP”).

FIG. 6B illustrates activation of hIL-18 receptor mediated signaling by fusion polypeptide WT IL-18, fusion polypeptide M12-DB7-Fc_N297A, and M12-DB7-Fc_N297A in the presence of IL-18 binding protein (“BP”).

FIG. 6C illustrates activation of hIL-18 receptor mediated signaling by fusion polypeptide WT IL-18, fusion polypeptide M12-DB9-Fc_N297A, and M12-DB9-Fc_N297A in the presence of IL-18 binding protein (“BP”).

FIG. 6D illustrates activation of hIL-18 receptor mediated signaling by fusion polypeptide WT IL-18, fusion polypeptide M12-DB10-Fc_N297A.

FIG. 7A illustrates activation of hIL-18 receptor mediated signaling by wild-type IL-18, wild-type IL-18 in the presence of IL-18 binding protein (“BP”), fusion polypeptide WT IL-18-DB6-Fc_N297A, and WT IL-18-DB6-Fc_N297A in the presence of IL-18 binding protein (“BP”).

FIG. 7B illustrates activation of hIL-18 receptor mediated signaling by wild-type IL-18, wild-type IL-18 in the presence of IL-18 binding protein (“BP”), fusion polypeptide MM5-DB6-Fc_N297A, and MM5-DB6-Fc_N297A in the presence of IL-18 binding protein (“BP”).

FIG. 7C illustrates activation of hIL-18 receptor mediated signaling by wild-type IL-18, and fusion polypeptide Fc_N297A-MM5-DB6.

FIG. 7D illustrates activation of hIL-18 receptor mediated signaling by wild-type IL-18, and fusion polypeptide Fc_N297A-M12-DB6.

### DETAILED DESCRIPTION OF THE APPLICATION

### Overview

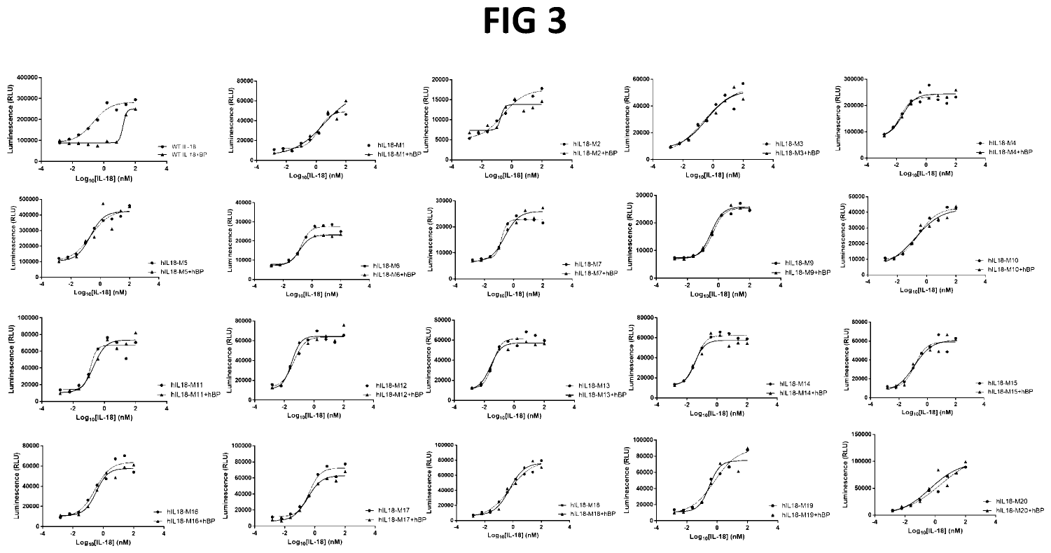

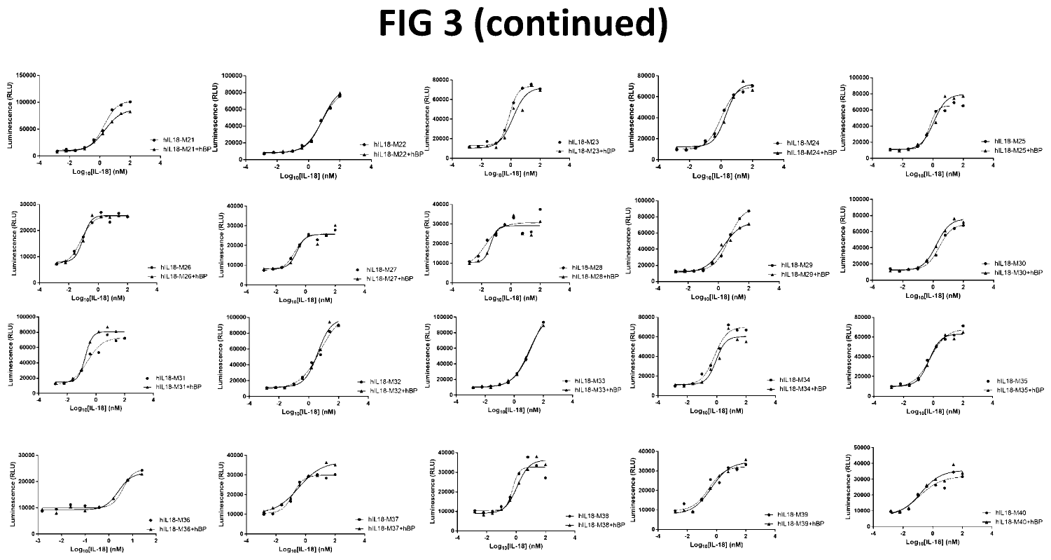

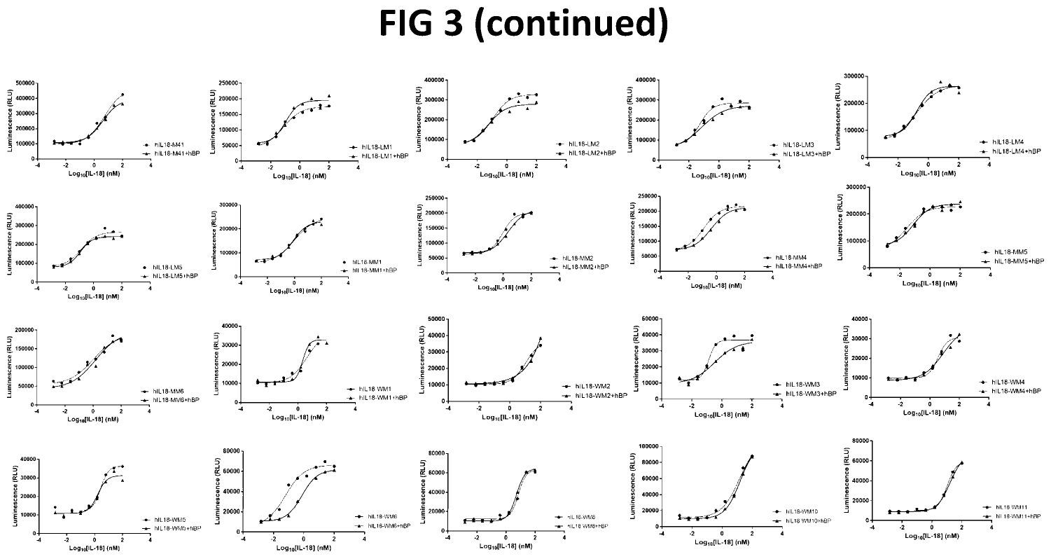

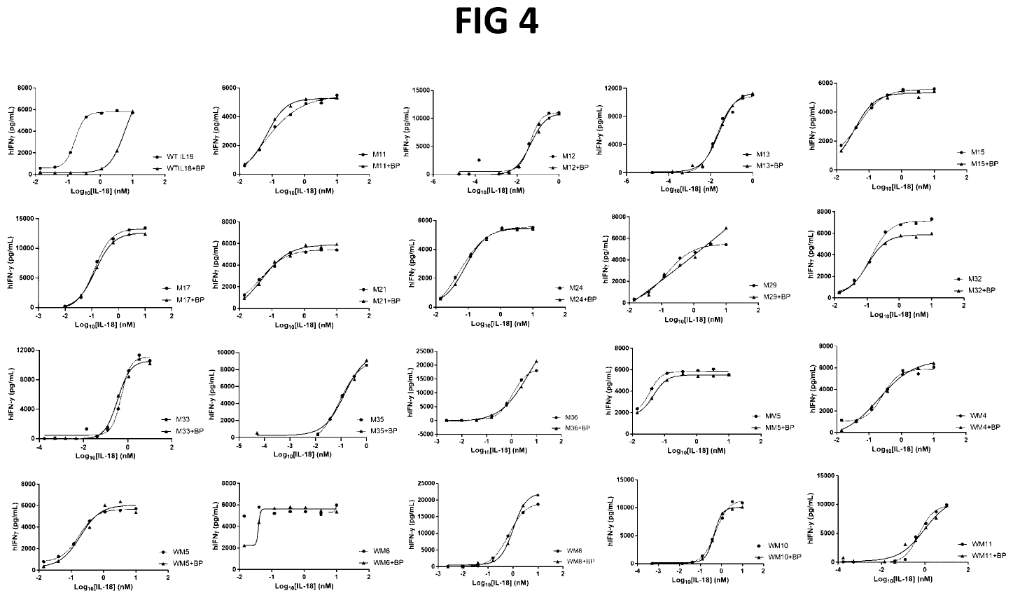

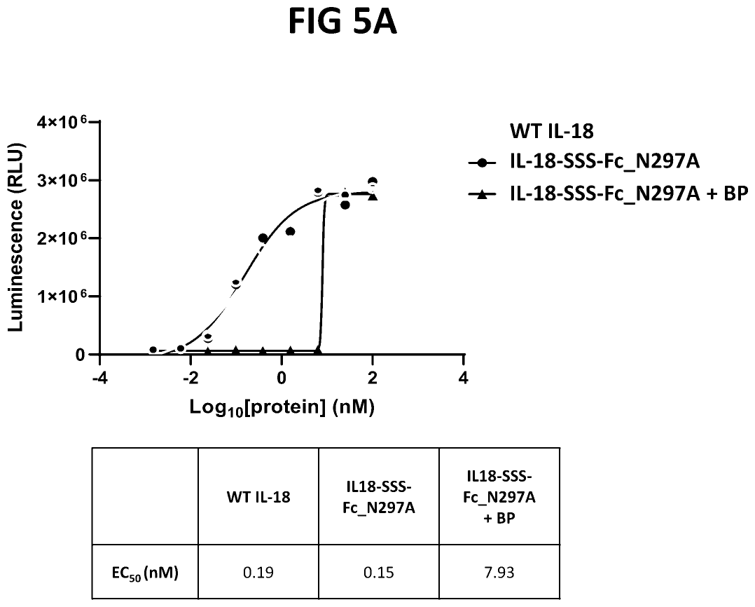

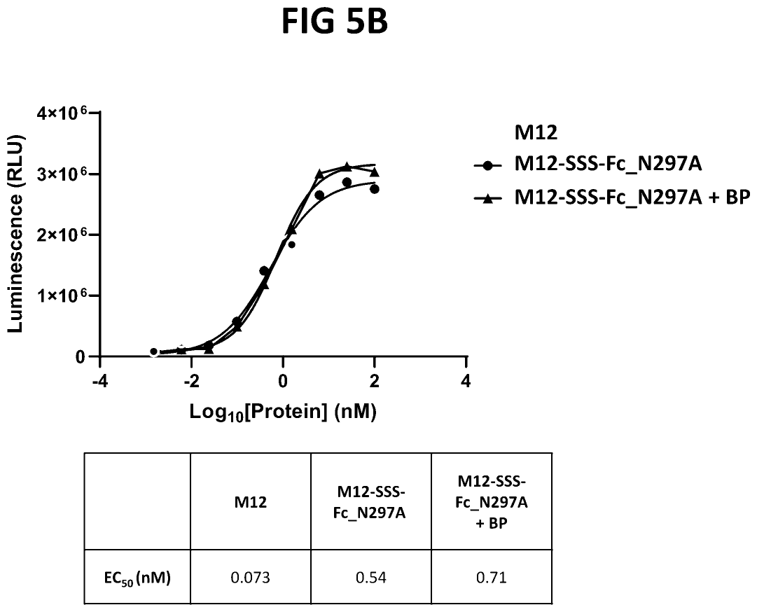

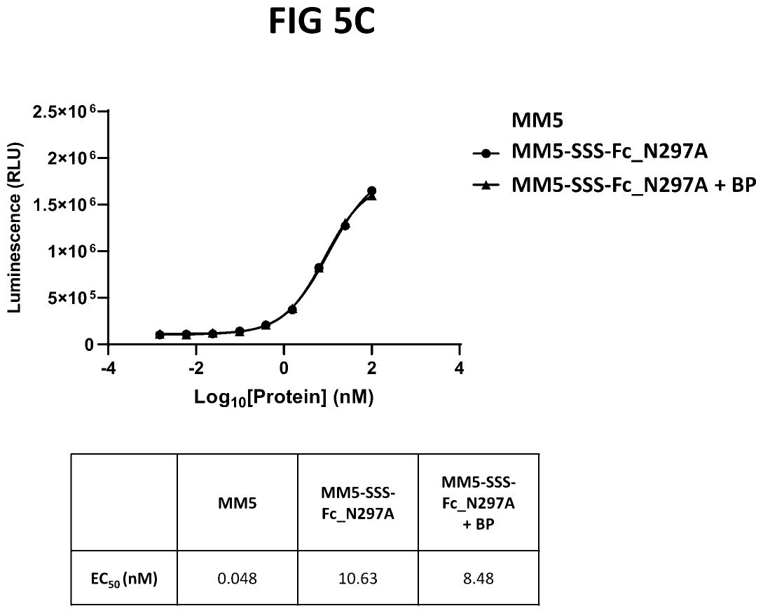

Components of the interleukin-18 (IL-18) pathway have been found to be upregulated on tumor infiltrating lymphocytes (TIL) (see, e.g., Zhou et al. (2020) Nature 583:609-614), suggesting that IL-18 therapy could enhance anti-tumor immunity. However, IL-18 has not demonstrated significant efficacy in clinical trials. IL-18BP, a high-affinity inhibitor of IL-18 (K_{D}<1 nM, see, e.g., Dinarello et al. (2013) Front Immunol 4:289, doi: 10.3389/fimmu.2013.00289), is frequently upregulated in diverse human and murine tumors and is believed to limit the anti-tumor activity of IL-18 in mice and clinical trial. The present application is based in part on Applicant's identification of IL-18 variant polypeptides that induce IL-18 receptor-mediated signaling, even in the presence of IL-18BP. It was found that by being substituted one or a set of amino acids (e.g., G3, e.g., E6), IL-18 variant polypeptides exhibit resistance to IL-18BP while still being able to bind to and activate IL-18 receptor. See, e.g., Example 6, FIGS. 3-4, Tables 3 and 4. It was also found that with one or more of amino acids at C38, C68, C76, S117, and/or C127 substituted, a higher yield, more purified, and/or more potent IL-18 variant polypeptides can be obtained. See, e.g., Example 7, FIGS. 5A-7D, Tables 5-9.

### Definitions

As used herein, the terms “specifically binds,” “specifically recognizing,” and “is specific for” refer to measurable and reproducible interactions, such as binding between a cytokine and a receptor thereof, which is determinative of the presence of the target in the presence of a heterogeneous population of molecules, including biological molecules. For example, a cytokine that specifically recognizes a receptor is the cytokine that binds this receptor with greater affinity, avidity, more readily, and/or with greater duration than its bindings to other targets. In some embodiments, the extent of binding of cytokine to an unrelated target is less than about 10% of the binding of the cytokine to the receptor thereof as measured, e.g., by a radioimmunoassay (RIA). In some embodiments, a cytokine that specifically binds the receptor thereof has a dissociation constant (K_{D}) of ≤10⁻⁵ M, ≤10⁻⁶ M, ≤10⁻⁷ M, ≤10⁻⁸ M, ≤10⁻⁹M, ≤10⁻¹⁰ M, ≤10⁻¹¹ M, or ≤10⁻¹² M. In some embodiments, said specific binding can include, but does not require exclusive binding. Binding specificity of the cytokine can be determined experimentally by methods known in the art. Such methods comprise, but are not limited to, e.g., Western blots, ELISA-, RIA-, ECL-, IRMA-, EIA-, BIACORE™-tests and peptide scans.

An “isolated” nucleic acid molecule encoding a polypeptides or a cytokine as described herein is a nucleic acid molecule that is identified and separated from at least one contaminant nucleic acid molecule with which it is ordinarily associated in the environment in which it was produced. Preferably, the isolated nucleic acid is free of association with all components associated with the production environment. In some embodiments, the isolated nucleic acid molecules encoding the polypeptides or the cytokine described herein is in a form other than in the form or setting in which it is found in nature.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, and may contain mutations. Mutant progeny that have the same function or biological activity as screened or selected for in the originally transformed cell are included herein.

As used herein, “treatment” or “treating” is an approach for obtaining beneficial or desired results, including clinical results. For purposes of this application, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delaying or slowing the progression of the disease, ameliorating the disease state, providing a remission (partial or total) of the disease, decreasing the dose of one or more other medications required to treat the disease, delaying the progression of the disease, increasing or improving the quality of life, increasing weight gain, and/or prolonging survival. Also encompassed by “treatment” is a reduction of pathological consequence of cancer (such as, for example, tumor volume). The methods of the application contemplate any one or more of these aspects of treatment.

In the context of cancer, the term “treating” includes any or all of: inhibiting growth of cancer cells, inhibiting replication of cancer cells, lessening of overall tumor burden and ameliorating one or more symptoms associated with the disease.

The terms “inhibition” or “inhibit” refer to a decrease or cessation of any phenotypic characteristic or to the decrease or cessation in the incidence, degree, or likelihood of that characteristic. To “reduce” or “inhibit” is to decrease, reduce or arrest an activity, function, and/or amount as compared to that of a reference. In certain embodiments, by “reduce” or “inhibit” is meant the ability to cause an overall decrease of 20% or greater. In another embodiment, by “reduce” or “inhibit” is meant the ability to cause an overall decrease of 50% or greater. In yet another embodiment, by “reduce” or “inhibit” is meant the ability to cause an overall decrease of 75%, 85%, 90%, 95%, or greater.

The terms “subject.” “individual,” and “patient” are used interchangeably herein to refer to a mammal, including, but not limited to, human, bovine, horse, feline, canine, rodent, or primate. In some embodiments, the individual is a human.

It is understood that embodiments of the application described herein include “consisting” and/or “consisting essentially of” embodiments.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter. For example, the method is not used to treat cancer of type X means the method is used to treat cancer of types other than X.

The term “about X-Y” used herein has the same meaning as “about X to about Y.”

As used herein and in the appended claims, the singular forms “a,” “or,” and “the” include plural referents unless the context clearly dictates otherwise.

### Interleukin 18 (IL-18) Variant Polypeptides

Interleukin-18 (IL-18, also known as interferon-gamma inducing factor) is a protein which in humans is encoded by the IL-18 gene. It is a proinflammatory cytokine which potentially can be produced by hematopoietic cells and non-hematopoietic cells. IL-18 can modulate both innate and adaptive immunity and its dysregulation can cause autoimmune or inflammatory diseases.

In one aspect, provided herein are interleukin 18 (“IL-18”) variant polypeptides comprising mutation(s) at one or more of F2, G3, E6, V11, Q24, L29, D54, A61, N91, K96, R107, K140, and I149, wherein amino acid positions are relative to a wild type (WT) human IL-18 set forth in SEQ ID NO:1. In some embodiments, the wild-type IL-18 is human IL-18 comprising the amino acid sequence of SEQ ID NO: 1.

(SEQ ID NO: 1)
YFGKLESKLSVIRNLNDQVLFIDQGNRPLFEDMTDSDCRDNAPRTIFIIS
MYKDSQPRGMAVTISVKCEKISTLSCENKIISFKEMNPPDNIKDTKSDII
FFQRSVPGHDNKMQFESSSYEGYFLACEKERDLFKLILKKEDELGDRSIM
FTVQNED

In some embodiments, the IL-18 variant polypeptide comprises mutations at two or more of F2, G3, E6, V11, Q24, L29, D54, A61, N91, K96, R107, K140, and I149, e.g., in any combination. In some embodiments, the two or more mutations are selected from the group consisting of: F2, G3, E6, V11, Q24, L29, D54, A61, N91, K96, R107, K140, and I149. In some embodiments, the IL-18 variant polypeptide comprises mutations at three or more of F2, G3, E6, V11, Q24, L29, D54, A61, N91, K96, R107, K140, and I149, e.g., in any combination. In some embodiments, the three or more mutations are selected from the group consisting of: F2, G3, E6, V11, Q24, L29, D54, A61, N91, K96, R107, K140, and I149. In some embodiments, the IL-18 variant polypeptide comprises mutations at four or more of F2, G3, E6, V11, Q24, L29, D54, A61, N91, K96, R107, K140, and I149, e.g., in any combination. In some embodiments, the four or more mutations are selected from the group consisting of: F2, G3, E6, V11, Q24, L29, D54, A61, N91, K96, R107, K140, and I149. In some embodiments, the IL-18 variant polypeptide comprises mutations at five or more of F2, G3, E6, V11, Q24, L29, D54, A61, N91, K96, R107, K140, and I149, e.g., in any combination. In some embodiments, the five or more mutations are selected from the group consisting of: F2, G3, E6, V11, Q24, L29, D54, A61, N91, K96, R107, K140, and I149. In some embodiments, the IL-18 variant polypeptide comprises mutations at six or more of F2, G3, E6, V11, Q24, L29, D54, A61, N91. K96, R107, K140, and I149, e.g., in any combination. In some embodiments, the six or more mutations are selected from the group consisting of: F2, G3, E6, V11, Q24, L29, D54, A61, N91, K96, R107, K140, and I149. In some embodiments, the IL-18 variant polypeptide comprises mutations at seven or more of F2, G3, E6, V11, Q24, L29, D54, A61, N91, K96, R107, K140, and I149, e.g., in any combination. In some embodiments, the seven or more mutations are selected from the group consisting of: F2, G3, E6, V11, Q24, L29, D54, A61, N91, K96, R107, K140, and I149. In some embodiments, the IL-18 variant polypeptide comprises mutations at eight or more of F2, G3, E6, V11, Q24, L29, D54, A61, N91, K96, R107, K140, and I149, e.g., in any combination. In some embodiments, the eight or more mutations are selected from the group consisting of: F2, G3, E6, V11, Q24, L29, D54, A61, N91, K96, R107, K140, and I149. In some embodiments, the IL-18 variant polypeptide comprises mutations at nine or more of F2, G3, E6, V11, Q24, L29, D54, A61, N91, K96, R107, K140, and I149, e.g., in any combination. In some embodiments, the nine or more mutations are selected from the group consisting of: F2, G3, E6, V11, Q24, L29, D54, A61, N91, K96, R107, K140, and I149. In some embodiments, the IL-18 variant polypeptide comprises mutations at ten or more of F2, G3, E6, V11, Q24, L29, D54, A61, N91, K96, R107, K140, and I149, e.g., in any combination. In some embodiments, the ten or more mutations are selected from the group consisting of: F2, G3, E6, V11, Q24, L29, D54, A61, N91, K96, R107, K140, and I149. In some embodiments, the IL-18 variant polypeptide comprises mutations at eleven or more of F2, G3, E6, V11, Q24, L29, D54, A61, N91, K96, R107, K140, and I149, e.g., in any combination. In some embodiments, the eleven or more mutations are selected from the group consisting of: F2, G3, E6, V11, Q24, L29, D54, A61, N91, K96, R107, K140, and I149. In some embodiments, the IL-18 variant polypeptide comprises mutations at twelve or more of F2, G3, E6, V11, Q24, L29, D54, A61, N91, K96, R107, K140, and I149, e.g., in any combination. In some embodiments, the twelve or more mutations are selected from the group consisting of: F2, G3, E6, V11, Q24, L29, D54, A61, N91, K96, R107, K140, and I149. In some embodiments, the IL-18 variant polypeptide comprises mutations at thirteen or more of F2, G3, E6, V11, Q24, L29, D54, A61, N91, K96. R107, K140, and I149, e.g., in any combination. In some embodiments, the thirteen or more mutations are selected from the group consisting of: F2, G3, E6, V11, Q24, L29, D54, A61, N91, K96, R107, K140, and I149. In some embodiments, the IL-18 variant polypeptide comprises mutations at F2, G3, E6, V11, Q24, L29, D54, A61, N91, K96, R107, K140, and I149. In some embodiments, the IL-18 variant polypeptide does not comprise mutations at positions other than F2, G3, E6, V11, Q24, L29, D54, A61, N91, K96, R107, K140, and I149. In some embodiments, the IL-18 variant comprises (or further comprises) mutations at positions other than at F2, G3, E6, V11, Q24, L29, D54, A61, N91, K96, R107, K140, and I149. In some embodiments, the IL-18 variant polypeptide comprises one or more mutations (such as any of 2, 3, 4, 5, 6, 7, 8, 9, 10, 11, 12, 13, 14, or 15 mutations), wherein at least one, at least 2, at least 3, or at least 4 mutations are selected from the group consisting of: F2, G3, E6, V11, Q24, L29. D54, A61, N91, K96, R107, K140, and I149. In some embodiments, the wild-type IL-18 is human IL-18 comprising the amino acid sequence of SEQ ID NO:1.

In some embodiments, the one or more mutations are selected from the group consisting of: G3, E6, D54, and N91. In some embodiments, the IL-18 variant polypeptide comprises mutations at two or more of G3, E6, D54, and N91, e.g., in any combination. In some embodiments, the two or more mutations are selected from the group consisting of: G3, E6, D54, and N91. In some embodiments, the IL-18 variant polypeptide comprises mutations at three or more of G3, E6, D54, and N91, e.g., in any combination. In some embodiments, the three or more mutations are selected from the group consisting of: G3, E6, D54, and N91. In some embodiments, the IL-18 variant polypeptide comprises mutations at G3, E6, D54, and N91. In some embodiments, the IL-18 variant polypeptide does not comprise mutations at positions other than G3, E6, D54, and/or N91. In some embodiments, the IL-18 variant comprises (or further comprises) mutations at positions other than at G3, E6, D54, and/or N91. In some embodiments, the IL-18 variant polypeptide comprises one or more mutations (such as any of 2, 3, 4, 5, 6, 7, 8, 9, 10, 11, 12, 13, 14, or 15 mutations), wherein at least one, at least 2, at least 3, or at least 4 mutations are selected from the group consisting of: G3, E6, D54, and N91. In some embodiments, the wild-type IL-18 is human IL-18 comprising the amino acid sequence of SEQ ID NO:1.

In some embodiments, the IL-18 variant polypeptide comprises (e.g., further comprises) at least one mutation at a residue selected from the group consisting of Q56, P57, M60, Q103, R104, M113, and N155, wherein the amino acid position is relative to a wild-type human IL-18 set forth in SEQ ID NO: 1.

In some embodiments, the IL-18 variant polypeptide comprises at least one mutation (or two, or three) at a residue selected from the group consisting of G3, E6, and M60, wherein the amino acid positions is relative to a wild-type human IL-18 set forth in SEQ ID NO: 1. In some embodiments, the IL-18 variant polypeptide comprises at least one of a) G3P, G3S, G3E, G3N, G3D, or G3A; b) E6K, E6T. E6R, E6A, E6M, E6L, E6G, E6H, E6S, or E6Y; or c) M60K. In some embodiments, the IL-18 variant polypeptide further comprises at least one or more (e.g., two, three, four, or five) mutations at a residue selected from the group consisting of V11, D54, Q56, P57, N91, K96, R104, R140, I149, and N155. In some embodiments, the IL-18 variant polypeptide further comprises at least one of a) D54H, D54W, D54Q, D54S, D54G, D54P, D54L, D54Y, D54F, D54R, or D54A; b) Q56D, Q56P, Q56H, Q56G, Q56T, Q56R, Q56L, Q56I, Q56S, Q56Y, Q56E, or Q56V; c) P57R, P57D, P57V, P57W, P57A, P57N, P57S, P57T, P57Q, P57G, P57K, P57H, P57L, P57I or P57E; d) N91V, N91G, N91A, N91S, N91T, N91R, N91K, N91P, N91I, or N91W; e) R104V, R104T, R104Y, R104F, R104T, R104L, R104S, R104A, R104E, R104I; f) N155K; g) V111; h) K96D or K96E; i) I149M; or j) K140R.

In some embodiments, the IL-18 variant polypeptide comprises a set of substitution combination comprising G3P, E6K, D54H, Q56D, P57R, N91V, and R104V, wherein the amino acid position(s) is relative to a wild-type human IL-18 set forth in SEQ ID NO: 1. In some embodiments, the IL-18 variant polypeptide does not comprise any other mutation relative to the wild-type human IL-18 set forth in SEQ ID NO: 1. In some embodiments, the IL-18 variant polypeptide further comprises one, two or three mutations or substitutions relative to the wild-type human IL-18 set forth in SEQ ID NO: 1, optionally wherein IL-18 variant polypeptide has a) a comparable or more potent binding affinity with IL-18 receptor (e.g., IL-18Rα), and/or b) a comparable or lower binding affinity with IL-18 binding protein, as compared to the corresponding IL-18 variant polypeptide without the one, two or three additional mutations or substitutions. In some embodiments, the IL-18 variant polypeptide further comprises a S117C substitution, optionally wherein the S117C substitution promotes a C76-C117 disulfide bond. In some embodiments, the IL-18 variant polypeptide further comprises a substitution at C38, optionally wherein the substitution is selected from the group consisting of C38I, C38V, C38L, and C38M, further optionally wherein the substitution is C38I or C38V. In some embodiments, the IL-18 variant polypeptide further comprises a substitution at C68, optionally wherein the substitution is selected from the group consisting of C68I, C68V, C68S, and C68D, further optionally wherein the substitution is C68I, C68L, or C38S. In some embodiments, the IL-18 variant polypeptide further comprises a substitution at C76 and/or C127, optionally wherein the C76 substitution is C76Y or C76V, and optionally wherein the C127 substitution is selected from the group consisting of C127A, C127I, C127Y, C127F, and C127L, further optionally wherein the C127 substitution is C127A or C127I. In some embodiments, the variant polypeptide further comprises C38S, C68S, and C76S substitutions, optionally wherein the variant polypeptide further comprises a C127S substitution. In some embodiments, the variant polypeptide further comprises C38I, C68S, and S117C. In some embodiments, the variant polypeptide further comprises C38V, C68I, S117C, and C127A. In some embodiments, the variant polypeptide further comprises C38S, C68I, S117C, and C127I. In some embodiments, the variant polypeptide further comprises C38I, C68I, C76V, and C127I. In some embodiments, the variant polypeptide further comprises C38I, C68L, and C76Y.

In some embodiments, the IL-18 variant polypeptide comprises a set of substitution combination comprising G3S, E6K, D54W, Q56P, P57D, N91G, and R104T, wherein the amino acid position(s) is relative to a wild-type human IL-18 set forth in SEQ ID NO: 1. In some embodiments, the IL-18 variant polypeptide does not comprise any other mutation relative to the wild-type human IL-18 set forth in SEQ ID NO: 1. In some embodiments, the IL-18 variant polypeptide further comprises one, two or three mutations or substitutions relative to the wild-type human IL-18 set forth in SEQ ID NO: 1, optionally wherein IL-18 variant polypeptide has a) a comparable or more potent binding affinity with IL-18 receptor (e.g., IL-18Rα), and/or b) a comparable or lower binding affinity with IL-18 binding protein, as compared to the corresponding IL-18 variant polypeptide without the one, two or three additional mutations or substitutions. In some embodiments, the IL-18 variant polypeptide further comprises a S117C substitution, optionally wherein the S117C substitution promotes a C76-C117 disulfide bond. In some embodiments, the IL-18 variant polypeptide further comprises a substitution at C38, optionally wherein the substitution is selected from the group consisting of C38I, C38V, C38L, and C38M, further optionally wherein the substitution is C38I or C38V. In some embodiments, the IL-18 variant polypeptide further comprises a substitution at C68, optionally wherein the substitution is selected from the group consisting of C68I, C68V, C68S, and C68D, further optionally wherein the substitution is C68I, C68L, or C38S. In some embodiments, the IL-18 variant polypeptide further comprises a substitution at C76 and/or C127, optionally wherein the C76 substitution is C76Y or C76V, and optionally wherein the C127 substitution is selected from the group consisting of C127A, C127I, C127Y, C127F, and C127L, further optionally wherein the C127 substitution is C127A or C127I. In some embodiments, the variant polypeptide further comprises C38S, C68S, and C76S substitutions, optionally wherein the variant polypeptide further comprises a C127S substitution. In some embodiments, the variant polypeptide further comprises C38I, C68S, and S117C. In some embodiments, the variant polypeptide further comprises C38V, C68I, S117C, and C127A. In some embodiments, the variant polypeptide further comprises C38S, C68I, S117C, and C127I. In some embodiments, the variant polypeptide further comprises C38I, C68I, C76V, and C127I. In some embodiments, the variant polypeptide further comprises C38I, C68L, and C76Y.

In some embodiments, the IL-18 variant polypeptide comprises a set of substitution combination comprising G3P, E6K, D54W, Q56H, P57V, N91A, and R104Y, wherein the amino acid position(s) is relative to a wild-type human IL-18 set forth in SEQ ID NO: 1. In some embodiments, the IL-18 variant polypeptide does not comprise any other mutation relative to the wild-type human IL-18 set forth in SEQ ID NO: 1. In some embodiments, the IL-18 variant polypeptide further comprises one, two optionally wherein IL-18 variant polypeptide has a) a comparable or more potent binding affinity with IL-18 receptor (e.g. IL-18Ra), and/or b) a comparable or lower binding affinity with IL-18 binding protein, compared to the corresponding IL-18 variant polypeptide without the one, two or three additional mutations or substitutions. In some embodiments, the IL-18 variant polypeptide further comprises a S117C substitution, optionally wherein the S117C substitution promotes a C76-C117 disulfide bond. In some embodiments, the IL-18 variant polypeptide further comprises a substitution at C38, optionally wherein the substitution is selected from the group consisting of C38I, C38V, C38L, and C38M, further optionally wherein the substitution is C38I or C38V. In some embodiments, the IL-18 variant polypeptide further comprises a substitution at C68, optionally wherein the substitution is selected from the group consisting of C68I, C68V, C68S, and C68D, further optionally wherein the substitution is C68I, C68L, or C38S. In some embodiments, the IL-18 variant polypeptide further comprises a substitution at C76 and/or C127, optionally wherein the C76 substitution is C76Y or C76V, and optionally wherein the C127 substitution is selected from the group consisting of C127A, C127I, C127Y, C127F, and C127L, further optionally wherein the C127 substitution is C127A or C127I. In some embodiments, the variant polypeptide further comprises C38S, C68S, and C76S substitutions, optionally wherein the variant polypeptide further comprises a C127S substitution. In some embodiments, the variant polypeptide further comprises C38I, C68S, and S117C. In some embodiments, the variant polypeptide further comprises C38V, C68I, S117C, and C127A. In some embodiments, the variant polypeptide further comprises C38S, C68I, S117C, and C127I. In some embodiments, the variant polypeptide further comprises C38I, C68I, C76V, and C127I. In some embodiments, the variant polypeptide further comprises C38I, C68L, and C76Y.

In some embodiments, the IL-18 variant polypeptide comprises a set of substitution combination comprising G3P, E6K, D54W, Q56G, P57V, N91V, and R104F, wherein the amino acid position(s) is relative to a wild-type human IL-18 set forth in SEQ ID NO: 1. In some embodiments, the IL-18 variant polypeptide does not comprise any other mutation relative to the wild-type human IL-18 set forth in SEQ ID NO: 1. In some embodiments, the IL-18 variant polypeptide further comprises one, two or three mutations or substitutions relative to the wild-type human IL-18 set forth in SEQ ID NO: 1, optionally wherein IL-18 variant polypeptide has a) a comparable or more potent binding affinity with IL-18 receptor (e.g., IL-18Rα), and/or b) a comparable or lower binding affinity with IL-18 binding protein, as compared to the corresponding IL-18 variant polypeptide without the one, two or three additional mutations or substitutions. In some embodiments, the IL-18 variant polypeptide further comprises a S117C substitution, optionally wherein the S117C substitution promotes a C76-C117 disulfide bond. In some embodiments, the IL-18 variant polypeptide further comprises a substitution at C38, optionally wherein the substitution is selected from the group consisting of C38I, C38V, C38L, and C38M, further optionally wherein the substitution is C38I or C38V. In some embodiments, the IL-18 variant polypeptide further comprises a substitution at C68, optionally wherein the substitution is selected from the group consisting of C68I, C68V, C68S, and C68D, further optionally wherein the substitution is C68I, C68L, or C38S. In some embodiments, the IL-18 variant polypeptide further comprises a substitution at C76 and/or C127, optionally wherein the C76 substitution is C76Y or C76V, and optionally wherein the C127 substitution is selected from the group consisting of C127A, C127I, C127Y, C127F, and C127L, further optionally wherein the C127 substitution is C127A or C127I. In some embodiments, the variant polypeptide further comprises C38S, C68S, and C76S substitutions, optionally wherein the variant polypeptide further comprises a C127S substitution. In some embodiments, the variant polypeptide further comprises C38I, C68S, and S117C. In some embodiments, the variant polypeptide further comprises C38V, C68I, S117C, and C127A. In some embodiments, the variant polypeptide further comprises C38S, C68I, S117C, and C127I. In some embodiments, the variant polypeptide further comprises C38I, C68I, C76V, and C127I. In some embodiments, the variant polypeptide further comprises C38I, C68L, and C76Y.

In some embodiments, the IL-18 variant polypeptide comprises a set of substitution combination comprising G3E, E6T, D54W, Q56P, P57W, N91V, R104T, and N155K, wherein the amino acid position(s) is relative to a wild-type human IL-18 set forth in SEQ ID NO: 1. In some embodiments, the IL-18 variant polypeptide does not comprise any other mutation relative to the wild-type human IL-18 set forth in SEQ ID NO: 1. In some embodiments, the IL-18 variant polypeptide further comprises one, two or three mutations or substitutions relative to the wild-type human IL-18 set forth in SEQ ID NO: 1, optionally wherein IL-18 variant polypeptide has a) a comparable or more potent binding affinity with IL-18 receptor (e.g., IL-18Rα), and/or b) a comparable or lower binding affinity with IL-18 binding protein, as compared to the corresponding IL-18 variant polypeptide without the one, two or three additional mutations or substitutions. In some embodiments, the IL-18 variant polypeptide further comprises a S117C substitution, optionally wherein the S117C substitution promotes a C76-C117 disulfide bond. In some embodiments, the IL-18 variant polypeptide further comprises a substitution at C38, optionally wherein the substitution is selected from the group consisting of C38I, C38V, C38L, and C38M, further optionally wherein the substitution is C38I or C38V. In some embodiments, the IL-18 variant polypeptide further comprises a substitution at C68, optionally wherein the substitution is selected from the group consisting of C68I, C68V, C68S, and C68D, further optionally wherein the substitution is C68I, C68L, or C38S. In some embodiments, the IL-18 variant polypeptide further comprises a substitution at C76 and/or C127, optionally wherein the C76 substitution is C76Y or C76V, and optionally wherein the C127 substitution is selected from the group consisting of C127A, C127I, C127Y, C127F, and C127L, further optionally wherein the C127 substitution is C127A or C127I. In some embodiments, the variant polypeptide further comprises C38S, C68S, and C76S substitutions, optionally wherein the variant polypeptide further comprises a C127S substitution. In some embodiments, the variant polypeptide further comprises C38I, C68S, and S117C. In some embodiments, the variant polypeptide further comprises C38V, C68I, S117C, and C127A. In some embodiments, the variant polypeptide further comprises C38S, C68I, S117C, and C127I. In some embodiments, the variant polypeptide further comprises C38I, C68I, C76V, and C127I. In some embodiments, the variant polypeptide further comprises C38I, C68L, and C76Y.

In some embodiments, the IL-18 variant polypeptide comprises a set of substitution combination comprising G3P, E6R, D54W, Q56T, N91V, and R104T, wherein the amino acid position(s) is relative to a wild-type human IL-18 set forth in SEQ ID NO: 1. In some embodiments, the IL-18 variant polypeptide does not comprise any other mutation relative to the wild-type human IL-18 set forth in SEQ ID NO: 1. In some embodiments, the IL-18 variant polypeptide further comprises one, two or three mutations or substitutions relative to the wild-type human IL-18 set forth in SEQ ID NO: 1, optionally wherein IL-18 variant polypeptide has a) a comparable or more potent binding affinity with IL-18 receptor (e.g., IL-18Rα), and/or b) a comparable or lower binding affinity with IL-18 binding protein, as compared to the corresponding IL-18 variant polypeptide without the one, two or three additional mutations or substitutions. In some embodiments, the IL-18 variant polypeptide further comprises a S117C substitution, optionally wherein the S117C substitution promotes a C76-C117 disulfide bond. In some embodiments, the IL-18 variant polypeptide further comprises a substitution at C38, optionally wherein the substitution is selected from the group consisting of C38I, C38V, C38L, and C38M, further optionally wherein the substitution is C38I or C38V. In some embodiments, the IL-18 variant polypeptide further comprises a substitution at C68, optionally wherein the substitution is selected from the group consisting of C68I, C68V, C68S, and C68D, further optionally wherein the substitution is C68I, C68L, or C38S. In some embodiments, the IL-18 variant polypeptide further comprises a substitution at C76 and/or C127, optionally wherein the C76 substitution is C76Y or C76V, and optionally wherein the C127 substitution is selected from the group consisting of C127A, C127I, C127Y, C127F, and C127L, further optionally wherein the C127 substitution is C127A or C127I. In some embodiments, the variant polypeptide further comprises C38S, C68S, and C76S substitutions, optionally wherein the variant polypeptide further comprises a C127S substitution. In some embodiments, the variant polypeptide further comprises C38I, C68S, and S117C. In some embodiments, the variant polypeptide further comprises C38V, C68I, S117C, and C127A. In some embodiments, the variant polypeptide further comprises C38S, C68I, S117C, and C127I. In some embodiments, the variant polypeptide further comprises C38I, C68I, C76V, and C127I. In some embodiments, the variant polypeptide further comprises C38I, C68L, and C76Y.

In some embodiments, the IL-18 variant polypeptide comprises a set of substitution combination comprising G3P, E6R, D54H, Q56T, P57A, and N91A, wherein the amino acid position(s) is relative to a wild-type human IL-18 set forth in SEQ ID NO: 1. In some embodiments, the IL-18 variant polypeptide does not comprise any other mutation relative to the wild-type human IL-18 set forth in SEQ ID NO: 1. In some embodiments, the IL-18 variant polypeptide further comprises one, two or three mutations or substitutions relative to the wild-type human IL-18 set forth in SEQ ID NO: 1, optionally wherein IL-18 variant polypeptide has a) a comparable or more potent binding affinity with IL-18 receptor (e.g., IL-18Rα), and/or b) a comparable or lower binding affinity with IL-18 binding protein, as compared to the corresponding IL-18 variant polypeptide without the one, two or three additional mutations or substitutions. In some embodiments, the IL-18 variant polypeptide further comprises a S117C substitution, optionally wherein the S117C substitution promotes a C76-C117 disulfide bond. In some embodiments, the IL-18 variant polypeptide further comprises a substitution at C38, optionally wherein the substitution is selected from the group consisting of C38I, C38V, C38L, and C38M, further optionally wherein the substitution is C38I or C38V. In some embodiments, the IL-18 variant polypeptide further comprises a substitution at C68, optionally wherein the substitution is selected from the group consisting of C68I, C68V, C68S, and C68D, further optionally wherein the substitution is C68I, C68L, or C38S. In some embodiments, the IL-18 variant polypeptide further comprises a substitution at C76 and/or C127, optionally wherein the C76 substitution is C76Y or C76V, and optionally wherein the C127 substitution is selected from the group consisting of C127A, C127I, C127Y, C127F, and C127L, further optionally wherein the C127 substitution is C127A or C127I. In some embodiments, the variant polypeptide further comprises C38S, C68S, and C76S substitutions, optionally wherein the variant polypeptide further comprises a C127S substitution. In some embodiments, the variant polypeptide further comprises C38I, C68S, and S117C. In some embodiments, the variant polypeptide further comprises C38V, C68I, S117C, and C127A. In some embodiments, the variant polypeptide further comprises C38S, C68I, S117C, and C127I. In some embodiments, the variant polypeptide further comprises C38I, C68I, C76V, and C127I. In some embodiments, the variant polypeptide further comprises C38I, C68L, and C76Y.

In some embodiments, the IL-18 variant polypeptide comprises a set of substitution combination comprising G3P, E6R, D54W, Q56P, P57A, N91A, and R104L, wherein the amino acid position(s) is relative to a wild-type human IL-18 set forth in SEQ ID NO: 1. In some embodiments, the IL-18 variant polypeptide does not comprise any other mutation relative to the wild-type human IL-18 set forth in SEQ ID NO: 1. In some embodiments, the IL-18 variant polypeptide further comprises one, two optionally wherein IL-18 variant polypeptide has a) a comparable or more potent binding affinity with IL-18 receptor (e.g., IL-18Rα), and/or b) a comparable or lower binding affinity with IL-18 binding protein, compared to the corresponding IL-18 variant polypeptide without the one, two or three additional mutations or substitutions. In some embodiments, the IL-18 variant polypeptide further comprises a S117C substitution, optionally wherein the S117C substitution promotes a C76-C117 disulfide bond. In some embodiments, the IL-18 variant polypeptide further comprises a substitution at C38, optionally wherein the substitution is selected from the group consisting of C38I, C38V, C38L, and C38M, further optionally wherein the substitution is C38I or C38V. In some embodiments, the IL-18 variant polypeptide further comprises a substitution at C68, optionally wherein the substitution is selected from the group consisting of C68I, C68V, C68S, and C68D, further optionally wherein the substitution is C68I, C68L, or C38S. In some embodiments, the IL-18 variant polypeptide further comprises a substitution at C76 and/or C127, optionally wherein the C76 substitution is C76Y or C76V, and optionally wherein the C127 substitution is selected from the group consisting of C127A, C127I, C127Y, C127F, and C127L, further optionally wherein the C127 substitution is C127A or C127I. In some embodiments, the variant polypeptide further comprises C38S, C68S, and C76S substitutions, optionally wherein the variant polypeptide further comprises a C127S substitution. In some embodiments, the variant polypeptide further comprises C38I, C68S, and S117C. In some embodiments, the variant polypeptide further comprises C38V, C68I, S117C, and C127A. In some embodiments, the variant polypeptide further comprises C38S, C68I, S117C, and C127I. In some embodiments, the variant polypeptide further comprises C38I, C68I, C76V, and C127I. In some embodiments, the variant polypeptide further comprises C38I, C68L, and C76Y.

In some embodiments, the IL-18 variant polypeptide comprises a set of substitution combination comprising G3P, E6R, D54W, Q56R, P57A, N91S, and R104S, wherein the amino acid position(s) is relative to a wild-type human IL-18 set forth in SEQ ID NO: 1. In some embodiments, the IL-18 variant polypeptide does not comprise any other mutation relative to the wild-type human IL-18 set forth in SEQ ID NO: 1. In some embodiments, the IL-18 variant polypeptide further comprises one, two or three mutations or substitutions relative to the wild-type human IL-18 set forth in SEQ ID NO: 1, optionally wherein IL-18 variant polypeptide has a) a comparable or more potent binding affinity with IL-18 receptor (e.g., IL-18Rα), and/or b) a comparable or lower binding affinity with IL-18 binding protein, as compared to the corresponding IL-18 variant polypeptide without the one, two or three additional mutations or substitutions. In some embodiments, the IL-18 variant polypeptide further comprises a S117C substitution, optionally wherein the S117C substitution promotes a C76-C117 disulfide bond. In some embodiments, the IL-18 variant polypeptide further comprises a substitution at C38, optionally wherein the substitution is selected from the group consisting of C38I, C38V, C38L, and C38M, further optionally wherein the substitution is C38I or C38V. In some embodiments, the IL-18 variant polypeptide further comprises a substitution at C68, optionally wherein the substitution is selected from the group consisting of C68I, C68V, C68S, and C68D, further optionally wherein the substitution is C68I, C68L, or C38S. In some embodiments, the IL-18 variant polypeptide further comprises a substitution at C76 and/or C127, optionally wherein the C76 substitution is C76Y or C76V, and optionally wherein the C127 substitution is selected from the group consisting of C127A, C127I, C127Y, C127F, and C127L, further optionally wherein the C127 substitution is C127A or C127I. In some embodiments, the variant polypeptide further comprises C38S, C68S, and C76S substitutions, optionally wherein the variant polypeptide further comprises a C127S substitution. In some embodiments, the variant polypeptide further comprises C38I, C68S, and S117C. In some embodiments, the variant polypeptide further comprises C38V, C68I, S117C, and C127A. In some embodiments, the variant polypeptide further comprises C38S, C68I, S117C, and C127I. In some embodiments, the variant polypeptide further comprises C38I, C68I, C76V, and C127I. In some embodiments, the variant polypeptide further comprises C38I, C68L, and C76Y.

In some embodiments, the IL-18 variant polypeptide comprises a set of substitution combination comprising G3P, E6R, D54Q, Q56L, P57W, and N91S, wherein the amino acid position(s) is relative to a wild-type human IL-18 set forth in SEQ ID NO: 1. In some embodiments, the IL-18 variant polypeptide does not comprise any other mutation relative to the wild-type human IL-18 set forth in SEQ ID NO: 1. In some embodiments, the IL-18 variant polypeptide further comprises one, two or three mutations or substitutions relative to the wild-type human IL-18 set forth in SEQ ID NO: 1, optionally wherein IL-18 variant polypeptide has a) a comparable or more potent binding affinity with IL-18 receptor (e.g., IL-18Rα), and/or b) a comparable or lower binding affinity with IL-18 binding protein, as compared to the corresponding IL-18 variant polypeptide without the one, two or three additional mutations or substitutions. In some embodiments, the IL-18 variant polypeptide further comprises a S117C substitution, optionally wherein the S117C substitution promotes a C76-C117 disulfide bond. In some embodiments, the IL-18 variant polypeptide further comprises a substitution at C38, optionally wherein the substitution is selected from the group consisting of C38I, C38V, C38L, and C38M, further optionally wherein the substitution is C38I or C38V. In some embodiments, the IL-18 variant polypeptide further comprises a substitution at C68, optionally wherein the substitution is selected from the group consisting of C68I, C68V, C68S, and C68D, further optionally wherein the substitution is C68I, C68L, or C38S. In some embodiments, the IL-18 variant polypeptide further comprises a substitution at C76 and/or C127, optionally wherein the C76 substitution is C76Y or C76V, and optionally wherein the C127 substitution is selected from the group consisting of C127A, C127I, C127Y, C127F, and C127L, further optionally wherein the C127 substitution is C127A or C127I. In some embodiments, the variant polypeptide further comprises C38S, C68S, and C76S substitutions, optionally wherein the variant polypeptide further comprises a C127S substitution. In some embodiments, the variant polypeptide further comprises C38I, C68S, and S117C. In some embodiments, the variant polypeptide further comprises C38V, C68I, S117C, and C127A. In some embodiments, the variant polypeptide further comprises C38S, C68I, S117C, and C127I. In some embodiments, the variant polypeptide further comprises C38I, C68I, C76V, and C127I. In some embodiments, the variant polypeptide further comprises C38I, C68L, and C76Y.

In some embodiments, the IL-18 variant polypeptide comprises a set of substitution combination comprising G3P, E6R, D54S, Q56R, P57N, N91G, and R104T, wherein the amino acid position(s) is relative to a wild-type human IL-18 set forth in SEQ ID NO: 1. In some embodiments, the IL-18 variant polypeptide does not comprise any other mutation relative to the wild-type human IL-18 set forth in SEQ ID NO: 1. In some embodiments, the IL-18 variant polypeptide further comprises one, two or three mutations or substitutions relative to the wild-type human IL-18 set forth in SEQ ID NO: 1, optionally wherein IL-18 variant polypeptide has a) a comparable or more potent binding affinity with IL-18 receptor (e.g., IL-18Rα), and/or b) a comparable or lower binding affinity with IL-18 binding protein, as compared to the corresponding IL-18 variant polypeptide without the one, two or three additional mutations or substitutions. In some embodiments, the IL-18 variant polypeptide further comprises a S117C substitution, optionally wherein the S117C substitution promotes a C76-C117 disulfide bond. In some embodiments, the IL-18 variant polypeptide further comprises a substitution at C38, optionally wherein the substitution is selected from the group consisting of C38I, C38V, C38L, and C38M, further optionally wherein the substitution is C38I or C38V. In some embodiments, the IL-18 variant polypeptide further comprises a substitution at C68, optionally wherein the substitution is selected from the group consisting of C68I, C68V, C68S, and C68D, further optionally wherein the substitution is C68I, C68L, or C38S. In some embodiments, the IL-18 variant polypeptide further comprises a substitution at C76 and/or C127, optionally wherein the C76 substitution is C76Y or C76V, and optionally wherein the C127 substitution is selected from the group consisting of C127A, C127I, C127Y, C127F, and C127L, further optionally wherein the C127 substitution is C127A or C127I. In some embodiments, the variant polypeptide further comprises C38S, C68S, and C76S substitutions, optionally wherein the variant polypeptide further comprises a C127S substitution. In some embodiments, the variant polypeptide further comprises C38I, C68S, and S117C. In some embodiments, the variant polypeptide further comprises C38V, C68I, S117C, and C127A. In some embodiments, the variant polypeptide further comprises C38S, C68I, S117C, and C127I. In some embodiments, the variant polypeptide further comprises C38I, C68I, C76V, and C127I. In some embodiments, the variant polypeptide further comprises C38I, C68L, and C76Y.

In some embodiments, the IL-18 variant polypeptide comprises a set of substitution combination comprising G3P, E6K, D54G, Q56G, P57A, and N91T, wherein the amino acid position(s) is relative to a wild-type human IL-18 set forth in SEQ ID NO: 1. In some embodiments, the IL-18 variant polypeptide does not comprise any other mutation relative to the wild-type human IL-18 set forth in SEQ ID NO: 1. In some embodiments, the IL-18 variant polypeptide further comprises one, two or three mutations or substitutions relative to the wild-type human IL-18 set forth in SEQ ID NO: 1, optionally wherein IL-18 variant polypeptide has a) a comparable or more potent binding affinity with IL-18 receptor (e.g., IL-18Rα), and/or b) a comparable or lower binding affinity with IL-18 binding protein, as compared to the corresponding IL-18 variant polypeptide without the one, two or three additional mutations or substitutions. In some embodiments, the IL-18 variant polypeptide further comprises a S117C substitution, optionally wherein the S117C substitution promotes a C76-C117 disulfide bond. In some embodiments, the IL-18 variant polypeptide further comprises a substitution at C38, optionally wherein the substitution is selected from the group consisting of C38I, C38V, C38L, and C38M, further optionally wherein the substitution is C38I or C38V. In some embodiments, the IL-18 variant polypeptide further comprises a substitution at C68, optionally wherein the substitution is selected from the group consisting of C68I, C68V, C68S, and C68D, further optionally wherein the substitution is C68I, C68L, or C38S. In some embodiments, the IL-18 variant polypeptide further comprises a substitution at C76 and/or C127, optionally wherein the C76 substitution is C76Y or C76V, and optionally wherein the C127 substitution is selected from the group consisting of C127A, C127I, C127Y, C127F, and C127L, further optionally wherein the C127 substitution is C127A or C127I. In some embodiments, the variant polypeptide further comprises C38S, C68S, and C76S substitutions, optionally wherein the variant polypeptide further comprises a C127S substitution. In some embodiments, the variant polypeptide further comprises C38I, C68S, and S117C. In some embodiments, the variant polypeptide further comprises C38V, C68I, S117C, and C127A. In some embodiments, the variant polypeptide further comprises C38S, C68I, S117C, and C127I. In some embodiments, the variant polypeptide further comprises C38I, C68I, C76V, and C127I. In some embodiments, the variant polypeptide further comprises C38I, C68L, and C76Y.

In some embodiments, the IL-18 variant polypeptide comprises a set of substitution combination comprising G3P, E6K, D54Q, Q56I, and P57W, wherein the amino acid position(s) is relative to a wild-type human IL-18 set forth in SEQ ID NO: 1. In some embodiments, the IL-18 variant polypeptide does not comprise any other mutation relative to the wild-type human IL-18 set forth in SEQ ID NO: 1. In some embodiments, the IL-18 variant polypeptide further comprises one, two or three mutations or substitutions relative to the wild-type human IL-18 set forth in SEQ ID NO: 1, optionally wherein IL-18 variant polypeptide has a) a comparable or more potent binding affinity with IL-18 receptor (e.g., IL-18Rα), and/or b) a comparable or lower binding affinity with IL-18 binding protein, as compared to the corresponding IL-18 variant polypeptide without the one, two or three additional mutations or substitutions. In some embodiments, the IL-18 variant polypeptide further comprises a S117C substitution, optionally wherein the S117C substitution promotes a C76-C117 disulfide bond. In some embodiments, the IL-18 variant polypeptide further comprises a substitution at C38, optionally wherein the substitution is selected from the group consisting of C38I, C38V, C38L, and C38M, further optionally wherein the substitution is C38I or C38V. In some embodiments, the IL-18 variant polypeptide further comprises a substitution at C68, optionally wherein the substitution is selected from the group consisting of C68I, C68V, C68S, and C68D, further optionally wherein the substitution is C68I, C68L, or C38S. In some embodiments, the IL-18 variant polypeptide further comprises a substitution at C76 and/or C127, optionally wherein the C76 substitution is C76Y or C76V, and optionally wherein the C127 substitution is selected from the group consisting of C127A, C127I, C127Y, C127F, and C127L, further optionally wherein the C127 substitution is C127A or C127I. In some embodiments, the variant polypeptide further comprises C38S, C68S, and C76S substitutions, optionally wherein the variant polypeptide further comprises a C127S substitution. In some embodiments, the variant polypeptide further comprises C38I, C68S, and S117C. In some embodiments, the variant polypeptide further comprises C38V, C68I, S117C, and C127A. In some embodiments, the variant polypeptide further comprises C38S, C68I, S117C, and C127I. In some embodiments, the variant polypeptide further comprises C38I, C68I, C76V, and C127I. In some embodiments, the variant polypeptide further comprises C38I, C68L, and C76Y.

In some embodiments, the IL-18 variant polypeptide comprises a set of substitution combination comprising G3N, E6K, D54P, Q56S, P57S, N91R, and R104A, wherein the amino acid position(s) is relative to a wild-type human IL-18 set forth in SEQ ID NO: 1. In some embodiments, the IL-18 variant polypeptide does not comprise any other mutation relative to the wild-type human IL-18 set forth in SEQ ID NO: 1. In some embodiments, the IL-18 variant polypeptide further comprises one, two or three mutations or substitutions relative to the wild-type human IL-18 set forth in SEQ ID NO: 1, optionally wherein IL-18 variant polypeptide has a) a comparable or more potent binding affinity with IL-18 receptor (e.g., IL-18Rα), and/or b) a comparable or lower binding affinity with IL-18 binding protein, as compared to the corresponding IL-18 variant polypeptide without the one, two or three additional mutations or substitutions. In some embodiments, the IL-18 variant polypeptide further comprises a S117C substitution, optionally wherein the S117C substitution promotes a C76-C117 disulfide bond. In some embodiments, the IL-18 variant polypeptide further comprises a substitution at C38, optionally wherein the substitution is selected from the group consisting of C38I, C38V, C38L, and C38M, further optionally wherein the substitution is C38I or C38V. In some embodiments, the IL-18 variant polypeptide further comprises a substitution at C68, optionally wherein the substitution is selected from the group consisting of C68I, C68V, C68S, and C68D, further optionally wherein the substitution is C68I, C68L, or C38S. In some embodiments, the IL-18 variant polypeptide further comprises a substitution at C76 and/or C127, optionally wherein the C76 substitution is C76Y or C76V, and optionally wherein the C127 substitution is selected from the group consisting of C127A, C127I, C127Y, C127F, and C127L, further optionally wherein the C127 substitution is C127A or C127I. In some embodiments, the variant polypeptide further comprises C38S, C68S, and C76S substitutions, optionally wherein the variant polypeptide further comprises a C127S substitution. In some embodiments, the variant polypeptide further comprises C38I, C68S, and S117C. In some embodiments, the variant polypeptide further comprises C38V, C68I, S117C, and C127A. In some embodiments, the variant polypeptide further comprises C38S, C68I, S117C, and C127I. In some embodiments, the variant polypeptide further comprises C38I, C68I, C76V, and C127I. In some embodiments, the variant polypeptide further comprises C38I, C68L, and C76Y.

In some embodiments, the IL-18 variant polypeptide comprises a set of substitution combination comprising G3P, E6R, D54S, Q56Y, P57T, and N91G, wherein the amino acid position(s) is relative to a wild-type human IL-18 set forth in SEQ ID NO: 1. In some embodiments, the IL-18 variant polypeptide does not comprise any other mutation relative to the wild-type human IL-18 set forth in SEQ ID NO: 1. In some embodiments, the IL-18 variant polypeptide further comprises one, two or three mutations or substitutions relative to the wild-type human IL-18 set forth in SEQ ID NO: 1, optionally wherein IL-18 variant polypeptide has a) a comparable or more potent binding affinity with IL-18 receptor (e.g., IL-18Rα), and/or b) a comparable or lower binding affinity with IL-18 binding protein, as compared to the corresponding IL-18 variant polypeptide without the one, two or three additional mutations or substitutions. In some embodiments, the IL-18 variant polypeptide further comprises a S117C substitution, optionally wherein the S117C substitution promotes a C76-C117 disulfide bond. In some embodiments, the IL-18 variant polypeptide further comprises a substitution at C38, optionally wherein the substitution is selected from the group consisting of C38I, C38V, C38L, and C38M, further optionally wherein the substitution is C38I or C38V. In some embodiments, the IL-18 variant polypeptide further comprises a substitution at C68, optionally wherein the substitution is selected from the group consisting of C68I, C68V, C68S, and C68D, further optionally wherein the substitution is C68I, C68L, or C38S. In some embodiments, the IL-18 variant polypeptide further comprises a substitution at C76 and/or C127, optionally wherein the C76 substitution is C76Y or C76V, and optionally wherein the C127 substitution is selected from the group consisting of C127A, C127I, C127Y, C127F, and C127L, further optionally wherein the C127 substitution is C127A or C127I. In some embodiments, the variant polypeptide further comprises C38S, C68S, and C76S substitutions, optionally wherein the variant polypeptide further comprises a C127S substitution. In some embodiments, the variant polypeptide further comprises C38I, C68S, and S117C. In some embodiments, the variant polypeptide further comprises C38V, C68I, S117C, and C127A. In some embodiments, the variant polypeptide further comprises C38S, C68I, S117C, and C127I. In some embodiments, the variant polypeptide further comprises C38I, C68I, C76V, and C127I. In some embodiments, the variant polypeptide further comprises C38I, C68L, and C76Y.

In some embodiments, the IL-18 variant polypeptide comprises a set of substitution combination comprising G3P, E6R, D54S, Q56R, P57N, N91G, and R104S, wherein the amino acid position(s) is relative to a wild-type human IL-18 set forth in SEQ ID NO: 1. In some embodiments, the IL-18 variant polypeptide does not comprise any other mutation relative to the wild-type human IL-18 set forth in SEQ ID NO: 1. In some embodiments, the IL-18 variant polypeptide further comprises one, two or three mutations or substitutions relative to the wild-type human IL-18 set forth in SEQ ID NO: 1, optionally wherein IL-18 variant polypeptide has a) a comparable or more potent binding affinity with IL-18 receptor (e.g., IL-18Rα), and/or b) a comparable or lower binding affinity with IL-18 binding protein, as compared to the corresponding IL-18 variant polypeptide without the one, two or three additional mutations or substitutions. In some embodiments, the IL-18 variant polypeptide further comprises a S117C substitution, optionally wherein the S117C substitution promotes a C76-C117 disulfide bond. In some embodiments, the IL-18 variant polypeptide further comprises a substitution at C38, optionally wherein the substitution is selected from the group consisting of C38I, C38V, C38L, and C38M, further optionally wherein the substitution is C38I or C38V. In some embodiments, the IL-18 variant polypeptide further comprises a substitution at C68, optionally wherein the substitution is selected from the group consisting of C68I, C68V, C68S, and C68D, further optionally wherein the substitution is C68I, C68L, or C38S. In some embodiments, the IL-18 variant polypeptide further comprises a substitution at C76 and/or C127, optionally wherein the C76 substitution is C76Y or C76V, and optionally wherein the C127 substitution is selected from the group consisting of C127A, C127I, C127Y, C127F, and C127L, further optionally wherein the C127 substitution is C127A or C127I. In some embodiments, the variant polypeptide further comprises C38S, C68S, and C76S substitutions, optionally wherein the variant polypeptide further comprises a C127S substitution. In some embodiments, the variant polypeptide further comprises C38I, C68S, and S117C. In some embodiments, the variant polypeptide further comprises C38V, C68I, S117C, and C127A. In some embodiments, the variant polypeptide further comprises C38S, C68I, S117C, and C127I. In some embodiments, the variant polypeptide further comprises C38I, C68I, C76V, and C127I. In some embodiments, the variant polypeptide further comprises C38I, C68L, and C76Y.

In some embodiments, the IL-18 variant polypeptide comprises a set of substitution combination comprising G3P, E6R, D54L, Q56T, P57A, and N91G, wherein the amino acid position(s) is relative to a wild-type human IL-18 set forth in SEQ ID NO: 1. In some embodiments, the IL-18 variant polypeptide does not comprise any other mutation relative to the wild-type human IL-18 set forth in SEQ ID NO: 1. In some embodiments, the IL-18 variant polypeptide further comprises one, two or three mutations or substitutions relative to the wild-type human IL-18 set forth in SEQ ID NO: 1, optionally wherein IL-18 variant polypeptide has a) a comparable or more potent binding affinity with IL-18 receptor (e.g., IL-18Rα), and/or b) a comparable or lower binding affinity with IL-18 binding protein, as compared to the corresponding IL-18 variant polypeptide without the one, two or three additional mutations or substitutions. In some embodiments, the IL-18 variant polypeptide further comprises a S117C substitution, optionally wherein the S117C substitution promotes a C76-C117 disulfide bond. In some embodiments, the IL-18 variant polypeptide further comprises a substitution at C38, optionally wherein the substitution is selected from the group consisting of C38I, C38V, C38L, and C38M, further optionally wherein the substitution is C38I or C38V. In some embodiments, the IL-18 variant polypeptide further comprises a substitution at C68, optionally wherein the substitution is selected from the group consisting of C68I, C68V, C68S, and C68D, further optionally wherein the substitution is C68I, C68L, or C38S. In some embodiments, the IL-18 variant polypeptide further comprises a substitution at C76 and/or C127, optionally wherein the C76 substitution is C76Y or C76V, and optionally wherein the C127 substitution is selected from the group consisting of C127A, C127I, C127Y, C127F, and C127L, further optionally wherein the C127 substitution is C127A or C127I. In some embodiments, the variant polypeptide further comprises C38S, C68S, and C76S substitutions, optionally wherein the variant polypeptide further comprises a C127S substitution. In some embodiments, the variant polypeptide further comprises C38I, C68S, and S117C. In some embodiments, the variant polypeptide further comprises C38V, C68I, S117C, and C127A. In some embodiments, the variant polypeptide further comprises C38S, C68I, S117C, and C127I. In some embodiments, the variant polypeptide further comprises C38I, C68I, C76V, and C127I. In some embodiments, the variant polypeptide further comprises C38I, C68L, and C76Y.

In some embodiments, the IL-18 variant polypeptide comprises a set of substitution combination comprising G3P, E6R, D54S, Q56R, P57R, N91G, and R104S, wherein the amino acid position(s) is relative to a wild-type human IL-18 set forth in SEQ ID NO: 1. In some embodiments, the IL-18 variant polypeptide does not comprise any other mutation relative to the wild-type human IL-18 set forth in SEQ ID NO: 1. In some embodiments, the IL-18 variant polypeptide further comprises one, two optionally wherein IL-18 variant polypeptide has a) a comparable or more potent binding affinity with IL-18 receptor (e.g., IL-18Rα), and/or b) a comparable or lower binding affinity with IL-18 binding protein, compared to the corresponding IL-18 variant polypeptide without the one, two or three additional mutations or substitutions. In some embodiments, the IL-18 variant polypeptide further comprises a S117C substitution, optionally wherein the S117C substitution promotes a C76-C117 disulfide bond. In some embodiments, the IL-18 variant polypeptide further comprises a substitution at C38, optionally wherein the substitution is selected from the group consisting of C38I, C38V, C38L, and C38M, further optionally wherein the substitution is C38I or C38V. In some embodiments, the IL-18 variant polypeptide further comprises a substitution at C68, optionally wherein the substitution is selected from the group consisting of C68I, C68V, C68S, and C68D, further optionally wherein the substitution is C68I, C68L, or C38S. In some embodiments, the IL-18 variant polypeptide further comprises a substitution at C76 and/or C127, optionally wherein the C76 substitution is C76Y or C76V, and optionally wherein the C127 substitution is selected from the group consisting of C127A, C127I, C127Y, C127F, and C127L, further optionally wherein the C127 substitution is C127A or C127I. In some embodiments, the variant polypeptide further comprises C38S, C68S, and C76S substitutions, optionally wherein the variant polypeptide further comprises a C127S substitution. In some embodiments, the variant polypeptide further comprises C38I, C68S, and S117C. In some embodiments, the variant polypeptide further comprises C38V, C68I, S117C, and C127A. In some embodiments, the variant polypeptide further comprises C38S, C68I, S117C, and C127I. In some embodiments, the variant polypeptide further comprises C38I, C68I, C76V, and C127I. In some embodiments, the variant polypeptide further comprises C38I, C68L, and C76Y.

In some embodiments, the IL-18 variant polypeptide comprises a set of substitution combination comprising G3P, E6K, D54H, Q56E, P57Q, and N91A, wherein the amino acid position(s) is relative to a wild-type human IL-18 set forth in SEQ ID NO: 1. In some embodiments, the IL-18 variant polypeptide does not comprise any other mutation relative to the wild-type human IL-18 set forth in SEQ ID NO: 1. In some embodiments, the IL-18 variant polypeptide further comprises one, two or three mutations or substitutions relative to the wild-type human IL-18 set forth in SEQ ID NO: 1, optionally wherein IL-18 variant polypeptide has a) a comparable or more potent binding affinity with IL-18 receptor (e.g., IL-18Rα), and/or b) a comparable or lower binding affinity with IL-18 binding protein, as compared to the corresponding IL-18 variant polypeptide without the one, two or three additional mutations or substitutions. In some embodiments, the IL-18 variant polypeptide further comprises a S117C substitution, optionally wherein the S117C substitution promotes a C76-C117 disulfide bond. In some embodiments, the IL-18 variant polypeptide further comprises a substitution at C38, optionally wherein the substitution is selected from the group consisting of C38I, C38V, C38L, and C38M, further optionally wherein the substitution is C38I or C38V. In some embodiments, the IL-18 variant polypeptide further comprises a substitution at C68, optionally wherein the substitution is selected from the group consisting of C68I, C68V, C68S, and C68D, further optionally wherein the substitution is C68I, C68L, or C38S. In some embodiments, the IL-18 variant polypeptide further comprises a substitution at C76 and/or C127, optionally wherein the C76 substitution is C76Y or C76V, and optionally wherein the C127 substitution is selected from the group consisting of C127A, C127I, C127Y, C127F, and C127L, further optionally wherein the C127 substitution is C127A or C127I. In some embodiments, the variant polypeptide further comprises C38S, C68S, and C76S substitutions, optionally wherein the variant polypeptide further comprises a C127S substitution. In some embodiments, the variant polypeptide further comprises C38I, C68S, and S117C. In some embodiments, the variant polypeptide further comprises C38V, C68I, S117C, and C127A. In some embodiments, the variant polypeptide further comprises C38S, C68I, S117C, and C127I. In some embodiments, the variant polypeptide further comprises C38I, C68I, C76V, and C127I. In some embodiments, the variant polypeptide further comprises C38I, C68L, and C76Y.

In some embodiments, the IL-18 variant polypeptide comprises a set of substitution combination comprising G3P, E6R, D54S, Q56R, P57S, N91G, and R104S, wherein the amino acid position(s) is relative to a wild-type human IL-18 set forth in SEQ ID NO: 1. In some embodiments, the IL-18 variant polypeptide does not comprise any other mutation relative to the wild-type human IL-18 set forth in SEQ ID NO: 1. In some embodiments, the IL-18 variant polypeptide further comprises one, two or three mutations or substitutions relative to the wild-type human IL-18 set forth in SEQ ID NO: 1, optionally wherein IL-18 variant polypeptide has a) a comparable or more potent binding affinity with IL-18 receptor (e.g., IL-18Rα), and/or b) a comparable or lower binding affinity with IL-18 binding protein, as compared to the corresponding IL-18 variant polypeptide without the one, two or three additional mutations or substitutions. In some embodiments, the IL-18 variant polypeptide further comprises a S117C substitution, optionally wherein the S117C substitution promotes a C76-C117 disulfide bond. In some embodiments, the IL-18 variant polypeptide further comprises a substitution at C38, optionally wherein the substitution is selected from the group consisting of C38I, C38V, C38L, and C38M, further optionally wherein the substitution is C38I or C38V. In some embodiments, the IL-18 variant polypeptide further comprises a substitution at C68, optionally wherein the substitution is selected from the group consisting of C68I, C68V, C68S, and C68D, further optionally wherein the substitution is C68I, C68L, or C38S. In some embodiments, the IL-18 variant polypeptide further comprises a substitution at C76 and/or C127, optionally wherein the C76 substitution is C76Y or C76V, and optionally wherein the C127 substitution is selected from the group consisting of C127A, C127I, C127Y, C127F, and C127L, further optionally wherein the C127 substitution is C127A or C127I. In some embodiments, the variant polypeptide further comprises C38S, C68S, and C76S substitutions, optionally wherein the variant polypeptide further comprises a C127S substitution. In some embodiments, the variant polypeptide further comprises C38I, C68S, and S117C. In some embodiments, the variant polypeptide further comprises C38V, C68I, S117C, and C127A. In some embodiments, the variant polypeptide further comprises C38S, C68I, S117C, and C127I. In some embodiments, the variant polypeptide further comprises C38I, C68I, C76V, and C127I. In some embodiments, the variant polypeptide further comprises C38I, C68L, and C76Y.

In some embodiments, the IL-18 variant polypeptide comprises a set of substitution combination comprising G3P, E6R, D54S, Q56S, P57T, N91G, and R104S, wherein the amino acid position(s) is relative to a wild-type human IL-18 set forth in SEQ ID NO: 1. In some embodiments, the IL-18 variant polypeptide does not comprise any other mutation relative to the wild-type human IL-18 set forth in SEQ ID NO: 1. In some embodiments, the IL-18 variant polypeptide further comprises one, two or three mutations or substitutions relative to the wild-type human IL-18 set forth in SEQ ID NO: 1, optionally wherein IL-18 variant polypeptide has a) a comparable or more potent binding affinity with IL-18 receptor (e.g., IL-18Rα), and/or b) a comparable or lower binding affinity with IL-18 binding protein, as compared to the corresponding IL-18 variant polypeptide without the one, two or three additional mutations or substitutions. In some embodiments, the IL-18 variant polypeptide further comprises a S117C substitution, optionally wherein the S117C substitution promotes a C76-C117 disulfide bond. In some embodiments, the IL-18 variant polypeptide further comprises a substitution at C38, optionally wherein the substitution is selected from the group consisting of C38I, C38V, C38L, and C38M, further optionally wherein the substitution is C38I or C38V. In some embodiments, the IL-18 variant polypeptide further comprises a substitution at C68, optionally wherein the substitution is selected from the group consisting of C68I, C68V, C68S, and C68D, further optionally wherein the substitution is C68I, C68L, or C38S. In some embodiments, the IL-18 variant polypeptide further comprises a substitution at C76 and/or C127, optionally wherein the C76 substitution is C76Y or C76V, and optionally wherein the C127 substitution is selected from the group consisting of C127A, C127I, C127Y, C127F, and C127L, further optionally wherein the C127 substitution is C127A or C127I. In some embodiments, the variant polypeptide further comprises C38S, C68S, and C76S substitutions, optionally wherein the variant polypeptide further comprises a C127S substitution. In some embodiments, the variant polypeptide further comprises C38I, C68S, and S117C. In some embodiments, the variant polypeptide further comprises C38V, C68I, S117C, and C127A. In some embodiments, the variant polypeptide further comprises C38S, C68I, S117C, and C127I. In some embodiments, the variant polypeptide further comprises C38I, C68I, C76V, and C127I. In some embodiments, the variant polypeptide further comprises C38I, C68L, and C76Y.

In some embodiments, the IL-18 variant polypeptide comprises a set of substitution combination comprising G3P, E6R, D54Y, Q56R, P57G, N91K, and R104S, wherein the amino acid position(s) is relative to a wild-type human IL-18 set forth in SEQ ID NO: 1. In some embodiments, the IL-18 variant polypeptide does not comprise any other mutation relative to the wild-type human IL-18 set forth in SEQ ID NO: 1. In some embodiments, the IL-18 variant polypeptide further comprises one, two or three mutations or substitutions relative to the wild-type human IL-18 set forth in SEQ ID NO: 1, optionally wherein IL-18 variant polypeptide has a) a comparable or more potent binding affinity with IL-18 receptor (e.g., IL-18Rα), and/or b) a comparable or lower binding affinity with IL-18 binding protein, as compared to the corresponding IL-18 variant polypeptide without the one, two or three additional mutations or substitutions. In some embodiments, the IL-18 variant polypeptide further comprises a S117C substitution, optionally wherein the S117C substitution promotes a C76-C117 disulfide bond. In some embodiments, the IL-18 variant polypeptide further comprises a substitution at C38, optionally wherein the substitution is selected from the group consisting of C38I, C38V, C38L, and C38M, further optionally wherein the substitution is C38I or C38V. In some embodiments, the IL-18 variant polypeptide further comprises a substitution at C68, optionally wherein the substitution is selected from the group consisting of C68I, C68V, C68S, and C68D, further optionally wherein the substitution is C68I, C68L, or C38S. In some embodiments, the IL-18 variant polypeptide further comprises a substitution at C76 and/or C127, optionally wherein the C76 substitution is C76Y or C76V, and optionally wherein the C127 substitution is selected from the group consisting of C127A, C127I, C127Y, C127F, and C127L, further optionally wherein the C127 substitution is C127A or C127I. In some embodiments, the variant polypeptide further comprises C38S, C68S, and C76S substitutions, optionally wherein the variant polypeptide further comprises a C127S substitution. In some embodiments, the variant polypeptide further comprises C38I, C68S, and S117C. In some embodiments, the variant polypeptide further comprises C38V, C68I, S117C, and C127A. In some embodiments, the variant polypeptide further comprises C38S, C68I, S117C, and C127I. In some embodiments, the variant polypeptide further comprises C38I, C68I, C76V, and C127I. In some embodiments, the variant polypeptide further comprises C38I, C68L, and C76Y.

In some embodiments, the IL-18 variant polypeptide comprises a set of substitution combination comprising G3P, E6R, D54Y, Q56T, and P57R, wherein the amino acid position(s) is relative to a wild-type human IL-18 set forth in SEQ ID NO: 1. In some embodiments, the IL-18 variant polypeptide does not comprise any other mutation relative to the wild-type human IL-18 set forth in SEQ ID NO: 1. In some embodiments, the IL-18 variant polypeptide further comprises one, two or three mutations or substitutions relative to the wild-type human IL-18 set forth in SEQ ID NO: 1, optionally wherein IL-18 variant polypeptide has a) a comparable or more potent binding affinity with IL-18 receptor (e.g., IL-18Rα), and/or b) a comparable or lower binding affinity with IL-18 binding protein, as compared to the corresponding IL-18 variant polypeptide without the one, two or three additional mutations or substitutions. In some embodiments, the IL-18 variant polypeptide further comprises a S117C substitution, optionally wherein the S117C substitution promotes a C76-C117 disulfide bond. In some embodiments, the IL-18 variant polypeptide further comprises a substitution at C38, optionally wherein the substitution is selected from the group consisting of C38I, C38V, C38L, and C38M, further optionally wherein the substitution is C38I or C38V. In some embodiments, the IL-18 variant polypeptide further comprises a substitution at C68, optionally wherein the substitution is selected from the group consisting of C68I, C68V, C68S, and C68D, further optionally wherein the substitution is C68I, C68L, or C38S. In some embodiments, the IL-18 variant polypeptide further comprises a substitution at C76 and/or C127, optionally wherein the C76 substitution is C76Y or C76V, and optionally wherein the C127 substitution is selected from the group consisting of C127A, C127I, C127Y, C127F, and C127L, further optionally wherein the C127 substitution is C127A or C127I. In some embodiments, the variant polypeptide further comprises C38S, C68S, and C76S substitutions, optionally wherein the variant polypeptide further comprises a C127S substitution. In some embodiments, the variant polypeptide further comprises C38I, C68S, and S117C. In some embodiments, the variant polypeptide further comprises C38V, C68I, S117C, and C127A. In some embodiments, the variant polypeptide further comprises C38S, C68I, S117C, and C127I. In some embodiments, the variant polypeptide further comprises C38I, C68I, C76V, and C127I. In some embodiments, the variant polypeptide further comprises C38I, C68L, and C76Y.

In some embodiments, the IL-18 variant polypeptide comprises a set of substitution combination comprising G3P, E6R, D54Y, Q56T, and P57S, wherein the amino acid position(s) is relative to a wild-type human IL-18 set forth in SEQ ID NO: 1. In some embodiments, the IL-18 variant polypeptide does not comprise any other mutation relative to the wild-type human IL-18 set forth in SEQ ID NO: 1. In some embodiments, the IL-18 variant polypeptide further comprises one, two or three mutations or substitutions relative to the wild-type human IL-18 set forth in SEQ ID NO: 1, optionally wherein IL-18 variant polypeptide has a) a comparable or more potent binding affinity with IL-18 receptor (e.g., IL-18Rα), and/or b) a comparable or lower binding affinity with IL-18 binding protein, as compared to the corresponding IL-18 variant polypeptide without the one, two or three additional mutations or substitutions. In some embodiments, the IL-18 variant polypeptide further comprises a S117C substitution, optionally wherein the S117C substitution promotes a C76-C117 disulfide bond. In some embodiments, the IL-18 variant polypeptide further comprises a substitution at C38, optionally wherein the substitution is selected from the group consisting of C38I, C38V, C38L, and C38M, further optionally wherein the substitution is C38I or C38V. In some embodiments, the IL-18 variant polypeptide further comprises a substitution at C68, optionally wherein the substitution is selected from the group consisting of C68I, C68V, C68S, and C68D, further optionally wherein the substitution is C68I, C68L, or C38S. In some embodiments, the IL-18 variant polypeptide further comprises a substitution at C76 and/or C127, optionally wherein the C76 substitution is C76Y or C76V, and optionally wherein the C127 substitution is selected from the group consisting of C127A, C127I, C127Y, C127F, and C127L, further optionally wherein the C127 substitution is C127A or C127I. In some embodiments, the variant polypeptide further comprises C38S, C68S, and C76S substitutions, optionally wherein the variant polypeptide further comprises a C127S substitution. In some embodiments, the variant polypeptide further comprises C38I, C68S, and S117C. In some embodiments, the variant polypeptide further comprises C38V, C68I, S117C, and C127A. In some embodiments, the variant polypeptide further comprises C38S, C68I, S117C, and C127I. In some embodiments, the variant polypeptide further comprises C38I, C68I, C76V, and C127I. In some embodiments, the variant polypeptide further comprises C38I, C68L, and C76Y.

In some embodiments, the IL-18 variant polypeptide comprises a set of substitution combination comprising G3P, E6R, D54L, Q56T, P57T, and N91R, wherein the amino acid position(s) is relative to a wild-type human IL-18 set forth in SEQ ID NO: 1. In some embodiments, the IL-18 variant polypeptide does not comprise any other mutation relative to the wild-type human IL-18 set forth in SEQ ID NO: 1. In some embodiments, the IL-18 variant polypeptide further comprises one, two or three mutations or substitutions relative to the wild-type human IL-18 set forth in SEQ ID NO: 1, optionally wherein IL-18 variant polypeptide has a) a comparable or more potent binding affinity with IL-18 receptor (e.g., IL-18Rα), and/or b) a comparable or lower binding affinity with IL-18 binding protein, as compared to the corresponding IL-18 variant polypeptide without the one, two or three additional mutations or substitutions. In some embodiments, the IL-18 variant polypeptide further comprises a S117C substitution, optionally wherein the S117C substitution promotes a C76-C117 disulfide bond. In some embodiments, the IL-18 variant polypeptide further comprises a substitution at C38, optionally wherein the substitution is selected from the group consisting of C38I, C38V, C38L, and C38M, further optionally wherein the substitution is C38I or C38V. In some embodiments, the IL-18 variant polypeptide further comprises a substitution at C68, optionally wherein the substitution is selected from the group consisting of C68I, C68V, C68S, and C68D, further optionally wherein the substitution is C68I, C68L, or C38S. In some embodiments, the IL-18 variant polypeptide further comprises a substitution at C76 and/or C127, optionally wherein the C76 substitution is C76Y or C76V, and optionally wherein the C127 substitution is selected from the group consisting of C127A, C127I, C127Y, C127F, and C127L, further optionally wherein the C127 substitution is C127A or C127I. In some embodiments, the variant polypeptide further comprises C38S, C68S, and C76S substitutions, optionally wherein the variant polypeptide further comprises a C127S substitution. In some embodiments, the variant polypeptide further comprises C38I, C68S, and S117C. In some embodiments, the variant polypeptide further comprises C38V, C68I, S117C, and C127A. In some embodiments, the variant polypeptide further comprises C38S, C68I, S117C, and C127I. In some embodiments, the variant polypeptide further comprises C38I, C68I, C76V, and C127I. In some embodiments, the variant polypeptide further comprises C38I, C68L, and C76Y.

In some embodiments, the IL-18 variant polypeptide comprises a set of substitution combination comprising G3P, E6R, D54H, Q56D, P57K, N91V, and R104Y, wherein the amino acid position(s) is relative to a wild-type human IL-18 set forth in SEQ ID NO: 1. In some embodiments, the IL-18 variant polypeptide does not comprise any other mutation relative to the wild-type human IL-18 set forth in SEQ ID NO: 1. In some embodiments, the IL-18 variant polypeptide further comprises one, two or three mutations or substitutions relative to the wild-type human IL-18 set forth in SEQ ID NO: 1, optionally wherein IL-18 variant polypeptide has a) a comparable or more potent binding affinity with IL-18 receptor (e.g., IL-18Rα), and/or b) a comparable or lower binding affinity with IL-18 binding protein, as compared to the corresponding IL-18 variant polypeptide without the one, two or three additional mutations or substitutions. In some embodiments, the IL-18 variant polypeptide further comprises a S117C substitution, optionally wherein the S117C substitution promotes a C76-C117 disulfide bond. In some embodiments, the IL-18 variant polypeptide further comprises a substitution at C38, optionally wherein the substitution is selected from the group consisting of C38I, C38V, C38L, and C38M, further optionally wherein the substitution is C38I or C38V. In some embodiments, the IL-18 variant polypeptide further comprises a substitution at C68, optionally wherein the substitution is selected from the group consisting of C68I, C68V, C68S, and C68D, further optionally wherein the substitution is C68I, C68L, or C38S. In some embodiments, the IL-18 variant polypeptide further comprises a substitution at C76 and/or C127, optionally wherein the C76 substitution is C76Y or C76V, and optionally wherein the C127 substitution is selected from the group consisting of C127A, C127I, C127Y, C127F, and C127L, further optionally wherein the C127 substitution is C127A or C127I. In some embodiments, the variant polypeptide further comprises C38S, C68S, and C76S substitutions, optionally wherein the variant polypeptide further comprises a C127S substitution. In some embodiments, the variant polypeptide further comprises C38I, C68S, and S117C. In some embodiments, the variant polypeptide further comprises C38V, C68I, S117C, and C127A. In some embodiments, the variant polypeptide further comprises C38S, C68I, S117C, and C127I. In some embodiments, the variant polypeptide further comprises C38I, C68I, C76V, and C127I. In some embodiments, the variant polypeptide further comprises C38I, C68L, and C76Y.

In some embodiments, the IL-18 variant polypeptide comprises a set of substitution combination comprising G3P, E6R, D54H, Q56Y, P57T, N91V, and R104Y, wherein the amino acid position(s) is relative to a wild-type human IL-18 set forth in SEQ ID NO: 1. In some embodiments, the IL-18 variant polypeptide does not comprise any other mutation relative to the wild-type human IL-18 set forth in SEQ ID NO: 1. In some embodiments, the IL-18 variant polypeptide further comprises one, two or three mutations or substitutions relative to the wild-type human IL-18 set forth in SEQ ID NO: 1, optionally wherein IL-18 variant polypeptide has a) a comparable or more potent binding affinity with IL-18 receptor (e.g., IL-18Rα), and/or b) a comparable or lower binding affinity with IL-18 binding protein, as compared to the corresponding IL-18 variant polypeptide without the one, two or three additional mutations or substitutions. In some embodiments, the IL-18 variant polypeptide further comprises a S117C substitution, optionally wherein the S117C substitution promotes a C76-C117 disulfide bond. In some embodiments, the IL-18 variant polypeptide further comprises a substitution at C38, optionally wherein the substitution is selected from the group consisting of C38I, C38V, C38L, and C38M, further optionally wherein the substitution is C38I or C38V. In some embodiments, the IL-18 variant polypeptide further comprises a substitution at C68, optionally wherein the substitution is selected from the group consisting of C68I, C68V, C68S, and C68D, further optionally wherein the substitution is C68I, C68L, or C38S. In some embodiments, the IL-18 variant polypeptide further comprises a substitution at C76 and/or C127, optionally wherein the C76 substitution is C76Y or C76V, and optionally wherein the C127 substitution is selected from the group consisting of C127A, C127I, C127Y, C127F, and C127L, further optionally wherein the C127 substitution is C127A or C127I. In some embodiments, the variant polypeptide further comprises C38S, C68S, and C76S substitutions, optionally wherein the variant polypeptide further comprises a C127S substitution. In some embodiments, the variant polypeptide further comprises C38I, C68S, and S117C. In some embodiments, the variant polypeptide further comprises C38V, C68I, S117C, and C127A. In some embodiments, the variant polypeptide further comprises C38S, C68I, S117C, and C127I. In some embodiments, the variant polypeptide further comprises C38I, C68I, C76V, and C127I. In some embodiments, the variant polypeptide further comprises C38I, C68L, and C76Y.

In some embodiments, the IL-18 variant polypeptide comprises a set of substitution combination comprising G3P, E6A, D54W, Q56G, P57G, N91V, and R104Y, wherein the amino acid position(s) is relative to a wild-type human IL-18 set forth in SEQ ID NO: 1. In some embodiments, the IL-18 variant polypeptide does not comprise any other mutation relative to the wild-type human IL-18 set forth in SEQ ID NO: 1. In some embodiments, the IL-18 variant polypeptide further comprises one, two optionally wherein IL-18 variant polypeptide has a) a comparable or more potent binding affinity with IL-18 receptor (e.g., IL-18Rα), and/or b) a comparable or lower binding affinity with IL-18 binding protein, compared to the corresponding IL-18 variant polypeptide without the one, two or three additional mutations or substitutions. In some embodiments, the IL-18 variant polypeptide further comprises a S117C substitution, optionally wherein the S117C substitution promotes a C76-C117 disulfide bond. In some embodiments, the IL-18 variant polypeptide further comprises a substitution at C38, optionally wherein the substitution is selected from the group consisting of C38I, C38V, C38L, and C38M, further optionally wherein the substitution is C38I or C38V. In some embodiments, the IL-18 variant polypeptide further comprises a substitution at C68, optionally wherein the substitution is selected from the group consisting of C68I, C68V, C68S, and C68D, further optionally wherein the substitution is C68I, C68L, or C38S. In some embodiments, the IL-18 variant polypeptide further comprises a substitution at C76 and/or C127, optionally wherein the C76 substitution is C76Y or C76V, and optionally wherein the C127 substitution is selected from the group consisting of C127A, C127I, C127Y, C127F, and C127L, further optionally wherein the C127 substitution is C127A or C127I. In some embodiments, the variant polypeptide further comprises C38S, C68S, and C76S substitutions, optionally wherein the variant polypeptide further comprises a C127S substitution. In some embodiments, the variant polypeptide further comprises C38I, C68S, and S117C. In some embodiments, the variant polypeptide further comprises C38V, C68I, S117C, and C127A. In some embodiments, the variant polypeptide further comprises C38S, C68I, S117C, and C127I. In some embodiments, the variant polypeptide further comprises C38I, C68I, C76V, and C127I. In some embodiments, the variant polypeptide further comprises C38I, C68L, and C76Y.

In some embodiments, the IL-18 variant polypeptide comprises a set of substitution combination comprising G3P, E6M, D54F, Q56D, P57R, and N91P, wherein the amino acid position(s) is relative to a wild-type human IL-18 set forth in SEQ ID NO: 1. In some embodiments, the IL-18 variant polypeptide does not comprise any other mutation relative to the wild-type human IL-18 set forth in SEQ ID NO: 1. In some embodiments, the IL-18 variant polypeptide further comprises one, two or three mutations or substitutions relative to the wild-type human IL-18 set forth in SEQ ID NO: 1, optionally wherein IL-18 variant polypeptide has a) a comparable or more potent binding affinity with IL-18 receptor (e.g., IL-18Rα), and/or b) a comparable or lower binding affinity with IL-18 binding protein, as compared to the corresponding IL-18 variant polypeptide without the one, two or three additional mutations or substitutions. In some embodiments, the IL-18 variant polypeptide further comprises a S117C substitution, optionally wherein the S117C substitution promotes a C76-C117 disulfide bond. In some embodiments, the IL-18 variant polypeptide further comprises a substitution at C38, optionally wherein the substitution is selected from the group consisting of C38I, C38V, C38L, and C38M, further optionally wherein the substitution is C38I or C38V. In some embodiments, the IL-18 variant polypeptide further comprises a substitution at C68, optionally wherein the substitution is selected from the group consisting of C68I, C68V, C68S, and C68D, further optionally wherein the substitution is C68I, C68L, or C38S. In some embodiments, the IL-18 variant polypeptide further comprises a substitution at C76 and/or C127, optionally wherein the C76 substitution is C76Y or C76V, and optionally wherein the C127 substitution is selected from the group consisting of C127A, C127I, C127Y, C127F, and C127L, further optionally wherein the C127 substitution is C127A or C127I. In some embodiments, the variant polypeptide further comprises C38S, C68S, and C76S substitutions, optionally wherein the variant polypeptide further comprises a C127S substitution. In some embodiments, the variant polypeptide further comprises C38I, C68S, and S117C. In some embodiments, the variant polypeptide further comprises C38V, C68I, S117C, and C127A. In some embodiments, the variant polypeptide further comprises C38S, C68I, S117C, and C127I. In some embodiments, the variant polypeptide further comprises C38I, C68I, C76V, and C127I. In some embodiments, the variant polypeptide further comprises C38I, C68L, and C76Y.

In some embodiments, the IL-18 variant polypeptide comprises a set of substitution combination comprising G3P, E6L, D54H, Q56T, P57V, and N91S, wherein the amino acid position(s) is relative to a wild-type human IL-18 set forth in SEQ ID NO: 1. In some embodiments, the IL-18 variant polypeptide does not comprise any other mutation relative to the wild-type human IL-18 set forth in SEQ ID NO: 1. In some embodiments, the IL-18 variant polypeptide further comprises one, two or three mutations or substitutions relative to the wild-type human IL-18 set forth in SEQ ID NO: 1, optionally wherein IL-18 variant polypeptide has a) a comparable or more potent binding affinity with IL-18 receptor (e.g., IL-18Rα), and/or b) a comparable or lower binding affinity with IL-18 binding protein, as compared to the corresponding IL-18 variant polypeptide without the one, two or three additional mutations or substitutions. In some embodiments, the IL-18 variant polypeptide further comprises a S117C substitution, optionally wherein the S117C substitution promotes a C76-C117 disulfide bond. In some embodiments, the IL-18 variant polypeptide further comprises a substitution at C38, optionally wherein the substitution is selected from the group consisting of C38I, C38V, C38L, and C38M, further optionally wherein the substitution is C38I or C38V. In some embodiments, the IL-18 variant polypeptide further comprises a substitution at C68, optionally wherein the substitution is selected from the group consisting of C68I, C68V, C68S, and C68D, further optionally wherein the substitution is C68I, C68L, or C38S. In some embodiments, the IL-18 variant polypeptide further comprises a substitution at C76 and/or C127, optionally wherein the C76 substitution is C76Y or C76V, and optionally wherein the C127 substitution is selected from the group consisting of C127A, C127I, C127Y, C127F, and C127L, further optionally wherein the C127 substitution is C127A or C127I. In some embodiments, the variant polypeptide further comprises C38S, C68S, and C76S substitutions, optionally wherein the variant polypeptide further comprises a C127S substitution. In some embodiments, the variant polypeptide further comprises C38I, C68S, and S117C. In some embodiments, the variant polypeptide further comprises C38V, C68I, S117C, and C127A. In some embodiments, the variant polypeptide further comprises C38S, C68I, S117C, and C127I. In some embodiments, the variant polypeptide further comprises C38I, C68I, C76V, and C127I. In some embodiments, the variant polypeptide further comprises C38I, C68L, and C76Y.

In some embodiments, the IL-18 variant polypeptide comprises a set of substitution combination comprising G3P, E6R, D54H, Q56I, P57H, N91I, and R104Y, wherein the amino acid position(s) is relative to a wild-type human IL-18 set forth in SEQ ID NO: 1. In some embodiments, the IL-18 variant polypeptide does not comprise any other mutation relative to the wild-type human IL-18 set forth in SEQ ID NO: 1. In some embodiments, the IL-18 variant polypeptide further comprises one, two or three mutations or substitutions relative to the wild-type human IL-18 set forth in SEQ ID NO: 1, optionally wherein IL-18 variant polypeptide has a) a comparable or more potent binding affinity with IL-18 receptor (e.g., IL-18Rα), and/or b) a comparable or lower binding affinity with IL-18 binding protein, as compared to the corresponding IL-18 variant polypeptide without the one, two or three additional mutations or substitutions. In some embodiments, the IL-18 variant polypeptide further comprises a S117C substitution, optionally wherein the S117C substitution promotes a C76-C117 disulfide bond. In some embodiments, the IL-18 variant polypeptide further comprises a substitution at C38, optionally wherein the substitution is selected from the group consisting of C38I, C38V, C38L, and C38M, further optionally wherein the substitution is C38I or C38V. In some embodiments, the IL-18 variant polypeptide further comprises a substitution at C68, optionally wherein the substitution is selected from the group consisting of C68I, C68V, C68S, and C68D, further optionally wherein the substitution is C68I, C68L, or C38S. In some embodiments, the IL-18 variant polypeptide further comprises a substitution at C76 and/or C127, optionally wherein the C76 substitution is C76Y or C76V, and optionally wherein the C127 substitution is selected from the group consisting of C127A, C127I, C127Y, C127F, and C127L, further optionally wherein the C127 substitution is C127A or C127I. In some embodiments, the variant polypeptide further comprises C38S, C68S, and C76S substitutions, optionally wherein the variant polypeptide further comprises a C127S substitution. In some embodiments, the variant polypeptide further comprises C38I, C68S, and S117C. In some embodiments, the variant polypeptide further comprises C38V, C68I, S117C, and C127A. In some embodiments, the variant polypeptide further comprises C38S, C68I, S117C, and C127I. In some embodiments, the variant polypeptide further comprises C38I, C68I, C76V, and C127I. In some embodiments, the variant polypeptide further comprises C38I, C68L, and C76Y.

In some embodiments, the IL-18 variant polypeptide comprises a set of substitution combination comprising G3P, E6G, D54S, Q56S, and P57R, wherein the amino acid position(s) is relative to a wild-type human IL-18 set forth in SEQ ID NO: 1. In some embodiments, the IL-18 variant polypeptide does not comprise any other mutation relative to the wild-type human IL-18 set forth in SEQ ID NO: 1. In some embodiments, the IL-18 variant polypeptide further comprises one, two or three mutations or substitutions relative to the wild-type human IL-18 set forth in SEQ ID NO: 1, optionally wherein IL-18 variant polypeptide has a) a comparable or more potent binding affinity with IL-18 receptor (e.g., IL-18Rα), and/or b) a comparable or lower binding affinity with IL-18 binding protein, as compared to the corresponding IL-18 variant polypeptide without the one, two or three additional mutations or substitutions. In some embodiments, the IL-18 variant polypeptide further comprises a S117C substitution, optionally wherein the S117C substitution promotes a C76-C117 disulfide bond. In some embodiments, the IL-18 variant polypeptide further comprises a substitution at C38, optionally wherein the substitution is selected from the group consisting of C38I, C38V, C38L, and C38M, further optionally wherein the substitution is C38I or C38V. In some embodiments, the IL-18 variant polypeptide further comprises a substitution at C68, optionally wherein the substitution is selected from the group consisting of C68I, C68V, C68S, and C68D, further optionally wherein the substitution is C68I, C68L, or C38S. In some embodiments, the IL-18 variant polypeptide further comprises a substitution at C76 and/or C127, optionally wherein the C76 substitution is C76Y or C76V, and optionally wherein the C127 substitution is selected from the group consisting of C127A, C127I, C127Y, C127F, and C127L, further optionally wherein the C127 substitution is C127A or C127I. In some embodiments, the variant polypeptide further comprises C38S, C68S, and C76S substitutions, optionally wherein the variant polypeptide further comprises a C127S substitution. In some embodiments, the variant polypeptide further comprises C38I, C68S, and S117C. In some embodiments, the variant polypeptide further comprises C38V, C68I, S117C, and C127A. In some embodiments, the variant polypeptide further comprises C38S, C68I, S117C, and C127I. In some embodiments, the variant polypeptide further comprises C38I, C68I, C76V, and C127I. In some embodiments, the variant polypeptide further comprises C38I, C68L, and C76Y.

In some embodiments, the IL-18 variant polypeptide comprises a set of substitution combination comprising G3E, E6H, D54R, Q56T, and P57H, wherein the amino acid position(s) is relative to a wild-type human IL-18 set forth in SEQ ID NO: 1. In some embodiments, the IL-18 variant polypeptide does not comprise any other mutation relative to the wild-type human IL-18 set forth in SEQ ID NO: 1. In some embodiments, the IL-18 variant polypeptide further comprises one, two or three mutations or substitutions relative to the wild-type human IL-18 set forth in SEQ ID NO: 1, optionally wherein IL-18 variant polypeptide has a) a comparable or more potent binding affinity with IL-18 receptor (e.g., IL-18Rα), and/or b) a comparable or lower binding affinity with IL-18 binding protein, as compared to the corresponding IL-18 variant polypeptide without the one, two or three additional mutations or substitutions. In some embodiments, the IL-18 variant polypeptide further comprises a S117C substitution, optionally wherein the S117C substitution promotes a C76-C117 disulfide bond. In some embodiments, the IL-18 variant polypeptide further comprises a substitution at C38, optionally wherein the substitution is selected from the group consisting of C38I, C38V, C38L, and C38M, further optionally wherein the substitution is C38I or C38V. In some embodiments, the IL-18 variant polypeptide further comprises a substitution at C68, optionally wherein the substitution is selected from the group consisting of C68I, C68V, C68S, and C68D, further optionally wherein the substitution is C68I, C68L, or C38S. In some embodiments, the IL-18 variant polypeptide further comprises a substitution at C76 and/or C127, optionally wherein the C76 substitution is C76Y or C76V, and optionally wherein the C127 substitution is selected from the group consisting of C127A, C127I, C127Y, C127F, and C127L, further optionally wherein the C127 substitution is C127A or C127I. In some embodiments, the variant polypeptide further comprises C38S, C68S, and C76S substitutions, optionally wherein the variant polypeptide further comprises a C127S substitution. In some embodiments, the variant polypeptide further comprises C38I, C68S, and S117C. In some embodiments, the variant polypeptide further comprises C38V, C68I, S117C, and C127A. In some embodiments, the variant polypeptide further comprises C38S, C68I, S117C, and C127I. In some embodiments, the variant polypeptide further comprises C38I, C68I, C76V, and C127I. In some embodiments, the variant polypeptide further comprises C38I, C68L, and C76Y.

In some embodiments, the IL-18 variant polypeptide comprises a set of substitution combination comprising G3P, E6R, D54H, Q56R, P57N, N91V, and R104E, wherein the amino acid position(s) is relative to a wild-type human IL-18 set forth in SEQ ID NO: 1. In some embodiments, the IL-18 variant polypeptide does not comprise any other mutation relative to the wild-type human IL-18 set forth in SEQ ID NO: 1. In some embodiments, the IL-18 variant polypeptide further comprises one, two or three mutations or substitutions relative to the wild-type human IL-18 set forth in SEQ ID NO: 1, optionally wherein IL-18 variant polypeptide has a) a comparable or more potent binding affinity with IL-18 receptor (e.g., IL-18Rα), and/or b) a comparable or lower binding affinity with IL-18 binding protein, as compared to the corresponding IL-18 variant polypeptide without the one, two or three additional mutations or substitutions. In some embodiments, the IL-18 variant polypeptide further comprises a S117C substitution, optionally wherein the S117C substitution promotes a C76-C117 disulfide bond. In some embodiments, the IL-18 variant polypeptide further comprises a substitution at C38, optionally wherein the substitution is selected from the group consisting of C38I, C38V, C38L, and C38M, further optionally wherein the substitution is C38I or C38V. In some embodiments, the IL-18 variant polypeptide further comprises a substitution at C68, optionally wherein the substitution is selected from the group consisting of C68I, C68V, C68S, and C68D, further optionally wherein the substitution is C68I, C68L, or C38S. In some embodiments, the IL-18 variant polypeptide further comprises a substitution at C76 and/or C127, optionally wherein the C76 substitution is C76Y or C76V, and optionally wherein the C127 substitution is selected from the group consisting of C127A, C127I, C127Y, C127F, and C127L, further optionally wherein the C127 substitution is C127A or C127I. In some embodiments, the variant polypeptide further comprises C38S, C68S, and C76S substitutions, optionally wherein the variant polypeptide further comprises a C127S substitution. In some embodiments, the variant polypeptide further comprises C38I, C68S, and S117C. In some embodiments, the variant polypeptide further comprises C38V, C68I, S117C, and C127A. In some embodiments, the variant polypeptide further comprises C38S, C68I, S117C, and C127I. In some embodiments, the variant polypeptide further comprises C38I, C68I, C76V, and C127I. In some embodiments, the variant polypeptide further comprises C38I, C68L, and C76Y.

In some embodiments, the IL-18 variant polypeptide comprises a set of substitution combination comprising G3P, E6R, D54G, Q56G, P57A, and N91G, wherein the amino acid position(s) is relative to a wild-type human IL-18 set forth in SEQ ID NO: 1. In some embodiments, the IL-18 variant polypeptide does not comprise any other mutation relative to the wild-type human IL-18 set forth in SEQ ID NO: 1. In some embodiments, the IL-18 variant polypeptide further comprises one, two or three mutations or substitutions relative to the wild-type human IL-18 set forth in SEQ ID NO: 1, optionally wherein IL-18 variant polypeptide has a) a comparable or more potent binding affinity with IL-18 receptor (e.g., IL-18Rα), and/or b) a comparable or lower binding affinity with IL-18 binding protein, as compared to the corresponding IL-18 variant polypeptide without the one, two or three additional mutations or substitutions. In some embodiments, the IL-18 variant polypeptide further comprises a S117C substitution, optionally wherein the S117C substitution promotes a C76-C117 disulfide bond. In some embodiments, the IL-18 variant polypeptide further comprises a substitution at C38, optionally wherein the substitution is selected from the group consisting of C38I, C38V, C38L, and C38M, further optionally wherein the substitution is C38I or C38V. In some embodiments, the IL-18 variant polypeptide further comprises a substitution at C68, optionally wherein the substitution is selected from the group consisting of C68I, C68V, C68S, and C68D, further optionally wherein the substitution is C68I, C68L, or C38S. In some embodiments, the IL-18 variant polypeptide further comprises a substitution at C76 and/or C127, optionally wherein the C76 substitution is C76Y or C76V, and optionally wherein the C127 substitution is selected from the group consisting of C127A, C127I, C127Y, C127F, and C127L, further optionally wherein the C127 substitution is C127A or C127I. In some embodiments, the variant polypeptide further comprises C38S, C68S, and C76S substitutions, optionally wherein the variant polypeptide further comprises a C127S substitution. In some embodiments, the variant polypeptide further comprises C38I, C68S, and S117C. In some embodiments, the variant polypeptide further comprises C38V, C68I, S117C, and C127A. In some embodiments, the variant polypeptide further comprises C38S, C68I, S117C, and C127I. In some embodiments, the variant polypeptide further comprises C38I, C68I, C76V, and C127I. In some embodiments, the variant polypeptide further comprises C38I, C68L, and C76Y.

In some embodiments, the IL-18 variant polypeptide comprises a set of substitution combination comprising G3P, E6S. D54A, Q56D, P57Q, and N91G, wherein the amino acid position(s) is relative to a wild-type human IL-18 set forth in SEQ ID NO: 1. In some embodiments, the IL-18 variant polypeptide does not comprise any other mutation relative to the wild-type human IL-18 set forth in SEQ ID NO: 1. In some embodiments, the IL-18 variant polypeptide further comprises one, two or three mutations or substitutions relative to the wild-type human IL-18 set forth in SEQ ID NO: 1, optionally wherein IL-18 variant polypeptide has a) a comparable or more potent binding affinity with IL-18 receptor (e.g., IL-18Rα), and/or b) a comparable or lower binding affinity with IL-18 binding protein, as compared to the corresponding IL-18 variant polypeptide without the one, two or three additional mutations or substitutions. In some embodiments, the IL-18 variant polypeptide further comprises a S117C substitution, optionally wherein the S117C substitution promotes a C76-C117 disulfide bond. In some embodiments, the IL-18 variant polypeptide further comprises a substitution at C38, optionally wherein the substitution is selected from the group consisting of C38I, C38V, C38L, and C38M, further optionally wherein the substitution is C38I or C38V. In some embodiments, the IL-18 variant polypeptide further comprises a substitution at C68, optionally wherein the substitution is selected from the group consisting of C68I, C68V, C68S, and C68D, further optionally wherein the substitution is C68I, C68L, or C38S. In some embodiments, the IL-18 variant polypeptide further comprises a substitution at C76 and/or C127, optionally wherein the C76 substitution is C76Y or C76V, and optionally wherein the C127 substitution is selected from the group consisting of C127A, C127I, C127Y, C127F, and C127L, further optionally wherein the C127 substitution is C127A or C127I. In some embodiments, the variant polypeptide further comprises C38S, C68S, and C76S substitutions, optionally wherein the variant polypeptide further comprises a C127S substitution. In some embodiments, the variant polypeptide further comprises C38I, C68S, and S117C. In some embodiments, the variant polypeptide further comprises C38V, C68I, S117C, and C127A. In some embodiments, the variant polypeptide further comprises C38S, C68I, S117C, and C127I. In some embodiments, the variant polypeptide further comprises C38I, C68I, C76V, and C127I. In some embodiments, the variant polypeptide further comprises C38I, C68L, and C76Y.

In some embodiments, the IL-18 variant polypeptide comprises a set of substitution combination comprising G3P, E6G, D54Q, Q56V, and P57W, wherein the amino acid position(s) is relative to a wild-type human IL-18 set forth in SEQ ID NO: 1. In some embodiments, the IL-18 variant polypeptide does not comprise any other mutation relative to the wild-type human IL-18 set forth in SEQ ID NO: 1. In some embodiments, the IL-18 variant polypeptide further comprises one, two or three mutations or substitutions relative to the wild-type human IL-18 set forth in SEQ ID NO: 1, optionally wherein IL-18 variant polypeptide has a) a comparable or more potent binding affinity with IL-18 receptor (e.g., IL-18Rα), and/or b) a comparable or lower binding affinity with IL-18 binding protein, as compared to the corresponding IL-18 variant polypeptide without the one, two or three additional mutations or substitutions. In some embodiments, the IL-18 variant polypeptide further comprises a S117C substitution, optionally wherein the S117C substitution promotes a C76-C117 disulfide bond. In some embodiments, the IL-18 variant polypeptide further comprises a substitution at C38, optionally wherein the substitution is selected from the group consisting of C38I, C38V, C38L, and C38M, further optionally wherein the substitution is C38I or C38V. In some embodiments, the IL-18 variant polypeptide further comprises a substitution at C68, optionally wherein the substitution is selected from the group consisting of C68I, C68V, C68S, and C68D, further optionally wherein the substitution is C68I, C68L, or C38S. In some embodiments, the IL-18 variant polypeptide further comprises a substitution at C76 and/or C127, optionally wherein the C76 substitution is C76Y or C76V, and optionally wherein the C127 substitution is selected from the group consisting of C127A, C127I, C127Y, C127F, and C127L, further optionally wherein the C127 substitution is C127A or C127I. In some embodiments, the variant polypeptide further comprises C38S, C68S, and C76S substitutions, optionally wherein the variant polypeptide further comprises a C127S substitution. In some embodiments, the variant polypeptide further comprises C38I, C68S, and S117C. In some embodiments, the variant polypeptide further comprises C38V, C68I, S117C, and C127A. In some embodiments, the variant polypeptide further comprises C38S, C68I, S117C, and C127I. In some embodiments, the variant polypeptide further comprises C38I, C68I, C76V, and C127I. In some embodiments, the variant polypeptide further comprises C38I, C68L, and C76Y.

In some embodiments, the IL-18 variant polypeptide comprises a set of substitution combination comprising G3P, E6S, D54W, Q56G, P57A, N91V, and R104I, wherein the amino acid position(s) is relative to a wild-type human IL-18 set forth in SEQ ID NO: 1. In some embodiments, the IL-18 variant polypeptide does not comprise any other mutation relative to the wild-type human IL-18 set forth in SEQ ID NO: 1. In some embodiments, the IL-18 variant polypeptide further comprises one, two optionally wherein IL-18 variant polypeptide has a) a comparable or more potent binding affinity with IL-18 receptor (e.g., IL-18Rα), and/or b) a comparable or lower binding affinity with IL-18 binding protein, compared to the corresponding IL-18 variant polypeptide without the one, two or three additional mutations or substitutions. In some embodiments, the IL-18 variant polypeptide further comprises a S117C substitution, optionally wherein the S117C substitution promotes a C76-C117 disulfide bond. In some embodiments, the IL-18 variant polypeptide further comprises a substitution at C38, optionally wherein the substitution is selected from the group consisting of C38I, C38V, C38L, and C38M, further optionally wherein the substitution is C38I or C38V. In some embodiments, the IL-18 variant polypeptide further comprises a substitution at C68, optionally wherein the substitution is selected from the group consisting of C68I, C68V, C68S, and C68D, further optionally wherein the substitution is C68I, C68L, or C38S. In some embodiments, the IL-18 variant polypeptide further comprises a substitution at C76 and/or C127, optionally wherein the C76 substitution is C76Y or C76V, and optionally wherein the C127 substitution is selected from the group consisting of C127A, C127I, C127Y, C127F, and C127L, further optionally wherein the C127 substitution is C127A or C127I. In some embodiments, the variant polypeptide further comprises C38S, C68S, and C76S substitutions, optionally wherein the variant polypeptide further comprises a C127S substitution. In some embodiments, the variant polypeptide further comprises C38I, C68S, and S117C. In some embodiments, the variant polypeptide further comprises C38V, C68I, S117C, and C127A. In some embodiments, the variant polypeptide further comprises C38S, C68I, S117C, and C127I. In some embodiments, the variant polypeptide further comprises C38I, C68I, C76V, and C127I. In some embodiments, the variant polypeptide further comprises C38I, C68L, and C76Y.

In some embodiments, the IL-18 variant polypeptide comprises a set of substitution combination comprising G3P, E6R, D54W, Q56P, P57G, N91V, and R104L, wherein the amino acid position(s) is relative to a wild-type human IL-18 set forth in SEQ ID NO: 1. In some embodiments, the IL-18 variant polypeptide does not comprise any other mutation relative to the wild-type human IL-18 set forth in SEQ ID NO: 1. In some embodiments, the IL-18 variant polypeptide further comprises one, two or three mutations or substitutions relative to the wild-type human IL-18 set forth in SEQ ID NO: 1, optionally wherein IL-18 variant polypeptide has a) a comparable or more potent binding affinity with IL-18 receptor (e.g., IL-18Rα), and/or b) a comparable or lower binding affinity with IL-18 binding protein, as compared to the corresponding IL-18 variant polypeptide without the one, two or three additional mutations or substitutions. In some embodiments, the IL-18 variant polypeptide further comprises a S117C substitution, optionally wherein the S117C substitution promotes a C76-C117 disulfide bond. In some embodiments, the IL-18 variant polypeptide further comprises a substitution at C38, optionally wherein the substitution is selected from the group consisting of C38I, C38V, C38L, and C38M, further optionally wherein the substitution is C38I or C38V. In some embodiments, the IL-18 variant polypeptide further comprises a substitution at C68, optionally wherein the substitution is selected from the group consisting of C68I, C68V, C68S, and C68D, further optionally wherein the substitution is C68I, C68L, or C38S. In some embodiments, the IL-18 variant polypeptide further comprises a substitution at C76 and/or C127, optionally wherein the C76 substitution is C76Y or C76V, and optionally wherein the C127 substitution is selected from the group consisting of C127A, C127I, C127Y, C127F, and C127L, further optionally wherein the C127 substitution is C127A or C127I. In some embodiments, the variant polypeptide further comprises C38S, C68S, and C76S substitutions, optionally wherein the variant polypeptide further comprises a C127S substitution. In some embodiments, the variant polypeptide further comprises C38I, C68S, and S117C. In some embodiments, the variant polypeptide further comprises C38V, C68I, S117C, and C127A. In some embodiments, the variant polypeptide further comprises C38S, C68I, S117C, and C127I. In some embodiments, the variant polypeptide further comprises C38I, C68I, C76V, and C127I. In some embodiments, the variant polypeptide further comprises C38I, C68L, and C76Y.

In some embodiments, the IL-18 variant polypeptide comprises a set of substitution combination comprising G3D, E6K, D54P, Q56S, P57W, and N91W, wherein the amino acid position(s) is relative to a wild-type human IL-18 set forth in SEQ ID NO: 1. In some embodiments, the IL-18 variant polypeptide does not comprise any other mutation relative to the wild-type human IL-18 set forth in SEQ ID NO: 1. In some embodiments, the IL-18 variant polypeptide further comprises one, two or three mutations or substitutions relative to the wild-type human IL-18 set forth in SEQ ID NO: 1, optionally wherein IL-18 variant polypeptide has a) a comparable or more potent binding affinity with IL-18 receptor (e.g., IL-18Rα), and/or b) a comparable or lower binding affinity with IL-18 binding protein, as compared to the corresponding IL-18 variant polypeptide without the one, two or three additional mutations or substitutions. In some embodiments, the IL-18 variant polypeptide further comprises a S117C substitution, optionally wherein the S117C substitution promotes a C76-C117 disulfide bond. In some embodiments, the IL-18 variant polypeptide further comprises a substitution at C38, optionally wherein the substitution is selected from the group consisting of C38I, C38V, C38L, and C38M, further optionally wherein the substitution is C38I or C38V. In some embodiments, the IL-18 variant polypeptide further comprises a substitution at C68, optionally wherein the substitution is selected from the group consisting of C68I, C68V, C68S, and C68D, further optionally wherein the substitution is C68I, C68L, or C38S. In some embodiments, the IL-18 variant polypeptide further comprises a substitution at C76 and/or C127, optionally wherein the C76 substitution is C76Y or C76V, and optionally wherein the C127 substitution is selected from the group consisting of C127A, C127I, C127Y, C127F, and C127L, further optionally wherein the C127 substitution is C127A or C127I. In some embodiments, the variant polypeptide further comprises C38S, C68S, and C76S substitutions, optionally wherein the variant polypeptide further comprises a C127S substitution. In some embodiments, the variant polypeptide further comprises C38I, C68S, and S117C. In some embodiments, the variant polypeptide further comprises C38V, C68I, S117C, and C127A. In some embodiments, the variant polypeptide further comprises C38S, C68I, S117C, and C127I. In some embodiments, the variant polypeptide further comprises C38I, C68I, C76V, and C127I. In some embodiments, the variant polypeptide further comprises C38I, C68L, and C76Y.

In some embodiments, the IL-18 variant polypeptide comprises one substitution (e.g., a single substitution) which is G3P, wherein the amino acid position(s) is relative to a wild-type human IL-18 set forth in SEQ ID NO: 1. In some embodiments, the IL-18 variant polypeptide does not comprise any other mutation relative to the wild-type human IL-18 set forth in SEQ ID NO: 1. In some embodiments, the IL-18 variant polypeptide further comprises one, two or three mutations or substitutions relative to the wild-type human IL-18 set forth in SEQ ID NO: 1, optionally wherein IL-18 variant polypeptide has a) a comparable or more potent binding affinity with IL-18 receptor (e.g., IL-18Rα), and/or b) a comparable or lower binding affinity with IL-18 binding protein, as compared to the corresponding IL-18 variant polypeptide without the one, two or three additional mutations or substitutions. In some embodiments, the IL-18 variant polypeptide further comprises a S117C substitution, optionally wherein the S117C substitution promotes a C76-C117 disulfide bond. In some embodiments, the IL-18 variant polypeptide further comprises a substitution at C38, optionally wherein the substitution is selected from the group consisting of C38I, C38V, C38L, and C38M, further optionally wherein the substitution is C38I or C38V. In some embodiments, the IL-18 variant polypeptide further comprises a substitution at C68, optionally wherein the substitution is selected from the group consisting of C68I, C68V, C68S, and C68D, further optionally wherein the substitution is C68I, C68L, or C38S. In some embodiments, the IL-18 variant polypeptide further comprises a substitution at C76 and/or C127, optionally wherein the C76 substitution is C76Y or C76V, and optionally wherein the C127 substitution is selected from the group consisting of C127A, C127I, C127Y, C127F, and C127L, further optionally wherein the C127 substitution is C127A or C127I. In some embodiments, the variant polypeptide further comprises C38S, C68S, and C76S substitutions, optionally wherein the variant polypeptide further comprises a C127S substitution. In some embodiments, the variant polypeptide further comprises C38I, C68S, and S117C. In some embodiments, the variant polypeptide further comprises C38V, C68I, S117C, and C127A. In some embodiments, the variant polypeptide further comprises C38S, C68I, S117C, and C127I. In some embodiments, the variant polypeptide further comprises C38I, C68I, C76V, and C127I. In some embodiments, the variant polypeptide further comprises C38I, C68L, and C76Y.

In some embodiments, the IL-18 variant polypeptide comprises a set of substitution combination comprising G3P, E6K, D54G, Q56G, and P57A, wherein the amino acid position(s) is relative to a wild-type human IL-18 set forth in SEQ ID NO: 1. In some embodiments, the IL-18 variant polypeptide does not comprise any other mutation relative to the wild-type human IL-18 set forth in SEQ ID NO: 1. In some embodiments, the IL-18 variant polypeptide further comprises one, two or three mutations or substitutions relative to the wild-type human IL-18 set forth in SEQ ID NO: 1, optionally wherein IL-18 variant polypeptide has a) a comparable or more potent binding affinity with IL-18 receptor (e.g., IL-18Rα), and/or b) a comparable or lower binding affinity with IL-18 binding protein, as compared to the corresponding IL-18 variant polypeptide without the one, two or three additional mutations or substitutions. In some embodiments, the IL-18 variant polypeptide further comprises a S117C substitution, optionally wherein the S117C substitution promotes a C76-C117 disulfide bond. In some embodiments, the IL-18 variant polypeptide further comprises a substitution at C38, optionally wherein the substitution is selected from the group consisting of C38I, C38V, C38L, and C38M, further optionally wherein the substitution is C38I or C38V. In some embodiments, the IL-18 variant polypeptide further comprises a substitution at C68, optionally wherein the substitution is selected from the group consisting of C68I, C68V, C68S, and C68D, further optionally wherein the substitution is C68I, C68L, or C38S. In some embodiments, the IL-18 variant polypeptide further comprises a substitution at C76 and/or C127, optionally wherein the C76 substitution is C76Y or C76V, and optionally wherein the C127 substitution is selected from the group consisting of C127A, C127I, C127Y, C127F, and C127L, further optionally wherein the C127 substitution is C127A or C127I. In some embodiments, the variant polypeptide further comprises C38S, C68S, and C76S substitutions, optionally wherein the variant polypeptide further comprises a C127S substitution. In some embodiments, the variant polypeptide further comprises C38I, C68S, and S117C. In some embodiments, the variant polypeptide further comprises C38V, C68I, S117C, and C127A. In some embodiments, the variant polypeptide further comprises C38S, C68I, S117C, and C127I. In some embodiments, the variant polypeptide further comprises C38I, C68I, C76V, and C127I. In some embodiments, the variant polypeptide further comprises C38I, C68L, and C76Y.

In some embodiments, the IL-18 variant polypeptide comprises a set of substitution combination comprising G3P, E6R, D54L, Q56G, P57S, and N91V, wherein the amino acid position(s) is relative to a wild-type human IL-18 set forth in SEQ ID NO: 1. In some embodiments, the IL-18 variant polypeptide does not comprise any other mutation relative to the wild-type human IL-18 set forth in SEQ ID NO: 1. In some embodiments, the IL-18 variant polypeptide further comprises one, two or three mutations or substitutions relative to the wild-type human IL-18 set forth in SEQ ID NO: 1, optionally wherein IL-18 variant polypeptide has a) a comparable or more potent binding affinity with IL-18 receptor (e.g., IL-18Rα), and/or b) a comparable or lower binding affinity with IL-18 binding protein, as compared to the corresponding IL-18 variant polypeptide without the one, two or three additional mutations or substitutions. In some embodiments, the IL-18 variant polypeptide further comprises a S117C substitution, optionally wherein the S117C substitution promotes a C76-C117 disulfide bond. In some embodiments, the IL-18 variant polypeptide further comprises a substitution at C38, optionally wherein the substitution is selected from the group consisting of C38I, C38V, C38L, and C38M, further optionally wherein the substitution is C38I or C38V. In some embodiments, the IL-18 variant polypeptide further comprises a substitution at C68, optionally wherein the substitution is selected from the group consisting of C68I, C68V, C68S, and C68D, further optionally wherein the substitution is C68I, C68L, or C38S. In some embodiments, the IL-18 variant polypeptide further comprises a substitution at C76 and/or C127, optionally wherein the C76 substitution is C76Y or C76V, and optionally wherein the C127 substitution is selected from the group consisting of C127A, C127I, C127Y, C127F, and C127L, further optionally wherein the C127 substitution is C127A or C127I. In some embodiments, the variant polypeptide further comprises C38S, C68S, and C76S substitutions, optionally wherein the variant polypeptide further comprises a C127S substitution. In some embodiments, the variant polypeptide further comprises C38I, C68S, and S117C. In some embodiments, the variant polypeptide further comprises C38V, C68I, S117C, and C127A. In some embodiments, the variant polypeptide further comprises C38S, C68I, S117C, and C127I. In some embodiments, the variant polypeptide further comprises C38I, C68I, C76V, and C127I. In some embodiments, the variant polypeptide further comprises C38I, C68L, and C76Y.

In some embodiments, the IL-18 variant polypeptide comprises a set of substitution combination comprising G3P, E6R, V11I, D54G, Q56G, P57A, and N91G, wherein the amino acid position(s) is relative to a wild-type human IL-18 set forth in SEQ ID NO: 1. In some embodiments, the IL-18 variant polypeptide does not comprise any other mutation relative to the wild-type human IL-18 set forth in SEQ ID NO: 1. In some embodiments, the IL-18 variant polypeptide further comprises one, two or three mutations or substitutions relative to the wild-type human IL-18 set forth in SEQ ID NO: 1, optionally wherein IL-18 variant polypeptide has a) a comparable or more potent binding affinity with IL-18 receptor (e.g., IL-18Rα), and/or b) a comparable or lower binding affinity with IL-18 binding protein, as compared to the corresponding IL-18 variant polypeptide without the one, two or three additional mutations or substitutions. In some embodiments, the IL-18 variant polypeptide further comprises a S117C substitution, optionally wherein the S117C substitution promotes a C76-C117 disulfide bond. In some embodiments, the IL-18 variant polypeptide further comprises a substitution at C38, optionally wherein the substitution is selected from the group consisting of C38I, C38V, C38L, and C38M, further optionally wherein the substitution is C38I or C38V. In some embodiments, the IL-18 variant polypeptide further comprises a substitution at C68, optionally wherein the substitution is selected from the group consisting of C68I, C68V, C68S, and C68D, further optionally wherein the substitution is C68I, C68L, or C38S. In some embodiments, the IL-18 variant polypeptide further comprises a substitution at C76 and/or C127, optionally wherein the C76 substitution is C76Y or C76V, and optionally wherein the C127 substitution is selected from the group consisting of C127A, C127I, C127Y, C127F, and C127L, further optionally wherein the C127 substitution is C127A or C127I. In some embodiments, the variant polypeptide further comprises C38S, C68S, and C76S substitutions, optionally wherein the variant polypeptide further comprises a C127S substitution. In some embodiments, the variant polypeptide further comprises C38I, C68S, and S117C. In some embodiments, the variant polypeptide further comprises C38V, C68I, S117C, and C127A. In some embodiments, the variant polypeptide further comprises C38S, C68I, S117C, and C127I. In some embodiments, the variant polypeptide further comprises C38I, C68I, C76V, and C127I. In some embodiments, the variant polypeptide further comprises C38I, C68L, and C76Y.

In some embodiments, the IL-18 variant polypeptide comprises a set of substitution combination comprising G3P, E6K, D54H, Q56Y, and P57S, wherein the amino acid position(s) is relative to a wild-type human IL-18 set forth in SEQ ID NO: 1. In some embodiments, the IL-18 variant polypeptide does not comprise any other mutation relative to the wild-type human IL-18 set forth in SEQ ID NO: 1. In some embodiments, the IL-18 variant polypeptide further comprises one, two or three mutations or substitutions relative to the wild-type human IL-18 set forth in SEQ ID NO: 1, optionally wherein IL-18 variant polypeptide has a) a comparable or more potent binding affinity with IL-18 receptor (e.g., IL-18Rα), and/or b) a comparable or lower binding affinity with IL-18 binding protein, as compared to the corresponding IL-18 variant polypeptide without the one, two or three additional mutations or substitutions. In some embodiments, the IL-18 variant polypeptide further comprises a S117C substitution, optionally wherein the S117C substitution promotes a C76-C117 disulfide bond. In some embodiments, the IL-18 variant polypeptide further comprises a substitution at C38, optionally wherein the substitution is selected from the group consisting of C38I, C38V, C38L, and C38M, further optionally wherein the substitution is C38I or C38V. In some embodiments, the IL-18 variant polypeptide further comprises a substitution at C68, optionally wherein the substitution is selected from the group consisting of C68I, C68V, C68S, and C68D, further optionally wherein the substitution is C68I, C68L, or C38S. In some embodiments, the IL-18 variant polypeptide further comprises a substitution at C76 and/or C127, optionally wherein the C76 substitution is C76Y or C76V, and optionally wherein the C127 substitution is selected from the group consisting of C127A, C127I, C127Y, C127F, and C127L, further optionally wherein the C127 substitution is C127A or C127I. In some embodiments, the variant polypeptide further comprises C38S, C68S, and C76S substitutions, optionally wherein the variant polypeptide further comprises a C127S substitution. In some embodiments, the variant polypeptide further comprises C38I, C68S, and S117C. In some embodiments, the variant polypeptide further comprises C38V, C68I, S117C, and C127A. In some embodiments, the variant polypeptide further comprises C38S, C68I, S117C, and C127I. In some embodiments, the variant polypeptide further comprises C38I, C68I, C76V, and C127I. In some embodiments, the variant polypeptide further comprises C38I, C68L, and C76Y.

In some embodiments, the IL-18 variant polypeptide comprises a set of substitution combination comprising E6R, D54W, Q56S, and P57Q, wherein the amino acid position(s) is relative to a wild-type human IL-18 set forth in SEQ ID NO: 1. In some embodiments, the IL-18 variant polypeptide does not comprise any other mutation relative to the wild-type human IL-18 set forth in SEQ ID NO: 1. In some embodiments, the IL-18 variant polypeptide further comprises one, two or three mutations or substitutions relative to the wild-type human IL-18 set forth in SEQ ID NO: 1, optionally wherein IL-18 variant polypeptide has a) a comparable or more potent binding affinity with IL-18 receptor (e.g., IL-18Rα), and/or b) a comparable or lower binding affinity with IL-18 binding protein, as compared to the corresponding IL-18 variant polypeptide without the one, two or three additional mutations or substitutions. In some embodiments, the IL-18 variant polypeptide further comprises a $117C substitution, optionally wherein the S117C substitution promotes a C76-C117 disulfide bond. In some embodiments, the IL-18 variant polypeptide further comprises a substitution at C38, optionally wherein the substitution is selected from the group consisting of C38I, C38V, C38L, and C38M, further optionally wherein the substitution is C38I or C38V. In some embodiments, the IL-18 variant polypeptide further comprises a substitution at C68, optionally wherein the substitution is selected from the group consisting of C68I, C68V, C68S, and C68D, further optionally wherein the substitution is C68I, C68L, or C38S. In some embodiments, the IL-18 variant polypeptide further comprises a substitution at C76 and/or C127, optionally wherein the C76 substitution is C76Y or C76V, and optionally wherein the C127 substitution is selected from the group consisting of C127A, C127I, C127Y, C127F, and C127L, further optionally wherein the C127 substitution is C127A or C127I. In some embodiments, the variant polypeptide further comprises C38S, C68S, and C76S substitutions, optionally wherein the variant polypeptide further comprises a C127S substitution. In some embodiments, the variant polypeptide further comprises C38I, C68S, and S117C. In some embodiments, the variant polypeptide further comprises C38V, C68I, S117C, and C127A. In some embodiments, the variant polypeptide further comprises C38S, C68I, S117C, and C127I. In some embodiments, the variant polypeptide further comprises C38I, C68I, C76V, and C127I. In some embodiments, the variant polypeptide further comprises C38I, C68L, and C76Y.

In some embodiments, the IL-18 variant polypeptide comprises a set of substitution combination comprising G3P, E6K, D54L, Q56T, P57Q, and N91V, wherein the amino acid position(s) is relative to a wild-type human IL-18 set forth in SEQ ID NO: 1. In some embodiments, the IL-18 variant polypeptide does not comprise any other mutation relative to the wild-type human IL-18 set forth in SEQ ID NO: 1. In some embodiments, the IL-18 variant polypeptide further comprises one, two or three mutations or substitutions relative to the wild-type human IL-18 set forth in SEQ ID NO: 1, optionally wherein IL-18 variant polypeptide has a) a comparable or more potent binding affinity with IL-18 receptor (e.g., IL-18Rα), and/or b) a comparable or lower binding affinity with IL-18 binding protein, as compared to the corresponding IL-18 variant polypeptide without the one, two or three additional mutations or substitutions. In some embodiments, the IL-18 variant polypeptide further comprises a S117C substitution, optionally wherein the S117C substitution promotes a C76-C117 disulfide bond. In some embodiments, the IL-18 variant polypeptide further comprises a substitution at C38, optionally wherein the substitution is selected from the group consisting of C38I, C38V, C38L, and C38M, further optionally wherein the substitution is C38I or C38V. In some embodiments, the IL-18 variant polypeptide further comprises a substitution at C68, optionally wherein the substitution is selected from the group consisting of C68I, C68V, C68S, and C68D, further optionally wherein the substitution is C68I, C68L, or C38S. In some embodiments, the IL-18 variant polypeptide further comprises a substitution at C76 and/or C127, optionally wherein the C76 substitution is C76Y or C76V, and optionally wherein the C127 substitution is selected from the group consisting of C127A, C127I, C127Y, C127F, and C127L, further optionally wherein the C127 substitution is C127A or C127I. In some embodiments, the variant polypeptide further comprises C38S, C68S, and C76S substitutions, optionally wherein the variant polypeptide further comprises a C127S substitution. In some embodiments, the variant polypeptide further comprises C38I, C68S, and S117C. In some embodiments, the variant polypeptide further comprises C38V, C68I, S117C, and C127A. In some embodiments, the variant polypeptide further comprises C38S, C68I, S117C, and C127I. In some embodiments, the variant polypeptide further comprises C38I, C68I, C76V, and C127I. In some embodiments, the variant polypeptide further comprises C38I, C68L, and C76Y.

In some embodiments, the IL-18 variant polypeptide comprises a set of substitution combination comprising G3A, E6Y, D54R, Q56S, P57L, and N91G, wherein the amino acid position(s) is relative to a wild-type human IL-18 set forth in SEQ ID NO: 1. In some embodiments, the IL-18 variant polypeptide does not comprise any other mutation relative to the wild-type human IL-18 set forth in SEQ ID NO: 1. In some embodiments, the IL-18 variant polypeptide further comprises one, two or three mutations or substitutions relative to the wild-type human IL-18 set forth in SEQ ID NO: 1, optionally wherein IL-18 variant polypeptide has a) a comparable or more potent binding affinity with IL-18 receptor (e.g., IL-18Rα), and/or b) a comparable or lower binding affinity with IL-18 binding protein, as compared to the corresponding IL-18 variant polypeptide without the one, two or three additional mutations or substitutions. In some embodiments, the IL-18 variant polypeptide further comprises a S117C substitution, optionally wherein the S117C substitution promotes a C76-C117 disulfide bond. In some embodiments, the IL-18 variant polypeptide further comprises a substitution at C38, optionally wherein the substitution is selected from the group consisting of C38I, C38V, C38L, and C38M, further optionally wherein the substitution is C38I or C38V. In some embodiments, the IL-18 variant polypeptide further comprises a substitution at C68, optionally wherein the substitution is selected from the group consisting of C68I, C68V, C68S, and C68D, further optionally wherein the substitution is C68I, C68L, or C38S. In some embodiments, the IL-18 variant polypeptide further comprises a substitution at C76 and/or C127, optionally wherein the C76 substitution is C76Y or C76V, and optionally wherein the C127 substitution is selected from the group consisting of C127A, C127I, C127Y, C127F, and C127L, further optionally wherein the C127 substitution is C127A or C127I. In some embodiments, the variant polypeptide further comprises C38S, C68S, and C76S substitutions, optionally wherein the variant polypeptide further comprises a C127S substitution. In some embodiments, the variant polypeptide further comprises C38I, C68S, and S117C. In some embodiments, the variant polypeptide further comprises C38V, C68I, S117C, and C127A. In some embodiments, the variant polypeptide further comprises C38S, C68I, S117C, and C127I. In some embodiments, the variant polypeptide further comprises C38I, C68I, C76V, and C127I. In some embodiments, the variant polypeptide further comprises C38I, C68L, and C76Y.

In some embodiments, the IL-18 variant polypeptide comprises a set of substitution combination comprising G3P, E6R, D54L, Q56T, P57I, and N91G, wherein the amino acid position(s) is relative to a wild-type human IL-18 set forth in SEQ ID NO: 1. In some embodiments, the IL-18 variant polypeptide does not comprise any other mutation relative to the wild-type human IL-18 set forth in SEQ ID NO: 1. In some embodiments, the IL-18 variant polypeptide further comprises one, two or three mutations or substitutions relative to the wild-type human IL-18 set forth in SEQ ID NO: 1, optionally wherein IL-18 variant polypeptide has a) a comparable or more potent binding affinity with IL-18 receptor (e.g., IL-18Rα), and/or b) a comparable or lower binding affinity with IL-18 binding protein, as compared to the corresponding IL-18 variant polypeptide without the one, two or three additional mutations or substitutions. In some embodiments, the IL-18 variant polypeptide further comprises a S117C substitution, optionally wherein the S117C substitution promotes a C76-C117 disulfide bond. In some embodiments, the IL-18 variant polypeptide further comprises a substitution at C38, optionally wherein the substitution is selected from the group consisting of C38I, C38V, C38L, and C38M, further optionally wherein the substitution is C38I or C38V. In some embodiments, the IL-18 variant polypeptide further comprises a substitution at C68, optionally wherein the substitution is selected from the group consisting of C68I, C68V, C68S, and C68D, further optionally wherein the substitution is C68I, C68L, or C38S. In some embodiments, the IL-18 variant polypeptide further comprises a substitution at C76 and/or C127, optionally wherein the C76 substitution is C76Y or C76V, and optionally wherein the C127 substitution is selected from the group consisting of C127A, C127I, C127Y, C127F, and C127L, further optionally wherein the C127 substitution is C127A or C127I. In some embodiments, the variant polypeptide further comprises C38S, C68S, and C76S substitutions, optionally wherein the variant polypeptide further comprises a C127S substitution. In some embodiments, the variant polypeptide further comprises C38I, C68S, and S117C. In some embodiments, the variant polypeptide further comprises C38V, C68I, S117C, and C127A. In some embodiments, the variant polypeptide further comprises C38S, C68I, S117C, and C127I. In some embodiments, the variant polypeptide further comprises C38I, C68I, C76V, and C127I. In some embodiments, the variant polypeptide further comprises C38I, C68L, and C76Y.

In some embodiments, the IL-18 variant polypeptide comprises a set of substitution combination comprising E6G, D54L, Q56T, P57E, N91G, and R104S, wherein the amino acid position(s) is relative to a wild-type human IL-18 set forth in SEQ ID NO: 1. In some embodiments, the IL-18 variant polypeptide does not comprise any other mutation relative to the wild-type human IL-18 set forth in SEQ ID NO: 1. In some embodiments, the IL-18 variant polypeptide further comprises one, two or three mutations or substitutions relative to the wild-type human IL-18 set forth in SEQ ID NO: 1, optionally wherein IL-18 variant polypeptide has a) a comparable or more potent binding affinity with IL-18 receptor (e.g., IL-18Rα), and/or b) a comparable or lower binding affinity with IL-18 binding protein, as compared to the corresponding IL-18 variant polypeptide without the one, two or three additional mutations or substitutions. In some embodiments, the IL-18 variant polypeptide further comprises a S117C substitution, optionally wherein the S117C substitution promotes a C76-C117 disulfide bond. In some embodiments, the IL-18 variant polypeptide further comprises a substitution at C38, optionally wherein the substitution is selected from the group consisting of C38I, C38V, C38L, and C38M, further optionally wherein the substitution is C38I or C38V. In some embodiments, the IL-18 variant polypeptide further comprises a substitution at C68, optionally wherein the substitution is selected from the group consisting of C68I, C68V, C68S, and C68D, further optionally wherein the substitution is C68I, C68L, or C38S. In some embodiments, the IL-18 variant polypeptide further comprises a substitution at C76 and/or C127, optionally wherein the C76 substitution is C76Y or C76V, and optionally wherein the C127 substitution is selected from the group consisting of C127A, C127I, C127Y, C127F, and C127L, further optionally wherein the C127 substitution is C127A or C127I. In some embodiments, the variant polypeptide further comprises C38S, C68S, and C76S substitutions, optionally wherein the variant polypeptide further comprises a C127S substitution. In some embodiments, the variant polypeptide further comprises C38I, C68S, and S117C. In some embodiments, the variant polypeptide further comprises C38V, C68I, S117C, and C127A. In some embodiments, the variant polypeptide further comprises C38S, C68I, S117C, and C127I. In some embodiments, the variant polypeptide further comprises C38I, C68I, C76V, and C127I. In some embodiments, the variant polypeptide further comprises C38I, C68L, and C76Y.

In some embodiments, the IL-18 variant polypeptide comprises a set of substitution combination comprising G3A, E6Y, D54R, Q56S, P57L, and N91A, wherein the amino acid position(s) is relative to a wild-type human IL-18 set forth in SEQ ID NO: 1. In some embodiments, the IL-18 variant polypeptide does not comprise any other mutation relative to the wild-type human IL-18 set forth in SEQ ID NO: 1. In some embodiments, the IL-18 variant polypeptide further comprises one, two or three mutations or substitutions relative to the wild-type human IL-18 set forth in SEQ ID NO: 1, optionally wherein IL-18 variant polypeptide has a) a comparable or more potent binding affinity with IL-18 receptor (e.g., IL-18Rα), and/or b) a comparable or lower binding affinity with IL-18 binding protein, as compared to the corresponding IL-18 variant polypeptide without the one, two or three additional mutations or substitutions. In some embodiments, the IL-18 variant polypeptide further comprises a S117C substitution, optionally wherein the S117C substitution promotes a C76-C117 disulfide bond. In some embodiments, the IL-18 variant polypeptide further comprises a substitution at C38, optionally wherein the substitution is selected from the group consisting of C38I, C38V, C38L, and C38M, further optionally wherein the substitution is C38I or C38V. In some embodiments, the IL-18 variant polypeptide further comprises a substitution at C68, optionally wherein the substitution is selected from the group consisting of C68I, C68V, C68S, and C68D, further optionally wherein the substitution is C68I, C68L, or C38S. In some embodiments, the IL-18 variant polypeptide further comprises a substitution at C76 and/or C127, optionally wherein the C76 substitution is C76Y or C76V, and optionally wherein the C127 substitution is selected from the group consisting of C127A, C127I, C127Y, C127F, and C127L, further optionally wherein the C127 substitution is C127A or C127I. In some embodiments, the variant polypeptide further comprises C38S, C68S, and C76S substitutions, optionally wherein the variant polypeptide further comprises a C127S substitution. In some embodiments, the variant polypeptide further comprises C38I, C68S, and S117C. In some embodiments, the variant polypeptide further comprises C38V, C68I, S117C, and C127A. In some embodiments, the variant polypeptide further comprises C38S, C68I, S117C, and C127I. In some embodiments, the variant polypeptide further comprises C38I, C68I, C76V, and C127I. In some embodiments, the variant polypeptide further comprises C38I, C68L, and C76Y.

In some embodiments, the IL-18 variant polypeptide comprises a set of substitution combination comprising M60K, and K96D, wherein the amino acid position(s) is relative to a wild-type human IL-18 set forth in SEQ ID NO: 1. In some embodiments, the IL-18 variant polypeptide does not comprise any other mutation relative to the wild-type human IL-18 set forth in SEQ ID NO: 1. In some embodiments, the IL-18 variant polypeptide further comprises one, two or three mutations or substitutions relative to the wild-type human IL-18 set forth in SEQ ID NO: 1, optionally wherein IL-18 variant polypeptide has a) a comparable or more potent binding affinity with IL-18 receptor (e.g., IL-18Rα), and/or b) a comparable or lower binding affinity with IL-18 binding protein, as compared to the corresponding IL-18 variant polypeptide without the one, two or three additional mutations or substitutions. In some embodiments, the IL-18 variant polypeptide further comprises a S117C substitution, optionally wherein the S117C substitution promotes a C76-C117 disulfide bond. In some embodiments, the IL-18 variant polypeptide further comprises a substitution at C38, optionally wherein the substitution is selected from the group consisting of C38I, C38V, C38L, and C38M, further optionally wherein the substitution is C38I or C38V. In some embodiments, the IL-18 variant polypeptide further comprises a substitution at C68, optionally wherein the substitution is selected from the group consisting of C68I, C68V, C68S, and C68D, further optionally wherein the substitution is C68I, C68L, or C38S. In some embodiments, the IL-18 variant polypeptide further comprises a substitution at C76 and/or C127, optionally wherein the C76 substitution is C76Y or C76V, and optionally wherein the C127 substitution is selected from the group consisting of C127A, C127I, C127Y, C127F, and C127L, further optionally wherein the C127 substitution is C127A or C127I. In some embodiments, the variant polypeptide further comprises C38S, C68S, and C76S substitutions, optionally wherein the variant polypeptide further comprises a C127S substitution. In some embodiments, the variant polypeptide further comprises C38I, C68S, and S117C. In some embodiments, the variant polypeptide further comprises C38V, C68I, S117C, and C127A. In some embodiments, the variant polypeptide further comprises C38S, C68I, S117C, and C127I. In some embodiments, the variant polypeptide further comprises C38I, C68I, C76V, and C127I. In some embodiments, the variant polypeptide further comprises C38I, C68L, and C76Y.

In some embodiments, the IL-18 variant polypeptide comprises a set of substitution combination comprising G3P, E6R, and K96E, wherein the amino acid position(s) is relative to a wild-type human IL-18 set forth in SEQ ID NO: 1. In some embodiments, the IL-18 variant polypeptide does not comprise any other mutation relative to the wild-type human IL-18 set forth in SEQ ID NO: 1. In some embodiments, the IL-18 variant polypeptide further comprises one, two or three mutations or substitutions relative to the wild-type human IL-18 set forth in SEQ ID NO: 1, optionally wherein IL-18 variant polypeptide has a) a comparable or more potent binding affinity with IL-18 receptor (e.g., IL-18Rα), and/or b) a comparable or lower binding affinity with IL-18 binding protein, as compared to the corresponding IL-18 variant polypeptide without the one, two or three additional mutations or substitutions. In some embodiments, the IL-18 variant polypeptide further comprises a S117C substitution, optionally wherein the S117C substitution promotes a C76-C117 disulfide bond. In some embodiments, the IL-18 variant polypeptide further comprises a substitution at C38, optionally wherein the substitution is selected from the group consisting of C38I, C38V, C38L, and C38M, further optionally wherein the substitution is C38I or C38V. In some embodiments, the IL-18 variant polypeptide further comprises a substitution at C68, optionally wherein the substitution is selected from the group consisting of C68I, C68V, C68S, and C68D, further optionally wherein the substitution is C68I, C68L, or C38S. In some embodiments, the IL-18 variant polypeptide further comprises a substitution at C76 and/or C127, optionally wherein the C76 substitution is C76Y or C76V, and optionally wherein the C127 substitution is selected from the group consisting of C127A, C127I, C127Y, C127F, and C127L, further optionally wherein the C127 substitution is C127A or C127I. In some embodiments, the variant polypeptide further comprises C38S, C68S, and C76S substitutions, optionally wherein the variant polypeptide further comprises a C127S substitution. In some embodiments, the variant polypeptide further comprises C38I, C68S, and S117C. In some embodiments, the variant polypeptide further comprises C38V, C68I, S117C, and C127A. In some embodiments, the variant polypeptide further comprises C38S, C68I, S117C, and C127I. In some embodiments, the variant polypeptide further comprises C38I, C68I, C76V, and C127I. In some embodiments, the variant polypeptide further comprises C38I, C68L, and C76Y.

In some embodiments, the IL-18 variant polypeptide comprises one substitution (e.g., a single substitution) comprising M60K, wherein the amino acid position(s) is relative to a wild-type human IL-18 set forth in SEQ ID NO: 1. In some embodiments, the IL-18 variant polypeptide does not comprise any other mutation relative to the wild-type human IL-18 set forth in SEQ ID NO: 1. In some embodiments, the IL-18 variant polypeptide further comprises one, two or three mutations or substitutions relative to the wild-type human IL-18 set forth in SEQ ID NO: 1, optionally wherein IL-18 variant polypeptide has a) a comparable or more potent binding affinity with IL-18 receptor (e.g., IL-18Rα), and/or b) a comparable or lower binding affinity with IL-18 binding protein, as compared to the corresponding IL-18 variant polypeptide without the one, two or three additional mutations or substitutions. In some embodiments, the IL-18 variant polypeptide further comprises a S117C substitution, optionally wherein the S117C substitution promotes a C76-C117 disulfide bond. In some embodiments, the IL-18 variant polypeptide further comprises a substitution at C38, optionally wherein the substitution is selected from the group consisting of C38I, C38V, C38L, and C38M, further optionally wherein the substitution is C38I or C38V. In some embodiments, the IL-18 variant polypeptide further comprises a substitution at C68, optionally wherein the substitution is selected from the group consisting of C68I, C68V, C68S, and C68D, further optionally wherein the substitution is C68I, C68L, or C38S. In some embodiments, the IL-18 variant polypeptide further comprises a substitution at C76 and/or C127, optionally wherein the C76 substitution is C76Y or C76V, and optionally wherein the C127 substitution is selected from the group consisting of C127A, C127I, C127Y, C127F, and C127L, further optionally wherein the C127 substitution is C127A or C127I. In some embodiments, the variant polypeptide further comprises C38S, C68S, and C76S substitutions, optionally wherein the variant polypeptide further comprises a C127S substitution. In some embodiments, the variant polypeptide further comprises C38I, C68S, and S117C. In some embodiments, the variant polypeptide further comprises C38V, C68I, S117C, and C127A. In some embodiments, the variant polypeptide further comprises C38S, C68I, S117C, and C127I. In some embodiments, the variant polypeptide further comprises C38I, C68I, C76V, and C127I. In some embodiments, the variant polypeptide further comprises C38I, C68L, and C76Y.

In some embodiments, the IL-18 variant polypeptide comprises a set of substitution combination comprising G3P and E6R, wherein the amino acid position(s) is relative to a wild-type human IL-18 set forth in SEQ ID NO: 1. In some embodiments, the IL-18 variant polypeptide does not comprise any other mutation relative to the wild-type human IL-18 set forth in SEQ ID NO: 1. In some embodiments, the IL-18 variant polypeptide further comprises one, two or three mutations or substitutions relative to the wild-type human IL-18 set forth in SEQ ID NO: 1, optionally wherein IL-18 variant polypeptide has a) a comparable or more potent binding affinity with IL-18 receptor (e.g., IL-18Rα), and/or b) a comparable or lower binding affinity with IL-18 binding protein, as compared to the corresponding IL-18 variant polypeptide without the one, two or three additional mutations or substitutions. In some embodiments, the IL-18 variant polypeptide further comprises a S117C substitution, optionally wherein the S117C substitution promotes a C76-C117 disulfide bond. In some embodiments, the IL-18 variant polypeptide further comprises a substitution at C38, optionally wherein the substitution is selected from the group consisting of C38I, C38V, C38L, and C38M, further optionally wherein the substitution is C38I or C38V. In some embodiments, the IL-18 variant polypeptide further comprises a substitution at C68, optionally wherein the substitution is selected from the group consisting of C68I, C68V, C68S, and C68D, further optionally wherein the substitution is C68I, C68L, or C38S. In some embodiments, the IL-18 variant polypeptide further comprises a substitution at C76 and/or C127, optionally wherein the C76 substitution is C76Y or C76V, and optionally wherein the C127 substitution is selected from the group consisting of C127A, C127I, C127Y, C127F, and C127L, further optionally wherein the C127 substitution is C127A or C127I. In some embodiments, the variant polypeptide further comprises C38S, C68S, and C76S substitutions, optionally wherein the variant polypeptide further comprises a C127S substitution. In some embodiments, the variant polypeptide further comprises C38I, C68S, and S117C. In some embodiments, the variant polypeptide further comprises C38V, C68I, S117C, and C127A. In some embodiments, the variant polypeptide further comprises C38S, C68I, S117C, and C127I. In some embodiments, the variant polypeptide further comprises C38I, C68I, C76V, and C127I. In some embodiments, the variant polypeptide further comprises C38I, C68L, and C76Y.

In some embodiments, the IL-18 variant polypeptide comprises a set of substitution combination comprising G3P and E6K, wherein the amino acid position(s) is relative to a wild-type human IL-18 set forth in SEQ ID NO: 1. In some embodiments, the IL-18 variant polypeptide does not comprise any other mutation relative to the wild-type human IL-18 set forth in SEQ ID NO: 1. In some embodiments, the IL-18 variant polypeptide further comprises one, two or three mutations or substitutions relative to the wild-type human IL-18 set forth in SEQ ID NO: 1, optionally wherein IL-18 variant polypeptide has a) a comparable or more potent binding affinity with IL-18 receptor (e.g., IL-18Rα), and/or b) a comparable or lower binding affinity with IL-18 binding protein, as compared to the corresponding IL-18 variant polypeptide without the one, two or three additional mutations or substitutions. In some embodiments, the IL-18 variant polypeptide further comprises a S117C substitution, optionally wherein the S117C substitution promotes a C76-C117 disulfide bond. In some embodiments, the IL-18 variant polypeptide further comprises a substitution at C38, optionally wherein the substitution is selected from the group consisting of C38I, C38V, C38L, and C38M, further optionally wherein the substitution is C38I or C38V. In some embodiments, the IL-18 variant polypeptide further comprises a substitution at C68, optionally wherein the substitution is selected from the group consisting of C68I, C68V, C68S, and C68D, further optionally wherein the substitution is C68I, C68L, or C38S. In some embodiments, the IL-18 variant polypeptide further comprises a substitution at C76 and/or C127, optionally wherein the C76 substitution is C76Y or C76V, and optionally wherein the C127 substitution is selected from the group consisting of C127A, C127I, C127Y, C127F, and C127L, further optionally wherein the C127 substitution is C127A or C127I. In some embodiments, the variant polypeptide further comprises C38S, C68S, and C76S substitutions, optionally wherein the variant polypeptide further comprises a C127S substitution. In some embodiments, the variant polypeptide further comprises C38I, C68S, and S117C. In some embodiments, the variant polypeptide further comprises C38V, C68I, S117C, and C127A. In some embodiments, the variant polypeptide further comprises C38S, C68I, S117C, and C127I. In some embodiments, the variant polypeptide further comprises C38I, C68I, C76V, and C127I. In some embodiments, the variant polypeptide further comprises C38I, C68L, and C76Y.

In some embodiments, the IL-18 variant polypeptide comprises a set of substitution combination comprising G3D, E6K, and N91S, wherein the amino acid position(s) is relative to a wild-type human IL-18 set forth in SEQ ID NO: 1. In some embodiments, the IL-18 variant polypeptide does not comprise any other mutation relative to the wild-type human IL-18 set forth in SEQ ID NO: 1. In some embodiments, the IL-18 variant polypeptide further comprises one, two or three mutations or substitutions relative to the wild-type human IL-18 set forth in SEQ ID NO: 1, optionally wherein IL-18 variant polypeptide has a) a comparable or more potent binding affinity with IL-18 receptor (e.g., IL-18Rα), and/or b) a comparable or lower binding affinity with IL-18 binding protein, as compared to the corresponding IL-18 variant polypeptide without the one, two or three additional mutations or substitutions. In some embodiments, the IL-18 variant polypeptide further comprises a S117C substitution, optionally wherein the S117C substitution promotes a C76-C117 disulfide bond. In some embodiments, the IL-18 variant polypeptide further comprises a substitution at C38, optionally wherein the substitution is selected from the group consisting of C38I, C38V, C38L, and C38M, further optionally wherein the substitution is C38I or C38V. In some embodiments, the IL-18 variant polypeptide further comprises a substitution at C68, optionally wherein the substitution is selected from the group consisting of C68I, C68V, C68S, and C68D, further optionally wherein the substitution is C68I, C68L, or C38S. In some embodiments, the IL-18 variant polypeptide further comprises a substitution at C76 and/or C127, optionally wherein the C76 substitution is C76Y or C76V, and optionally wherein the C127 substitution is selected from the group consisting of C127A, C127I, C127Y, C127F, and C127L, further optionally wherein the C127 substitution is C127A or C127I. In some embodiments, the variant polypeptide further comprises C38S, C68S, and C76S substitutions, optionally wherein the variant polypeptide further comprises a C127S substitution. In some embodiments, the variant polypeptide further comprises C38I, C68S, and S117C. In some embodiments, the variant polypeptide further comprises C38V, C68I, S117C, and C127A. In some embodiments, the variant polypeptide further comprises C38S, C68I, S117C, and C127I. In some embodiments, the variant polypeptide further comprises C38I, C68I, C76V, and C127I. In some embodiments, the variant polypeptide further comprises C38I, C68L, and C76Y.

In some embodiments, the IL-18 variant polypeptide comprises a set of substitution combination comprising G3S, and I149M, wherein the amino acid position(s) is relative to a wild-type human IL-18 set forth in SEQ ID NO: 1. In some embodiments, the IL-18 variant polypeptide does not comprise any other mutation relative to the wild-type human IL-18 set forth in SEQ ID NO: 1. In some embodiments, the IL-18 variant polypeptide further comprises one, two or three mutations or substitutions relative to the wild-type human IL-18 set forth in SEQ ID NO: 1, optionally wherein IL-18 variant polypeptide has a) a comparable or more potent binding affinity with IL-18 receptor (e.g., IL-18Rα), and/or b) a comparable or lower binding affinity with IL-18 binding protein, as compared to the corresponding IL-18 variant polypeptide without the one, two or three additional mutations or substitutions. In some embodiments, the IL-18 variant polypeptide further comprises a $117C substitution, optionally wherein the S117C substitution promotes a C76-C117 disulfide bond. In some embodiments, the IL-18 variant polypeptide further comprises a substitution at C38, optionally wherein the substitution is selected from the group consisting of C38I, C38V, C38L, and C38M, further optionally wherein the substitution is C38I or C38V. In some embodiments, the IL-18 variant polypeptide further comprises a substitution at C68, optionally wherein the substitution is selected from the group consisting of C68I, C68V, C68S, and C68D, further optionally wherein the substitution is C68I, C68L, or C38S. In some embodiments, the IL-18 variant polypeptide further comprises a substitution at C76 and/or C127, optionally wherein the C76 substitution is C76Y or C76V, and optionally wherein the C127 substitution is selected from the group consisting of C127A, C127I, C127Y, C127F, and C127L, further optionally wherein the C127 substitution is C127A or C127I. In some embodiments, the variant polypeptide further comprises C38S, C68S, and C76S substitutions, optionally wherein the variant polypeptide further comprises a C127S substitution. In some embodiments, the variant polypeptide further comprises C38I, C68S, and S117C. In some embodiments, the variant polypeptide further comprises C38V, C68I, S117C, and C127A. In some embodiments, the variant polypeptide further comprises C38S, C68I, S117C, and C127I. In some embodiments, the variant polypeptide further comprises C38I, C68I, C76V, and C127I. In some embodiments, the variant polypeptide further comprises C38I, C68L, and C76Y.

In some embodiments, the IL-18 variant polypeptide comprises a set of substitution combination comprising G3P, E6R, and N91S, wherein the amino acid position(s) is relative to a wild-type human IL-18 set forth in SEQ ID NO: 1. In some embodiments, the IL-18 variant polypeptide does not comprise any other mutation relative to the wild-type human IL-18 set forth in SEQ ID NO: 1. In some embodiments, the IL-18 variant polypeptide further comprises one, two or three mutations or substitutions relative to the wild-type human IL-18 set forth in SEQ ID NO: 1, optionally wherein IL-18 variant polypeptide has a) a comparable or more potent binding affinity with IL-18 receptor (e.g., IL-18Rα), and/or b) a comparable or lower binding affinity with IL-18 binding protein, as compared to the corresponding IL-18 variant polypeptide without the one, two or three additional mutations or substitutions. In some embodiments, the IL-18 variant polypeptide further comprises a $117C substitution, optionally wherein the S117C substitution promotes a C76-C117 disulfide bond. In some embodiments, the IL-18 variant polypeptide further comprises a substitution at C38, optionally wherein the substitution is selected from the group consisting of C38I, C38V, C38L, and C38M, further optionally wherein the substitution is C38I or C38V. In some embodiments, the IL-18 variant polypeptide further comprises a substitution at C68, optionally wherein the substitution is selected from the group consisting of C68I, C68V, C68S, and C68D, further optionally wherein the substitution is C68I, C68L, or C38S. In some embodiments, the IL-18 variant polypeptide further comprises a substitution at C76 and/or C127, optionally wherein the C76 substitution is C76Y or C76V, and optionally wherein the C127 substitution is selected from the group consisting of C127A, C127I, C127Y, C127F, and C127L, further optionally wherein the C127 substitution is C127A or C127I. In some embodiments, the variant polypeptide further comprises C38S, C68S, and C76S substitutions, optionally wherein the variant polypeptide further comprises a C127S substitution. In some embodiments, the variant polypeptide further comprises C38I, C68S, and S117C. In some embodiments, the variant polypeptide further comprises C38V, C68I, S117C, and C127A. In some embodiments, the variant polypeptide further comprises C38S, C68I, S117C, and C127I. In some embodiments, the variant polypeptide further comprises C38I, C68I, C76V, and C127I. In some embodiments, the variant polypeptide further comprises C38I, C68L, and C76Y.

In some embodiments, the IL-18 variant polypeptide comprises one substitution (e.g., a single substitution) comprising E6R, wherein the amino acid position(s) is relative to a wild-type human IL-18 set forth in SEQ ID NO: 1. In some embodiments, the IL-18 variant polypeptide does not comprise any other mutation relative to the wild-type human IL-18 set forth in SEQ ID NO: 1. In some embodiments, the IL-18 variant polypeptide further comprises one, two or three mutations or substitutions relative to the wild-type human IL-18 set forth in SEQ ID NO: 1, optionally wherein IL-18 variant polypeptide has a) a comparable or more potent binding affinity with IL-18 receptor (e.g., IL-18Rα), and/or b) a comparable or lower binding affinity with IL-18 binding protein, as compared to the corresponding IL-18 variant polypeptide without the one, two or three additional mutations or substitutions. In some embodiments, the IL-18 variant polypeptide further comprises a S117C substitution, optionally wherein the S117C substitution promotes a C76-C117 disulfide bond. In some embodiments, the IL-18 variant polypeptide further comprises a substitution at C38, optionally wherein the substitution is selected from the group consisting of C38I, C38V, C38L, and C38M, further optionally wherein the substitution is C38I or C38V. In some embodiments, the IL-18 variant polypeptide further comprises a substitution at C68, optionally wherein the substitution is selected from the group consisting of C68I, C68V, C68S, and C68D, further optionally wherein the substitution is C68I, C68L, or C38S. In some embodiments, the IL-18 variant polypeptide further comprises a substitution at C76 and/or C127, optionally wherein the C76 substitution is C76Y or C76V, and optionally wherein the C127 substitution is selected from the group consisting of C127A, C127I, C127Y, C127F, and C127L, further optionally wherein the C127 substitution is C127A or C127I. In some embodiments, the variant polypeptide further comprises C38S, C68S, and C76S substitutions, optionally wherein the variant polypeptide further comprises a C127S substitution. In some embodiments, the variant polypeptide further comprises C38I, C68S, and S117C. In some embodiments, the variant polypeptide further comprises C38V, C68I, S117C, and C127A. In some embodiments, the variant polypeptide further comprises C38S, C68I, S117C, and C127I. In some embodiments, the variant polypeptide further comprises C38I, C68I, C76V, and C127I. In some embodiments, the variant polypeptide further comprises C38I, C68L, and C76Y.

In some embodiments, the IL-18 variant polypeptide comprises a set of substitution combination comprising E6R and N91S, wherein the amino acid position(s) is relative to a wild-type human IL-18 set forth in SEQ ID NO: 1. In some embodiments, the IL-18 variant polypeptide does not comprise any other mutation relative to the wild-type human IL-18 set forth in SEQ ID NO: 1. In some embodiments, the IL-18 variant polypeptide further comprises one, two or three mutations or substitutions relative to the wild-type human IL-18 set forth in SEQ ID NO: 1, optionally wherein IL-18 variant polypeptide has a) a comparable or more potent binding affinity with IL-18 receptor (e.g., IL-18Rα), and/or b) a comparable or lower binding affinity with IL-18 binding protein, as compared to the corresponding IL-18 variant polypeptide without the one, two or three additional mutations or substitutions. In some embodiments, the IL-18 variant polypeptide further comprises a S117C substitution, optionally wherein the S117C substitution promotes a C76-C117 disulfide bond. In some embodiments, the IL-18 variant polypeptide further comprises a substitution at C38, optionally wherein the substitution is selected from the group consisting of C38I, C38V, C38L, and C38M, further optionally wherein the substitution is C38I or C38V. In some embodiments, the IL-18 variant polypeptide further comprises a substitution at C68, optionally wherein the substitution is selected from the group consisting of C68I, C68V, C68S, and C68D, further optionally wherein the substitution is C68I, C68L, or C38S. In some embodiments, the IL-18 variant polypeptide further comprises a substitution at C76 and/or C127, optionally wherein the C76 substitution is C76Y or C76V, and optionally wherein the C127 substitution is selected from the group consisting of C127A, C127I, C127Y, C127F, and C127L, further optionally wherein the C127 substitution is C127A or C127I. In some embodiments, the variant polypeptide further comprises C38S, C68S, and C76S substitutions, optionally wherein the variant polypeptide further comprises a C127S substitution. In some embodiments, the variant polypeptide further comprises C38I, C68S, and S117C. In some embodiments, the variant polypeptide further comprises C38V, C68I, S117C, and C127A. In some embodiments, the variant polypeptide further comprises C38S, C68I, S117C, and C127I. In some embodiments, the variant polypeptide further comprises C38I, C68I, C76V, and C127I. In some embodiments, the variant polypeptide further comprises C38I, C68L, and C76Y.

In some embodiments, the IL-18 variant polypeptide comprises one substitution (e.g., a single substitution) comprising V111, wherein the amino acid position(s) is relative to a wild-type human IL-18 set forth in SEQ ID NO: 1. In some embodiments, the IL-18 variant polypeptide does not comprise any other mutation relative to the wild-type human IL-18 set forth in SEQ ID NO: 1. In some embodiments, the IL-18 variant polypeptide further comprises one, two or three mutations or substitutions relative to the wild-type human IL-18 set forth in SEQ ID NO: 1, optionally wherein IL-18 variant polypeptide has a) a comparable or more potent binding affinity with IL-18 receptor (e.g., IL-18Rα), and/or b) a comparable or lower binding affinity with IL-18 binding protein, as compared to the corresponding IL-18 variant polypeptide without the one, two or three additional mutations or substitutions. In some embodiments, the IL-18 variant polypeptide further comprises a S117C substitution, optionally wherein the S117C substitution promotes a C76-C117 disulfide bond. In some embodiments, the IL-18 variant polypeptide further comprises a substitution at C38, optionally wherein the substitution is selected from the group consisting of C38I, C38V, C38L, and C38M, further optionally wherein the substitution is C38I or C38V. In some embodiments, the IL-18 variant polypeptide further comprises a substitution at C68, optionally wherein the substitution is selected from the group consisting of C68I, C68V, C68S, and C68D, further optionally wherein the substitution is C68I, C68L, or C38S. In some embodiments, the IL-18 variant polypeptide further comprises a substitution at C76 and/or C127, optionally wherein the C76 substitution is C76Y or C76V, and optionally wherein the C127 substitution is selected from the group consisting of C127A, C127I, C127Y, C127F, and C127L, further optionally wherein the C127 substitution is C127A or C127I. In some embodiments, the variant polypeptide further comprises C38S, C68S, and C76S substitutions, optionally wherein the variant polypeptide further comprises a C127S substitution. In some embodiments, the variant polypeptide further comprises C38I, C68S, and S117C. In some embodiments, the variant polypeptide further comprises C38V, C68I, S117C, and C127A. In some embodiments, the variant polypeptide further comprises C38S, C68I, S117C, and C127I. In some embodiments, the variant polypeptide further comprises C38I, C68I, C76V, and C127I. In some embodiments, the variant polypeptide further comprises C38I, C68L, and C76Y.

In some embodiments, the IL-18 variant polypeptide comprises a set of substitution combination comprising G3S and K140R, wherein the amino acid position(s) is relative to a wild-type human IL-18 set forth in SEQ ID NO: 1. In some embodiments, the IL-18 variant polypeptide does not comprise any other mutation relative to the wild-type human IL-18 set forth in SEQ ID NO: 1. In some embodiments, the IL-18 variant polypeptide further comprises one, two or three mutations or substitutions relative to the wild-type human IL-18 set forth in SEQ ID NO: 1, optionally wherein IL-18 variant polypeptide has a) a comparable or more potent binding affinity with IL-18 receptor (e.g., IL-18Rα), and/or b) a comparable or lower binding affinity with IL-18 binding protein, as compared to the corresponding IL-18 variant polypeptide without the one, two or three additional mutations or substitutions. In some embodiments, the IL-18 variant polypeptide further comprises a S117C substitution, optionally wherein the S117C substitution promotes a C76-C117 disulfide bond. In some embodiments, the IL-18 variant polypeptide further comprises a substitution at C38, optionally wherein the substitution is selected from the group consisting of C38I, C38V, C38L, and C38M, further optionally wherein the substitution is C38I or C38V. In some embodiments, the IL-18 variant polypeptide further comprises a substitution at C68, optionally wherein the substitution is selected from the group consisting of C68I, C68V, C68S, and C68D, further optionally wherein the substitution is C68I, C68L, or C38S. In some embodiments, the IL-18 variant polypeptide further comprises a substitution at C76 and/or C127, optionally wherein the C76 substitution is C76Y or C76V, and optionally wherein the C127 substitution is selected from the group consisting of C127A, C127I, C127Y, C127F, and C127L, further optionally wherein the C127 substitution is C127A or C127I. In some embodiments, the variant polypeptide further comprises C38S, C68S, and C76S substitutions, optionally wherein the variant polypeptide further comprises a C127S substitution. In some embodiments, the variant polypeptide further comprises C38I, C68S, and S117C. In some embodiments, the variant polypeptide further comprises C38V, C68I, S117C, and C127A. In some embodiments, the variant polypeptide further comprises C38S, C68I, S117C, and C127I. In some embodiments, the variant polypeptide further comprises C38I, C68I, C76V, and C127I. In some embodiments, the variant polypeptide further comprises C38I, C68L, and C76Y.

In some embodiments, the IL-18 variant polypeptide comprises an amino acid sequence set forth in any one of SEQ ID NOs: 1-4, 8-9, 11-12, 17, 21-22, 26-28, 35, 38-39, 41, 43-44, 49-50, 54-56, 58-59, 65-66, 72-74, 81, 91, 94, 99, 104-105, 114, 116-117, 121-125, 131-134, 139-145, 147, and 149-153.

In some embodiments, the IL-18 variant polypeptide comprises an amino acid sequence set forth in any one of SEQ ID NOs: 2-4, 8-9, 11-12, 17, 21, 22, 26-28, 35, 38, 39, 41, 43, 44, 49, 50, 54-56, 58-59, 65-66, 72-74, 81, 91, 94, 99, 104-105, 114, 116-117, 121-125, 131-134, 139-145, 147, and 149-150.

In some embodiments, the IL-18 variant polypeptide comprises an amino acid sequence set forth in any one of 3, 9, 17, 27, 39, 43, 49, 94, 105, 116-117, 133, and 143-150.

In some embodiments, the IL-18 variant polypeptide comprises (or further comprises) at least one, at least two, at least three, at least four, at least five, or six mutation at residues selected from the group consisting of C38, C68, C76, D98, S117, and C127, wherein the amino acid position is relative to a wild-type human IL-18 set forth in SEQ ID NO: 1. In some embodiments, the IL-18 variant polypeptide comprises (or further comprises) a substitution at C38 (i.e., wherein the wild-type cysteine at position 38 is substituted with any amino acid), a substitution at C68 (i.e., wherein the wild-type cysteine at position 68 is substituted with any amino acid), and an S117C substitution. In some embodiments, the IL-18 variant polypeptide comprises (or further comprises) a C38I, C38V, C38L, C38M or C38S mutation. Additionally or alternatively, in some embodiments, the IL-18 variant polypeptide comprises (or further comprises) a C68S, C68I, C68D, C68V or C68L mutation. Additionally or alternatively, in some embodiments, the IL-18 variant polypeptide comprises (or further comprises) a C76S, C76V, or C76Y mutation. Additionally or alternatively, in some embodiments, the IL-18 variant polypeptide comprises (or further comprises) an S117C mutation. Additionally or alternatively, in some embodiments, the IL-18 variant polypeptide comprises (or further comprises) a C127A, C127Y, C127F, C127L or C127I mutation. In some embodiments, the IL-18 variant polypeptide comprises (or further comprises) at least one, at least two, at least three, or four mutations selected from the group consisting of C38S, C68S. C76S, and C127S, wherein the amino acid position is relative to a wild-type human IL-18 set forth in SEQ ID NO: 1. In some embodiments, the IL-18 variant polypeptide comprises (or further comprises) C38S, C68S, and C76S mutations. In some embodiments, the IL-18 variant polypeptide comprises (or further comprises or consists of) C38I, C68S, and S117C mutations. In some embodiments, the IL-18 variant polypeptide comprises (or further comprises or consists of) C38V, C68I, S117C, and C127A mutations. In some embodiments, the IL-18 variant polypeptide comprises (or further comprises or consists of) C38S, C68I, S117C, and C127I mutations. In some embodiments, the IL-18 variant polypeptide comprises (or further comprises or consists of) C38I, C68I, C76V, and C127I mutations. In some embodiments, the IL-18 variant polypeptide comprises (or further comprises or consists of) C38I, C68L, and C76Y mutations.

In some embodiments, the IL-18 variant polypeptide further comprises mutation(s) at one or more of Q56, P57, and R104. In some embodiments, the IL-18 variant polypeptide further comprises mutations at two or more of Q56, P57, and R104. In some embodiments, the IL-18 variant polypeptide further comprises mutations at Q56, P57, and R104. In some embodiments, the IL-18 variant polypeptide does not comprise mutations at positions other than G3, E6, D54, Q56, P57, N91, and/or R104. In some embodiments, the IL-18 variant comprises (or further comprises) mutations at positions other than G3, E6, D54, Q56, P57, N91, and/or R104. In some embodiments, the IL-18 variant polypeptide comprises one or more mutations (such as any of 2, 3, 4, 5, 6, 7, 8, 9, 10, 11, 12, 13, 14, or 15 mutations), wherein at least one, at least 2, at least 3, at least 4, at least 5, at least 6, or at least 7 mutations are selected from the group consisting of: G3, E6, D54, Q56, P57, N91, and R104. In some embodiments, the IL-18 variant polypeptide does not comprise substitutions at Q56 and P57, wherein the amino acid positions are relative to SEQ ID NO: 1. In some embodiments, the wild-type IL-18 is human IL-18 comprising the amino acid sequence of SEQ ID NO:1.

In some embodiments, the IL-18 variant polypeptide specifically binds to IL-18 receptor α (“IL-18Rα”), e.g., human IL-18Rα or “hIL-18a,” and exhibits substantially reduced binding to IL-18 binding protein (“IL-18BP”), e.g., human IL-18 or “hIL-18BP.” In some embodiments, the IL-18 variant polypeptide exhibits substantially reduced binding to IL-18BP, relative to the WT human IL-18 set forth in SEQ ID NO: 1. In some embodiments, the affinity of the IL-18 variant polypeptide for IL-18Rα (e.g., hIL-18Rα) is comparable to the affinity of WT human IL-18 set forth in SEQ ID NO: 1 for IL-18Rα (e.g., hIL-18Rα). In some embodiments, the affinity of the IL-18 variant polypeptide for IL-18Rα (e.g., hIL-18Rα) is increased as compared to the affinity of WT human IL-18 set forth in SEQ ID NO: 1 for IL-18Rα (e.g., hIL-18Rα).

In some embodiments, the IL-18 variant polypeptide binds to IL-18Rα with a K_{D} of less than about 5×10⁻⁵ M, less than about 5×10⁻⁶ M, less than about 5×10⁻⁷ M, less than about 5×10⁻⁸ M, less than about 5×10⁻⁹ M, less than about 5×10⁻¹⁰ M, or less than about 5×10⁻¹¹ M, including any range in between these values. In some embodiments, the IL-18 variant polypeptide binds to IL-18Rα with a K_{D} of between about 5×10⁻⁵ and about 5×10⁻¹¹ M. In some embodiments, the IL-18 variant polypeptide binds to IL-18Rα with a K_{D} of between about 5×10⁻⁷ and about 5×10⁻¹¹ M. In some embodiments, the IL-18 variant polypeptide binds to IL-18Rα with a K_{D} of between about 5×10⁻⁷ and about 5×10⁻¹⁰ M. In some embodiments, the IL-18 variant polypeptide binds to IL-18Rα with a K_{D} of between about 5×10⁻⁷ and about 5×10⁻⁹ M. In some embodiments, the IL-18 variant polypeptide binds to IL-18Rα with a K_{D} of between about 5×10⁻⁸ and about 5×10⁻¹¹ M. In some embodiments, the IL-18 variant polypeptide binds to IL-18Rα with a K_{D} of between about 5×10⁻⁹ and about 5×10⁻¹¹ M. In some embodiments, the IL-18 variant polypeptide binds to IL-18Rα with a K_{D} of between about 5×10⁻⁸ and about 5×10⁻¹⁰ M. In some embodiments, the IL-18 variant polypeptide binds to IL-18Rα with a K_{D} of between about 5×10⁻⁷ and about 5×10⁻¹⁰ M. In some embodiments, the IL-18 variant polypeptide binds to IL-18Rα with a K_{D} of between about 5×10⁻⁹ and about 5×10⁻¹⁰ M. In some embodiments, the IL-18 variant polypeptide binds to IL-18Rα with a K_{D} of between about 5×10⁻⁷ and about 5×10⁻⁹ M. In some embodiments, the IL-18 variant polypeptide binds to IL-18Rα with a K_{D} of between about 5×10⁻⁸ and about 5×10⁻⁹ M. In some embodiments, the IL-18 variant binds IL-18Rα (e.g., hIL-18Rα) with a K_{D} between about 5×10⁻⁵ and 5×10⁻¹¹ M, e.g., about any one of 5×10⁻⁵, 6×10⁻⁵, 7×10⁻⁵, 8×10⁻⁵, 9×10⁻⁵, 1×10⁻⁶, 2×10⁻⁶, 3×10⁻⁶, 4×10⁻⁶, 5×10⁻⁶, 6×10⁻⁶, 7×10⁻⁶, 8×10⁻⁶, 9×10⁻⁶, 1×10⁻⁷, 2×10⁻⁷, 3×10⁻⁷, 4×10⁻⁷, 5×10⁻⁷, 6×10⁻⁷, 7×10⁻⁷, 8×10⁻⁷, 9×10⁻⁷, 1×10⁻⁸, 2×10⁻⁸, 3×10⁻⁸, 4×10⁻⁸, 5×10⁻⁸, 6×10⁻⁸, 7×10⁻⁸, 8×10⁻⁸, 9×10⁻⁸, 1×10⁻⁹, 2×10⁻⁹, 3×10⁻⁹, 4×10⁻⁹, 5×10⁻⁹, 6×10⁻⁹, 7×10⁻⁹, 8×10⁻⁹, 9×10⁻⁹, 1×10⁻¹⁰, 2×10⁻¹⁰, 3×10⁻¹⁰, 4×10⁻¹⁰, 5×10⁻¹⁰, 6×10⁻¹⁰, 7×10⁻¹⁰, 8×10⁻¹⁰, 9×10⁻¹⁰, 1×10⁻¹¹, 2×10⁻¹¹, 3×10⁻¹¹, 4×10⁻¹¹, or 5×10⁻¹¹ M, including any range in between these values. In some embodiments, the affinity of the IL-18 variant polypeptide for IL-18Rα (e.g., hIL-18Rα) is higher than the affinity of the wild-type human IL-18 set forth in SEQ ID NO: 1 for IL-18Rα (e.g., hIL-18Rα). In some embodiments, the affinity of the IL-18 variant polypeptide for IL-18Rα (e.g., hIL-18Rα) is comparable to (e.g., about the same as) the affinity of the wild-type human IL-18 set forth in SEQ ID NO: 1 for IL-18Rα (e.g., hIL-18Rα).

In some embodiments, the IL-18 variant polypeptide exhibits substantially reduced binding to IL-18 binding protein (IL-18BP) relative to the wild-type IL-18. In some embodiments, the IL-18 variant polypeptide binds to IL-18BP with a K_{D} of more than 5×10⁻⁹ M. In some embodiments, the IL-18 variant polypeptide binds to IL-18BP with a K_{D} of more than 5×10⁻⁸ M. In some embodiments, the IL-18 variant polypeptide binds to IL-18BP with a K_{D} of more than 5×10⁻⁷ M. In some embodiments, the IL-18 variant polypeptide binds to IL-18BP with a K_{D} of more than 5×10⁻⁶ M. In some embodiments, the IL-18 variant polypeptide binds to IL-18BP with a K_{D} of more than 5×10⁻⁵ M. In some embodiments, the IL-18 variant polypeptide exhibits no binding (such as no detectable binding) to IL-18BP (e.g., hIL-18BP). In some embodiments, an IL-18 variant polypeptide that exhibits no binding (e.g., no detectable binding) to IL-18BP (e.g., hIL-18BP) binds to IL-18BP (e.g., hIL-18BP) with a K_{D} greater than 10⁻³ M.

The affinities of the IL-18 described herein for IL-18Rα and/or IL-18BP can be determined experimentally by methods known in the art. Such methods include, but are not limited to, e.g., Western blots, enzyme-linked immunosorbent assay (ELISA), radioimmunoassay (RIA), electrochemiluminescence (ECL) assay, immunoradiometric (IRMA) assay, enzyme immunoassay (EIA), surface plasmon resonance (SPR), peptide scans, and fluorescence activated cell sorting (FACS)-based kinetic competition screening.

In some embodiments, the IL-18 variant polypeptide comprises a mutation at residue G3, wherein the mutation is selected from the group consisting of G3P, G3D, G3E, G3N, and G3S. As used herein, “G3X” means that G, i.e., the wild-type amino acid at position 3 of SEQ ID NO: 1, has been replaced with amino acid X. In some embodiments, the mutation is G3P, i.e., the wild type G at position 3 of SEQ ID NO: 1 has been replaced with amino acid P.

In some embodiments, the variant polypeptide comprises (such as further comprises) a mutation at residue E6, wherein the mutation is selected from the group consisting of E6R, E6K, E6G, E6T, E6A, E6S, E6E, E6L, E6M, and E6N. In some embodiments, the mutation is selected from the group consisting of E6R, E6K, E6G, E6T, E6A, and E6S. In some embodiments, the mutation is selected from the group consisting of E6R and E6K.

In some embodiments, the variant polypeptide comprises (such as further comprises) a mutation at residue D54, wherein the mutation is selected from the group consisting of D54W, D54H, D54S, D54Q, D54L, D54Y, D54P, D54A, D54F, D54G, and D54T. In some embodiments, the mutation is selected from the group consisting of D54W, D54H, D54S, D54Q, D54L, and D54Y.

In some embodiments, the variant polypeptide comprises (such as further comprises) a mutation at residue N91, wherein the mutation is selected from the group consisting of N91V, N91A, N91G, N91S, N91I, N91P, N91R, N91T, N91C, N91K, and N91W. In some embodiments, the mutation is selected from the group consisting of N91V, N91A, N91G, and N91S.

In some embodiments, the variant polypeptide further comprises a mutation at residue R104, wherein the mutation is selected from the group consisting of R104S, R104Y, R104T, R104L, R104V, R104A, R104F, R104H, R104I, and R104N. In some embodiments, the mutation is selected from the group consisting of R104S, R104Y, and R104T. In some embodiments, the IL-18 variant polypeptide comprises no mutation at residue R104.

In some embodiments, wherein the IL-18 variant polypeptide comprises (such as further comprises) a mutation at residue Q56, the mutation is selected from the group consisting of Q56T, Q56G, Q56R, Q56S, Q56D, Q56P, Q56I, Q56Y, Q56H, Q56L, Q56E, Q56F, Q56N, and Q56V. In some embodiments, the mutation is selected from the group consisting of Q56T, Q56G, Q56R, Q56S, Q56D, Q56P, Q56I, and Q56Y.

In some embodiments, wherein the IL-18 variant polypeptide comprises (such as further comprises) a mutation at residue P57, the mutation is selected from the group consisting of P57A, P57G, P57R, P57W, P57S, P57T, P57V, P57Q, P57H, P57K, P57N, P57Y, and P57D. In some embodiments, the mutation is selected from the group consisting of P57A, P57G, P57R, P57W, P57S, P57T, and P57V.

In some embodiments, the IL-18 variant polypeptide comprises mutations at one or more of G3, E6, D54, and N91. In some embodiments, the IL-18 variant polypeptide comprises mutations at two or more of G3, E6. D54, and N91. In some embodiments, the IL-18 variant polypeptide comprises mutations at three or more of G3, E6, D54, and N91. In some embodiments, the IL-18 variant polypeptide comprises mutations at G3, E6, D54, and N91. In some embodiments, wherein the IL-18 variant polypeptide comprises a G3P mutation. Additionally or alternatively, in some embodiments, the IL-18 variant polypeptide comprises a E6R or E6K mutation. Additionally or alternatively, in some embodiments, the IL-18 variant polypeptide comprises a D54W, D54H, D54S, or D54Q mutation. Additionally or alternatively, in some embodiments, the IL-18 variant polypeptide comprises a N91V, N91A, N91G, or N91S mutation. In some embodiments, the IL-18 variant polypeptide does not comprise mutations at positions other than G3, E6, D54, and/or N91. In some embodiments, the IL-18 variant further comprises mutations at positions other than at G3, E6, D54, and/or N91. In some embodiments, the IL-18 variant polypeptide comprises one or more mutations (such as any of 2, 3, 4, 5, 6, 7, 8, 9, 10, 11, 12, 13, 14, or 15 mutations), wherein at least one, at least 2, at least 3, or at least 4 mutations are selected from the group consisting of: G3, E6, D54, and N91. In some embodiments, the IL-18 variant polypeptide further comprises mutations at Q56 and/or P57. In some embodiments, the IL-18 variant polypeptide does not comprise mutations at positions other than G3, E6, D54, N91, Q56 and/or P57. In some embodiments, the IL-18 variant polypeptide further comprises mutations at positions other than at G3, E6, D54, N91, Q56 and/or P57. In some embodiments, the IL-18 variant polypeptide comprises one or more mutations (such as any of 2, 3, 4, 5, 6, 7, 8, 9, 10, 11, 12, 13, 14, or 15 mutations), wherein at least one, at least 2, at least 3, at least 4 mutations, at least 5 mutations, or at least 6 mutations are selected from the group consisting of: G3, E6, D54, N91, Q56, and P57.

In some embodiments, the IL-18 variant polypeptide comprises an amino acid sequence having at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity with the sequence of any one of SEQ ID NOs: 2-299, 307-318. In some embodiments, the IL-18 variant polypeptide comprises an amino acid sequence set forth in any one of SEQ ID NOs: 2-299, 307-318. The amino acid sequences of SEQ ID NOs: 2-299, 307-318 are provided in the Sequence Summary Table below the Examples.

### Fusion Polypeptides

In some embodiments, an activatable IL-18 polypeptide of the present disclosure comprises a fusion polypeptide comprising wild-type IL-18 or an IL-18 variant polypeptide described herein. In some embodiments, the fusion polypeptide comprises (1) a wild-type IL-18 or an IL-18 variant polypeptide and (2) a dimerization domain. In some embodiments, the fusion polypeptide comprises (1) two or more wild-type IL-18s, two or more IL-18 variant polypeptides, or two or more of a wild-type IL-18 and an IL-18 variant polypeptide in any combination, and (2) a dimerization domain. In some embodiments, the dimerization domain is or comprises a leucine zipper (LZ) element. Leucine zippers have been identified, generally, as stretches of about 35 amino acids containing 4-5 leucine residues separated from each other by six amino acids (Maniatis and Abel (1989) Nature 341:24-25). Exemplary leucine zippers occur in a variety of eukaryotic DNA binding proteins, such as GCN4, C/EBP, c-Fos, c-Jun, c-Myc and c-Max. In some embodiments, the dimerization domain is or comprises a helix-loop-helix domain (Murre, C. et al. (1989) Cell 58:537-544). Dimerization domains may also be selected from other proteins, such as the retinoic acid receptor, the thyroid hormone receptor or other nuclear hormone receptors (Kurokawa et al. (1993) Genes Dev. 7:1423-1435) or from the yeast transcription factors GAL4 and HAP1 (Marmonstein et al. (1992) Nature 356:408-414; Zhang et al. (1993) Proc. Natl. Acad. Sci. USA 90:2851-2855). Dimerization domains are further described in U.S. Pat. No. 5,624,818 by Eisenman. In some embodiments, the dimerization domain is an antibody Fc domain.

In some embodiments, the fusion polypeptide comprises a wild-type IL-18 (e.g., the wild-type IL-18 who amino acid sequence is set forth in SEQ ID NO: 1) and an antibody Fc domain. In some embodiments, the fusion polypeptide comprises a IL-18 variant polypeptide (e.g., an IL-18 variant polypeptide described herein) and an antibody Fc domain. In some embodiments the C-terminus of the wild-type IL-18 or the C-terminus of the IL-18 variant polypeptide is fused to the N-terminus of the antibody Fc domain. In some embodiments the C-terminus of the antibody Fc domain is fused to the N-terminus of the wild-type IL-18 or the N-terminus of the IL-18 variant polypeptide. In some embodiments, the Fc domain is a human Fc domain or a variant thereof comprising one or more amino acid substitutions. In some embodiments, the Fc domain is an human IgG Fc domain or variant thereof, e.g., a human IgG1, IgG2, or IgG4 Fc domain or a variant of any of the preceding. In some embodiments, the fusion polypeptide comprises, N-terminus-to-C-terminus, an IL-18 variant polypeptide described herein and a human IgG1 Fc variant that comprises an N297A mutation, wherein the amino acid numbering is according to the EU numbering system, also called the EU index, as described in Kabat et al., Sequences of Proteins of Immunological Interest, 5th Ed. Public Health Service, National Institutes of Health, Bethesda, MD. 1991. In some embodiments, the fusion polypeptide comprises, N-terminus-to-C-terminus, a wild-type IL-18 and a human IgG1 Fc variant that comprises an N297A mutation. In some embodiments, the fusion polypeptide comprises the amino acid sequence of any one of SEQ ID NOs: 319-332 and 336-347.

### Nucleic Acids, Vectors, Host Cell, and Methods of Producing IL-18 Variant Polypeptides

Nucleic acid molecules encoding the IL-18 variant polypeptides described herein or fusion polypeptides described herein are also contemplated. In some embodiments, provided is a nucleic acid encoding an IL-18 variant polypeptide or a fusion polypeptide described herein. In some embodiments, the nucleic acid comprises a polynucleotide sequence having at least 70%, at least 75%, at least 80%, at least 85%, at least 90%, at least 95%, at least 96%, at least 97%, at least 98%, or at least 99% identity with a sequence selected from the group consisting of SEQ ID NO 301-306 and 333-335. In some embodiments, the nucleic acid comprises a polynucleotide sequence selected from the group consisting of SEQ ID NOs: 301-306 and 333-355. The nucleic acid sequences of SEQ ID NOs: 301-306 and 333-335 are provided in the Sequence Summary Table below the Examples.

Also provided are vectors in which a nucleic acid described herein is inserted.

In brief summary, the expression of an IL-18 variant polypeptide or a fusion polypeptide described herein by a natural or synthetic nucleic acid encoding the IL-18 variant polypeptide or fusion polypeptide can be achieved by inserting the nucleic acid into an appropriate expression vector, such that the nucleic acid is operably linked to 5′ and 3′ regulatory elements, including for example a promoter (e.g., a constitutive, regulatable, tissue-specific promoter) and a 3′ untranslated region (UTR). The vectors can be suitable for replication and integration in eukaryotic host cells. Typical cloning and expression vectors contain transcription and translation terminators, initiation sequences, and promoters useful for regulation of the expression of the desired nucleic acid sequence.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers (see, e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In some embodiments, lentivirus vectors are used. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity.

Additional promoter elements, e.g., enhancers, regulate the frequency of transcriptional initiation. Typically, these are located in the region 30-110 base pairs (bp) upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In some embodiments, the expression of the nucleic acid(s) encoding the IL-18 variant polypeptide or fusion polypeptide is inducible. In some embodiments, the nucleic acid(s) encoding the IL-18 variant polypeptide or fusion polypeptide is operably linked to an inducible promoter, including any inducible promoter known in the art. In some embodiments, the nucleic acid(s) encoding an IL-18 variant polypeptide or fusion polypeptide described herein has been engineered to encode an epitope tag, e.g., to facilitate purification or detection of the polypeptide. Exemplary epitope tags include, but are not limited to, e.g., 6×His (also known as His-tag or hexahistidine tag), FLAG, HA, Myc, V5, GFP (green fluorescent protein, e.g., enhanced green fluorescent protein or EGFP), SUMO (Small ubiquitin-like modifier), GST (glutathione-S-transferase), β-GAL (β-galactosidase), Luciferase, MBP (Maltose Binding Protein), RFP (Red Fluorescence Protein), and VSV-G (Vesicular Stomatitis Virus Glycoprotein).

An IL-18 variant polypeptide or fusion polypeptide of the present disclosure may be produced by any means known in the art. Exemplary techniques for polypeptide production are described below; however, these exemplary techniques are provided for illustrative purposes only and are not intended to be limiting.

An IL-18 variant polypeptide or fusion polypeptide described herein can be produced using recombinant methods. For recombinant production of an IL-18 variant polypeptide or fusion polypeptide, a nucleic acid encoding the IL-18 variant polypeptide or fusion polypeptide is isolated and inserted into a replicable vector for further cloning (amplification of the DNA) or for expression. DNA encoding the IL-18 variant polypeptide or fusion polypeptide may be readily isolated and sequenced using conventional procedures (e.g., by using oligonucleotide probes that are capable of binding specifically to genes encoding the IL-18 variant polypeptide or fusion polypeptide). Many vectors are available. The vector components generally include, but are not limited to, one or more of the following: a signal sequence, an origin of replication, one or more marker genes, an enhancer element, a promoter, and a transcription termination sequence.

Both expression and cloning vectors contain a nucleic acid sequence that enables the vector to replicate in one or more selected host cells, e.g., to allow the vector to replicate independently of the host chromosomal DNA. This sequence can include origins of replication or autonomously replicating sequences. Such sequences are well known for a variety of bacteria, yeast, and viruses. Generally, the origin of replication component is not needed for mammalian expression vectors (the SV40 origin may be used because it contains the early promoter).

Expression and cloning vectors can contain a selection gene or selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media. Examples of dominant selection use the drugs neomycin, mycophenolic acid and hygromycin. Another example of suitable selectable markers for mammalian cells are those that enable the identification of cells competent to take up IL-18 variant polypeptide- or fusion polypeptide-encoding nucleic acid, such as DHFR, glutamine synthetase (GS), thymidine kinase, metallothionein-I and -II, preferably primate metallothionein genes, adenosine deaminase, ornithine decarboxylase, and the like. For example, a Chinese hamster ovary (CHO) cell line deficient in endogenous DHFR activity transformed with the DHFR gene is identified by culturing the transformants in a culture medium containing methotrexate (Mtx), a competitive antagonist of DHFR.

Alternatively, host cells (particularly wild-type hosts that contain endogenous DHFR) transformed or co-transformed with DNA sequences encoding an IL-18 variant polypeptide or fusion polypeptide of interest, wild-type DHFR gene, and another selectable marker such as aminoglycoside 3′-phosphotransferase (APH) can be selected by cell growth in medium containing a selection agent for the selectable marker such as an aminoglycosidic antibiotic, e.g., kanamycin, neomycin, or G418.

Expression and cloning vectors generally contain a promoter that is recognized by the host organism and is operably linked to nucleic acid encoding an IL-18 variant polypeptide or fusion polypeptide. Promoters suitable for use with prokaryotic hosts include the phoA promoter, β-lactamase and lactose promoter systems, alkaline phosphatase promoter, a tryptophan (trp) promoter system, and hybrid promoters such as the tac promoter. However, other known bacterial promoters are suitable. Promoter sequences are known for eukaryotes. Yeast promoters are well known in the art and can include inducible promoters/enhancers regulated by growth conditions. Virtually all eukaryotic genes have an AT-rich region located approximately 25 to 30 bases upstream from the site where transcription is initiated. Examples include without limitation the promoters for 3-phosphoglycerate kinase or other glycolytic enzymes, such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase. The transcription of an IL-18 variant polypeptide or fusion polypeptide from vectors in mammalian host cells can be controlled, for example, by promoters obtained from the genomes of viruses. The early and late promoters of the SV40 virus are conveniently obtained as an SV40 restriction fragment that also contains the SV40 viral origin of replication. The immediate early promoter of the human cytomegalovirus is conveniently obtained as a HindIII restriction fragment. Alternatively, the Rous Sarcoma Virus long terminal repeat can be used as the promoter.

Transcription of a DNA encoding an IL-18 variant polypeptide or fusion polypeptide described herein by higher eukaryotes is often increased by inserting an enhancer sequence into the vector. Many enhancer sequences are now known from mammalian genes (globin, elastase, albumin, α-fetoprotein, and insulin). Typically, however, one will use an enhancer from a eukaryotic cell virus.

Expression vectors used in eukaryotic host cells (yeast, fungi, insect, plant, animal, human, or nucleated cells from other multicellular organisms) will also contain sequences necessary for the termination of transcription and for stabilizing the mRNA.

Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, etc. In addition to prokaryotes, eukaryotic microbes such as filamentous fungi or yeast are suitable cloning or expression hosts for IL-18 polypeptide variant- or fusion polypeptide-encoding vectors. Saccharomyces cerevisiae, or common baker's yeast, is the most commonly used among lower eukaryotic host microorganisms. Certain fungi and yeast strains may be selected in which glycosylation pathways have been “humanized,” resulting in the production of an IL-18 variant polypeptide or fusion polypeptide with a partially or fully human glycosylation pattern. See, e.g., Li et al., Nat. Biotech. 24:210-215 (2006).

Plant cell cultures of cotton, corn, potato, soybean, petunia, tomato, duckweed (Leninaceae), alfalfa (M. truncatula), and tobacco can also be utilized as hosts.

Suitable host cells for the expression of a glycosylated IL-18 variant polypeptide or fusion polypeptide are also derived from multicellular organisms (invertebrates and vertebrates). Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified.

Vertebrate cells may be used as hosts, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2). Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as NS0 and Sp2/0. For a review of certain mammalian host cell lines suitable for production, see, e.g., Yazaki and Wu, Methods in Molecular Biology, Vol. 248 (B. K. C. Lo, ed., Humana Press, Totowa, N.J., 2003), pp. 255-268.

The host cells of the present disclosure may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to one of skill in the art.

When using recombinant techniques, the IL-18 variant polypeptide or fusion polypeptide can be produced intracellularly, in the periplasmic space, or directly secreted into the medium. If the polypeptide is produced intracellularly, as a first step, the particulate debris, either host cells or lysed fragments, are removed, for example, by centrifugation or ultrafiltration. Carter et al., Bio/Technology 10:163-167 (1992) describe a procedure for isolating antibodies which are secreted to the periplasmic space of E. coli.

The polypeptide composition prepared from the cells can be purified using, for example, hydroxyapatite chromatography, hydrophobic interaction chromatography, gel electrophoresis, dialysis, and affinity chromatography, with affinity chromatography being among one of the typically preferred purification steps. In some embodiments, an IL-18 variant polypeptide or fusion polypeptide described herein comprises an epitope tag (e.g., a tag attached to the IL-18 variant polypeptide or fusion polypeptide via a cleavable linker) to facilitate purification. Exemplary epitope tags include, but are not limited to, e.g., 6×His (also known as His-tag or hexahistidine tag), FLAG, HA, Myc, V5, GFP (green fluorescent protein, e.g., enhanced green fluorescent protein or EGFP), SUMO (Small ubiquitin-like modifier), GST (glutathione-S-transferase), β-GAL (β-galactosidase), Luciferase, MBP (Maltose Binding Protein), RFP (Red Fluorescence Protein), and VSV-G (Vesicular Stomatitis Virus Glycoprotein.

Methods of Treating Disease, Activating hIL-18 Receptor-Mediated Signaling, and Stimulating Antigen-Experienced T-Cells or NK Cells

Also provided here are methods of treating a disease or condition in an individual. The methods comprise administering an IL-18 variant polypeptide or fusion polypeptide described herein, a nucleic acid described herein, a vector described herein, and/or a pharmaceutical composition described herein to an individual having the disease or condition. In some embodiments, the disease or condition is a proliferative disorder. In some embodiments the proliferative disorder is cancer.

In some embodiments, provided are methods of activating hIL-18 receptor mediated signal in an individual, which methods comprise administering an IL-18 variant polypeptide or fusion polypeptide described herein, a nucleic acid described herein, a vector described herein, and/or a pharmaceutical composition described herein to the individual. In some embodiments, provided herein are methods of stimulating antigen-experienced T cells or NK cells in an individual, which methods comprise administering an IL-18 variant polypeptide or fusion polypeptide described herein, a nucleic acid described herein, a vector described herein, and/or a pharmaceutical composition described herein to the individual. In some embodiments of any of the methods herein, the individual is a mammal (e.g., human, non-human primate, rat, mouse, cow, horse, pig, sheep, goat, dog, cat, etc.). In some embodiments, the individual is a human. In some embodiments, the individual is a clinical patient, a clinical trial volunteer, an experimental animal, etc.

### Compositions, Kits and Articles of Manufacture

Also provided herein are compositions (such as formulations) comprising an IL-18 variant polypeptide, a fusion polypeptide, a nucleic acid, a vector, or a host cell described herein.

Suitable composition be obtained by mixing the IL-18 variant polypeptide, the fusion polypeptide, the nucleic acid, the vector, or the host cell having the desired degree of purity with optional pharmaceutically acceptable carriers, excipients or stabilizers (Remington's Pharmaceutical Sciences 16th edition, Osol, A. Ed. (1980)).

Also provided are kits comprising an IL-18 variant polypeptide, a fusion polypeptide, a nucleic acid, a vector, or a host cell comprising a nucleic acid or vector described herein. The kits may be useful for any of the methods of treatment described herein.

The kits of the present application are in suitable packaging. Suitable packaging includes, but is not limited to, vials, bottles, jars, flexible packaging (e.g., sealed Mylar or plastic bags), and the like. Kits may optionally provide additional components such as buffers and interpretative information.

The present application thus also provides articles of manufacture. The article of manufacture can comprise a container and a label or package insert on or associated with the container. Suitable containers include vials (such as sealed vials), bottles, jars, flexible packaging, and the like. Generally, the container holds a composition, and may have a sterile access port (for example the container may be an intravenous solution bag or a vial having a stopper pierceable by a hypodermic injection needle).

Those skilled in the art will recognize that several embodiments are possible within the scope and spirit of this invention. The invention will now be described in greater detail by reference to the following non-limiting examples. The following examples further illustrate the invention but, of course, should not be construed as in any way limiting its scope.

### EXEMPLARY EMBODIMENTS

Embodiment 1. An interleukin 18 (IL-18) variant polypeptide comprising at least one mutation at a residue selected from the group consisting of: F2, G3, E6, V11, Q24, L29, D54, A61, N91, K96, R107, K140, and I149, wherein the amino acid position is relative to a wild-type human IL-18 set forth in SEQ ID NO: 1.

Embodiment 2. The IL-18 variant polypeptide of embodiment 1, comprising at least one mutation at a residue selected from the group consisting of: G3, E6, D54, and N91, wherein the amino acid positions is relative to a wild-type human IL-18 set forth in SEQ ID NO: 1.

Embodiment 3. The IL-18 variant polypeptide of embodiment 1 or 2, further comprising at least one mutation at a residue selected from the group consisting of Q56, P57, M60, Q103, R104, M113, and N155, wherein the amino acid position is relative to a wild-type human IL-18 set forth in SEQ ID NO: 1.

Embodiment 4. The IL-18 variant polypeptide of any one of embodiments 1-3, further comprising at least one mutation at a residue selected from the group consisting of: C38, C68, C76, D98, S117, and C127, wherein the amino acid position is relative to a wild-type human IL-18 set forth in SEQ ID NO: 1.

Embodiment 5. The IL-18 variant polypeptide of embodiment 4, wherein the at least one mutation comprises a substitution at C38, a substitution at C68, and an S117C substitution.

Embodiment 6. The IL-18 variant polypeptide of embodiment 4, wherein the at least one mutation is selected from the group consisting of: C38S, C68S, C76S, and C127S.

Embodiment 7. The IL-18 variant polypeptide of embodiment 4, wherein the variant polypeptide further comprises C38S, C68S, and C76S mutations.

Embodiment 8. The IL-18 variant polypeptide of embodiment 4, wherein the variant polypeptide further comprises a set of mutations selected from the group consisting of: (a) C38I, C68S, and S117C; (b) C38V, C68I, S117C, and C127A; (c) C38S, C68I, S117C, and C127I; (d) C38I, C68I, C76V, and C127I; and (e) C38I, C68L, and C76Y.

Embodiment 9. An interleukin 18 (IL-18) variant polypeptide comprising at least one mutation at a residue selected from the group consisting of: C38, C68, C76, D98, S117, and C127, wherein the amino acid position is relative to a wild-type human IL-18 set forth in SEQ ID NO: 1.

Embodiment 10. The IL-18 variant polypeptide of embodiment 9, wherein the at least one mutation comprises a substitution at C38, a substitution at C68, and an S117C substitution.

Embodiment 11. The IL-18 variant polypeptide of embodiment 9, wherein the at least one mutation is selected from the group consisting of: C38S, C68S, C76S, and C127S.

Embodiment 12. The IL-18 variant polypeptide of embodiment 10, wherein the variant polypeptide comprises C38S, C68S, and C76S mutations.

Embodiment 13. The IL-18 variant polypeptide of embodiment 9, wherein the variant polypeptide comprises a set of mutations selected from the group consisting of: (a) C38I, C68S, and S117C; (b) C38V, C68I, S117C, and C127A; (c) C38S, C68I, S117C, and C127I; (d) C38I, C68I, C76V, and C127I; and (e) C38I, C68L, and C76Y.

Embodiment 14. The IL-18 variant polypeptide of any one of embodiments 1-13, wherein the wild-type IL-18 is human IL-18 comprising the amino acid sequence of SEQ ID NO:1.

Embodiment 15. The IL-18 variant polypeptide of any one of embodiments 1-14, wherein the IL-18 variant polypeptide specifically binds to IL-18 receptor α (IL-18Rα) and exhibits substantially reduced binding to IL-18 binding protein (IL-18BP) relative to the wild-type IL-18.

Embodiment 16. The IL-18 variant polypeptide of any one of embodiments 1-15, wherein the IL-18 variant polypeptide exhibits an increased binding to IL-18Rα relative to the wild-type IL-18.

Embodiment 17. The IL-18 variant polypeptide of any one of embodiments 1-16, wherein the IL-18 variant polypeptide binds to IL-18Rα with a K_{D} of less than about 5×10⁻⁵ M.

Embodiment 18. The IL-18 variant polypeptide of any one of embodiments 1-17, wherein the IL-18 variant polypeptide binds to IL-18Rα with a K_{D} of between about 5×10⁻⁵ and about 5×10⁻¹¹ M.

Embodiment 19. The IL-18 variant polypeptide of any one of embodiments 1-18, wherein the IL-18 variant polypeptide binds to IL-18BP with a K_{D} of more than 5×10⁻⁹ M.

Embodiment 20. The IL-18 variant polypeptide of any one of embodiments 1-19, wherein the IL-18 variant polypeptide exhibits no binding to IL-18BP.

Embodiment 21. The IL-18 variant polypeptide of any one of embodiments 1-20, wherein the variant polypeptide comprises a mutation at residue G3, wherein the mutation is selected from the group consisting of: G3P, G3D, G3E, G3F, G3K, G3T, G3W, G3N, and G3S.

Embodiment 22. The IL-18 variant polypeptide of embodiment 21, wherein the mutation is G3P.

Embodiment 23. The IL-18 variant polypeptide of any one of embodiments 1-22, wherein the variant polypeptide comprises a mutation at residue E6, wherein the mutation is selected from the group consisting of: E6R, E6K, E6G, E6T, E6A, E6S, E6H, E6L, E6M, E6N, E6P, E6Q, E6V, E6W, and E6Y.

Embodiment 24. The IL-18 variant polypeptide of embodiment 23, wherein the mutation is selected from the group consisting of: E6R, E6K, E6G, E6T, E6A, and E6S.

Embodiment 25. The IL-18 variant polypeptide of embodiment 24, wherein the mutation is selected from the group consisting of: E6R and E6K.

Embodiment 26. The IL-18 variant polypeptide of any one of embodiments 1-25, wherein the variant polypeptide comprises a mutation at residue D54, wherein the mutation is selected from the group consisting of: D54W, D54H, D54I, D54S, D54Q, D54L, D54M, D54Y, D54P, D54R, D54A, D54F, D54G, D54V, and D54T.

Embodiment 27. The IL-18 variant polypeptide of embodiment 26, wherein the mutation is selected from the group consisting of: D54W, D54H, D54S, D54Q, D54L, and D54Y.

Embodiment 28. The IL-18 variant polypeptide of any one of embodiments 1-27, wherein the variant polypeptide comprises a mutation at residue N91, wherein the mutation is selected from the group consisting of: N91V, N91A, N91D, N91F, N91G, N91S, N91I, N91P, N91R, N91L, N91T, N91C, N91K, N91Y and N91W.

Embodiment 29. The IL-18 variant polypeptide of embodiment 28, wherein the mutation is selected from the group consisting of: N91V, N91A, N91G, and N91S.

Embodiment 30. The IL-18 variant polypeptide of any one of embodiments 1-29, wherein the variant polypeptide further comprises a mutation at residue R104, wherein the mutation is selected from the group consisting of: R104S, R104Y, R104T, R104L, R104M, R104V, R104A, R104C, R104E, R104G, R104F, R104H, R104I, and R104N.

Embodiment 31. The IL-18 variant polypeptide of embodiment 30, wherein the mutation is selected from the group consisting of: R104S, R104Y, and R104T.

Embodiment 32. The IL-18 variant polypeptide of any one of embodiments 1-29, wherein the variant polypeptide comprises no mutation at residue R104.

Embodiment 33. The IL-18 variant polypeptide of any one of embodiments 1-32, wherein the variant polypeptide comprises a mutation at residue Q56, wherein the mutation is selected from the group consisting of: Q56A, Q56T, Q56G, Q56R, Q56S, Q56D, Q56P, Q56I, Q56Y, Q56H, Q56I, Q56K, Q56W, Q56L, Q56E. Q56F, Q56N, and Q56V.

Embodiment 34. The IL-18 variant polypeptide of embodiment 33, wherein the mutation is selected from the group consisting of: Q56T, Q56G, Q56R, Q56S, Q56D, Q56P, Q56I, and Q56Y.

Embodiment 35. The IL-18 variant polypeptide of any one of embodiments 1-32, wherein the variant polypeptide comprises no mutation at residue Q56.

Embodiment 36. The IL-18 variant polypeptide of any one of embodiments 1-35, wherein the variant polypeptide comprises a mutation at residue P57, wherein the mutation is selected from the group consisting of: P57A, P57E, P57F, P57G, P57R, P57W, P57S, P57T, P57V, P57Q, P57H, P57I, P57K, P57L, P57N, P57Y, and P57D.

Embodiment 37. The IL-18 variant polypeptide of embodiment 36, wherein the mutation is selected from the group consisting of: P57A, P57G, P57R, P57W, P57S, P57T, and P57V.

Embodiment 38. The IL-18 variant polypeptide of any one of embodiments 1-35, wherein the variant polypeptide comprises no mutation at residue P57.

Embodiment 39. The IL-18 variant polypeptide of any one of embodiments 1-38, wherein the variant polypeptide comprises mutations at G3, E6, D54, and N91.

Embodiment 40. The IL-18 variant polypeptide of embodiment 39, wherein the variant polypeptide comprises mutations: (i) G3P; (ii) E6R or E6K; (iii) D54W, D54H, D54S, or D54Q; and (iv) N91V, N91A, N91G, or N91S.

Embodiment 41. The IL-18 variant polypeptide of any one of embodiments 1-34, 36-37, and 39-40, wherein the variant polypeptide further comprises mutations at Q56 and P57.

Embodiment 42. The IL-18 variant polypeptide of any one of embodiments 1-41, wherein the variant polypeptide comprises the amino acid sequence set forth in any one of SEQ ID NOs: 2-299 and 307-318.

Embodiment 43. A fusion polypeptide comprising the IL-18 variant polypeptide of any one of embodiments 1-42 and a human IgG Fc domain or variant thereof.

Embodiment 44. The fusion polypeptide of embodiment 43, comprising a human IgG1 Fc domain variant that comprises an N297A mutation (EU numbering).

Embodiment 45. The fusion polypeptide of embodiment 43 or 44, wherein the C-terminus of the IL-18 variant polypeptide is fused to the N-terminus of the human IgG Fc domain or variant thereof.

Embodiment 46. The fusion polypeptide of embodiment 43 or 44, wherein the C-terminus of the human IgG Fc domain or variant thereof is fused to the N-terminus of the IL-18 variant polypeptide.

Embodiment 47. The fusion polypeptide of any one of embodiments 43-46, comprising the amino acid sequence of any one of SEQ ID NOs: 319-332 and 336-347.

Embodiment 48. A dimer comprising two fusion polypeptides of any one of embodiments 43-47.

Embodiment 49. The dimer of embodiment 48, wherein the dimer is a homodimer.

Embodiment 50. The dimer of embodiment 48, wherein the dimer is a heterodimer.

Embodiment 51. A nucleic acid encoding the IL-18 variant polypeptide of any one of embodiments 1-42 or the fusion polypeptide of any one of embodiments 43-47.

Embodiment 52. The nucleic acid of embodiment 51, comprising a polynucleotide sequence having at least 80% identity to any one of SEQ ID NOs: 301-306 and 333-335.

Embodiment 53. A vector comprising the nucleic acid of embodiment 51 or 52.

Embodiment 54. A host cell comprising the nucleic acid of embodiment 51 or 52 or the vector of embodiment 53.

Embodiment 55. A method of producing an IL-18 variant polypeptide or fusion polypeptide, comprising: (a) culturing the host cell of embodiment 54 under conditions where the IL-18 variant polypeptide or fusion polypeptide is expressed; and (b) recovering the IL-18 variant polypeptide or fusion polypeptide produced by the host cell.

Embodiment 56. The method of embodiment 55, further comprising purifying the IL-18 variant polypeptide or the fusion polypeptide.

Embodiment 57. A pharmaceutical composition comprising the IL-18 variant polypeptide of any one of embodiments 1-42, the fusion polypeptide of any one of embodiments 43-47, the nucleic acid of embodiment 51 or 52, or the vector of embodiment 53.

Embodiment 58. A method of treating a disease in an individual, comprising administering to the individual an effective amount of the pharmaceutical composition of embodiment 57.

Embodiment 59. The method of embodiment 58, wherein the disease is cancer.

Embodiment 60. A method of activating hIL-18 receptor mediated signal in an individual, comprising administering to the individual an effective amount of the pharmaceutical composition of embodiment 57.

Embodiment 61. A method of stimulating antigen-experienced immune cells in an individual in need thereof, comprising administering to the individual an effective amount of the pharmaceutical composition of embodiment 57.

Embodiment 62. The method of embodiment 61, wherein the stimulation comprises increasing activity and/or number of the antigen-experienced immune cells.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. The examples below are intended to be purely exemplary of the application and should therefore not be considered to limit the application in any way. The following examples and detailed description are offered by way of illustration and not by way of limitation.

### Example 1: Construction of a Yeast Expression Library of IL-18 Variant Polypeptides that do not Bind IL-18BP

Interleukin 18 (“IL-18”) stimulates T and NK cells by binding to its heterodimer receptor complex, which is composed of IL-18Rα and IL-18Rβ subunits. IL-18 binding protein (“IL-18BP”) is a natural inhibitor of IL-18 activity. IL-18BP has much higher affinity for IL-18 than IL-18Rα, and the binding of IL-18BP to IL-18 blocks IL-18's interaction with IL-18Rα. To develop variants of human IL-18 (“hIL-18”) that do not bind IL-18BP or have reduced binding affinity for IL-18BP, an artificial-intelligence guided molecular design method was utilized to identify amino acid positions in wild-type hIL-18 for mutations. Next, a yeast library displaying hIL-18 variant polypeptides comprising identified mutation(s) relative to wild-type IL-18 (SEQ ID NO: 1) was constructed using NNK mutagenesis, and the final library diversity was ˜2.72×10⁻⁸. A c-myc tag was fused to each of the IL-18 variants for expression testing.

### Example 2: Screening for IL-18 Variant Polypeptides that Bind Human IL-18Rα

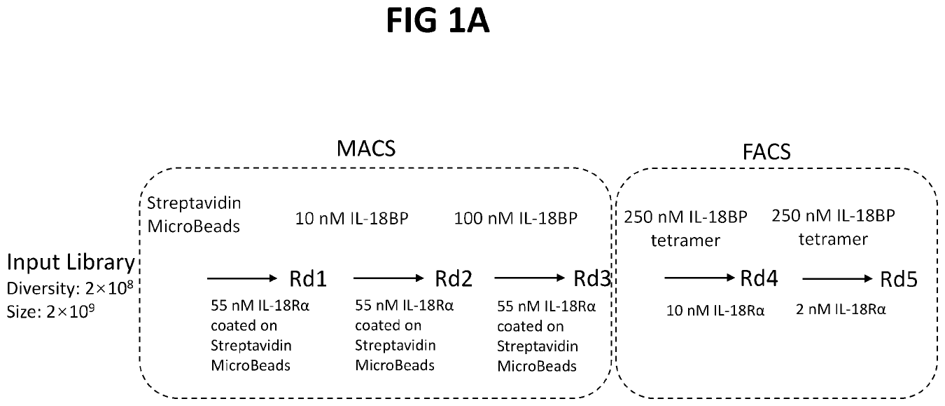

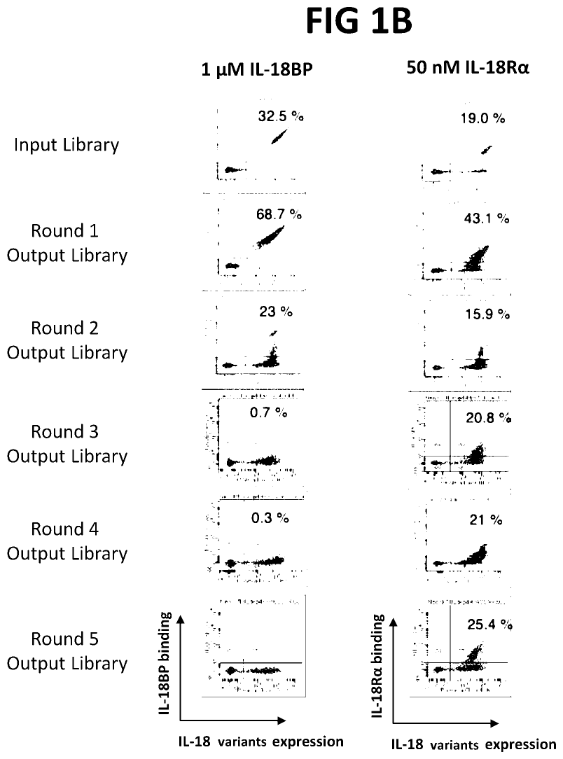

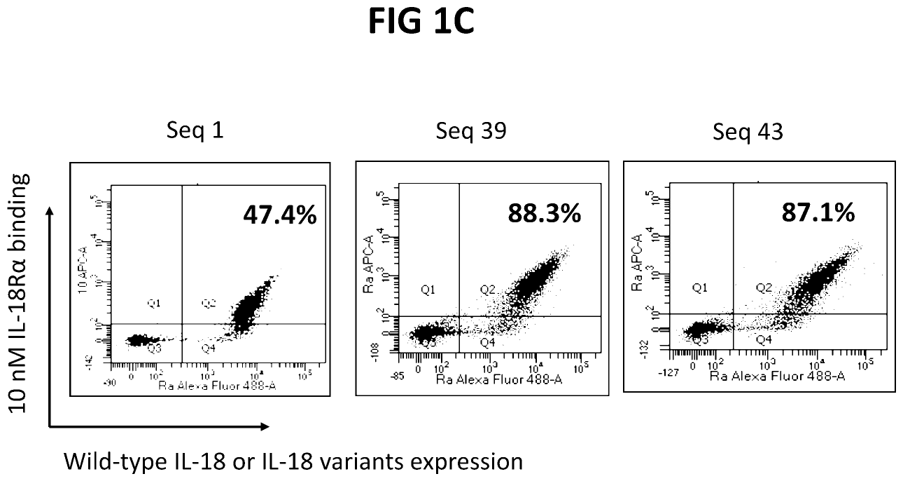

A positive/negative selection strategy was used to screen the library constructed in Example 1 to identify IL-18 variant polypeptides that bind to hIL-18Rα, but not to hIL-18BP and IL-18 variant polypeptides that have reduced binding affinity for hIL-18BP, as compared to wild-type IL-18. Briefly, recombinant hIL-18Rα extracellular domain (“hIL-18Rα ECD”) was labeled with biotin and immobilized on a first set of streptavidin (SA) magnetic microbeads or anti-biotin magnetic microbeads for magnetic activated cell sorting (MACS)-based screening. Recombinant hIL-18BP was immobilized on a second set of SA magnetic microbeads or anti-biotin magnetic microbeads. The hIL-18Rα ECD-bound beads and the hIL-18BP-bound beads were used to perform three rounds of selection on the library constructed in Example 1, as illustrated in FIG. 1A.

Next, two additional rounds of FACS-based kinetic competition screening were performed (i.e., rounds 4 and 5 as shown in FIG. 1A) to further enrich IL-18 variant polypeptides that exhibited: (a) higher or comparable affinities for hIL-18Rα as compared to WT hIL-18 and (b) no detectable or reduced binding to hIL-18BP (e.g., with a K_{D} for hIL-18BP of >10⁻³ M). Then, single yeast cells from round 4 and round 5 output pools were isolated. Plasmids encoding the isolated IL-18 variant polypeptides were extracted from the yeast and sequenced via next generation sequencing (NGS).

After each round of screening, output library size was determined by FACS. The binding ability of output library to 50 nM hIL-18Rα ECD or 1 μM hIL-18BP were also confirmed by FACS. As shown in FIG. 1B, after above screening, most of the displayed IL-18 variants did not bind to hIL-18BP or exhibited reduced binding to hIL-18BP, and meanwhile the binding ability to hIL-18Rα ECD was maintained or increased.

### Example 3: Single Clone Fluorescence-Activated Cell Sorting (FACS) of Yeast Cells Displaying IL-18 Variant Polypeptides

Following the screens described in Example 2, single clone FACS analysis was performed to verify the binding of selected yeast cell surface-expressed IL-18 variant polypeptides from Round 4 and Round 5 to 10 nM hIL-18Rα-ECD. Briefly, each yeast clone at the same density was incubated with 10 nM IL-18Rα-ECD labeled by fluorescent probe, respectively, followed by washing step. Then the ratio of fluorescent positive IL-18Rα ECD binding yeasts in the population of each clone were analyzed.

As exemplarily shown in Table 1 and FIG. 1C (which shows WT IL-18 (SEQ ID NO. 1), and clones M12 (SEQ ID NO: 39) and M17 (SEQ ID NO: 43) as examples), among the 192 IL-18 variant clones tested, 117 (i.e., SEQ ID NOs 2-118) were identified as positive clones (>0.1% cells with fluorescence in the population of the clone). SEQ ID NO: 1 is wild-type human IL-18. Table 1:

TABLE 1
Single-Clone FACS Results for 117 Clones

| | % of fluorescence positive cells in the population of the |
|---|---|
| SEQ ID NO. | yeast clone after incubation with 10 nM hIL-18 Rα ECD |
| 1(WT hIL-18) | 47.4% |
| 2 | 96.72 |
| 3 | 96.44 |
| 4 | 96.01 |
| 5 | 95.63 |
| 6 | 95.54 |
| 7 | 95.52 |
| 8 | 95.38 |
| 9 | 95.01 |
| 10 | 94.75 |
| 11 | 93.85 |
| 12 | 93.29 |
| 13 | 93.25 |
| 14 | 93.21 |
| 15 | 93.05 |
| 16 | 93 |
| 17 | 92.88 |
| 18 | 91.99 |
| 19 | 97.88 |
| 20 | 91.71 |
| 21 | 91.63 |
| 22 | 91.62 |
| 23 | 90.77 |
| 24 | 90.1 |
| 25 | 90 |
| 26 | 89.96 |
| 27 | 89.94 |
| 28 | 89.84 |
| 29 | 89.73 |
| 30 | 89.66 |
| 31 | 89.49 |
| 32 | 88.93 |
| 33 | 88.78 |
| 34 | 88.61 |
| 35 | 88.55 |
| 36 | 88.52 |
| 37 | 88.43 |
| 38 | 88.3 |
| 39 | 88.29 |
| 40 | 87.91 |
| 41 | 87.54 |
| 42 | 87.25 |
| 43 | 87.14 |
| 44 | 86.18 |
| 45 | 86.08 |
| 46 | 84.89 |
| 47 | 84.68 |
| 48 | 83.85 |
| 49 | 83.57 |
| 50 | 83.44 |
| 51 | 83.06 |
| 52 | 83 |
| 53 | 82.42 |
| 54 | 80.87 |
| 55 | 79.54 |
| 56 | 79 |
| 57 | 79 |
| 58 | 77.74 |
| 59 | 76.5 |
| 60 | 76.49 |
| 61 | 76.11 |
| 62 | 73.4 |
| 63 | 72.57 |
| 64 | 70.78 |
| 65 | 69.05 |
| 66 | 68.1 |
| 67 | 66.36 |
| 68 | 60.9 |
| 69 | 60.87 |
| 70 | 51.29 |
| 71 | 45.08 |
| 72 | 44.9 |
| 73 | 39.56 |
| 74 | 36.09 |
| 75 | 30 |
| 76 | 29.68 |
| 77 | 28.39 |
| 78 | 27.78 |
| 79 | 21.5 |
| 80 | 18.56 |
| 81 | 14.81 |
| 82 | 14.47 |
| 83 | 13.53 |
| 84 | 11.48 |
| 85 | 10.77 |
| 86 | 9.28 |
| 87 | 6.12 |
| 88 | 5.45 |
| 89 | 5.27 |
| 90 | 5.15 |
| 91 | 4.83 |
| 92 | 4.48 |
| 93 | 4.48 |
| 94 | 4.21 |
| 95 | 3.45 |
| 96 | 2.79 |
| 97 | 2.66 |
| 98 | 2.36 |
| 99 | 2.36 |
| 100 | 2.16 |
| 101 | 1.91 |
| 102 | 1.64 |
| 103 | 1.44 |
| 104 | 1.41 |
| 105 | 1.36 |
| 106 | 1.34 |
| 107 | 1.23 |
| 108 | 1.08 |
| 109 | 0.98 |
| 110 | 0.71 |
| 111 | 0.69 |
| 112 | 0.69 |
| 113 | 0.61 |
| 114 | 0.46 |
| 115 | 0.35 |
| 116 | 0.18 |
| 117 | 0.17 |
| 118 | 0.15 |

### Example 4: Recombinant hIL-18 Expression and Purification

WT hIL-18 and selected IL-18 variant polypeptides described above were expressed in an E. coli system. Briefly, PET30a plasmids comprising codon optimized sequences encoding WT hIL-18 or hIL-18 variant polypeptides were transfected into E. coli BL21 (DE3) cells. Then the transfected E. coli cells were expanded in culture medium, and the expressed polypeptide were harvested and purified.

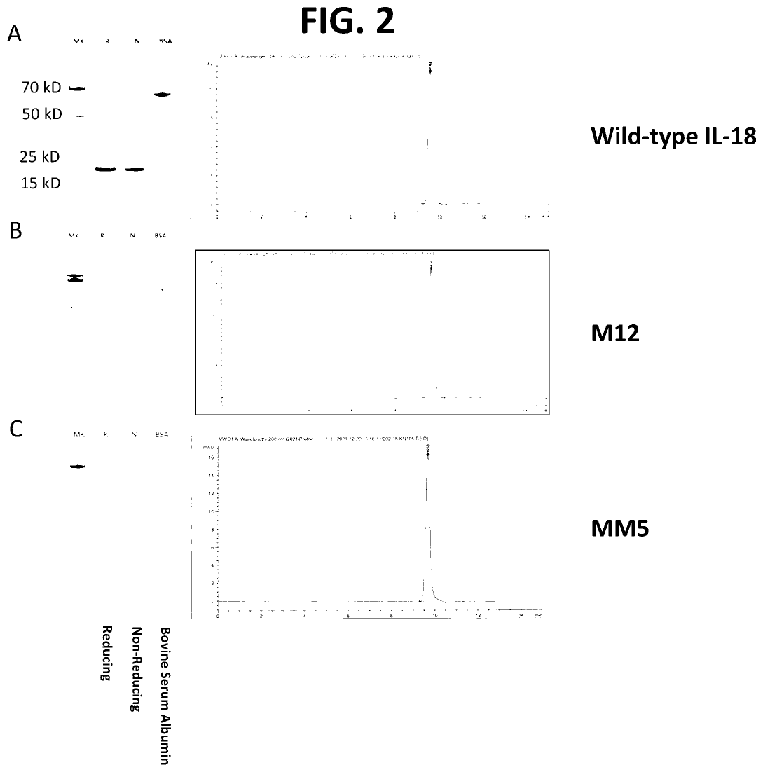

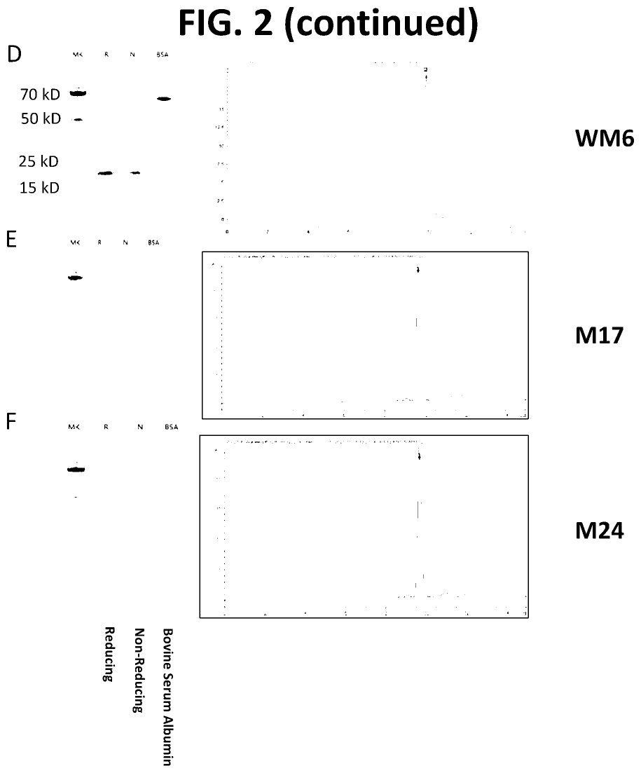

FIGS. 2A-2F illustrate WT hIL-18 and five exemplary variants (M12, MM5, WM6, M17 and M24) with purity of over 95%, as determined via SDS-PAGE and SEC-HPLC.

### Example 5: Affinities of IL-18 Variant Polypeptides for hIL-18Rα

The affinities of IL-18 variant polypeptides for hIL-18Rα were assessed by Biolayer Interferometry (BLI) on Octet RED96 using Protein A sensor coated with hIL-18Rα ECD-Fc fusion protein, and WT hIL-18 was used as a control. The binding affinities of WT hIL-18 and IL-18 variant polypeptides for hIL-18Rα ECD-Fc were as shown in Table 2A.

TABLE 2A
Affinities of IL-18 variant polypeptides
for hIL-18Rα (as determined by BLI)

| | SEQ | Response | | | |
|---|---|---|---|---|---|
| Sample ID | ID NO: | (nm) | K_{D} (M) | kₒₙ(1/M · s) | k_{dis}(1/s) |
| WT hIL-18 | 1 | 0.3082 | 6.46E−09 | 1.83E+05 | 1.18E−03 |
| M1 | 28 | 0.0186 | 2.03E−06 | 5.44E+04 | 1.11E−01 |
| M2 | 21 | 0.0184 | 1.22E−05 | 4.09E+03 | 4.99E−02 |
| M3 | 12 | 0.057 | 2.14E−06 | 2.01E+04 | 4.31E−02 |
| M4 | 72 | 0.0326 | 3.91E−08 | 4.36E+05 | 1.70E−02 |
| M5 | 4 | 0.0468 | 3.88E−08 | 3.83E+05 | 1.49E−02 |
| M6 | 56 | <0.02 | >1E−06 | | |
| M7 | 11 | 0.0222 | 1.19E−08 | 2.27E+06 | 2.71E−02 |
| M9 | 74 | <0.02 | 4.88E−09 | 2.21E+06 | 1.08E−02 |
| M10 | 66 | 0.0788 | 4.86E−08 | 4.83E+05 | 2.35E−02 |
| M11 | 49 | 0.08 | 6.82E−08 | 2.77E+05 | 1.89E−02 |
| M12 | 39 | 0.1862 | 2.49E−08 | 4.27E+05 | 1.07E−02 |
| M13 | 3 | 0.3096 | 1.16E−08 | 3.27E+05 | 3.80E−03 |
| M14 | 38 | 0.2185 | 2.36E−08 | 2.50E+05 | 5.88E−03 |
| M15 | 9 | 0.1223 | 3.67E−08 | 4.99E+05 | 1.83E−02 |
| M16 | 35 | 0.1113 | 4.26E−08 | 4.15E+05 | 1.77E−02 |
| M17 | 43 | 0.0784 | 6.77E−08 | 3.40E+05 | 2.31E−02 |
| M18 | 54 | 0.1735 | 3.31E−08 | 4.04E+05 | 1.34E−02 |
| M19 | 26 | 0.025 | 1.12E−07 | 4.95E+05 | 5.54E−02 |
| M20 | 44 | 0.1364 | 3.51E−08 | 4.76E+05 | 1.67E−02 |
| M21 | 27 | 0.0791 | 6.49E−08 | 3.40E+05 | 2.21E−02 |
| M22 | 91 | 0.0592 | 4.08E−06 | 9.74E+03 | 3.98E−02 |
| M23 | 99 | 0.0522 | 3.68E−07 | 8.23E+04 | 3.02E−02 |
| M24 | 105 | 0.0434 | 9.30E−07 | 4.85E+04 | 4.51E−02 |
| M25 | 104 | 0.0798 | 4.41E−08 | 5.81E+05 | 2.56E−02 |
| M26 | 2 | 0.0917 | 5.71E−08 | 2.07E+05 | 1.19E−02 |
| M27 | 58 | 0.0836 | 4.59E−08 | 1.65E+05 | 7.56E−03 |
| M28 | 50 | 0.178 | 3.04E−08 | 3.18E+05 | 9.66E−03 |
| M29 | 116 | 0.0323 | 8.39E−08 | 7.30E+05 | 6.12E−02 |
| M30 | 114 | 0.0275 | 3.02E−08 | 1.29E+06 | 3.89E−02 |
| M31 | 65 | 0.0947 | 5.41E−08 | 3.49E+05 | 1.89E−02 |
| M32 | 94 | 0.0381 | 1.73E−08 | 1.36E+06 | 2.34E−02 |
| M33 | 117 | 0.0257 | 5.95E−07 | 1.61E+05 | 9.57E−02 |
| M34 | 41 | 0.084 | 3.28E−08 | 5.05E+05 | 1.66E−02 |
| M35 | 17 | 0.1287 | 3.41E−08 | 4.56E+05 | 1.56E−02 |
| M36 | 81 | <0.02 | >1E−06 | | |
| M37 | 8 | 0.2219 | 2.18E−08 | 4.04E+05 | 8.83E−03 |
| M38 | 55 | 0.0307 | 1.20E−07 | 2.78E+05 | 3.34E−02 |
| M39 | 59 | 0.0605 | 2.06E−08 | 1.07E+06 | 2.21E−02 |
| M40 | 73 | 0.1609 | 3.26E−08 | 3.71E+05 | 1.21E−02 |
| M41 | 139 | <0.02 | >1E−06 | | |
| LM1 | 121 | 0.0618 | 8.62E−08 | 5.45E+05 | 4.70E−02 |
| LM2 | 122 | 0.0338 | 3.79E−08 | 1.37E+06 | 5.20E−02 |
| LM3 | 123 | 0.0422 | 1.46E−08 | 2.24E+06 | 3.28E−02 |
| LM4 | 124 | 0.0262 | 4.17E−09 | 4.06E+06 | 1.69E−02 |
| LM5 | 125 | 0.038 | 1.96E−08 | 2.10E+06 | 4.11E−02 |
| MM4 | 132 | 0.0238 | 9.82E−09 | 3.82E+06 | 3.75E−02 |
| MM5 | 133 | 0.0326 | 1.04E−07 | 7.43E+05 | 7.70E−02 |
| WM1 | 140 | 0.1596 | 2.82E−08 | 2.39E+05 | 6.75E−03 |
| WM2 | 141 | <0.02 | >1E−06 | | |
| WM3 | 142 | 0.3045 | 1.01E−08 | 3.94E+05 | 3.98E−03 |
| WM4 | 143 | 0.0373 | 2.20E−08 | 2.86E+05 | 6.29E−03 |
| WM5 | 144 | 0.0471 | 1.06E−07 | 7.24E+04 | 7.71E−03 |
| WM6 | 145 | 0.3038 | 1.01E−08 | 1.96E+05 | 1.99E−03 |
| WM7 | 146 | 0.3213 | 7.71E−09 | 2.38E+05 | 1.83E−03 |
| WM8 | 147 | <0.02 | >1E−06 | | |
| WM9 | 148 | 0.3886 | 2.22E−09 | 5.04E+05 | 1.12E−03 |
| WM10 | 149 | <0.02 | 1.29E−08 | 5.43E+05 | 7.00E−03 |
| WM11 | 150 | <0.02 | 2.09E−08 | 5.98E+05 | 1.25E−02 |
| WM12 | 151 | 0.3823 | 2.66E−09 | 4.46E+05 | 1.18E−03 |
| WM13 | 152 | 0.3084 | 8.23E−09 | 2.07E+05 | 1.70E−03 |
| WM14 | 153 | 0.3357 | 7.05E−09 | 3.07E+05 | 2.16E−03 |

The mutations of theses IL-18 variant polypeptides (i.e., M1-M41, LM1-LM5, MM1-MM2, MM4-MM6, WM1-WM6, WM8, WM10-WM14, corresponding to SEQ ID NOs: 1-4, 8-9, 11-12, 17, 21-22, 26-28, 35, 38-39, 41, 43-44, 49-50, 54-56, 58-59, 65-66, 72-74, 81, 91, 94, 99, 104-105, 114, 116-117, 121-125, 131-134, 139-145, 147, 149-153) were shown in Table 2B.

TABLE 2B

| | | | | | | | | Other |
|---|---|---|---|---|---|---|---|---|
| | G3 | E6 | D54 | Q56 | P57 | N91 | R104 | mutations |
| M1 | P | K | H | D | R | V | V | |
| M2 | S | K | W | P | D | G | T | |
| M3 | P | K | W | H | V | A | Y | |
| M4 | P | K | W | G | V | V | F | |
| M5 | E | T | W | P | W | V | T | N155K |
| M6 | P | R | W | T | | V | T | |
| M7 | P | R | H | T | A | A | | |
| M8 | P | R | W | P | A | A | L | |
| M9 | P | R | W | R | A | S | S | |
| M10 | P | R | Q | L | W | S | | |
| M11 | P | R | S | R | N | G | T | |
| M12 | P | K | G | G | A | T | | |
| M13 | P | K | Q | I | W | | | |
| M14 | N | K | P | S | S | R | A | |
| M15 | P | R | S | Y | T | G | | |
| M16 | P | R | S | R | N | G | S | |
| M17 | P | R | L | T | A | G | | |
| M18 | P | R | S | R | R | G | S | |
| M19 | P | K | H | E | Q | A | | |
| M20 | P | R | S | R | S | G | S | |
| M21 | P | R | S | S | T | G | S | |
| M22 | P | R | Y | R | G | K | S | |
| M23 | P | R | Y | T | R | | | |
| M24 | P | R | Y | T | S | | | |
| M25 | P | R | L | T | T | R | | |
| M26 | P | R | H | D | K | V | Y | |
| M27 | P | R | H | Y | | V | Y | |
| M28 | P | A | W | G | G | V | Y | |
| M29 | P | M | F | D | R | P | | |
| M30 | P | L | H | T | V | S | | |
| M31 | P | R | H | I | H | I | Y | |
| M32 | P | G | S | S | R | | | |
| M33 | E | H | R | T | H | | | |
| M34 | P | R | H | R | N | V | E | |
| M35 | P | R | G | G | A | G | | |
| M36 | P | S | A | D | Q | G | | |
| M37 | P | G | Q | V | W | | | |
| M38 | P | S | W | G | A | V | I | |
| M39 | P | R | W | P | G | V | L | |
| M40 | D | K | P | S | W | W | | |
| M41 | P | | | | | | | |
| LM1 | P | K | G | G | A | | | |
| LM2 | P | R | L | G | S | V | | |
| LM3 | P | R | G | G | A | G | | V11I |
| LM4 | P | K | H | Y | S | | | |
| LM5 | | R | W | S | Q | | | |
| MM1 | P | K | L | T | Q | | | |
| MM2 | A | Y | R | S | L | G | | |
| MM4 | P | R | L | T | I | G | | |
| MM5 | | G | L | T | E | G | S | |
| MM6 | A | Y | R | S | L | A | | |
| WM1 | | | | | | | | M60K, |
| | | | | | | | | K96D |
| WM2 | P | R | | | | | | K96E |
| WM3 | | | | | | | | M60K |
| WM4 | P | R | | | | | | |
| WM5 | P | K | | | | | | |
| WM6 | D | K | | | | S | | |
| WM8 | S | | | | | | | I149M |
| WM10 | P | R | | | | S | | |
| WM11 | | R | | | | | | |
| WM12 | | R | | | | S | | |
| WM13 | | | | | | | | V11I |
| WM14 | S | | | | | | | K140R |

### Example 6: Functional Characterization of IL-18 Variant Polypeptides

6.1 Activation of hIL-18 Receptor-Mediated Signaling by IL-18 Variant Polypeptides

An NFκB-luc/hIL-18RαRβ HEK293 cell line stably expressing hIL-18Rα, hIL-18Rβ and an NFκB luciferase reporter gene was used as the reporter cell to determine the abilities of IL-18 variant polypeptides in activating hIL-18 receptor mediated signaling.

Briefly, the reporter cells were first seeded in the wells of a white 96-well plate at a density of 3.0×10⁴ cells per well. WT hIL-18 or IL-18 variant polypeptides were serially diluted in medium in (a) the presence and (b) the absence of 1 μg/mL hIL-18 BP-hFc. Luminescence intensity was measured after 16 hours of incubation at 37° C.

As shown in FIG. 3 and Table 3, all the IL-18 variant polypeptides tested showed potency of activating IL-18 receptor-mediated signaling in a dose dependent manner. Unlike WT hIL-18, the activity of which was greatly decreased (by about 80-fold) in the presence of 1 μg/mL hIL-18BP-hFc, the IL-18 variant polypeptides (i.e., M1-M7, M9-M41, LM1-LM5, MM1-MM2, MM4-MM6, WM1-WM6, WM8, WM10-11, corresponding to SEQ ID NOs: 2-4, 8-9, 11-12, 17, 21, 22, 26-28, 35, 38, 39, 41, 43, 44, 49, 50, 54-56, 58-59, 65-66, 72-74, 81, 91, 94, 99, 104-105, 114, 116-117, 121-125, 131-134, 139-145, 147, 149-150) demonstrated Hil-18 BP-resistant activity with changes of EC₅₀ by no more than 3 folds, indicating that the IL-18 variant polypeptides were significantly less affected by Hil-18BP in view of Hil-18 receptor-mediated signaling activation. One exception is WM6, EC₅₀ of which shows ˜12-fold potency loss in the presence of 1 μg/MI Hil-18BP-hFc, which is still a lot better than the WT hIL-18.

TABLE 3
EC₅₀s of IL-18 Variant Polypeptides in hIL-18 Reporter Assay,
in the Presence or Absence of 1 μg/mL hIL-18BP-hFc

| | SEQ | | EC₅₀ (nM) in the | EC₅₀ Fold |
|---|---|---|---|---|
| | ID | EC₅₀ | Presence of 1 μg/ | Change |
| Protein ID | NO | (nM) | mL hIL-18BP-hFc | (+BP/−BP) |
| WT hIL-18 | 1 | 0.1-0.3 | 18-25 | ~83.33 |
| M1 | 28 | 1.45 | 3.45 | 2.38 |
| M2 | 21 | 0.27 | 0.19 | 0.70 |
| M3 | 12 | 0.24 | 0.37 | 1.54 |
| M4 | 72 | 0.018 | 0.025 | 1.39 |
| M5 | 4 | 0.22 | 0.21 | 0.95 |
| M6 | 56 | 0.18 | 0.14 | 0.78 |
| M7 | 11 | 0.15 | 0.25 | 1.67 |
| M9 | 74 | 0.43 | 0.33 | 0.77 |
| M10 | 66 | 0.29 | 0.17 | 0.59 |
| M11 | 49 | 0.13 | 0.21 | 1.62 |
| M12 | 39 | 0.04 | 0.03 | 0.75 |
| M13 | 3 | 0.035 | 0.026 | 0.74 |
| M14 | 38 | 0.04 | 0.03 | 0.75 |
| M15 | 9 | 0.13 | 0.12 | 0.92 |
| M16 | 35 | 0.29 | 0.34 | 1.17 |
| M17 | 43 | 0.41 | 0.32 | 0.78 |
| M18 | 54 | 0.58 | 0.56 | 0.97 |
| M19 | 26 | 0.66 | 0.31 | 0.47 |
| M20 | 44 | 2.2 | 0.5 | 0.23 |
| M21 | 27 | 1.9 | 2.1 | 1.11 |
| M22 | 91 | 7.2 | 7.5 | 1.04 |
| M23 | 99 | 0.8 | 1.4 | 1.75 |
| M24 | 105 | 0.99 | 1.99 | 2.01 |
| M25 | 104 | 0.58 | 1.15 | 1.98 |
| M26 | 2 | 0.068 | 0.094 | 1.38 |
| M27 | 58 | 0.18 | 0.26 | 1.44 |
| M28 | 50 | 0.018 | 0.042 | 2.33 |
| M29 | 116 | 4.8 | 1.9 | 0.40 |
| M30 | 114 | 2.5 | 1.7 | 0.68 |
| M31 | 65 | 0.25 | 0.18 | 0.72 |
| M32 | 94 | 5.9 | 4.1 | 0.69 |
| M33 | 117 | 18.4 | 10.7 | 0.58 |
| M34 | 41 | 0.71 | 0.9 | 1.27 |
| M35 | 17 | 0.62 | 0.51 | 0.82 |
| M36 | 81 | 3.9 | 2.5 | 0.64 |
| M37 | 8 | 0.14 | 0.33 | 2.36 |
| M38 | 55 | 0.57 | 1.2 | 2.11 |
| M39 | 59 | 0.3 | 0.44 | 1.47 |
| M40 | 73 | 0.12 | 0.16 | 1.33 |
| M41 | 139 | 5.17 | 4 | 0.77 |
| LM1 | 121 | 0.076 | 0.11 | 1.45 |
| LM2 | 122 | 0.11 | 0.055 | 0.50 |
| LM3 | 123 | 0.052 | 0.069 | 1.33 |
| LM4 | 124 | 0.16 | 0.17 | 1.06 |
| LM5 | 125 | 0.13 | 0.12 | 0.92 |
| MM1 | 22 | 0.97 | 0.81 | 0.84 |
| MM2 | 131 | 0.93 | 2.24 | 2.41 |
| MM4 | 132 | 0.13 | 0.43 | 3.31 |
| MM5 | 133 | 0.028 | 0.08 | 2.86 |
| MM6 | 134 | 1 | 1.5 | 1.50 |
| WM1 | 140 | 4.27 | 2.1 | 0.49 |
| WM2 | 141 | 10.71 | 14.19 | 1.32 |
| WM3 | 142 | 0.13 | 0.33 | 2.54 |
| WM4 | 143 | 3.5 | 7.7 | 2.20 |
| WM5 | 144 | 2.7 | 1.7 | 0.63 |
| WM6 | 145 | 0.06 | 0.74 | 12.33 |
| WM8 | 147 | 7.0 | 5.2 | 0.74 |
| WM10 | 149 | 14.7 | 15.6 | 1.06 |
| WM11 | 150 | 10.2 | 15.7 | 1.54 |

6.2 Human PBMC-Based hIFNγ Release Assays

PBMC-based assays were performed to test abilities of IL-18 variant polypeptides in activating T/NK cells and inducing hIFNγ release. Briefly, human PBMC cells were seeded in the wells of 96-well plate at a density of 5.0×10⁵ cells per well and cultured for 5 hours before treatment. WT hIL-18 or IL-18 variant polypeptides were serially diluted with 1 ng/ml human IL-12 in (a) the presence or (b) the absence of 1 μg/mL hIL-18 BP-hFc, and then supplemented to the wells. Culture medium was collected from each well after 16 hour incubation at 37° C., and hIFNγ concentration was measured via FRET using a kit (62HIFNGPEG, Cisbio) according to manufacturer's instructions.

As shown in FIG. 4, the tested IL-18 variant polypeptides showed potency of inducing hIFNγ release from PBMC in a dose dependent manner. In addition, as shown in Table 4, the activity of WT hIL-18 was greatly attenuated in the presence of 1 μg/mL hIL-18BP-hFc, with a decrease of EC₅₀ by about 35 folds. In contrast, the IL-18 variant polypeptides (i.e., M11-M13, M15, M17, M21, M24, M29, M32-M33, M35-M36, MM5, WM4-WM6, WM8, WM10-11, corresponding to SEQ ID NOs: 3, 9, 17, 27, 39, 43, 49, 94, 105, 116-117, 133, 143-150) demonstrated hIL-18 BP-resistant activity with changes of EC₅₀ by no more than 2 folds, indicating that the IL-18 variant polypeptides were significantly less affected by hIL-18BP in view of hIFNγ induction.

TABLE 4
EC₅₀s of IL-18 Variant Polypeptides in PBMC Assay,
in the Presence or Absence of 1 μg/mL hIL-18BP

| | | EC50 (nM) in the | |
|---|---|---|---|
| | | Presence of 1 μg/mL | EC50 Fold Change |
| Protein ID | EC50 (nM) | hIL-18BP-hFc | (+BP/−BP) |
| WT hIL-18 | 0.17 | 5.9 | 34.71 |
| M11 | 0.044 | 0.068 | 1.55 |
| M12 | 0.043 | 0.045 | 1.05 |
| M13 | 0.02 | 0.025 | 1.25 |
| M15 | 0.049 | 0.036 | 0.73 |
| M17 | 0.13 | 0.13 | 1.00 |
| M21 | 0.045 | 0.049 | 1.09 |
| M24 | 0.054 | 0.08 | 1.48 |
| M29 | 0.14 | 0.096 | 0.69 |
| M32 | 0.13 | 0.1 | 0.77 |
| M33 | 0.51 | 0.35 | 0.69 |
| M35 | 0.085 | 0.14 | 1.65 |
| M36 | 1.1 | 0.8 | 0.73 |
| MM5 | 0.035 | 0.045 | 1.29 |
| WM4 | 0.26 | 0.2 | 0.77 |
| WM5 | 0.18 | 0.19 | 1.06 |
| WM6 | NA | ~0.04 | TBD |
| WM8 | 0.68 | 1.02 | 1.50 |
| WM10 | 0.53 | 0.44 | 0.83 |
| WM11 | 0.54 | 0.95 | 1.76 |

6.3 Mouse Splenocyte-Based mIFNγ Release Assay

To assess in vivo efficacy of the IL-18 variant polypeptides of the present application, mouse splenocyte-based murine IFNγ (“mIFNγ”) release assays are performed as follows.

Briefly, fresh isolated mouse splenocytes are seeded in the wells of a 96-well plate at a density of 6.5×10⁻⁵ cells per well. WT hIL-18 or IL-18 variant polypeptides were serially diluted with 1 ng/mL mouse IL-12 in (a) the presence or (b) the absence of 1 μg/mL mouse IL-18BP-Fc, and then supplemented to the wells. Culture medium is collected from each well after 16 hour incubation at 37° C. and then mIFNγ concentration in the medium is measured via FRET using a kit (62MIFNGPEG, Cisbio) according to manufacturer's instructions.

### Example 7: Generation and Characterization of IL-18 Variant Polypeptides Comprising Serine→Cystine and/or Cystine→Serine Substitutions

### 7.1 Wild-Type IL-18 or IL-18 Polypeptide Variants Comprising Cystine→Serine Substitutions

Disulfide bond engineering can improve a polypeptide's stability and yield. The following experiments were performed to assess the effects of Cys to Ser (“C→S”) mutations on the druggability of IL-18 variants and on the expression of IL-18 variants in mammalian cells, such as CHO cells and 293F cells. Wild-type IL-18 and IL-18 variant polypeptides M12 and MM5 were modified (or further modified) to comprise C38S, C68S, and C76S substitutions to generate new variants comprising the amino acid sequences below:

IL-18 VARIANT COMPRISING C38S, C68S, and C76S
substitutions in WT hIL-18 background (“IL-18-SSS”):
(SEQ ID NO: 307)
YFGKLESKLSVIRNLNDQVLFIDQGNRPLFEDMTDSDSRDNAPRTIFIIS
MYKDSQPRGMAVTISVKSEKISTLSSENKIISFKEMNPPDNIKDTKSDII
FFQRSVPGHDNKMQFESSSYEGYFLACEKERDLFKLILKKEDELGDRSIM
FTVQNED
IL-18 VARIANT COMPRISING C38S, C68S, and C76S
substitutions in M12 background (“M12-SSS”):
(SEQ ID NO: 308)
YFPKLKSKLSVIRNLNDQVLFIDQGNRPLFEDMTDSDSRDNAPRTIFIIS
MYKGSGARGMAVTISVKSEKISTLSSENKIISFKEMNPPDTIKDTKSDII
FFQRSVPGHDNKMQFESSSYEGYFLACEKERDLFKLILKKEDELGDRSIM
FTVQNED
IL-18 VARIANT COMPRISING C38S, C68S, and C76S
substitutions in MM5 background (“MM5-SSS”):
(SEQ ID NO: 309)
YFGKLGSKLSVIRNLNDQVLFIDQGNRPLFEDMTDSDSRDNAPRTIFIIS
MYKLSTERGMAVTISVKSEKISTLSSENKIISFKEMNPPDGIKDTKSDII
FFQSSVPGHDNKMQFESSSYEGYFLACEKERDLFKLILKKEDELGDRSIM
FTVQNED

Next, the C-termini of each of IL-18-SSS, M12-SSS, and M5-SSS were fused to the N-terminus of a human IgG1 Fc comprising an N297A substitution (EU numbering) to generate IL-18-SSS-Fc_N297A (SEQ ID NO: 319), M12-SSS_N297A (SEQ ID NO: 320), and M5-SSS_N-297A (SEQ ID NO: 321). The fusion polypeptides were expressed in CHO cells, harvested, and purified via Protein A chromatography. The yield of IL-18-SSS-Fc_N297A obtained following one step Protein A purification was 14.7 mg/L, with 65% purity. By contrast, the yield of WT IL-18-Fc_N297A obtained following one step Protein A purification was ˜16 mg/L, with 11% purity. The yield of M12-SSS-Fc_N297A obtained following one step Protein A purification was 5.6 mg/ml, with 72% purity. By contrast, M12-Fc_N297A was not expressed at a detectable level in 100 mL CHO host cells. The yield of MM5-SSS-Fc_N297A obtained following one step Protein A chromatography was 24.5 mg/L, with 46% purity. Following a further purification step by gel filtration, the yield of MM5-SSS-Fc_N297 was 3.4 mg/L, with 99% purity. By contrast, MM5-Fc_N297A was not expressed at a detectable level in 100 mL CHO host cells.

M12-SSS was further modified to comprise a C127S substitution to generate M12-SSSS:

(SEQ ID NO: 310)
YFPKLKSKLSVIRNLNDQVLFIDQGNRPLFEDMTDSDSRDNAPRTIFIIS
MYKGSGARGMAVTISVKSEKISTLSSENKIISFKEMNPPDTIKDTKSDII
FFQRSVPGHDNKMQFESSSYEGYFLASEKERDLFKLILKKEDELGDRSIM
FTVQNED

The C-terminus of M12-SSSS was fused to the N-terminus of a human IgG1 Fc variant comprising an N297A substitution (EU numbering) to generate M12-SSSS-Fc_N297A (SEQ ID NO: 322). M12-SSSS-Fc_N297A was expressed in CHO cells, harvested, and purified via Protein A chromatography. Introduction of the C127S substitution (i.e., a fourth C→S substitution) led to decreased yield and purity of M12-SSSS-Fc_N297A as compared to the yield and purity of M12-SSS-Fc_N297A (data not shown).

Next, the potencies of IL-18-SSS-Fc_N297A and M12-SSS-Fc_N297A in activating IL-18 receptor-mediated signaling were characterized using the in vitro assay described in Section 6.1 of Example 6. The potency of IL-18-SSS-Fc_N297A showed was comparable to that of wild-type IL-18 (see FIG. 5A), whereas M12-SSS-Fc_N297A showed a ˜7-fold potency loss as compared to M12 (see FIG. 5B). The signaling activity of IL-18-SSS-Fc_N297A decreased in the presence of 1 μg/ml hIL-18 BP-hFc (“BP”), whereas M12-SSS-Fc_N297A demonstrated hIL-18 BP-resistant activity in the presence of 1 μg/ml hIL-18BP-hFc. See FIGS. 5A and 5B. As shown in FIG. 5C, MM5-SSS-Fc_N297A showed a ˜220-fold potency loss as compared to MM5, but demonstrated hIL-18 BP-resistant activity in the presence of 1 μg/ml hIL-18BP-hFc. Such results indicate that that the 3 cystine→serine substitutions do not affect IL-18 BP resistance. These data suggests that C38, C68, and C76 are critical for improving the yield of IL-18 and of IL-18 polypeptide-Fc fusion protein during production and following purification, with improved yield and purity. MM5-SSS-Fc_N297A may exhibit a higher half-life than MM5.

7.2 IL-18 Polypeptide Variants Derived from M12 Comprising Amino Acid Substitutions that Remove and/or Introduce Cysteine Residues

The following experiments were performed to assess the effects of substitutions that delete inherent cystines (i.e., naturally occurring cysteines in a polypeptide sequence) and/or introduce a disulfide bond on the druggability, expression, and purification of IL-18 variant polypeptides. First, in silico screening was performed based on molecular dynamic simulation, and following AI-based stability assessment, mutation hot spots on each of the four cystines (C38, C68, C76, and C127) of M12 were identified as listed. See Table 5. In addition, structure-guided design was used to identify amino acids in spatial positions which, when substituted to Cys residue(s), would permit the formation of new, non-native disulfide bond(s). The mutation strategies shown in Table 5 (i.e., DB6, DB7, DB8, DB9, and DB10) were proposed.

TABLE 5
M12-Derived IL-18 Polypeptide Variants

| Wild-Type | | | | | | |
|---|---|---|---|---|---|---|
| Amino | Mutation | | | | | |
| Acid | Hot Spot | DB6 | DB7 | DB8 | DB9 | DB10 |
| C38 | IVLM | I | V | S | I | I |
| C68 | IVSD | S | I | I | I | L |
| C76 | YV | | | | V | Y |
| S117 | | C | C | C | | |
| C127 | AIYFL | | A | I | I | |

The mutation strategies in Table 5 were introduced into IL-18 polypeptide variant M12 to generate new variants M12-DB6, M12-DB7, M12-DB8, M12-DB9, and M12-DB10.

M12-DB6
(SEQ ID NO: 311)
YFPKLKSKLSVIRNLNDQVLFIDQGNRPLFEDMTDSDIRDNAPRTIFIIS
MYKGSGARGMAVTISVKSEKISTLSCENKIISFKEMNPPDTIKDTKSDII
FFQRSVPGHDNKMQFECSSYEGYFLACEKERDLFKLILKKEDELGDRSIM
FTVQNED
M12-DB7
(SEQ ID NO: 312)
YFPKLKSKLSVIRNLNDQVLFIDQGNRPLFEDMTDSDVRDNAPRTIFIIS
MYKGSGARGMAVTISVKIEKISTLSCENKIISFKEMNPPDTIKDTKSDII
FFQRSVPGHDNKMQFECSSYEGYFLAAEKERDLFKLILKKEDELGDRSIM
FTVQNED
M12-DB8
(SEQ ID NO: 313)
YFPKLKSKLSVIRNLNDQVLFIDQGNRPLFEDMTDSDSRDNAPRTIFIIS
MYKGSGARGMAVTISVKIEKISTLSCENKIISFKEMNPPDTIKDTKSDII
FFQRSVPGHDNKMQFECSSYEGYFLAIEKERDLFKLILKKEDELGDRSIM
FTVQNED
M12-DB9
(SEQ ID NO: 314)
YFPKLKSKLSVIRNLNDQVLFIDQGNRPLFEDMTDSDIRDNAPRTIFIIS
MYKGSGARGMAVTISVKIEKISTLSVENKIISFKEMNPPDTIKDTKSDII
FFQRSVPGHDNKMQFESSSYEGYFLAIEKERDLFKLILKKEDELGDRSIM
FTVQNED
M12-DB10
(SEQ ID NO: 315)
YFPKLKSKLSVIRNLNDQVLFIDQGNRPLFEDMTDSDIRDNAPRTIFIIS
MYKGSGARGMAVTISVKLEKISTLSYENKIISFKEMNPPDTIKDTKSDII
FFQRSVPGHDNKMQFESSSYEGYFLACEKERDLFKLILKKEDELGDRSIM
FTVQNED

The C-termini of each of M12-DB6, M12-DB7, M12-DB8, M12-DB9, and M12-DB10 variant was fused to a human IgG1 Fc variant comprising an N297A substitution (EU numbering). The resulting fusion polypeptides, i.e., M12-DB6-Fc_N297A (SEQ ID NO: 323), M12-DB7-Fc_N297A (SEQ ID NO: 324), M12-DB8-Fc_N297A (SEQ ID NO: 325), M12-DB9-Fc_N297A (SEQ ID NO: 326), and M12-DB10-Fc_N297A (SEQ ID NO: 327), were expressed in CHO cells, harvested, and purified via Protein A chromatography. The purified preparations were analyzed via SEC and SDS PAGE (data not shown). See Table 6 below.

TABLE 6
Expression and Purification of M12-DB6, M12-DB7, M12-
DB8, M12-DB9, and M12-DB10 Fc_N297A fusion proteins

| IL-18 Variant | | Purity following Protein A (%) |
|---|---|---|
| Polypeptide Fusion | Yield (mg/L) | (as determined by SEC-HPLC) |
| M12-DB6-Fc_N297A | 313 | 89.779% |
| M12-DB7-Fc_N297A | 166 | 88.644% |
| M12-DB8-Fc_N297A | / | / |
| M12-DB9- Fc_N297A | 1.2 | 90.807% |
| M12-DB10-Fc_N297A | 31.4 | 56.272% |

M12-DB8-Fc_N297A was not expressed at detectable levels. M12-DB6-Fc_N297A, M12-DB7-Fc_N297A, M12-DB9-Fc_N297A, and M12-DB10-Fc_N297A showed greatly improved expression yields or purity (see Table 6) than either M12-Fc_N297A or M12-SSS-Fc_N297A. Among M12-DB6-Fc_N297A, M12-DB7-Fc_N297A, M12-DB9-Fc_N297A, and M12-DB10-Fc_N297A, M12-DB6-Fc_N297A exhibited the highest expression yield (313 mg/L) with 89.8% purity (as determined by SEC-HPLC) after one-step Protein A purification. The expression yield of M12-DB7-Fc_N297A was about 50% of the yield of M12-DB6-Fc_N297A. M12-DB7-Fc_N297A showed similar purity with M12-DB6-Fc_N297A following Protein A chromatography, such result indicates that a C127X substitution does not further improve polypeptide production in mammalian cells.

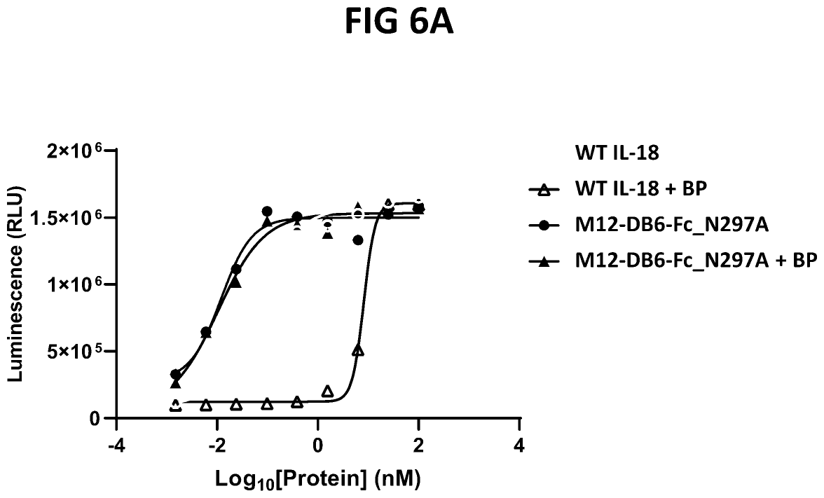

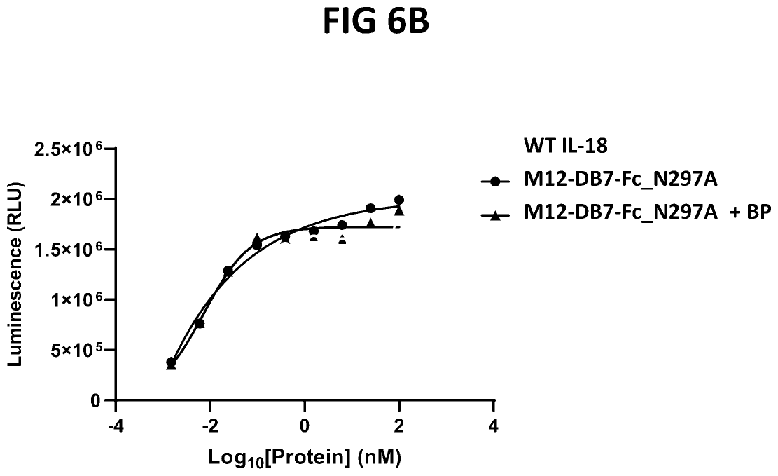

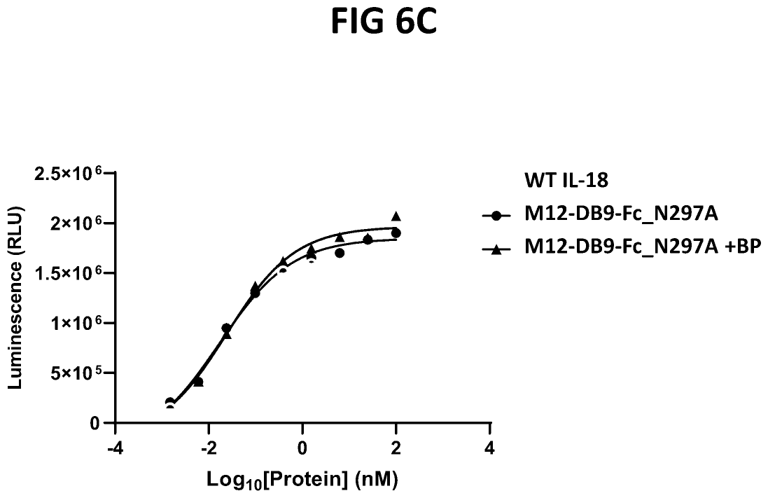

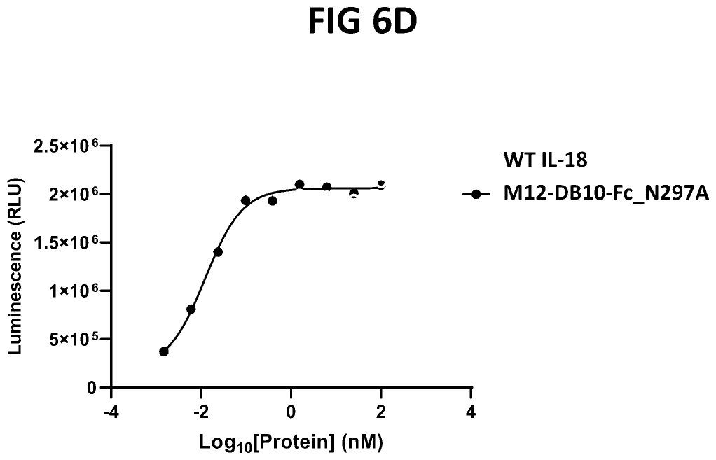

Next, the potencies of M12-DB6-Fc_N297A, M12-DB7-Fc_N297A, M12-DB9-Fc_N297A, and M12-DB10-Fc_N297A in activating IL-18 receptor-mediated signaling were characterized using the in vitro assay described in Section 6.1 of Example 6. As shown in FIGS. 6A-6D and in Table 7 below, M12-DB6-Fc_N297A (FIG. 6A), M12-DB7-Fc_N297A (FIG. 6B), M12-DB9-Fc_N297A (FIG. 6C), and M12-DB10-Fc_N297A (FIG. 6D) showed much higher potency in activating IL-18 receptor-mediated signaling than WT IL-18. The signaling activities of M12-DB6-Fc_N297A (FIG. 6A), M12-DB7-Fc_N297A (FIG. 6B), and M12-DB9-Fc_N297A (FIG. 6C) tested were not inhibited in the presence of 1 μg/ml hIL-18BP-hFc. See Table 7.

TABLE 7
EC50s of M12-DB6-Fc_N297A, M12-DB7-Fc_N297A,
M12-DB9-Fc_N297A, and M12-DB10-Fc_N297A
in an in vitro IL-18 Receptor Mediated Signaling Assay

| | | EC50 (nM) + |
|---|---|---|
| IL-18 Variant-Fc Fusion | | 1 μg/ml |
| Polypeptide | EC50 (nM) | hIL-18BP-hFc |
| WT IL-18 (control) | 0.066 | 8.2 |
| M12-DB6-Fc_N297A | 0.012 | 0.01 |
| M12-DB7-Fc_N297A | ~0.007 | 0.007 |
| M12-DB9-Fc_N297A | 0.017 | 0.025 |
| M12-DB10-Fc_N297A | 0.012 | / |

Next, “DB6” substitutions, i.e., C38I, C68S, and S117C, were introduced into variant MM5 to generate MM5-DB6:

MM5-DB6
(SEQ ID NO: 316)
YFGKLGSKLSVIRNLNDQVLFIDQGNRPLFEDMTDSDIRDNAPRTIFIIS
MYKLSTERGMAVTISVKSEKISTLSCENKIISFKEMNPPDGIKDTKSDII
FFQSSVPGHDNKMQFECSSYEGYFLACEKERDLFKLILKKEDELGDRSIM
FTVQNED

“DB6” substitutions were also introduced into wild-type IL-18 to generate WT IL18-DB6:

WT IL18-DB6
(SEQ ID NO: 317)
YFGKLESKLSVIRNLNDQVLFIDQGNRPLFEDMTDSDIRDNAPRTIFIIS
MYKDSQPRGMAVTISVKSEKISTLSCENKIISFKEMNPPDNIKDTKSDII
FFQRSVPGHDNKMQFECSSYEGYFLACEKERDLFKLILKKEDELGDRSIM
FTVQNED

An additional variant, WT-DBo was designed by introducing an S117C substitution into wild-type IL-18:

WT IL-18-DBo
(SEQ ID NO: 318)
YFGKLESKLSVIRNLNDQVLFIDQGNRPLFEDMTDSDCRDNAPRTIFIIS
MYKDSQPRGMAVTISVKCEKISTLSCENKIISFKEMNPPDNIKDTKSDII
FFQRSVPGHDNKMQFECSSYEGYFLACEKERDLFKLILKKEDELGDRSIM
FTVQNED

The C-termini of each of WT IL-18-DB6, WT IL-18-DBo, and MM5-DB6 was fused to the N-terminus of a human IgG1 Fc variant comprising an N297A substitution (EU numbering). The resulting fusion polypeptides, WT IL18-DB6-Fc_N297A (SEQ ID NO: 328), WT IL-18-DBo-Fc_N297A (SEQ ID NO: 329), and MM5-DB6-Fc_N297A (SEQ ID NO: 330), were expressed in CHO cells, harvested, and purified via Protein A chromatography. The purified preparations were analyzed via SEC and SDS PAGE (data not shown). As shown in Table 8 below, WT IL18-DBo-Fc_N297A was not expressed at detectable levels. The expression yield of WT IL18-DB6-Fc was 157 mg/L, and WT IL18-DB6-Fc_N297A was 91.6% pure (as determined by SEC-HPLC) following one-step Protein A chromatography (see Table 8). Such data indicate that engineering a disulfide bond between C76 and C117 is not sufficient to improve fusion polypeptide expression in mammalian cells. Improved expression in mammalian cells is achieved with fusion polypeptides comprising an IL-18 variant that comprises (i) an engineered C76-C117 disulfide bond, (ii) a C38 substitution (see Table 5), and (iii) a C68 substitution (see Table 5). MM5-DB6-Fc_N297A shows much higher expression yield than WT IL18-DB6-Fc_N297A with similar purity (see Table 8). Such data also suggests that the “DB6” substitutions are suitable for improving the yield of WT IL-18 and IL-18 variant polypeptides during production in mammalian cells and following purification.

Next, fusion polypeptides in which the C-terminus of a human IgG1 Fc comprising an N297A substitution (EU numbering) was fused to the N termini of each of variants M12-DB6 and MM5-DB6 were designed. The resulting fusion polypeptides, i.e., Fc_N297A-MM5-DB6 (SEQ ID NO: 332) and Fc_N297A-M12-DB6 (SEQ ID NO: 331) were expressed in CHO cells, harvested and purified via Protein A chromatography. As shown in Table 8, both Fc_N297A-MM5-DB6 and Fc_N297A-M12-DB6 demonstrated high expression yield in CHO cells. Following one-step Protein A chromatography purification, Fc_N297A-MM5-DB6 was ˜97% pure (as determined by SEC-HPLC), and Fc_N297A-M12-DB6 was 89% pure (as determined by SEC-HPLC).

TABLE 8
Yield and Purity of WT IL18-DB6-Fc_N297A,
WT IL18-DBo-Fc_N297A, MM5-DB6-Fc_N297A, Fc_N297A-
MM5-DB6, and Fc_N297A-M12-DB6 following expression
in CHO cells and Protein A Chromatography

| | Yield | Purity (%) |
|---|---|---|
| Fusion Polypeptide | (mg/L) | (as determined by SEC-HPLC) |
| WT IL18-DB6-Fc_N297A | 157 | 91.63 |
| WT IL18-DBo-Fc_N297A | 0 | / |
| MM5-DB6-Fc_N297A | 349 | 91.31 |
| Fc_N297A-MM5-DB6 | 236 | 97 |
| Fc_N297A-M12-DB6 | 284 | 88.6 |

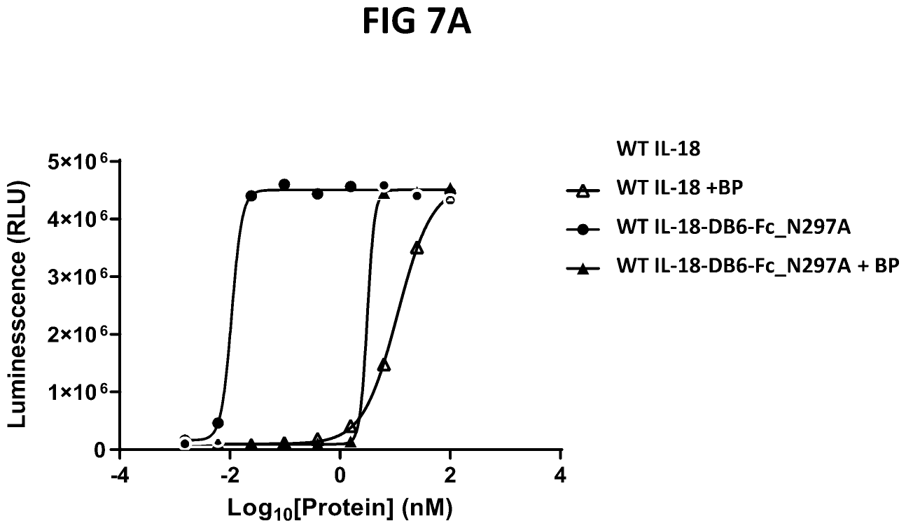

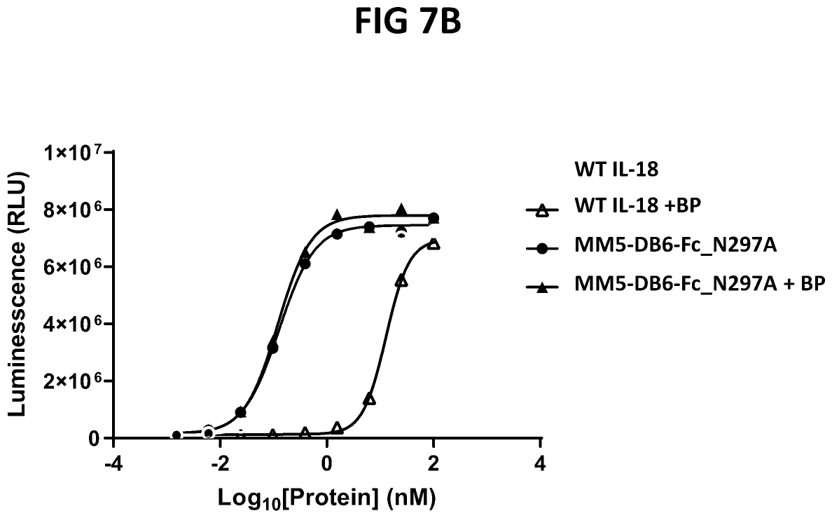

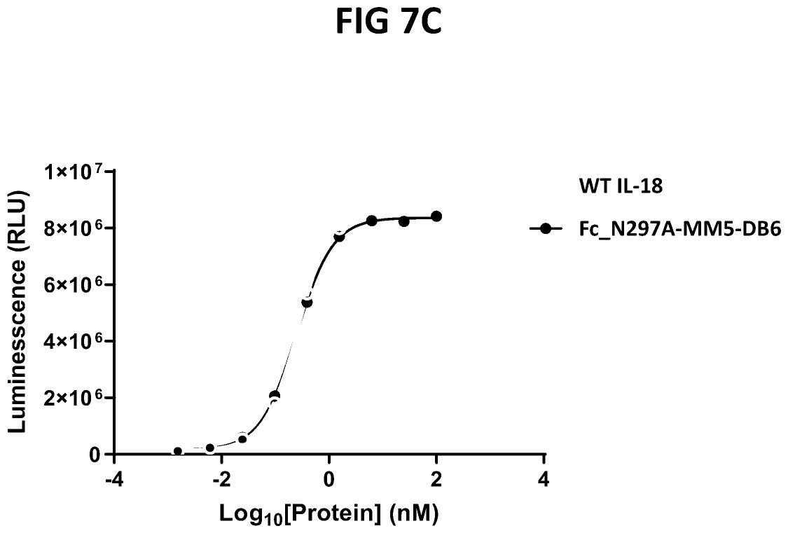

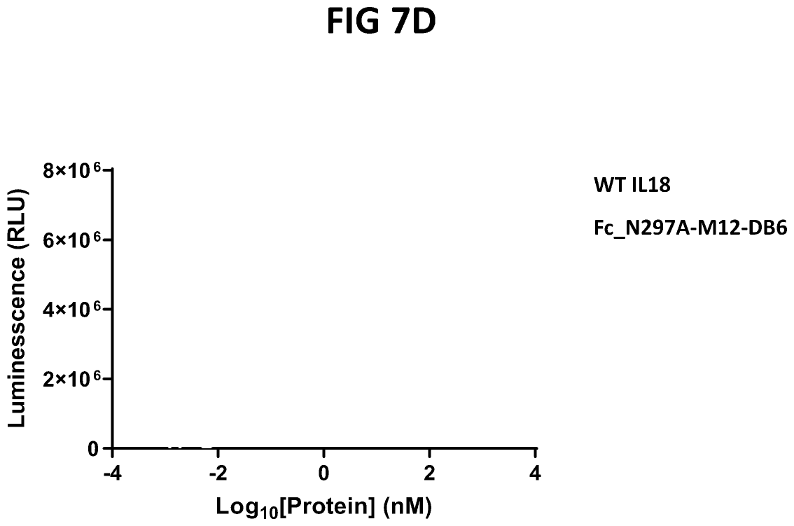

Next, the potencies of WT IL18-DB6-Fc_N297A, MM5-DB6-Fc_N297A, Fc_N297A-MM5-DB6 and Fc_N297A-M12-DB6 in activating IL-18 receptor-mediated signaling were characterized using the in vitro assay described in Section 6.1 of Example 6. Both WT IL18-DB6-Fc_N297A and MMS-DB6-Fc_N297A exhibited potent IL-18 receptor cell activation signal, stronger than that of WT IL-18 (see FIGS. 7A, 7B, and Table 9). Fc_N297A-MMS-DB6 shown similar potency with WT IL-18, while Fc_N297A-M12-DB6 resulted in much stronger activation signal to the IL-18 reporter cell (see FIGS. 7C, 7D, and Table 9). In the presence of 1 μg/mL IL-18BP-hFc, activity of WT IL18-DB6-Fc_N297A was greatly decreased, whereas the activity of MM5-DB6-Fc_N297A was resistant to inhibition by IL-18 BP (see FIGS. 7A, 7B, and Table 9). Such results demonstrate that introducing the “DB6” set of mutations (i.e., C38I, C68S, and S117C, see Table 5) into a WT IL-18-Fc fusion polypeptide or into an IL-18 variant-Fc fusion polypeptide improves the expression yield of the fusion polypeptide during production in mammalian cells and improves the yield of purified fusion polypeptide following chromatography. Further, WT IL-18 and IL-18 variants that have been engineered to comprise the “DB6” set of mutations exhibit higher potencies of activating IL-18 receptor-mediated signaling. Such results were observed with fusion polypeptides in which the C-terminus of the Fc region was fused to the N-terminus of the IL-18 variant, as well as with fusion polypeptide in which the C-terminus of the IL-18 variant was fused to the N-terminus of the Fc domain.

TABLE 9
EC50s of WT IL-18-DB6-Fc_N297A, MM5-DB6-Fc_N297A,
Fc_N297A-MM5-DB6, and -Fc_N297A-M12-DB6 in an
in vitro IL-18 Receptor Mediated Signaling Assay

| | | EC50 (nM) + 1 μg/ml |
|---|---|---|
| Polypeptide | EC50 (nM) | IL-18 BP-hFc |
| WT IL-18 | 0.15 to 0.31 | 11.1 to 12.9 |
| WT IL-18-DB6-Fc_N297A | 0.011 | 3.13 |
| MM5-DB6-Fc_N297A | 0.13 | 0.12 |
| Fc_N297A-MM5-DB6 | 0.25 | / |
| Fc_N297A-M12-DB6 | 0.06 | / |

The present disclosure is not to be limited in scope by the specific embodiments described which are intended as single illustrations of individual aspects of the disclosure, and any compositions or methods which are functionally equivalent are within the scope of this disclosure. It will be apparent to those skilled in the art that various modifications and variations can be made in the methods and compositions of the present disclosure without departing from the spirit or scope of the disclosure. Thus, it is intended that the present disclosure cover the modifications and variations of this disclosure provided they come within the scope of the appended claims and their equivalents.

All publications and patent applications mentioned in this specification are herein incorporated by reference to the same extent as if each individual publication or patent application was specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

SEQUENCE SUMMARY TABLE

| SEQ ID NO: | NAME | SEQUENCE |
|---|---|---|
| 1 | WT hIL-18 | YFGKLESKLSVIRNLNDQVLFIDQGNRPLFEDMTDSDCRDNAPRTIFIISMYKD |
| | | SQPRGMAVTISVKCEKISTLSCENKIISFKEMNPPDNIKDTKSDIIFFQRSVPGH |
| | | DNKMQFESSSYEGYFLACEKERDLFKLILKKEDELGDRSIMFTVQNED |
| 2 | M26 | YFPKLRSKLSVIRNLNDQVLFIDQGNRPLFEDMTDSDCRDNAPRTIFIISMYKH |
| | | SDKRGMAVTISVKCEKISTLSCENKIISFKEMNPPDVIKDTKSDIIFFQYSVPGH |
| | | DNKMQFESSSYEGYFLACEKERDLFKLILKKEDELGDRSIMFTVQNED |
| 3 | M13 | YFPKLKSKLSVIRNLNDQVLFIDQGNRPLFEDMTDSDCRDNAPRTIFIISMYKQ |
| | | SIWRGMAVTISVKCEKISTLSCENKIISFKEMNPPDNIKDTKSDIIFFQRSVPGH |
| | | DNKMQFESSSYEGYFLACEKERDLFKLILKKEDELGDRSIMFTVQNED |
| 4 | M5 | YFEKLTSKLSVIRNLNDQVLFIDQGNRPLFEDMTDSDCRDNAPRTIFIISMYKW |
| | | SPWRGMAVTISVKCEKISTLSCENKIISFKEMNPPDVIKDTKSDIIFFQTSVPGH |
| | | DNKMQFESSSYEGYFLACEKERDLFKLILKKEDELGDRSIMFTVQKED |
| 5 | | YFPKLRSKLSVIRNLNDQVLFIDQGNRPLFEDMTDSDCRDNAPRTIFIISMYKH |
| | | STVRGMAVTISVKCEKISTLSCENKIISFKEMNPPDVIKDTKSDIIFFQRSVPGH |
| | | DNKMQFESSSYEGYFLACEKERDLFKLILKKEDELGDRSIMFTVQNED |
| 6 | | YFPKLSSKLSVIRNLNDQVLFIDQGNRPLFEDMTDSDCRDNAPRTIFIISMYKW |
| | | SPWRGMAVTISVKCEKISTLSCENKIISFKEMNPPDDIKDTKSDIIFFQRSVPGH |
| | | DNKMQFESSSYEGYFLACEKERDLFKLILKKEDELGDRSIMFTVQNED |
| 7 | | YFPKLKSKLSVIRNLNDQVLFIDQGNRPLFEDMTDSDCRDNAPRTIFIISMYKH |
| | | STVRGMAVTISVKCEKISTLSCENKIISFKEMNPPDVIKDTKSDIIFFQRSVPGH |
| | | DNKMQFESSSYEGYFLACEKERDLFKLILKKEDELGDRSIMFTVQNED |
| 8 | M37 | YFPKLGSKLSVIRNLNDQVLFIDQGNRPLFEDMTDSDCRDNAPRTIFIISMYKQ |
| | | SVWRGMAVTISVKCEKISTLSCENKIISFKEMNPPDNIKDTKSDIIFFQRSVPGH |
| | | DNKMQFESSSYEGYFLACEKERDLFKLILKKEDELGDRSIMFTVQNED |
| 9 | M15 | YFPKLRSKLSVIRNLNDQVLFIDQGNRPLFEDMTDSDCRDNAPRTIFIISMYKS |
| | | SYTRGMAVTISVKCEKISTLSCENKIISFKEMNPPDGIKDTKSDIIFFQRSVPGH |
| | | DNKMQFESSSYEGYFLACEKERDLFKLILKKEDELGDRSIMFTVQNED |
| 10 | | YFTKLKSKLSVIRNLNDQVLFIDQGNRPLFEDMTDSDCRDNAPRTIFIISMYK |
| | | WSSDRGMAVTISVKCEKISTLSCENKIISFKEMNPPDAIKDTKSDIIFFQVSVPG |
| | | HDNKMQFESSSYEGYFLACEKERDLFKLILKKEDELGDRSIMFTVQNED |
| 11 | M7 | YFPKLRSKLSVIRNLNDQVLFIDQGNRPLFEDMTDSDCRDNAPRTIFIISMYKH |
| | | STARGMAVTISVKCEKISTLSCENKIISFKEMNPPDAIKDTKSDIIFFQRSVPGH |
| | | DNKMQFESSSYEGYFLACEKERDLFKLILKKEDELGDRSIMFTVQNED |
| 12 | M3 | YFPKLKSKLSVIRNLNDQVLFIDQGNRPLFEDMTDSDCRDNAPRTIFIISMYKW |
| | | SHVRGMAVTISVKCEKISTLSCENKIISFKEMNPPDAIKDTKSDIIFFQYSVPGH |
| | | DNKMQFESSSYEGYFLACEKERDLFKLILKKEDELGDRSIMFTVQNED |
| 13 | | YFPKLRSKLSVIRNLNDQVLFIDQGNRPLFEDMTDSDCRDNAPRTIFIISMYKW |
| | | SKTRGMAVTISVKCEKISTLSCENKIISFKEMNPPDVIKDTKSDIIFFQYSVPGH |
| | | DNKMQFESSSYEGYFLACEKERDLFKLILKKEDELGDRSIMFTVQNED |
| 14 | | YFPKLRSKLSVIRNLNDQVLFIDQGNRPLFEDMTDSDCRDNAPRTIFIISMYKY |
| | | STARGMAVTISVKCEKISTLSCENKIISFKEMNPPDVIKDTKSDIIFFQHSVPGH |
| | | DNKMQFESSSYEGYFLACEKERDLFKLILKKEDELGDRSIMFTVQNED |
| 15 | | YFPKLRSKLSVIRNLNDQVLFIDQGNRPLFEDMTDSDCRDNAPRTIFIISMYKS |
| | | SRGRGMAVTISVKCEKISTLSCENKIISFKEMNPPDVIKDTKSDIIFFQTSVPGH |
| | | DNKMQFESSSYEGYFLACEKERDLFKLILKKEDELGDRSIMFTVQNED |
| 16 | | YFPKLTSKLSVIRNLNDQVLFIDQGNRPLFEDMTDSDCRDNAPRTIFIISMYKH |
| | | STYRGMAVTISVKCEKISTLSCENKIISFKEMNPPDAIKDTKSDIIFFQRSVPGH |
| | | DNKMQFESSSYEGYFLACEKERDLFKLILKKEDELGDRSIMFTVQKED |
| 17 | M35 | YFPKLRSKLSVIRNLNDQVLFIDQGNRPLFEDMTDSDCRDNAPRTIFIISMYKG |
| | | SGARGMAVTISVKCEKISTLSCENKIISFKEMNPPDGIKDTKSDIIFFQRSVPGH |
| | | DNKMQFESSSYEGYFLACEKERDLFKLILKKEDELGDRSIMFTVQNED |
| 18 | | YLPKLRSKLSVIRNLNDQVLFIDQGNRPLFEDMTDSDCRDNAPRTIFIISMYKW |
| | | STSRGMAVTISVKCEKISTLSCENKIISFKEMNPPDSIKDTKSDIIFFQSSVPGHD |
| | | NKMQFESSSYEGYFLACEKERDLFKLILKKEDELGDRSIMFTVQKED |
| 19 | | YFPKLRSKLSVIRNLNDQVLFIDQGNRPLFEDMTDSDCRDNAPRTIFIISMYKW |
| | | SYPRGMAVTISVKCEKISTLSCENKIISFKEMNPPDSIKDTKSDIIFFQSSVPGHD |
| | | NKMQFESSSYEGYFLACEKERDLFKLILKKEDELGDRSIMFTVQNED |
| 20 | | YFPKLKSKLSVIRNLNDQVLFIDQGNRPLFEDMTDSDCRDNAPRTIFIISMYKF |
| | | STVRGMAVTISVKCEKISTLSCENKIISFKEMNPPDGIKDTKSDIIFFQHSVPGH |
| | | DNKMQFESSSYEGYFLACEKERDLFKLILKKEDELGDRSIMFTVQNED |
| 21 | M2 | YFSKLKSKLSVIRNLNDQVLFIDQGNRPLFEDMTDSDCRDNAPRTIFIISMYKW |
| | | SPDRGMAVTISVKCEKISTLSCENKIISFKEMNPPDGIKDTKSDIIFFQTSVPGH |
| | | DNKMQFESSSYEGYFLACEKERDLFKLILKKEDELGDRSIMFTVQNED |
| 22 | MM1 | YFPKLKSKLSVIRNLNDQVLFIDQGNRPLFEDMTDSDCRDNAPRTIFIISMYKL |
| | | STQRGMAVTISVKCEKISTLSCENKIISFKEMNPPDVIKDTKSDIIFFQRSVPGH |
| | | DNKMQFESSSYEGYFLACEKERDLFKLILKKEDELGDRSIMFTVQNED |
| 23 | | YFPKLKSKLSVIRNLNDQVLFIDQGNRPLFEDMTDSDCRDNAPRTIFIISMYKG |
| | | SGTRGMAVTISVKCEKISTLSCENKIISFKEMNPPDAIKDTKSDIIFFQRSVPGH |
| | | DNKMQFESSSYEGYFLACEKERDLFKLILKKEDELGDRSIMFTVQNED |
| 24 | | YFDKLGSKLSVIRNLNDQVLFIDQGNRPLFEDMTDSDCRDNAPRTIFIISMYK |
| | | WSPWRGMAVTISVKCEKISTLSCENKIISFKEMNPPDNIKDTKSDIIFFQRSVPG |
| | | HDNKMQFESSSYEGYFLACEKERDLFKLILKKEDELGDRSIMFTVQNED |
| 25 | | YFPKLKSKLSVIRNLNDQVLFIDQGNRPLFEDMTDSDCRDNAPRTIFIISMYKL |
| | | SGVRGMAVTISVKCEKISTLSCENKIISFKEMNPPDTIKDTKSDIIFFQRSVPGH |
| | | DNKMQFESSSYEGYFLACEKERDLFKLILKKEDELGDRSIMFTVQNED |
| 26 | M19 | YFPKLKSKLSVIRNLNDQVLFIDQGNRPLFEDMTDSDCRDNAPRTIFIISMYKH |
| | | SEQRGMAVTISVKCEKISTLSCENKIISFKEMNPPDAIKDTKSDIIFFQRSVPGH |
| | | DNKMQFESSSYEGYFLACEKERDLFKLILKKEDELGDRSIMFTVQNED |
| 27 | M21 | YFPKLRSKLSVIRNLNDQVLFIDQGNRPLFEDMTDSDCRDNAPRTIFIISMYKS |
| | | SSTRGMAVTISVKCEKISTLSCENKIISFKEMNPPDGIKDTKSDIIFFQSSVPGHD |
| | | NKMQFESSSYEGYFLACEKERDLFKLILKKEDELGDRSIMFTVQNED |
| 28 | M1 | YFPKLKSKLSVIRNLNDQVLFIDQGNRPLFEDMTDSDCRDNAPRTIFIISMYKH |
| | | SDRRGMAVTISVKCEKISTLSCENKIISFKEMNPPDVIKDTKSDIIFFQVSVPGH |
| | | DNKMQFESSSYEGYFLACEKERDLFKLILKKEDELGDRSIMFTVQNED |
| 29 | | YFPKLTSKLSVIRNLNDQVLFIDQGNRPLFEDMTDSDCRDNAPRTIFIISMYKH |
| | | SYARGMAVTISVKCEKISTLSCENKIISFKEMNPPDVIKDTKSDIIFFQRSVPGH |
| | | DNKMQFESSSYEGYFLACEKERDLFKLILKKEDELGDRSIMFTVQNED |
| 30 | | YFPKLKSKLSVIRNLNDQVLFIDQGNRPLFEDMTDSDCRDNAPRTIFIISMYKH |
| | | SIHRGMAVTISVKCEKISTLSCENKIISFKEMNPPDVIKDTKSDIIFFQVSVPGHD |
| | | NKMQFESSSYEGYFLACEKERDLFKLILKKEDELGDRSIMFTVQNED |
| 31 | | YFPKLKSKLSVIRNLNDQVLFIDQGNRPLFEDMTDSDCRDNAPRTIFIISMYKW |
| | | SARRGMAVTISVKCEKISTLSCENKIISFKEMNPPDPIKDTKSDIIFFQSSVPGHD |
| | | NKMQFESSSYEGYFLACEKERDLFKLILKKEDELGDRSIMFTVQNED |
| 32 | | YFPKLRSKLSVIRNLNDQVLFIDQGNRPLFEDMTDSDCRDNAPRTIFIISMYKH |
| | | SSRRGMAVTISVKCEKISTLSCENKIISFKEMNPPDIIKDTKSDIIFFQYSVPGHD |
| | | NKMQFESSSYEGYFLACEKERDLFKLILKKEDELGDRSIMFTVQNED |
| 33 | | YFGKLKSKLSVIRNLNDQVLFIDQGNRPLFEDMTDSDCRDNAPRTIFIISMYKL |
| | | STRRGMAVTISVKCEKISTLSCENKIISFKEMNPPDPIKDTKSDIIFFQRSVPGHD |
| | | NKMQFESSSYEGYFLACEKERDLFKLILKKEDELGDRSIMFTVQNED |
| 34 | | YFPKLKSKLSVIRNLNDQVLFIDQGNRPLFEDMTDSDCRDNAPRTIFIISMYKQ |
| | | STWRGMAVTISVKCEKISTLSCENKIISFKEMNPPDCIKDTKSDIIFFQRSVPGH |
| | | DNKMQFESSSYEGYFLACEKERDLFKLILKKEDELGDRSIMFTVQNED |
| 35 | M16 | YFPKLRSKLSVIRNLNDQVLFIDQGNRPLFEDMTDSDCRDNAPRTIFIISMYKS |
| | | SRNRGMAVTISVKCEKISTLSCENKIISFKEMNPPDGIKDTKSDIIFFQSSVPGH |
| | | DNKMQFESSSYEGYFLACEKERDLFKLILKKEDELGDRSIMFTVQNED |
| 36 | | YFPKLRSKLSVIRNLNDQVLFIDQGNRPLFEDMTDSDCRDNAPRTIFIISMYKW |
| | | SRPRGMAVTISVKCEKISTLSCENKIISFKEMNPPDSIKDTKSDIIFFQYSVPGHD |
| | | NKMQFESSSYEGYFLACEKERDLFKLILKKEDELGDRSIMFTVQNED |
| 37 | | YFPKLRSKLSVIRNLNDQVLFIDQGNRPLFEDMTDSDCRDNAPRTIFIISMYKG |
| | | SGIRGMAVTISVKCEKISTLSCENKIISFKEMNPPDGIKDTKSDIIFFQRSVPGHD |
| | | NKMQFESSSYEGYFLACEKERDLFKLILKKEDELGDRSIMFTVQNED |
| 38 | M14 | YFNKLKSKLSVIRNLNDQVLFIDQGNRPLFEDMTDSDCRDNAPRTIFIISMYKP |
| | | SSSRGMAVTISVKCEKISTLSCENKIISFKEMNPPDRIKDTKSDIIFFQASVPGHD |
| | | NKMQFESSSYEGYFLACEKERDLFKLILKKEDELGDRSIMFTVQNED |
| 39 | M12 | YFPKLKSKLSVIRNLNDQVLFIDQGNRPLFEDMTDSDCRDNAPRTIFIISMYKG |
| | | SGARGMAVTISVKCEKISTLSCENKIISFKEMNPPDTIKDTKSDIIFFQRSVPGH |
| | | DNKMQFESSSYEGYFLACEKERDLFKLILKKEDELGDRSIMFTVQNED |
| 40 | | YFPKLKSKLSVIRNLNDQVLFIDQGNRPLFEDMTDSDCRDNAPRTIFIISMYKL |
| | | SGARGMAVTISVKCEKISTLSCENKIISFKEMNPPDVIKDTKSDIIFFQRSVPGH |
| | | DNKMQFESSSYEGYFLACEKERDLFKLILKKEDELGDRSIMFTVQNED |
| 41 | M34 | YFPKLRSKLSVIRNLNDQVLFIDQGNRPLFEDMTDSDCRDNAPRTIFIISMYKH |
| | | SRNRGMAVTISVKCEKISTLSCENKIISFKEMNPPDVIKDTKSDIIFFQESVPGH |
| | | DNKMQFESSSYEGYFLACEKERDLFKLILKKEDELGDRSIMFTVQNED |
| 42 | | YFEKLRSKLSVIRNLNDQVLFIDQGNRPLFEDMTDSDCRDNAPRTIFIISMYKW |
| | | SPDRGMAVTISVKCEKISTLSCENKIISFKEMNPPDVIKDTKSDIIFFQVSVPGH |
| | | DNKMQFESSSYEGYFLACEKERDLFKLILKKEDELGDRSIMFTVQNED |
| 43 | M17 | YFPKLRSKLSVIRNLNDQVLFIDQGNRPLFEDMTDSDCRDNAPRTIFIISMYKL |
| | | STARGMAVTISVKCEKISTLSCENKIISFKEMNPPDGIKDTKSDIIFFQRSVPGH |
| | | DNKMQFESSSYEGYFLACEKERDLFKLILKKEDELGDRSIMFTVQNED |
| 44 | M20 | YFPKLRSKLSVIRNLNDQVLFIDQGNRPLFEDMTDSDCRDNAPRTIFIISMYKS |
| | | SRSRGMAVTISVKCEKISTLSCENKIISFKEMNPPDGIKDTKSDIIFFQSSVPGHD |
| | | NKMQFESSSYEGYFLACEKERDLFKLILKKEDELGDRSIMFTVQNED |
| 45 | | YFPKLRSKLSVIRNLNDQVLFIDQGNRPLFEDMTDSDCRDNAPRTIFIISMYKH |
| | | SYGRGMAVTISVKCEKISTLSCENKIISFKEMNPPDAIKDTKSDIIFFQTSVPGH |
| | | DNKMQFESSSYEGYFLACEKERDLFKLILKKEDELGDRSIMFTVQNED |
| 46 | | YFPKLGSKLSVIRNLNDQVLFIDQGNRPLFEDMTDSDCRDNAPRTIFIISMYKH |
| | | SDPRGMAVTISVKCEKISTLSCENKIISFKEMNPPDVIKDTKSDIIFFQMSVPGH |
| | | DNKMQFESSSYEGYFLACEKERDLFKLILKKEDELGDRSIMFTVQNED |
| 47 | | YFPKLRSKLSVIRNLNDQVLFIDQGNRPLFEDMTDSDCRDNAPRTIFIISMYKL |
| | | STVRGMAVTISVKCEKISTLSCENKIISFKEMNPPDGIKDTKSDIIFFQHSVPGH |
| | | DNKMQFESSSYEGYFLACEKERDLFKLILKKEDELGDRSIMFTVQNED |
| 48 | | YFFKLKSKLSVIRNLNDQVLFIDQGNRPLFEDMTDSDCRDNAPRTIFIISMYKP |
| | | SPPRGMAVTISVKCEKISTLSCENKIISFKEMNPPDTIKDTKSDIIFFQYSVPGHD |
| | | NKMQFESSSYEGYFLACEKERDLFKLILKKEDELGDRSIMFTVQNED |
| 49 | M11 | YFPKLRSKLSVIRNLNDQVLFIDQGNRPLFEDMTDSDCRDNAPRTIFIISMYKS |
| | | SRNRGMAVTISVKCEKISTLSCENKIISFKEMNPPDGIKDTKSDIIFFQTSVPGH |
| | | DNKMQFESSSYEGYFLACEKERDLFKLILKKEDELGDRSIMFTVQNED |
| 50 | M28 | YFPKLASKLSVIRNLNDQVLFIDQGNRPLFEDMTDSDCRDNAPRTIFIISMYKW |
| | | SGGRGMAVTISVKCEKISTLSCENKIISFKEMNPPDVIKDTKSDIIFFQYSVPGH |
| | | DNKMQFESSSYEGYFLACEKERDLFKLILKKEDELGDRSIMFTVQNED |
| 51 | | YFPKLRSKLSVIRNLNDQVLFIDQGNRPLFEDMTDSDCRDNAPRTIFIISMYKW |
| | | SAVRGMAVTISVKCEKISTLSCENKIISFKEMNPPDSIKDTKSDIIFFQSSVPGHD |
| | | NKMQFESSSYEGYFLACEKERDLFKLILKKEDELGDRSIMFTVQNED |
| 52 | | YFWKLKSKLSVIRNLNDQVLFIDQGNRPLFEDMTDSDCRDNAPRTIFIISMYKP |
| | | SSHRGMAVTISVKCEKISTLSCENKIISFKEMNPPDVIKDTKSDIIFFQLSVPGH |
| | | DNKMQFESSSYEGYFLACEKERDLFKLILKKEDELGDRSIMFTVQNED |
| 53 | | YFPKLRSKLSVIRNLNDQVLFIDQGNRPLFEDMTDSDCRDNAPRTIFIISMYKL |
| | | SGWRGMAVTISVKCEKISTLSCENKIISFKEMNPPDVIKDTKSDIIFFQRSVPGH |
| | | DNKMQFESSSYEGYFLACEKERDLFKLILKKEDELGDRSIMFTVQNED |
| 54 | M18 | YFPKLRSKLSVIRNLNDQVLFIDQGNRPLFEDMTDSDCRDNAPRTIFIISMYKS |
| | | SRRRGMAVTISVKCEKISTLSCENKIISFKEMNPPDGIKDTKSDIIFFQSSVPGH |
| | | DNKMQFESSSYEGYFLACEKERDLFKLILKKEDELGDRSIMFTVQNED |
| 55 | M38 | YFPKLSSKLSVIRNLNDQVLFIDQGNRPLFEDMTDSDCRDNAPRTIFIISMYKW |
| | | SGARGMAVTISVKCEKISTLSCENKIISFKEMNPPDVIKDTKSDIIFFQISVPGHD |
| | | NKMQFESSSYEGYFLACEKERDLFKLILKKEDELGDRSIMFTVQNED |
| 56 | M6 | YFPKLRSKLSVIRNLNDQVLFIDQGNRPLFEDMTDSDCRDNAPRTIFIISMYKW |
| | | STPRGMAVTISVKCEKISTLSCENKIISFKEMNPPDVIKDTKSDIIFFQTSVPGHD |
| | | NKMQFESSSYEGYFLACEKERDLFKLILKKEDELGDRSIMFTVQNED |
| 57 | | YFPKLKSKLSVIRNLNDQVLFIDQGNRPLFEDMTDSDCRDNAPRTIFIISMYKH |
| | | SRARGMAVTISVKCEKISTLSCENKIISFKEMNPPDSIKDTKSDIIFFQTSVPGHD |
| | | NKMQFESSSYEGYFLACEKERDLFKLILKKEDELGDRSIMFTVQNED |
| 58 | M27 | YFPKLRSKLSVIRNLNDQVLFIDQGNRPLFEDMTDSDCRDNAPRTIFIISMYKH |
| | | SYTRGMAVTISVKCEKISTLSCENKIISFKEMNPPDVIKDTKSDIIFFQYSVPGH |
| | | DNKMQFESSSYEGYFLACEKERDLFKLILKKEDELGDRSIMFTVQNED |
| 59 | M39 | YFPKLRSKLSVIRNLNDQVLFIDQGNRPLFEDMTDSDCRDNAPRTIFIISMYKW |
| | | SPGRGMAVTISVKCEKISTLSCENKIISFKEMNPPDVIKDTKSDIIFFQLSVPGH |
| | | DNKMQFESSSYEGYFLACEKERDLFKLILKKEDELGDRSIMFTVQNED |
| 60 | | YFPKLKSKLSVIRNLNDQVLFIDQGNRPLFEDMTDSDCRDNAPRTIFIISMYKH |
| | | SSTRGMAVTISVKCEKISTLSCENKIISFKEMNPPDVIKDTKSDIIFFQCSVPGHD |
| | | NKMQFESSSYEGYFLACEKERDLFKLILKKEDELGDRSIMFTVQNED |
| 61 | | YFPKLKSKLSVIRNLNDQVLFIDQGNRPLFEDMTDSDCRDNAPRTIFIISMYKF |
| | | STERGMAVTISVKCEKISTLSCENKIISFKEMNPPDPIKDTKSDIIFFQRSVPGHD |
| | | NKMQFESSSYEGYFLACEKERDLFKLILKKEDELGDRSIMFTVQNED |
| 62 | M8 | YFPKLRSKLSVIRNLNDQVLFIDQGNRPLFEDMTDSDCRDNAPRTIFIISMYKW |
| | | SPARGMAVTISVKCEKISTLSCENKIISFKEMNPPDAIKDTKSDIIFFQLSVPGH |
| | | DNKMQFESSSYEGYFLACEKERDLFKLILKKEDELGDRSIMFTVQNED |
| 63 | | YFPKLQSKLSVIRNLNDQVLFIDQGNRPLFEDMTDSDCRDNAPRTIFIISMYKL |
| | | STPRGMAVTISVKCEKISTLSCENKIISFKEMNPPDAIKDTKSDIIFFQRSVPGHD |
| | | NKMQFESSSYEGYFLACEKERDLFKLILKKEDELGDRSIMFTVQNED |
| 64 | | YFPKLNSKLSVIRNLNDQVLFIDQGNRPLFEDMTDSDCRDNAPRTIFIISMYKV |
| | | PTARGMAVTISVKCEKISTLSCENKIISFKEMNPPDAIKDTKSDIIFFQRSVPGH |
| | | DNKMQFESSSYEGYFLACEKERDLFKLILKKEDELGDRSIMFTVQNED |
| 65 | M31 | YFPKLRSKLSVIRNLNDQVLFIDQGNRPLFEDMTDSDCRDNAPRTIFIISMYKH |
| | | SIHRGMAVTISVKCEKISTLSCENKIISFKEMNPPDIIKDTKSDIIFFQYSVPGHD |
| | | NKMQFESSSYEGYFLACEKERDLFKLILKKEDELGDRSIMFTVQNED |
| 66 | M10 | YFPKLRSKLSVIRNLNDQVLFIDQGNRPLFEDMTDSDCRDNAPRTIFIISMYKQ |
| | | SLWRGMAVTISVKCEKISTLSCENKIISFKEMNPPDSIKDTKSDIIFFQRSVPGH |
| | | DNKMQFESSSYEGYFLACEKERDLFKLILKKEDELGDRSIMFTVQNED |
| 67 | | YFTKLRSKLSVIRNLNDQVLFIDQGNRPLFEDMTDSDCRDNAPRTIFIISMYKL |
| | | SHLRGIAVTISVKCEKISTLSCENKIISFKEMNPPDAIKDTKSDIIFFQSSVPGHD |
| | | NKMQFESSSYEGYFLACEKERDLFKLILKKEDELGDRSIMFTVQNED |
| 68 | | YFPKLKSKLSVIRNLNDQVLFIDQGNRPLFEDMTDSDCRDNAPRTIFIISMYKW |
| | | SPWRGMAVTISVKCEKISTLSCENKIISFKEMNPPDNIKDTKSDIIFFQTSVPGH |
| | | DNKMQFESSSYEGYFLACEKERDLFKLILKKEDELGDRSIMFTVQNED |
| 69 | | YFPKLVSKLSVIRNLNDQVLFIDQGNRPLFEDMTDSDCRDNAPRTIFIISMYKW |
| | | SAIRGMAVTISVKCEKISTLSCENKIISFKEMNPPDVIKDTKSDIIFFQTSVPGHD |
| | | NKMQFESSSYEGYFLACEKERDLFKLILKKEDELGDRSIMFTVQNED |
| 70 | | YFPKLRSKLSVIRNLNDQVLFIDQGNRPLFEDMTDSDCRDNAPRTIFIISMYKW |
| | | SGGRGMAVTISVKCEKISTLSCENKIISFKEMNPPDAIKDTKSDIIFFQLSVPGH |
| | | DNKMQFESSSYEGYFLACEKERDLFKLILKKEDELGDRSIMFTVQNED |
| 71 | | YFPKLYSKLSVIRNLNDQVLFIDQGNRPLFEDMTDSDCRDNAPRTIFIISMYKH |
| | | STNRGMAVTISVKCEKISTLSCENKIISFKEMNPPDGIKDTKSDIIFFQRSVPGH |
| | | DNKMQFESSSYEGYFLACEKERDLFKLILKKEDELGDRSIMFTVQNED |
| 72 | M4 | YFPKLKSKLSVIRNLNDQVLFIDQGNRPLFEDMTDSDCRDNAPRTIFIISMYKW |
| | | SGVRGMAVTISVKCEKISTLSCENKIISFKEMNPPDVIKDTKSDIIFFQFSVPGH |
| | | DNKMQFESSSYEGYFLACEKERDLFKLILKKEDELGDRSIMFTVQNED |
| 73 | M40 | YFDKLKSKLSVIRNLNDQVLFIDQGNRPLFEDMTDSDCRDNAPRTIFIISMYKP |
| | | SSWRGMAVTISVKCEKISTLSCENKIISFKEMNPPDWIKDTKSDIIFFQRSVPGH |
| | | DNKMQFESSSYEGYFLACEKERDLFKLILKKEDELGDRSIMFTVQNED |
| 74 | M9 | YFPKLRSKLSVIRNLNDQVLFIDQGNRPLFEDMTDSDCRDNAPRTIFIISMYKW |
| | | SRARGMAVTISVKCEKISTLSCENKIISFKEMNPPDSIKDTKSDIIFFQSSVPGHD |
| | | NKMQFESSSYEGYFLACEKERDLFKLILKKEDELGDRSIMFTVQNED |
| 75 | | YFPKLGSKLSVIRNLNDQVLFIDQGNRPLFEDMTDSDCRDNAPRTIFIISMYKH |
| | | SSRRGMAVTISVKCEKISTLSCENKIISFKEMNPPDVIKDTKSDIIFFQNSVPGH |
| | | DNKMQFESSSYEGYFLACEKERDLFKLILKKEDELGDRSIMFTVQNED |
| 76 | | YFPKLKSKLSVIRNLNDQVLFIDQGNRPLFEDMTDSDCRDNAPRTIFIISMYKW |
| | | SWARGMAVTISVKCEKISTLSCENKIISFKEMNPPDLIKDTKSDIIFFQYSVPGH |
| | | DNKMQFESSSYEGYFLACEKERDLFKLILKKEDELGDRSIMFTVQNED |
| 77 | | YFPKLRSKLSVIRNLNDQVLFIDQGNRPLFEDMTDSDCRDNAPRTIFIISMYKH |
| | | SYTRGMAVTISVKCEKISTLSCENKIISFKEMNPPDIIKDTKSDIIFFQYSVPGHD |
| | | NKMQFESSSYEGYFLACEKERDLFKLILKKEDELGDRSIMFTVQNED |
| 78 | | YFKKLPSKLSVIHNLNDQVLFIDQGNRPLFEDMTDSDCRDNAPRTIFIISMYKL |
| | | SSERGMAVTISVKCEKISTLSCENKIISFKEMNPPDNIKDTKSDIIFFQRSVPGHD |
| | | NKMQFESSSYEGYFLACEKERDLFKLILKKEDELGDRSIMFTVQNED |
| 79 | | YFPKLGSKLSVIRNLNDQVLFIDQGNRPLFEDMTDSDCRDNAPRTIFIISMYKW |
| | | SQPRGMAVTISVKCEKISTLSCENKIISFKEMNPPDVIKDTKSDIIFFQYSVPGH |
| | | DNKMQFESSSYEGYFLACEKERDLFKLILKKEDELGDRSIMFTVQNED |
| 80 | | YFPKLRSKLSVIRNLNDQVLFIDQGNRPLFEDMTDSDCRDNAPRTIFIISMYKW |
| | | SYPRGMAVTISVKCEKISTLSCENKIISFKEMNPPDGIKDTKSDIIFFQFSVPGHD |
| | | NKMQFESSSYEGYFLACEKERDLFKLILKKEDELGDRSIMFTVQNED |
| 81 | M36 | YFPKLSSKLSVIRNLNDQVLFIDQGNRPLFEDMTDSDCRDNAPRTIFIISMYKA |
| | | SDQRGMAVTISVKCEKISTLSCENKIISFKEMNPPDGIKDTKSDIIFFQRSVPGH |
| | | DNKMQFESSSYEGYFLACEKERDLFKLILKKEDELGDRSIMFTVQNED |
| 82 | | YFPKLRSKLSVIRNLNDQVLFIDQGNRPLFEDMTDSDCRDNAPRTIFIISMYKL |
| | | STIRGMAVTISVKCEKISTLSCENKIISFKEMNPPDYIKDTKSDIIFFQRSVPGHD |
| | | NKMQFESSSYEGYFLACEKERDLFKLILKKEDELGDRSIMFTVQNED |
| 83 | | YFWKLWSKLSVIRNLNDQVLFIDQGNRPLFEDMTDSDCRDNAPRTIFIISMYK |
| | | PSNFRGMAVTISVKCEKISTLSCENKIISFKEMNPPDVIKDTKSDIIFFQGSVPGH |
| | | DNKMQFESSSYEGYFLACEKERDLFKLILKKEDELGDRSIMFTVQNED |
| 84 | | YFPKLKSKLSVIRNLNDQVLFIDQGNRPLFEDMTDSDCRDNAPRTIFIISMYKM |
| | | SSARGMAVTISVKCEKISTLSCENKIISFKEMNPPDGIKDTKSDIIFFQSSVPGHD |
| | | NKMQFESSSYEGYFLACEKERDLFKLILKKEDELGDRSIMFTVQNED |
| 85 | | YFPKLKSKLSVIRNLNDQVLFIDQGNRPLFEDMTDSDCRDNAPRTIFIISMYKW |
| | | SGGRGMAVTISVKCEKISTLSCENKIISFKEMNPPDSIKDTKSDIIFFQFSVPGHD |
| | | NKMQFESSSYEGYFLACEKERDLFKLILKKEDELGDRSIMFTVQNED |
| 86 | | YFPKLNSKLSVIRNLNDQVLFIDQGNRPLFEDMTDSDCRDNAPRTIFIISMYKL |
| | | STVRGMAVTISVKCEKISTLSCENKIISFKEMNPPDPIKDTKSDIIFFQHSVPGH |
| | | DNKMQFESSSYEGYFLACEKERDLFKLILKKEDELGDRSIMFTVQNED |
| 87 | | YFPKLGSKLSVIRNLNDQVLFIDQGNRPLFEDMTDSDCRDNAPRTIFIISMYKG |
| | | SHFRGMAVTISVKCEKISTLSCENKIISFKEMNPPDGIKDTKSDIIFFQSSVPGHD |
| | | NKMQFESSSYEGYFLACEKERDLFKLILKKEDELGDRSIMFTVQNED |
| 88 | | YFPKLSSKLSVIRNLNDQVLFIDQGNRPLFEDMTDSDCRDNAPRTIFIISMYKH |
| | | SSRRGMAVTISVKCEKISTLSCENKIISFKEMNPPDVIKDTKSDIIFFQASVPGH |
| | | DNKMQFESSSYEGYFLACEKERDLFKLILKKEDELGDRSIMFTVQNED |
| 89 | | YFPKLASKLSVIRNLNDQVLFIDQGNRPLFEDMTDSDCRDNAPRTIFIISMYKH |
| | | SSKRGMAVTISVKCEKISTLSCENKIISFKEMNPPDVIKDTKSDIIFFQVSVPGH |
| | | DNKMQFESSSYEGYFLACEKERDLFKLILKKEDELGDRSIMFTVQNED |
| 90 | | YFPKLRSKLSVIRNLNDQVLFIDQGNRPLFEDMTDSDCRDNAPRTIFIISMYKS |
| | | SLKRGMAVTISVKCEKISTLSCENKIISFKEMNPPDTIKDTKSDIIFFQSSVPGHD |
| | | NKMQFESSSYEGYFLACEKERDLFKLILKKEDELGDRSIMFTVQNED |
| 91 | M22 | YFPKLRSKLSVIRNLNDQVLFIDQGNRPLFEDMTDSDCRDNAPRTIFIISMYKY |
| | | SRGRGMAVTISVKCEKISTLSCENKIISFKEMNPPDKIKDTKSDIIFFQSSVPGH |
| | | DNKMQFESSSYEGYFLACEKERDLFKLILKKEDELGDRSIMFTVQNED |
| 92 | | YFWKLSSKLSVIRNLNDQVLFIDQGNRPLFEDMTDSDCRDNAPRTIFIISMYKQ |
| | | SLRRGMAVTISVKCEKISTLSCENKIISFKEMNPPDRIKDTKSDIIFFQASVPGH |
| | | DNKMQFESSSYEGYFLACEKERDLFKLILKKEDELGDRSIMFTVQNED |
| 93 | | YFGKLPSKLSVIRNLNDQVLFIDQGNRPLFEDMTDSDCRDNAPRTIFIISMYKL |
| | | SNRRGMAVTISVKCEKISTLSCENKIISFKEMNPPDGIKDTKSDIIFFQRSVPGH |
| | | DNKMQFESSSYEGYFLACEKERDLFKLILKKEDELGDRSIMFTVQNED |
| 94 | M32 | YFPKLGSKLSVIRNLNDQVLFIDQGNRPLFEDMTDSDCRDNAPRTIFIISMYKS |
| | | SSRRGMAVTISVKCEKISTLSCENKIISFKEMNPPDNIKDTKSDIIFFQRSVPGH |
| | | DNKMQFESSSYEGYFLACEKERDLFKLILKKEDELGDRSIMFTVQNED |
| 95 | | YFWKLSSKLSVIRNLNDQVLFIDQGNRPLFEDMTDSDCRDNAPRTIFIISMYKL |
| | | STWRGMAVTISVKCEKISTLSCENKIISFKEMNPPDYIKDTKSDIIFFQRSVPGH |
| | | DNKMQFESSSYEGYFLACEKERDLFKLILKKEDELGDRSIMFTVQNED |
| 96 | | YFPKLHSKLSVIRNLNDQVLFIDQGNRPLFEDMTDSDCRDNAPRTIFIISMYKR |
| | | STLRGMAVTISVKCEKISTLSCENKIISFKEMNPPDGIKDTKSDIIFFQRSVPGH |
| | | DNKMQFESSSYEGYFLACEKERDLFKLILKKEDELGDRSIMFTVQNED |
| 97 | | YFPKLKSKLSVIRNLNDQVLFIDQGNRPLFEDMTDSDCRDNAPRTIFIISMYKT |
| | | SFNRGMAVTISVKCEKISTLSCENKIISFKEMNPPDVIKDTKSDIIFFQRSVPGH |
| | | DNKMQFESSSYEGYFLACEKERDLFKLILKKEDELGDRSIMFTVQNED |
| 98 | | YFPKLTSKLSVIRNLNDQVLFIDQGNRPLFEDMTDSDCRDNAPRTIFIISMYKY |
| | | STRRGMAVTISVKCEKISTLSCENKIISFKEMNPPDNIKDTKSDIIFFQRSVPGH |
| | | DNKMQFESSSYEGYFLACEKERDLFKLILKKEDELGDRSIMFTVQNED |
| 99 | M23 | YFPKLRSKLSVIRNLNDQVLFIDQGNRPLFEDMTDSDCRDNAPRTIFIISMYKY |
| | | STRRGMAVTISVKCEKISTLSCENKIISFKEMNPPDNIKDTKSDIIFFQRSVPGH |
| | | DNKMQFESSSYEGYFLACEKERDLFKLILKKEDELGDRSIMFTVQNED |
| 100 | | YFSKLPSKLSVIRNLNDQVLFIDQGNRPLFEDMTDSDCRDNAPRTIFIISMYKY |
| | | STWRGMAVTISVKCEKISTLSCENKIISFKEMNPPDFIKDTKSDIIFFQSSVPGH |
| | | DNKMQFESSSYEGYFLACEKERDLFKLILKKEDELGDRSIMFTVQNED |
| 101 | | YFPKLKSKLSVIRNLNDQVLFIDQGNRPLFEDMTDSDCRDNAPRTIFIISMYKL |
| | | SRTRGMAVTISVKCEKISTLSCENKIISFKEMNPPDFIKDTKSDIIFFQSSVPGHD |
| | | NKMQFESSSYEGYFLACEKERDLFKLILKKEDELGDRSIMFTVQNED |
| 102 | | YFPKLRSKLSVIRNLNDQVLFIDQGNRPLFEDMTDSDCRDNAPRTIFIISMYKF |
| | | SNFRGMAVTISVKCEKISTLSCENKIISFKEMNPPDAIKDTKSDIIFFQSSVPGHD |
| | | NKMQFESSSYEGYFLACEKERDLFKLILKKEDELGDRSIMFTVQNED |
| 103 | | YFSKLPSKLSVIRNLNDQVLFIDQGNRPLFEDMTDSDCRDNAPRTIFIISMYKW |
| | | SPRRGMAVTISVKCEKISTLSCENKIISFKEMNPPDIIKDTKSDIIFFQSSVPGHD |
| | | NKMQFESSSYEGYFLACEKERDLFKLILKKEDELGDRSIMFTVQNED |
| 104 | M25 | YFPKLRSKLSVIRNLNDQVLFIDQGNRPLFEDMTDSDCRDNAPRTIFIISMYKL |
| | | STTRGMAVTISVKCEKISTLSCENKIISFKEMNPPDRIKDTKSDIIFFQRSVPGHD |
| | | NKMQFESSSYEGYFLACEKERDLFKLILKKEDELGDRSIMFTVQNED |
| 105 | M24 | YFPKLRSKLSVIRNLNDQVLFIDQGNRPLFEDMTDSDCRDNAPRTIFIISMYKY |
| | | STSRGMAVTISVKCEKISTLSCENKIISFKEMNPPDNIKDTKSDIIFFQRSVPGHD |
| | | NKMQFESSSYEGYFLACEKERDLFKLILKKEDELGDRSIMFTVQNED |
| 106 | | YFPKLSSKLSVIRNLNDQVLFIDQGNRPLFEDMTDSDCRDNAPRTIFIISMYKA |
| | | SSLRGMAVTISVKCEKISTLSCENKIISFKEMNPPDSIKDTKSDIIFFQRSVPGHD |
| | | NKMQFESSSYEGYFLACEKERDLFKLILKKEDELGDRSIMFTVQNED |
| 107 | | YFPKLTSKLSVIRNLNDQVLFIDQGNRPLFEDMTDSDCRDNAPRTIFIISMYKT |
| | | SYERGMAVTISVKCEKISTLSCENKIISFKEMNPPDVIKDTKSDIIFFQRSVPGH |
| | | DNKMQFESSSYEGYFLACEKERDLFKLILKKEDELGDRSIMFTVQNED |
| 108 | | YFPKLGSKLSVIRNLNDQVLFIDQGNRPLFEDMTDSDCRDNAPRTIFIISMYKQ |
| | | SHYRGMAVTISVKCEKISTLSCENKIISFKEMNPPDNIKDTKSDIIFFQRSVPGH |
| | | DNKMQFESSSYEGYFLACEKERDLFKLILKKEDELGDRSIMFTVQNED |
| 109 | | YFKKLPSKLSVIRNLNDQVLFIDQGNRPLFEDMTDSDCRDNAPRTIFIISMYKL |
| | | SSERGMAVTISVKCEKISTLSCENKIISFKEMNPPDNIKDTKSDIIFFQRSVPGHD |
| | | NKMQFESSSYEGYFLACEKERDLFKLILKKEDELGDRSIMFTVQNED |
| 110 | | YFPKLNSKLSVIRNLNDQVLFIDQGNRPLFEDMTDSDCRDNAPRTIFIISMYKL |
| | | SPRRGMAVTISVKCEKISTLSCENKIISFKEMNPPDNIKDTKSDIIFFQRSVPGH |
| | | DNKMQFESSSYEGYFLACEKERDLFKLILKKEDELGDRSIMFTVQNED |
| 111 | | YFPKLESKLSVIRNLNDQVLFIDQGNRPLFEDMTDSDCRDNAPRTIFIISMYKY |
| | | SSKRGMAVTISVKCEKISTLSCENKIISFKEMNPPDGIKDTKSDIIFFQRSVPGH |
| | | DNKMQFESSSYEGYFLACEKERDLFKLILKKEDELGDRSIMFTVQNED |
| 112 | | YFPKLRSKLSVIRNLNDQVLFIDQGNRPLFEDMTDSDCRDNAPRTIFIISMYKH |
| | | STNRGMAVTISVKCEKISTLSCENKIISFKEMNPPDIIKDTKSDIIFFQISVPGHD |
| | | NKMQFESSSYEGYFLACEKERDLFKLILKKEDELGDRSIMFTVQNED |
| 113 | | YFPKLRSKLSVIRNLNDQVLFIDQGNRPLFEDMTDSDCRDNAPRTIFIISMYKIS |
| | | ISRGMAVTISVKCEKISTLSCENKIISFKEMNPPDNIKDTKSDIIFFQRSVPGHDN |
| | | KMQFESSSYEGYFLACEKERDLFKLILKKEDELGDRSIMFTVQNED |
| 114 | M30 | YFPKLLSKLSVIRNLNDQVLFIDQGNRPLFEDMTDSDCRDNAPRTIFIISMYKH |
| | | STVRGMAVTISVKCEKISTLSCENKIISFKEMNPPDSIKDTKSDIIFFQRSVPGHD |
| | | NKMQFESSSYEGYFLACEKERDLFKLILKKEDELGDRSIMFTVQNED |
| 115 | | YFPKLGSKLSVIRNLNDQVLFIDQGNRPLFEDMTDSDCRDNAPRTIFIISMYKIS |
| | | HDRGMAVTISVKCEKISTLSCENKIISFKEMNPPDTIKDTKSDIIFFQSSVPGHD |
| | | NKMQFESSSYEGYFLACEKERDLFKLILKKEDELGDRSIMFTVQNED |
| 116 | M29 | YFPKLMSKLSVIRNLNDQVLFIDQGNRPLFEDMTDSDCRDNAPRTIFIISMYKF |
| | | SDRRGMAVTISVKCEKISTLSCENKIISFKEMNPPDPIKDTKSDIIFFQRSVPGH |
| | | DNKMQFESSSYEGYFLACEKERDLFKLILKKEDELGDRSIMFTVQNED |
| 117 | M33 | YFEKLHSKLSVIRNLNDQVLFIDQGNRPLFEDMTDSDCRDNAPRTIFIISMYKR |
| | | STHRGMAVTISVKCEKISTLSCENKIISFKEMNPPDNIKDTKSDIIFFQRSVPGH |
| | | DNKMQFESSSYEGYFLACEKERDLFKLILKKEDELGDRSIMFTVQNED |
| 118 | | YFPKLASKLSVIRNLNDQVLFIDQGNRPLFEDMTDSDCRDNAPRTIFIISMYKW |
| | | SNARGMAVTISVKCEKISTLSCENKIISFKEMNPPDSIKDTKSDIIFFQRSVPGH |
| | | DNKMQFESSSYEGYFLACEKERDLFKLILKKEDELGDRSIMFTVQNED |
| 119 | | YFPKLKSKLSVIRNLNDQVLFIDQGNRPLFEDMTDSDCRDNAPRTIFIISMYKY |
| | | SCARGMAVTISVKCEKISTLSCENKIISFKEMNPPDAIKDTKSDIIFFQRSVPGH |
| | | DNKMQFESSSYEGYFLACEKERDLFKLILKKEDELGDRSIMFTVQNED |
| 120 | | YFPKLVSKLSVIRNLNDQVLFIDQGNRPLFEDMTDSDCRDNAPRTIFIISMYKW |
| | | SPWRGMAVTISVKCEKISTLSCENKIISFKEMNPPDSIKDTKSDIIFFQRSVPGH |
| | | DNKMQFESSSYEGYFLACEKERDLFKLILKKEDELGDRSIMFTVQNED |
| 121 | LM1 | YFPKLKSKLSVIRNLNDQVLFIDQGNRPLFEDMTDSDCRDNAPRTIFIISMYKG |
| | | SGARGMAVTISVKCEKISTLSCENKIISFKEMNPPDNIKDTKSDIIFFQRSVPGH |
| | | DNKMQFESSSYEGYFLACEKERDLFKLILKKEDELGDRSIMFTVQNED |
| 122 | LM2 | YFPKLRSKLSVIRNLNDQVLFIDQGNRPLFEDMTDSDCRDNAPRTIFIISMYKL |
| | | SGSRGMAVTISVKCEKISTLSCENKIISFKEMNPPDVIKDTKSDIIFFQRSVPGH |
| | | DNKMQFESSSYEGYFLACEKERDLFKLILKKEDELGDRSIMFTVQNED |
| 123 | LM3 | YFPKLRSKLSIIRNLNDQVLFIDQGNRPLFEDMTDSDCRDNAPRTIFIISMYKGS |
| | | GARGMAVTISVKCEKISTLSCENKIISFKEMNPPDGIKDTKSDIIFFQRSVPGHD |
| | | NKMQFESSSYEGYFLACEKERDLFKLILKKEDELGDRSIMFTVQNED |
| 124 | LM4 | YFPKLKSKLSVIRNLNDQVLFIDQGNRPLFEDMTDSDCRDNAPRTIFIISMYKH |
| | | SYSRGMAVTISVKCEKISTLSCENKIISFKEMNPPDNIKDTKSDIIFFQRSVPGH |
| | | DNKMQFESSSYEGYFLACEKERDLFKLILKKEDELGDRSIMFTVQNED |
| 125 | LM5 | YFGKLRSKLSVIRNLNDQVLFIDQGNRPLFEDMTDSDCRDNAPRTIFIISMYK |
| | | WSSQRGMAVTISVKCEKISTLSCENKIISFKEMNPPDNIKDTKSDIIFFQRSVPG |
| | | HDNKMQFESSSYEGYFLACEKERDLFKLILKKEDELGDRSIMFTVQNED |
| 126 | | YFPKLVSKLSVIRNLNDQVLFIDQGNRPLFEDMTDSDCRDNAPRTIFIISMYKR |
| | | SSLRGMAVTISVKCEKISTLSCENKIISFKEMNPPDDIKDTKSDIIFFQFSVPGHD |
| | | NKMQFESSSYEGYFLACEKERDLFKLILKKEDELGDRSIMFTVQNED |
| 127 | | YFAKLYSKLSVIRNLNDQVLFIDQGNRPLFEDMTDSDCRDNAPRTIFIISMYKY |
| | | STARGMAVTISVKCEKISTLSCENKIISFKEMNPPDVIKDTKSDIIFFQHSVPGH |
| | | DNKMQFESSSYEGYFLACEKERDLFKLILKKEDELGDRSIMFTVQNED |
| 128 | | YFPKLRSKLSVIRNLNDQVLFIDQGNRPLFEDMTDSDCRDNAPRTIFIISMYKG |
| | | SGARGMAVTISVKCEKISTLSCENKIISFKEMNPPDVIKDTKSDIIFFQRSVPGH |
| | | DNKMQFESSSYEGYFLACEKERDLFKLILKKEDELGDRSIMFTVQNED |
| 129 | | YFPKLRSKLSVIRNLNDQVLFIDQGNRPLFEDMTDSDCRDNAPRTIFIISMYKF |
| | | SDVRGMAVTISVKCEKISTLSCENKIISFKEMNPPDAIKDTKSDIIFFQRSVPGH |
| | | DNKMQFESSSYEGYFLACEKERDLFKLILKKEDELGDRSIMFTVQNED |
| 130 | | YFPKLRSKLSVIRNLNDQVLFIDQGNRPLFEDMTDSDCRDNAPRTIFIISMYKL |
| | | SPWRGMAVTISVKCEKISTLSCENKIISFKEMNPPDVIKDTKSDIIFFQRSVPGH |
| | | DNKMQFESSSYEGYFLACEKERDLFKLILKKEDELGDRSIMFTVQNED |
| 131 | MM2 | YFAKLYSKLSVIRNLNDQVLFIDQGNRPLFEDMTDSDCRDNAPRTIFIISMYKR |
| | | SSLRGMAVTISVKCEKISTLSCENKIISFKEMNPPDGIKDTKSDIIFFQRSVPGHD |
| | | NKMQFESSSYEGYFLACEKERDLFKLILKKEDELGDRSIMFTVQNED |
| 132 | MM4 | YFPKLRSKLSVIRNLNDQVLFIDQGNRPLFEDMTDSDCRDNAPRTIFIISMYKL |
| | | STIRGMAVTISVKCEKISTLSCENKIISFKEMNPPDGIKDTKSDIIFFQRSVPGHD |
| | | NKMQFESSSYEGYFLACEKERDLFKLILKKEDELGDRSIMFTVQNED |
| 133 | MM5 | YFGKLGSKLSVIRNLNDQVLFIDQGNRPLFEDMTDSDCRDNAPRTIFIISMYKL |
| | | STERGMAVTISVKCEKISTLSCENKIISFKEMNPPDGIKDTKSDIIFFQSSVPGHD |
| | | NKMQFESSSYEGYFLACEKERDLFKLILKKEDELGDRSIMFTVQNED |
| 134 | MM6 | YFAKLYSKLSVIRNLNDQVLFIDQGNRPLFEDMTDSDCRDNAPRTIFIISMYKR |
| | | SSLRGMAVTISVKCEKISTLSCENKIISFKEMNPPDAIKDTKSDIIFFQRSVPGHD |
| | | NKMQFESSSYEGYFLACEKERDLFKLILKKEDELGDRSIMFTVQNED |
| 135 | | YFPKLRSKLSVIRNLNDQVLFIDQGNRPLFEDMTDSDCRDNAPRTIFIISMYKH |
| | | SWARGMAVTISVKCEKISTLSCENKIISFKEMNPPDGIKDTKSDIIFFQRSVPGH |
| | | DNKMQFESSSYEGYFLACEKERDLFKLILKKEDELGDRSIMFTVQNED |
| 136 | | YFPKLSSKLSVIRNLNDQVLFIDQGNRPLFEDMTDSDCRDNAPRTIFIISMYKR |
| | | SSLRGMAVTISVKCEKISTLSCENKIISFKEMNPPDDIKDTKSDIIFFQFSVPGHD |
| | | NKMQFESSSYEGYFLACEKERDLFKLILKKEDELGDRSIMFTVQNED |
| 137 | | YFPKLRSKLSVIRNLNDQVLFIDQGNRPLFEDMTDSDCRDNAPRTIFIISMYKH |
| | | STSRGMAVTISVKCEKISTLSCENKIISFKEMNPPDNIKDTKSDIIFFQRSVPGHD |
| | | NKMQFESSSYEGYFLACEKERDLFKLILKKEDELGDRSIMFTVQNED |
| 138 | | YFAKLYSKLSVIRNLNDQVLFIDQGNRPLFEDMTDSDCRDNAPRTIFIISMYKR |
| | | SSLRGMAVTISVKCEKISTLSCENKIISFKEMNPPDYIKDTKSDIIFFQRSVPGHD |
| | | NKMQFESSSYEGYFLACEKERDLFKLILKKEDELGDRSIMFTVQNED |
| 139 | M41 | YFPKLESKLSVIRNLNDQVLFIDQGNRPLFEDMTDSDCRDNAPRTIFIISMYKD |
| | | SQPRGMAVTISVKCEKISTLSCENKIISFKEMNPPDNIKDTKSDIIFFQRSVPGH |
| | | DNKMQFESSSYEGYFLACEKERDLFKLILKKEDELGDRSIMFTVQNED |
| 140 | WM1 | YFGKLESKLSVIRNLNDQVLFIDQGNRPLFEDMTDSDCRDNAPRTIFIISMYKD |
| | | SQPRGKAVTISVKCEKISTLSCENKIISFKEMNPPDNIKDTDSDIIFFQRSVPGHD |
| | | NKMQFESSSYEGYFLACEKERDLFKLILKKEDELGDRSIMFTVQNED |
| 141 | WM2 | YFPKLRSKLSVIRNLNDQVLFIDQGNRPLFEDMTDSDCRDNAPRTIFIISMYKD |
| | | SQPRGMAVTISVKCEKISTLSCENKIISFKEMNPPDNIKDTESDIIFFQRSVPGHD |
| | | NKMQFESSSYEGYFLACEKERDLFKLILKKEDELGDRSIMFTVQNED |
| 142 | WM3 | YFGKLESKLSVIRNLNDQVLFIDQGNRPLFEDMTDSDCRDNAPRTIFIISMYKD |
| | | SQPRGKAVTISVKCEKISTLSCENKIISFKEMNPPDNIKDTKSDIIFFQRSVPGHD |
| | | NKMQFESSSYEGYFLACEKERDLFKLILKKEDELGDRSIMFTVQNED |
| 143 | WM4 | YFPKLRSKLSVIRNLNDQVLFIDQGNRPLFEDMTDSDCRDNAPRTIFIISMYKD |
| | | SQPRGMAVTISVKCEKISTLSCENKIISFKEMNPPDNIKDTKSDIIFFQRSVPGH |
| | | DNKMQFESSSYEGYFLACEKERDLFKLILKKEDELGDRSIMFTVQNED |
| 144 | WM5 | YFPKLKSKLSVIRNLNDQVLFIDQGNRPLFEDMTDSDCRDNAPRTIFIISMYKD |
| | | SQPRGMAVTISVKCEKISTLSCENKIISFKEMNPPDNIKDTKSDIIFFQRSVPGH |
| | | DNKMQFESSSYEGYFLACEKERDLFKLILKKEDELGDRSIMFTVQNED |
| 145 | WM6 | YFDKLKSKLSVIRNLNDQVLFIDQGNRPLFEDMTDSDCRDNAPRTIFIISMYKD |
| | | SQPRGMAVTISVKCEKISTLSCENKIISFKEMNPPDSIKDTKSDIIFFQRSVPGHD |
| | | NKMQFESSSYEGYFLACEKERDLFKLILKKEDELGDRSIMFTVQNED |
| 146 | WM7 | YFSKLESKLSVIRNLNDQVLFIDQGNRPLFEDMTDSDCRDNAPRTIFIISMYKD |
| | | SQPRGMAVTISVKCEKISTLSCENKIISFKEMNPPDNIKDTKSDIIFFQRSVPGH |
| | | DNKMQFESSSYEGYFLACEKERDLFKLILKKEDELGDRSMMFTVQNED |
| 147 | WM8 | YFPKLRSKLSVIRNLNDQVLFIDQGNRPLFEDMTDSDCRDNAPRTIFIISMYKD |
| | | SQPRGMAVTISVKCEKISTLSCENKIISFKEMNPPDSIKDTKSDIIFFQRSVPGHD |
| | | NKMQFESSSYEGYFLACEKERDLFKLILKKEDELGDRSIMFTVQNED |
| 148 | WM9 | YFGKLRSKLSVIRNLNDQVLFIDQGNRPLFEDMTDSDCRDNAPRTIFIISMYKD |
| | | SQPRGMAVTISVKCEKISTLSCENKIISFKEMNPPDNIKDTKSDIIFFQRSVPGH |
| | | DNKMQFESSSYEGYFLACEKERDLFKLILKKEDELGDRSIMFTVQNED |
| 149 | WM10 | YFPKLKSKLSVIRNLNDQVLFIDQGNRPLFEDMTDSDCRDNAPRTIFIISMYKD |
| | | SQPRGMAVTISVKCEKISTLSCENKIISFKEMNPPDSIKDTKSDIIFFQRSVPGHD |
| | | NKMQFESSSYEGYFLACEKERDLFKLILKKEDELGDRSIMFTVQNED |
| 150 | WM11 | YFPKLNSKLSVIRNLNDQVLFIDQGNRPLFEDMTDSDCRDNAPRTIFIISMYKD |
| | | SQPRGMAVTISVKCEKISTLSCENKIISFKEMNPPDNIKDTKSDIIFFQRSVPGH |
| | | DNKMQFESSSYEGYFLACEKERDLFKLILKKEDELGDRSIMFTVQNED |
| 151 | WM12 | YFGKLRSKLSVIRNLNDQVLFIDQGNRPLFEDMTDSDCRDNAPRTIFIISMYKD |
| | | SQPRGMAVTISVKCEKISTLSCENKIISFKEMNPPDSIKDTKSDIIFFQRSVPGHD |
| | | NKMQFESSSYEGYFLACEKERDLFKLILKKEDELGDRSIMFTVQNED |
| 152 | WM13 | YFGKLESKLSIIRNLNDQVLFIDQGNRPLFEDMTDSDCRDNAPRTIFIISMYKDS |
| | | QPRGMAVTISVKCEKISTLSCENKIISFKEMNPPDNIKDTKSDIIFFQRSVPGHD |
| | | NKMQFESSSYEGYFLACEKERDLFKLILKKEDELGDRSIMFTVQNED |
| 153 | WM14 | YFSKLESKLSVIRNLNDQVLFIDQGNRPLFEDMTDSDCRDNAPRTIFIISMYKD |
| | | SQPRGMAVTISVKCEKISTLSCENKIISFKEMNPPDNIKDTKSDIIFFQRSVPGH |
| | | DNKMQFESSSYEGYFLACEKERDLFKLILKREDELGDRSIMFTVQNED |
| 154 | | YFPKLGSKLSVIRNLNDQVLFIDQGNRPLFEDMTDSDCRDNAPRTIFIISMYKW |
| | | SQPRGMAVTISVKCEKISTLSCENKIISFKEMNPPDVIKDTKSDIIFFQYSVPGH |
| | | DNKMQFESSSYEGYFLACEKERDLFKLILKKEDELGDRSIMFTVQIED |
| 155 | | YFRKLLSKLSVIRNLNDQVLFIDQGNRPLFEDMTDSDCRDNAPRTIFIISMYKD |
| | | SQPRGMAVTISVKCEKISTLSCENKIISFKEMNPPDNIKDTKSDIIFFQRSVPGH |
| | | DNKMQFESSSYEGYFLACEKERDLFKLILKKEDELGDRSIMFTVQNED |
| 156 | | YFSKLESKLSIIRNLNDQVLFIDQGNRPLFEDMTDSDCRDNAPRTIFIISMYKDS |
| | | QPRGMAVTISVKCEKISTLSCENKIISFKEMNPPDNIKDTKSDIIFFQRSVPGHD |
| | | NKMQFESSSYEGYFLACEKERDLFKLILKKEDELGDRSIMFTVQNED |
| 157 | | YFPKLNSKLSVIRNLNDQVLFIDQGNRPLFEDMTDSDCRDNAPRTIFIISMYKD |
| | | SQPRGMAVTISVKCEKISTLSCENKIISFKEMNPPDSIKDTKSDIIFFQRSVPGHD |
| | | NKMQFESSSYEGYFLACEKERDLFKLILKKEDELGDRSIMFTVQNED |
| 158 | | YFGKLESKLSVIRNLNDQVLFIDQGNRPLFEDMTDSDCRDNAPRTIFIISMYKD |
| | | SQPRGMAVTISVKCEKISTLSCENKIISFKEMNPPDNIKDTKSDIIFFQRSVPGH |
| | | DNKVQFESSSYEGYFLACEKERDLFKLILKKEDELGDRSIMFTVQNED |
| 159 | | YFSKLESKLSVIRNLNDQVLFIDQGNRPLFEDMTDSDCRDNAPRTIFIISMYKD |
| | | SQPRGMAVTISVKCEKISTLSCENKIISFKEMNPPDNIKDTKSDIIFFQRSVPGH |
| | | DNKMQFESSSYEGYFLACEKERDLFKLILKKEDELGDRSIMFTVQNED |
| 160 | | YFGKLESKLSVIRNLNDQVLFIDQGNRPLFEDMTDSDCRDNAPRTIFIISMYKD |
| | | SQPRGMAVTISVKCEKISTLSCENKIISFKEMNPPDGIKDTKSDIIFFQRSVPGH |
| | | DNKMQFESSSYEGYFLACEKERDLFKLILKKEDELGDRSIMFTVQNED |
| 161 | | YFGKLESKLSVIRNLNDQVLFIDQGNRPFFEDMTDSDCRDNAPRTIFIISMYKD |
| | | SQPRGMAVTISVKCEKISTLSCENKIISFKEMNPPDNIKDTKSDIIFFQRSVPGH |
| | | DNKMQFESSSYEGYFLACEKERDLFKLILKKEDELGDRSIMFTVQNED |
| 162 | | YFSKLESKLSVIRNLNDQVLFIDQGNRPLFEDMTDSDCRDNAPRTIFIISMYKD |
| | | SQPRGMAVTISVKCEKISTLSCENKIISFKEMNPPDNIKDTKSDIIFFQRSVPGH |
| | | DNKVQFESSSYEGYFLACEKERDLFKLILKKEDELGDRSIMFTVQNED |
| 163 | | YFGKLESKLSVIRNLNDQVLFIDQGNRPLFEDMTDSDCRDNAPRTIFIISMYKD |
| | | SQPRGMAVTISVKCEKISTLSCENKIISFKEMNPPDSIKDTKSDIIFFQRSVPGHD |
| | | NKMQFESSSYEGYFLACEKERDLFKLILKKEDELGDRSIMFTVQNED |
| 164 | | YFSKLESKLSVIRNLNDQVLFIDHGNRPLFEDMTDSDCRDNAPRTIFIISMYKD |
| | | SQPRGMAVTISVKCEKISTLSCENKIISFKEMNPPDNIKDTKSDIIFFQRSVPGH |
| | | DNKMQFESSSYEGYFLACEKERDLFKLILKKEDELGDRSIMFTVQNED |
| 165 | | YFSKLESKLSVIRNLNDQVLFIDQGNRPFFEDMTDSDCRDNAPRTIFIISMYKD |
| | | SQPRGMAVTISVKCEKISTLSCENKIISFKEMNPPDNIKDTKSDIIFFQRSVPGH |
| | | DNKMQFESSSYEGYFLACEKERDLFKLILKKEDELGDRSIMFTVQNED |
| 166 | | YFTKLRSKLSVIRNLNDQVLFIDQGNRPLFEDMTDSDCRDNAPRTIFIISMYKD |
| | | SQPRGMAVTISVKCEKISTLSCENKIISFKEMNPPDNIKDTKSDIIFFQRSVPGH |
| | | DNKMQFESSSYEGYFLACEKERDLFKLILKKEDELGDRSIMFTVQNED |
| 167 | | YFGKLESKLSVIRNLNDQVLFIDQGNRPLFEDMTDSDCRDNAPRTIFIISMYKD |
| | | SQPRGMAVTISVKCEKISTLSCENKIISFKEMNPPDAIKDTKSDIIFFQRSVPGH |
| | | DNKMQFESSSYEGYFLACEKERDLFKLILKKEDELGDRSIMFTVQNED |
| 168 | | YFSKLESKLSVIRNLNDQVLFIDQGNRPLFEDMTDSDCRDNAPRTIFIISMYKD |
| | | SQPRGMAVTISVKCEKISTLSCENKIISFKEMNPPDAIKDTKSDIIFFQRSVPGH |
| | | DNKMQFESSSYEGYFLACEKERDLFKLILKKEDELGDRSIMFTVQNED |
| 169 | | YFPKLLSKLSVIRNLNDQVLFIDQGNRPLFEDMTDSDCRDNAPRTIFIISMYKD |
| | | SQPRGMAVTISVKCEKISTLSCENKIISFKEMNPPDNIKDTKSDIIFFQRSVPGH |
| | | DNKMQFESSSYEGYFLACEKERDLFKLILKKEDELGDRSIMFTVQNED |
| 170 | | YFAKLSSKLSVIRNLNDQVLFIDQGNRPLFEDMTDSDCRDNAPRTIFIISMYKD |
| | | SQPRGMAVTISVKCEKISTLSCENKIISFKEMNPPDNIKDTKSDIIFFQRSVPGH |
| | | DNKMQFESSSYEGYFLACEKERDLFKLILKKEDELGDRSIMFTVQNED |
| 171 | | YFSKLESKLSVIRNLNDQVLFIDQGNRPLFEDMTDSDCRDNAPRTIFIISMYKD |
| | | SQPRGMAVTISVKCEKISTLSCENKIISFKEMNPPDNIKDTKSDIIFFQRSVPGH |
| | | DNKMQFESSSYEGYFLACEKERDLFKLILKMEDELGDRSIMFTVQNED |
| 172 | | YFGKLESKLSVIRNLNDQVLFIDQGNRPLFEDMTDSDCRDNAPRTIFIISMYKD |
| | | SQPRGMAVTISVKCEKISTLSCENKIISFKEMNPPDVIKDTKSDIIFFQRSVPGH |
| | | DNKMQFESSSYEGYFLACEKERDLFKLILKKEDELGDRSIMFTVQNED |
| 173 | | YFGKLESKLSVIRNLNDQVLFIDQGNRPLFEDMTDSDCRDNAPRTIFIISMYKD |
| | | SQPRGMAVTISVKCEKISTLSCENKIISFKEMNPPDFIKDTKSDIIFFQRSVPGHD |
| | | NKMQFESSSYEGYFLACEKERDLFKLILKKEDELGDRSIMFTVQNED |
| 174 | | YFGKLESKLSVIRNLNDQVLFIDQGNRPLFEDMTDSDCRDNAPRTIFIISMYKD |
| | | SQPRGMAVTISVKCEKISTLSCENKIISFKEMNPPDNIKDTKSDIIFFQSSVPGHD |
| | | NKMQFESSSYEGYFLACEKERDLFKLILKKEDELGDRSIMFTVQNED |
| 175 | | YFPKLVSKLSVIRNLNDQVLFIDQGNRPLFEDMTDSDCRDNAPRTIFIISMYKD |
| | | SQPRGMAVTISVKCEKISTLSCENKIISFKEMNPPDNIKDTKSDIIFFQRSVPGH |
| | | DNKMQFESSSYEGYFLACEKERDLFKLILKKEDELGDRSIMFTVQNED |
| 176 | | YFPKLGSKLSVIRNLNDQVLFIDQGNRPLFEDMTDSDCRDNAPRTIFIISMYKW |
| | | SQPRGMAVTISVKCEKISTLSCENKIISFKEMNPPDNIKDTKSDIIFFQRSVPGH |
| | | DNKMQFESSSYEGYFLACEKERDLFKLILKKEDELGDRSIMFTVQNED |
| 177 | | YFSKLESKLSVIRNLNDQVLFIDQGNRPLFEDMTDSDCRDNAPRTIFIISMYKD |
| | | SQPRGMAVTISVKCEKISTLSCENKIISFKEMNPPDNIKDTKSDIIFFQSSVPGHD |
| | | NKMQFESSSYEGYFLACEKERDLFKLILKKEDELGDRSIMFTVQNED |
| 178 | | YFGKLESKLSVIRNLNDQVLFIDQGNRPLFEDMTDSDCRDNAPRTIFIISMYKD |
| | | SQPRGMAVTISVKCEKISTLSCENKIISFKEMNPPDSIKDTKSDIIFFQSSVPGHD |
| | | NKMQFESSSYEGYFLACEKERDLFKLILKKEDELGDRSIMFTVQNED |
| 179 | | YFPKLRSKLSVIRNLNDQVLFIDQGNRPLFEDMTDSDCRDNAPRTIFIISMYKD |
| | | SQPRGMAVTISVKCEKISTLSCENKIISFKEMNPPDNIKDTKSDIIFFQTSVPGH |
| | | DNKMQFESSSYEGYFLACEKERDLFKLILKKEDELGDRSIMFTVQNED |
| 180 | | YFPKLGSKLSVIRNLNDQVLFIDQGNRPLFEDMTDSDCRDNAPRTIFIISMYKW |
| | | SQPRGMAVTISVKCEKISTLSCENKIISFKEMNPPDGIKDTKSDIIFFQRSVPGH |
| | | DNKMQFESSSYEGYFLACEKERDLFKLILKKEDELGDRSIMFTVQNED |
| 181 | | YFPKLKSKLSVIRNLNDQVLFIDQGNRPLFEDMTDSDCRDNAPRTIFIISMYKD |
| | | SQPRGMAVTISVKCEKISTLSCENKIISFKEMNPPDNIKDTKSDIIFFQTSVPGH |
| | | DNKMQFESSSYEGYFLACEKERDLFKLILKKEDELGDRSIMFTVQNED |
| 182 | | YFPKLGSKLSVIRNLNDQVLFIDQGNRPLFEDMTDSDCRDNAPRTIFIISMYKW |
| | | SQPRGMAVTISVKCEKISTLSCENKIISFKEMNPPDSIKDTKSDIIFFQRSVPGHD |
| | | NKMQFESSSYEGYFLACEKERDLFKLILKKEDELGDRSIMFTVQNED |
| 183 | | YFGKLESKLSVIRNLNDQVLFIDQGNRPLFEDMTDSDCRDNAPRTIFIISMYKD |
| | | SQPRGMAVTISVKCEKISTLSCENKIISFKEMNPPDPIKDTKSDIIFFQHSVPGHD |
| | | NKMQFESSSYEGYFLACEKERDLFKLILKKEDELGDRSIMFTVQNED |
| 184 | | YFGKLESKLSVIRNLNDQVLFIDQGNRPLFEDMTDSDCRDNAPRTIFIISMYKD |
| | | SQPRGMAVTISVKCEKISTLSCENKIISFKEMNPPDAIKDTKSDIIFFQSSVPGHD |
| | | NKMQFESSSYEGYFLACEKERDLFKLILKKEDELGDRSIMFTVQNED |
| 185 | | YFTKLMSKLSVIRNLNDQVLFIDQGNRPLFEDMTDSDCRDNAPRTIFIISMYKD |
| | | SQPRGMAVTISVKCEKISTLSCENKIISFKEMNPPDNIKDTKSDIIFFQRSVPGH |
| | | DNKMQFESSSYEGYFLACEKERDLFKLILKKEDELGDRSIMFTVQNED |
| 186 | | YFWKLKSKLSVIRNLNDQVLFIDQGNRPLFEDMTDSDCRDNAPRTIFIISMYK |
| | | DSQPRGMAVTISVKCEKISTLSCENKIISFKEMNPPDNIKDTKSDIIFFQRSVPG |
| | | HDNKMQFESSSYEGYFLACEKERDLFKLILKKEDELGDRSIMFTVQNED |
| 187 | | YFPKLGSKLSVIRNLNDQVLFIDQGNRPLFEDMTDSDCRDNAPRTIFIISMYKW |
| | | SQPRGMAVTISVKCEKISTLSCENKIISFKEMNPPDAIKDTKSDIIFFQRSVPGH |
| | | DNKMQFESSSYEGYFLACEKERDLFKLILKKEDELGDRSIMFTVQNED |
| 188 | | YFAKLYSKLSVIRNLNDQVLFIDQGNRPLFEDMTDSDCRDNAPRTIFIISMYKD |
| | | SQPRGMAVTISVKCEKISTLSCENKIISFKEMNPPDNIKDTKSDIIFFQRSVPGH |
| | | DNKMQFESSSYEGYFLACEKERDLFKLILKKEDELGDRSIMFTVQNED |
| 189 | | YFAKLYSKLSVIRNLNDQVLFIDQGNRPLFEDMTDSDCRDNAPRTIFIISMYKD |
| | | SQPRGMAVTISVKCEKISTLSCENKIISFKEMNPPDSIKDTKSDIIFFQRSVPGHD |
| | | NKMQFESSSYEGYFLACEKERDLFKLILKKEDELGDRSIMFTVQNED |
| 190 | | YFVKLPSKLSVIRNLNDQVLFIDQGNRPLFEDMTDSDCRDNAPRTIFIISMYKD |
| | | SQPRGMAVTISVKCEKISTLSCENKIISFKEMNPPDNIKDTKSDIIFFQRSVPGH |
| | | DNKMQFESSSYEGYFLACEKERDLFKLILKKEDELGDRSIMFTVQNED |
| 191 | | YFTKLRSKLSVIRNLNDQVLFIDQGNRPLFEDMTDSDCRDNAPRTIFIISMYKD |
| | | SQPRGMAVTISVKCEKISTLSCENKIISFKEMNPPDNIKDTKSDIIFFQTSVPGH |
| | | DNKMQFESSSYEGYFLACEKERDLFKLILKREDELGDRSIMFTVQNED |
| 192 | | YFTKLRSKLSIIRNLNDQVLFIDQGNRPLFEDMTDSDCRDNAPRTIFIISMYKDS |
| | | QPRGMAVTISVKCEKISTLSCENKIISFKEMNPPDNIKDTKSDIIFFQTSVPGHD |
| | | NKMQFESSSYEGYFLACEKERDLFKLILKKEDELGDRSIMFTVQNED |
| 193 | | YFPKLGSKLSVIRNLNDQVLFIDQGNRPLFEDMTDSDCRDNAPRTIFIISMYKW |
| | | SQPRGMAVTISVKCEKISTLSCENKIISFKEMNPPDVIKDTKSDIIFFQRSVPGH |
| | | DNKMQFESSSYEGYFLACEKERDLFKLILKKEDELGDRSIMFTVQNED |
| 194 | | YFTKLRSKLSVIRNLNDQVLFIDQGNRPLFEDMTDSDCRDNAPRTIFIISMYKD |
| | | SQPRGMAVTISVKCEKISTLSCENKIISFKEMNPPDNIKDTKSDIIFFQTSVPGH |
| | | DNKMQFESSSYEGYFLACEKERDLFKLILKKEDELGDRSIMFTVQNED |
| 195 | | YFTKLRSKLSVIRNLNDQVLFIDQGNRPLFEDMTDSDCRDNAPRTIFIISMYKD |
| | | SQPRGMAVTISVKCEKISTLSCENKIISFKEMNPPDNIKDTKSDIIFFQTSVPGH |
| | | DNKVQFESSSYEGYFLACEKERDLFKLILKKEDELGDRSIMFTVQNED |
| 196 | | YFTKLRSKLSVIRNLNDQVLFIDHGNRPLFEDMTDSDCRDNAPRTIFIISMYKD |
| | | SQPRGMAVTISVKCEKISTLSCENKIISFKEMNPPDNIKDTKSDIIFFQTSVPGH |
| | | DNKMQFESSSYEGYFLACEKERDLFKLILKKEDELGDRSIMFTVQNED |
| 197 | | YFTKLRSKLSVIRNLNDQVLFIDQGNRPFFEDMTDSDCRDNAPRTIFIISMYKD |
| | | SQPRGMAVTISVKCEKISTLSCENKIISFKEMNPPDNIKDTKSDIIFFQTSVPGH |
| | | DNKMQFESSSYEGYFLACEKERDLFKLILKKEDELGDRSIMFTVQNED |
| 198 | | YFWKLSSKLSVIRNLNDQVLFIDQGNRPLFEDMTDSDCRDNAPRTIFIISMYKD |
| | | SQPRGMAVTISVKCEKISTLSCENKIISFKEMNPPDNIKDTKSDIIFFQRSVPGH |
| | | DNKMQFESSSYEGYFLACEKERDLFKLILKKEDELGDRSIMFTVQNED |
| 199 | | YFPKLGSKLSVIRNLNDQVLFIDQGNRPLFEDMTDSDCRDNAPRTIFIISMYKW |
| | | SQPRGMAVTISVKCEKISTLSCENKIISFKEMNPPDGIKDTKSDIIFFQSSVPGHD |
| | | NKMQFESSSYEGYFLACEKERDLFKLILKKEDELGDRSIMFTVQNED |
| 200 | | YFPKLGSKLSVIRNLNDQVLFIDQGNRPLFEDMTDSDCRDNAPRTIFIISMYKW |
| | | SQPRGMAVTISVKCEKISTLSCENKIISFKEMNPPDSIKDTKSDIIFFQSSVPGHD |
| | | NKMQFESSSYEGYFLACEKERDLFKLILKKEDELGDRSIMFTVQNED |
| 201 | | YFWKLSSKLSVIRNLNDQVLFIDQGNRPLFEDMTDSDCRDNAPRTIFIISMYKD |
| | | SQPRGMAVTISVKCEKISTLSCENKIISFKEMNPPDSIKDTKSDIIFFQRSVPGHD |
| | | NKMQFESSSYEGYFLACEKERDLFKLILKKEDELGDRSIMFTVQNED |
| 202 | | YFPKLGSKLSVIRNLNDQVLFIDQGNRPLFEDMTDSDCRDNAPRTIFIISMYKW |
| | | SQPRGMAVTISVKCEKISTLSCENKIISFKEMNPPDPIKDTKSDIIFFQHSVPGHD |
| | | NKMQFESSSYEGYFLACEKERDLFKLILKKEDELGDRSIMFTVQNED |
| 203 | | YFGKLESKLSVIRNLNDQVLFIDQGNRPLFEDMTDSDCRDNAPRTIFIISMYKD |
| | | SQPRGMAVTISVKCEKISTLSCENKIISFKEMNPPDNIKDTKSDIIFFQCSVPGH |
| | | DNKMQFESSSYEGYFLACEKERDLFKLILKKEDELGDRSIMFTVQNED |
| 204 | | YFPKLGSKLSVIRNLNDQVLFIDQGNRPLFEDMTDSDCRDNAPRTIFIISMYKW |
| | | SQPRGMAVTISVKCEKISTLSCENKIISFKEMNPPDAIKDTKSDIIFFQSSVPGHD |
| | | NKMQFESSSYEGYFLACEKERDLFKLILKKEDELGDRSIMFTVQNED |
| 205 | | YFPKLGSKLSVIRNLNDQVLFIDQGNRPLFEDMTDSDCRDNAPRTIFIISMYKW |
| | | SQPRGMAVTISVKCEKISTLSCENKIISFKEMNPPDGIKDTKSDIIFFQTSVPGH |
| | | DNKMQFESSSYEGYFLACEKERDLFKLILKKEDELGDRSIMFTVQNED |
| 206 | | YFPKLGSKLSVIRNLNDQVLFIDQGNRPLFEDMTDSDCRDNAPRTIFIISMYKW |
| | | SQPRGMAVTISVKCEKISTLSCENKIISFKEMNPPDVIKDTKSDIIFFQGSVPGH |
| | | DNKMQFESSSYEGYFLACEKERDLFKLILKKEDELGDRSIMFTVQNED |
| 207 | | YFPKLGSKLSVIRNLNDQVLFIDQGNRPLFEDMTDSDCRDNAPRTIFIISMYKW |
| | | SQPRGMAVTISVKCEKISTLSCENKIISFKEMNPPDVIKDTKSDIIFFQSSVPGHD |
| | | NKMQFESSSYEGYFLACEKERDLFKLILKKEDELGDRSIMFTVQNED |
| 208 | | YFPKLFSKLSVIRNLNDQVLFIDQGNRPLFEDMTDSDCRDNAPRTIFIISMYKH |
| | | SQPRGMAVTISVKCEKISTLSCENKIISFKEMNPPDAIKDTKSDIIFFQSSVPGHD |
| | | NKMQFESSSYEGYFLACEKERDLFKLILKKEDELGDRSIMFTVQNED |
| 209 | | YFPKLRSKLSVIRNLNDQVLFIDQGNRPLFEDMTDSDCRDNAPRTIFIISMYKD |
| | | SQPRGMAVTISVKCEKISTLSCENKIISFKEMNPPDNIKDTKSDIIFFQISVPGHD |
| | | NKMQFESSSYEGYFLACEKERDLFKLILKKEDELGDRSIMFTVQNED |
| 210 | | YFGKLESKLSVIRNLNDQVLFIDQGNRPLFEDMTDSDCRDNAPRTIFIISMYKD |
| | | SQPRGMAVTISVKCEKISTLSCENKIISFKEMNPPDNIKDTKSDIIFFQLSVPGH |
| | | DNKMQFESSSYEGYFLACEKERDLFKLILKKEDELGDRSIMFTVQNED |
| 211 | | YFGKLESKLSVIRNLNDQVLFIDQGNRPLFEDMTDSDCRDNAPRTIFIISMYKD |
| | | SQPRGMAVTISVKCEKISTLSCENKIISFKEMNPPDNIKDTKSDIIFFQISVPGHD |
| | | NKMQFESSSYEGYFLACEKERDLFKLILKKEDELGDRSMMFTVQNED |
| 212 | | YFPKLKSKLSVIRNLNDQVLFIDQGNRPLFEDMTDSDCRDNAPRTIFIISMYKD |
| | | SQPRGMAVTISVKCEKISTLSCENKIISFKEMNPPDNIKDTKSDIIFFQISVPGHD |
| | | NKMQFESSSYEGYFLACEKERDLFKLILKKEDELGDRSIMFTVQNED |
| 213 | | YFGKLESKLSVIRNLNDQVLFIDQGNRPLFEDMTDSDCRDNAPRTIFIISMYKD |
| | | SQPRGMAVTISVKCEKISTLSCENKIISFKEMNPPDGIKDTKSDIIFFQLSVPGH |
| | | DNKMQFESSSYEGYFLACEKERDLFKLILKKEDELGDRSIMFTVQNED |
| 214 | | YFPKLGSKLSVIRNLNDQVLFIDQGNRPLFEDMTDSDCRDNAPRTIFIISMYKW |
| | | SQPRGMAVTISVKCEKISTLSCENKIISFKEMNPPDVIKDTKSDIIFFQHSVPGH |
| | | DNKMQFESSSYEGYFLACEKERDLFKLILKKEDELGDRSIMFTVQNED |
| 215 | | YFGKLRSKLSVIRNLNDQVLFIDQGNRPLFEDMTDSDCRDNAPRTIFIISMYKD |
| | | SQPRGMAVTISVKCEKISTLSCENKIISFKEMNPPDNIKDTKSDIIFFQISVPGHD |
| | | NKMQFESSSYEGYFLACEKERDLFKLILKKEDELGDRSIMFTVQNED |
| 216 | | YFGKLESKLSVIRNLNDQVLFIDQGNRPLFEDMTDSDCRDNAPRTIFIISMYKD |
| | | SQPRGMAVTISVKCEKISTLSCENKIISFKEMNPPDDIKDTKSDIIFFQFSVPGHD |
| | | NKMQFESSSYEGYFLACEKERDLFKLILKKEDELGDRSIMFTVQNED |
| 217 | | YFGKLESKLSVIRNLNDQVLFIDQGNRPLFEDMTDSDCRDNAPRTIFIISMYKD |
| | | SQPRGMAVTISVKCEKISTLSCENKIISFKEMNPPDNIKDTKSDIIFFQISVPGHD |
| | | NKMQFESSSYEGYFLACEKERDLFKLILKREDELGDRSIMFTVQNED |
| 218 | | YFPKLGSKLSVIRNLNDQVLFIDQGNRPLFEDMTDSDCRDNAPRTIFIISMYKD |
| | | SQPRGMAVTISVKCEKISTLSCENKIISFKEMNPPDNIKDTKSDIIFFQISVPGHD |
| | | NKMQFESSSYEGYFLACEKERDLFKLILKKEDELGDRSIMFTVQNED |
| 219 | | YFPKLSSKLSVIRNLNDQVLFIDQGNRPLFEDMTDSDCRDNAPRTIFIISMYKD |
| | | SQPRGMAVTISVKCEKISTLSCENKIISFKEMNPPDNIKDTKSDIIFFQISVPGHD |
| | | NKMQFESSSYEGYFLACEKERDLFKLILKKEDELGDRSIMFTVQNED |
| 220 | | YFTKLRSKLSVIRNLNDQVLFIDQGNRPLFEDMTDSDCRDNAPRTIFIISMYKD |
| | | SQPRGMAVTISVKCEKISTLSCENKIISFKEMNPPDNIKDTKSDIIFFQLSVPGH |
| | | DNKMQFESSSYEGYFLACEKERDLFKLILKKEDELGDRSIMFTVQNED |
| 221 | | YFGKLESKLSIIRNLNDQVLFIDQGNRPLFEDMTDSDCRDNAPRTIFIISMYKDS |
| | | QPRGMAVTISVKCEKISTLSCENKIISFKEMNPPDNIKDTKSDIIFFQISVPGHDN |
| | | KMQFESSSYEGYFLACEKERDLFKLILKKEDELGDRSIMFTVQNED |
| 222 | | YFGKLESKLSVIRNLNDQVLFIDQGNRPLFEDMTDSDCRDNAPRTIFIISMYKD |
| | | SQPRGMAVTISVKCEKISTLSCENKIISFKEMNPPDNIKDTKSDIIFFQYSVPGH |
| | | DNKMQFESSSYEGYFLACEKERDLFKLILKKEDELGDRSIMFTVQNED |
| 223 | | YFSKLESKLSVIRNLNDQVLFIDQGNRPLFEDMTDSDCRDNAPRTIFIISMYKD |
| | | SQPRGMAVTISVKCEKISTLSCENKIISFKEMNPPDDIKDTKSDIIFFQFSVPGHD |
| | | NKMQFESSSYEGYFLACEKERDLFKLILKKEDELGDRSIMFTVQNED |
| 224 | | YFGKLESKLSVIRNLNDQVLFIDQGNRPLFEDMTDSDCRDNAPRTIFIISMYKD |
| | | SQPRGMAVTISVKCEKISTLSCENKIISFKEMNPPDNIKDTKSDIIFFQISVPGHD |
| | | NKMQFESSSYEGYFLACEKERDLFKLILKKEDELGDRSIMFTVQNED |
| 225 | | YFPKLGSKLSVIRNLNDQVLFIDQGNRPLFEDMTDSDCRDNAPRTIFIISMYKW |
| | | SQPRGMAVTISVKCEKISTLSCENKIISFKEMNPPDVIKDTKSDIIFFQTSVPGH |
| | | DNKMQFESSSYEGYFLACEKERDLFKLILKKEDELGDRSIMFTVQNED |
| 226 | | YFSKLESKLSVIRNLNDQVLFIDQGNRPLFEDMTDSDCRDNAPRTIFIISMYKD |
| | | SQPRGMAVTISVKCEKISTLSCENKIISFKEMNPPDNIKDTKSDIIFFQYSVPGH |
| | | DNKMQFESSSYEGYFLACEKERDLFKLILKKEDELGDRSIMFTVQNED |
| 227 | | YFGKLESKLSVIRNLNDQVLFIDQGNRPLFEDMTDSDCRDNAPRTIFIISMYKD |
| | | SQPRGMAVTISVKCEKISTLSCENKIISFKEMNPPDNIKDTKSDIIFFQISVPGHD |
| | | NKVQFESSSYEGYFLACEKERDLFKLILKKEDELGDRSIMFTVQNED |
| 228 | | YFPKLKSKLSVIRNLNDQVLFIDQGNRPLFEDMTDSDCRDNAPRTIFIISMYKD |
| | | SQPRGMAVTISVKCEKISTLSCENKIISFKEMNPPDVIKDTKSDIIFFQLSVPGH |
| | | DNKMQFESSSYEGYFLACEKERDLFKLILKKEDELGDRSIMFTVQNED |
| 229 | | YFGKLESKLSVIRNLNDQVLFIDHGNRPLFEDMTDSDCRDNAPRTIFIISMYKD |
| | | SQPRGMAVTISVKCEKISTLSCENKIISFKEMNPPDNIKDTKSDIIFFQISVPGHD |
| | | NKMQFESSSYEGYFLACEKERDLFKLILKKEDELGDRSIMFTVQNED |
| 230 | | YFGKLESKLSVIRNLNDQVLFIDQGNRPFFEDMTDSDCRDNAPRTIFIISMYKD |
| | | SQPRGMAVTISVKCEKISTLSCENKIISFKEMNPPDNIKDTKSDIIFFQISVPGHD |
| | | NKMQFESSSYEGYFLACEKERDLFKLILKKEDELGDRSIMFTVQNED |
| 231 | | YFPKLTSKLSVIRNLNDQVLFIDQGNRPLFEDMTDSDCRDNAPRTIFIISMYKD |
| | | SQPRGMAVTISVKCEKISTLSCENKIISFKEMNPPDNIKDTKSDIIFFQISVPGHD |
| | | NKMQFESSSYEGYFLACEKERDLFKLILKKEDELGDRSIMFTVQNED |
| 232 | | YFPKLRSKLSVIRNLNDQVLFIDQGNRPLFEDMTDSDCRDNAPRTIFIISMYKD |
| | | SQPRGMAVTISVKCEKISTLSCENKIISFKEMNPPDMIKDTKSDIIFFQISVPGHD |
| | | NKMQFESSSYEGYFLACEKERDLFKLILKKEDELGDRSIMFTVQNED |
| 233 | | YFPKLGSKLSVIRNLNDQVLFIDQGNRPLFEDMTDSDCRDNAPRTIFIISMYKW |
| | | SQPRGMAVTISVKCEKISTLSCENKIISFKEMNPPDVIKDTKSDIIFFPYSVPGHD |
| | | NKMQFESSSYEGYFLACEKERDLFKLILKKEDELGDRSIMFTVQNED |
| 234 | | YFGKLESKLSVIRNLNDQVLFIDQGNRPLFEDMTDSDCRDNAPRTIFIISMYKD |
| | | SQPRGMAVTISVKCEKISTLSCENKIISFKEMNPPDNIKDTKSDIIFFQVSVPGH |
| | | DNKMQFESSSYEGYFLACEKERDLFKLILKKEDELGDRSIMFTVQNED |
| 235 | | YFPKLRSKLSVIRNLNDQVLFIDQGNRPLFEDMTDSDCRDNAPRTIFIISMYKQ |
| | | SQPRGMAVTISVKCEKISTLSCENKIISFKEMNPPDAIKDTKSDIIFFQISVPGHD |
| | | NKMQFESSSYEGYFLACEKERDLFKLILKKEDELGDRSIMFTVQNED |
| 236 | | YFPKLRSKLSVIRNLNDQVLFIDQGNRPLFEDMTDSDCRDNAPRTIFIISMYKH |
| | | SQPRGMAVTISVKCEKISTLSCENKIISFKEMNPPDHIKDTKSDIIFFQYSVPGH |
| | | DNKMQFESSSYEGYFLACEKERDLFKLILKKEDELGDRSIMFTVQNED |
| 237 | | YFPKLKSKLSVIRNLNDQVLFIDQGNRPLFEDMTDSDCRDNAPRTIFIISMYKQ |
| | | SQPRGMAVTISVKCEKISTLSCENKIISFKEMNPPDAIKDTKSDIIFFQISVPGHD |
| | | NKMQFESSSYEGYFLACEKERDLFKLILKKEDELGDRSIMFTVQNED |
| 238 | | YFPKLGSKLSVIRNLNDQVLFIDQGNRPLFEDMTDSDCRDNAPRTIFIISMYKW |
| | | SQPRGMAVTISVKCEKISTLSCENKIISFKEMNPPDVIKDTKSDIIFFKYSVPGH |
| | | DNKMQFESSSYEGYFLACEKERDLFKLILKKEDELGDRSIMFTVQNED |
| 239 | | YFPKLGSKLSVIRNLNDQVLFIDQGNRPLFEDMTDSDCRDNAPRTIFIISMYKW |
| | | SQPRGMAVTISVKCEKISTLSCENKIISFKEMNPPDAIKDTKSDIIFFQCSVPGH |
| | | DNKMQFESSSYEGYFLACEKERDLFKLILKKEDELGDRSIMFTVQNED |
| 240 | | YFGKLESKLSVIRNLNDQVLFIDQGNRPLFEDMTDSDCRDNAPRTIFIISMYKD |
| | | SQPRGMAVTISVKCEKISTLSCENKIISFKEMNPPDVIKDTKSDIIFFQLSVPGH |
| | | DNKMQFESSSYEGYFLACEKERDLFKLILKKEDELGDRSIMFTVQNED |
| 241 | | YFGKLESKLSVIRNLNDQVLFIDQGNRPLFEDMTDSDCRDNAPRTIFIISMYKD |
| | | SQPRGMAVTISVKCEKISTLSCENKIISFKEMNPPDNIKDTKSDIIFFQFSVPGHD |
| | | NKMQFESSSYEGYFLACEKERDLFKLILKKEDELGDRSIMFTVQNED |
| 242 | | YFPKLGSKLSVIRNLNDQVLFIDQGNRPLFEDMTDSDCRDNAPRTIFIISMYKR |
| | | SQPRGMAVTISVKCEKISTLSCENKIISFKEMNPPDVIKDTKSDIIFFQYSVPGH |
| | | DNKMQFESSSYEGYFLACEKERDLFKLILKKEDELGDRSIMFTVQNED |
| 243 | | YFPKLLSKLSVIRNLNDQVLFIDQGNRPLFEDMTDSDCRDNAPRTIFIISMYKD |
| | | SQPRGMAVTISVKCEKISTLSCENKIISFKEMNPPDNIKDTKSDIIFFQISVPGHD |
| | | NKMQFESSSYEGYFLACEKERDLFKLILKKEDELGDRSIMFTVQNED |
| 244 | | YFSKLESKLSVIRNLNDQVLFIDQGNRPLFEDMTDSDCRDNAPRTIFIISMYKD |
| | | SQPRGMAVTISVKCEKISTLSCENKIISFKEMNPPDVIKDTKSDIIFFQLSVPGH |
| | | DNKMQFESSSYEGYFLACEKERDLFKLILKKEDELGDRSIMFTVQNED |
| 245 | | YFTKLRSKLSVIRNLNDQVLFIDQGNRPLFEDMTDSDCRDNAPRTIFIISMYKD |
| | | SQPRGMAVTISVKCEKISTLSCENKIISFKEMNPPDAIKDTKSDIIFFQYSVPGH |
| | | DNKMQFESSSYEGYFLACEKERDLFKLILKKEDELGDRSIMFTVQNED |
| 246 | | YFGKLESKLSVIRNLNDQVLFIDQGNRPLFEDMTDSDCRDNAPRTIFIISMYKD |
| | | SQPRGMAVTISVKCEKISTLSCENKIISFKEMNPPDNIKDTKSDIIFFQISVPGHD |
| | | NKMQFESSSYEGYFLACEKERDLFKLILKMEDELGDRSIMFTVQNED |
| 247 | | YFPKLGSKLSVIRNLNDQVLFIDQGNRPLFEDMTDSDCRDNAPRTIFIISMYKW |
| | | SQPRGMAVTISVKCEKISTLSCENKIISFKEMNPPDGIKDTKSDIIFFQLSVPGH |
| | | DNKMQFESSSYEGYFLACEKERDLFKLILKKEDELGDRSIMFTVQNED |
| 248 | | YFPKLTSKLSIIRNLNDQVLFIDQGNRPLFEDMTDSDCRDNAPRTIFIISMYKQS |
| | | QPRGMAVTISVKCEKISTLSCENKIISFKEMNPPDAIKDTKSDIIFFQISVPGHDN |
| | | KMQFESSSYEGYFLACEKERDLFKLILKKEDELGDRSIMFTVQNED |
| 249 | | YFTKLRSKLSVIRNLNDQVLFIDQGNRPLFEDMTDSDCRDNAPRTIFIISMYKD |
| | | SQPRGMAVTISVKCEKISTLSCENKIISFKEMNPPDVIKDTKSDIIFFQLSVPGH |
| | | DNKMQFESSSYEGYFLACEKERDLFKLILKKEDELGDRSIMFTVQNED |
| 250 | | YFGKLESKLSVIRNLNDQVLFIDQGNRPLFEDMTDSDCRDNAPRTIFIISMYKD |
| | | SQPRGMAVTISVKCEKISTLSCENKIISFKEMNPPDVIKDTKSDIIFFQYSVPGH |
| | | DNKMQFESSSYEGYFLACEKERDLFKLILKKEDELGDRSIMFTVQNED |
| 251 | | YFPKLGSKLSVIRNLNDQVLFIDQGNRPLFEDMTDSDCRDNAPRTIFIISMYKW |
| | | SQPRGMAVTISVKCEKISTLSCENKIISFKEMNPPDVIKDTKSDIIFFQCSVPGH |
| | | DNKMQFESSSYEGYFLACEKERDLFKLILKKEDELGDRSIMFTVQNED |
| 252 | | YFPKLTSKLSVIRNLNDQVLFIDQGNRPLFEDMTDSDCRDNAPRTIFIISMYKQ |
| | | SQPRGMAVTISVKCEKISTLSCENKIISFKEMNPPDAIKDTKSDIIFFQISVPGHD |
| | | NKMQFESSSYEGYFLACEKERDLFKLILKKEDELGDRSIMFTVQNED |
| 253 | | YFSKLESKLSVIRNLNDQVLFIDQGNRPLFEDMTDSDCRDNAPRTIFIISMYKD |
| | | SQPRGMAVTISVKCEKISTLSCENKIISFKEMNPPDVIKDTKSDIIFFQYSVPGH |
| | | DNKMQFESSSYEGYFLACEKERDLFKLILKKEDELGDRSIMFTVQNED |
| 254 | | YFGKLESKLSVIRNLNDQVLFIDQGNRPLFEDMTDSDCRDNAPRTIFIISMYKY |
| | | SQPRGMAVTISVKCEKISTLSCENKIISFKEMNPPDNIKDTKSDIIFFQISVPGHD |
| | | NKMQFESSSYEGYFLACEKERDLFKLILKKEDELGDRSIMFTVQNED |
| 255 | | YFPKLGSKLSVIRNLNDQVLFIDQGNRPLFEDMTDSDCRDNAPRTIFIISMYKW |
| | | SQPRGMAVTISVKCEKISTLSCENKIISFKEMNPPDAIKDTKSDIIFFQLSVPGH |
| | | DNKMQFESSSYEGYFLACEKERDLFKLILKKEDELGDRSIMFTVQNED |
| 256 | | YFPKLGSKLSVIRNLNDQVLFIDQGNRPLFEDMTDSDCRDNAPRTIFIISMYKW |
| | | SQPRGMAVTISVKCEKISTLSCENKIISFKEMNPPDGIKDTKSDIIFFQYSVPGH |
| | | DNKMQFESSSYEGYFLACEKERDLFKLILKKEDELGDRSIMFTVQNED |
| 257 | | YFGKLESKLSVIRNLNDQVLFIDQGNRPLFEDMTDSDCRDNAPRTIFIISMYKD |
| | | SQPRGMAVTISVKCEKISTLSCENKIISFKEMNPPDVIKDTKSDIIFFQVSVPGH |
| | | DNKMQFESSSYEGYFLACEKERDLFKLILKKEDELGDRSIMFTVQNED |
| 258 | | YFTKLRSKLSVIRNLNDQVLFIDQGNRPLFEDMTDSDCRDNAPRTIFIISMYKD |
| | | SQPRGMAVTISVKCEKISTLSCENKIISFKEMNPPDVIKDTKSDIIFFQYSVPGH |
| | | DNKMQFESSSYEGYFLACEKERDLFKLILKKEDELGDRSIMFTVQNED |
| 259 | | YFPKLRSKLSVIRNLNDQVLFIDQGNRPLFEDMTDSDCRDNAPRTIFIISMYKW |
| | | SQPRGMAVTISVKCEKISTLSCENKIISFKEMNPPDVIKDTKSDIIFFQLSVPGH |
| | | DNKMQFESSSYEGYFLACEKERDLFKLILKKEDELGDRSIMFTVQNED |
| 260 | | YFPKLKSKLSVIRNLNDQVLFIDQGNRPLFEDMTDSDCRDNAPRTIFIISMYKW |
| | | SQPRGMAVTISVKCEKISTLSCENKIISFKEMNPPDVIKDTKSDIIFFQLSVPGH |
| | | DNKMQFESSSYEGYFLACEKERDLFKLILKKEDELGDRSIMFTVQNED |
| 261 | | YFSKLESKLSVIRNLNDQVLFIDQGNRPLFEDMTDSDCRDNAPRTIFIISMYKD |
| | | SQPRGMAVTISVKCEKISTLSCENKIISFKEMNPPDVIKDTKSDIIFFQVSVPGH |
| | | DNKMQFESSSYEGYFLACEKERDLFKLILKKEDELGDRSIMFTVQNED |
| 262 | | YLPKLRSKLSVIRNLNDQVLFIDQGNRPLFEDMTDSDCRDNAPRTIFIISMYKW |
| | | SQPRGMAVTISVKCEKISTLSCENKIISFKEMNPPDVIKDTKSDIIFFQYSVPGH |
| | | DNKMQFESSSYEGYFLACEKERDLFKLILKKEDELGDRSIMFTVQNED |
| 263 | | YFPKLGSKLSVIRNLNDQVLFIDQGNRPLFEDMTDSDCRDNAPRTIFIISMYKW |
| | | SQPRGMAVTISVKCEKISTLSCENKIISFKEMNPPDAIKDTKSDIIFFQYSVPGH |
| | | DNKMQFESSSYEGYFLACEKERDLFKLILKKEDELGDRSIMFTVQNED |
| 264 | | YFTKLRSKLSVIRNLNDQVLFIDQGNRPLFEDMTDSDCRDNAPRTIFIISMYKD |
| | | SQPRGMAVTISVKCEKISTLSCENKIISFKEMNPPDVIKDTKSDIIFFQVSVPGH |
| | | DNKMQFESSSYEGYFLACEKERDLFKLILKKEDELGDRSIMFTVQNED |
| 265 | | YFPKLGSKLSVIRNLNDQVLFIDQGNRPLFEDMTDSDCRDNAPRTIFIISMYKW |
| | | SQPRGMAVTISVKCEKISTLSCENKIISFKEMNPPDVIKDTKSDIIFFQLSVPGH |
| | | DNKMQFESSSYEGYFLACEKERDLFKLILKKEDELGDRSIMFTVQNED |
| 266 | | YFPKLRSKLSIIRNLNDQVLFIDQGNRPLFEDMTDSDCRDNAPRTIFIISMYKW |
| | | SQPRGMAVTISVKCEKISTLSCENKIISFKEMNPPDVIKDTKSDIIFFQYSVPGH |
| | | DNKMQFESSSYEGYFLACEKERDLFKLILKKEDELGDRSIMFTVQNED |
| 267 | | YFPKLGSKLSVIRNLNDQVLFIDQGNRPLFEDMTDSDCRDNAPRTIFIISMYKW |
| | | SQPRGMAVTISVKCEKISTLSCENKIISFKEMNPPDVIKDTKSDIIFFQYSVPGH |
| | | DNKMQFESSSYEGYFLACEKERDLFKLILKKEDELGDRSMMFTVQNED |
| 268 | | YFPKLRSKLSVIRNLNDQVLFIDQGNRPLFEDMTDSDCRDNAPRTIFIISMYKW |
| | | SQPRGMAVTISVKCEKISTLSCENKIISFKEMNPPDVIKDTKSDIIFFQYSVPGH |
| | | DNKMQFESSSYEGYFLACEKERDLFKLILKKEDELGDRSIMFTVQNED |
| 269 | | YFPKLGSKLSVIRNLNDQVLFIDQGNRPLFEDMTDSDCRDNAPRTIFIISMYKL |
| | | SQPRGMAVTISVKCEKISTLSCENKIISFKEMNPPDVIKDTKSDIIFFQYSVPGH |
| | | DNKMQFESSSYEGYFLACEKERDLFKLILKKEDELGDRSIMFTVQNED |
| 270 | | YFAKLYSKLSVIRNLNDQVLFIDQGNRPLFEDMTDSDCRDNAPRTIFIISMYKD |
| | | SQPRGMAVTISVKCEKISTLSCENKIISFKEMNPPDNIKDTKSDIIFFQISVPGHD |
| | | NKMQFESSSYEGYFLACEKERDLFKLILKKEDELGDRSIMFTVQNED |
| 271 | | YFPKLKSKLSVIRNLNDQVLFIDQGNRPLFEDMTDSDCRDNAPRTIFIISMYKW |
| | | SQPRGMAVTISVKCEKISTLSCENKIISFKEMNPPDVIKDTKSDIIFFQYSVPGH |
| | | DNKMQFESSSYEGYFLACEKERDLFKLILKKEDELGDRSIMFTVQNED |
| 272 | | YFPKLGSKLSVIRNLNDQVLFIDQGNRPLFEDMTDSDCRDNAPRTIFIISMYKW |
| | | SQPRGMAVTISVKCEKISTLSCENKIISFKEMNPPDMIKDTKSDIIFFQYSVPGH |
| | | DNKMQFESSSYEGYFLACEKERDLFKLILKKEDELGDRSIMFTVQNED |
| 273 | | YFPKLGSKLSVIRNLNDQVLFIDQGNRPLFEDMTDSDCRDNAPRTIFIISMYKW |
| | | SQPRGMAVTISVKCEKISTLSCENKIISFKEMNPPDAIKDTKSDIIFFQVSVPGH |
| | | DNKMQFESSSYEGYFLACEKERDLFKLILKKEDELGDRSIMFTVQNED |
| 274 | | YFGKLRSKLSVIRNLNDQVLFIDQGNRPLFEDMTDSDCRDNAPRTIFIISMYK |
| | | WSQPRGMAVTISVKCEKISTLSCENKIISFKEMNPPDVIKDTKSDIIFFQYSVPG |
| | | HDNKMQFESSSYEGYFLACEKERDLFKLILKKEDELGDRSIMFTVQNED |
| 275 | | YFPKLGSKLSVIRNLNDQVLFIDQGNRPLFEDMTDSDCRDNAPRTIFIISMYKC |
| | | SQPRGMAVTISVKCEKISTLSCENKIISFKEMNPPDVIKDTKSDIIFFQYSVPGH |
| | | DNKMQFESSSYEGYFLACEKERDLFKLILKKEDELGDRSIMFTVQNED |
| 276 | | YFPKLGSKLSVIRNLNDQVLFIDQGNRPLFEDMTDSDCRDNAPRTIFIISMYKW |
| | | SQPRGMAVTISVKCEKISTLSCENKIISFKEMNPPDVIKDTKSDIIFFQYSVPGH |
| | | DNKMQFESSSYEGYFLACEKERDLFKLILKREDELGDRSIMFTVQNED |
| 277 | | YFPKLGSKLSVIRNLNDQVLFIDQGNRPLFEDMTDSDCRDNAPRTIFIISMYKW |
| | | SQPRGMAVTISVKCEKISTLSCENKIISFKEMNPPDIIKDTKSDIIFFQYSVPGHD |
| | | NKMQFESSSYEGYFLACEKERDLFKLILKKEDELGDRSIMFTVQNED |
| 278 | | YFPKLGSKLSIIRNLNDQVLFIDQGNRPLFEDMTDSDCRDNAPRTIFIISMYKW |
| | | SQPRGMAVTISVKCEKISTLSCENKIISFKEMNPPDVIKDTKSDIIFFQYSVPGH |
| | | DNKMQFESSSYEGYFLACEKERDLFKLILKKEDELGDRSIMFTVQNED |
| 279 | | YFPKLNSKLSVIRNLNDQVLFIDQGNRPLFEDMTDSDCRDNAPRTIFIISMYKW |
| | | SQPRGMAVTISVKCEKISTLSCENKIISFKEMNPPDVIKDTKSDIIFFQYSVPGH |
| | | DNKMQFESSSYEGYFLACEKERDLFKLILKKEDELGDRSIMFTVQNED |
| 280 | | YFSKLKSKLSVIRNLNDQVLFIDQGNRPLFEDMTDSDCRDNAPRTIFIISMYKW |
| | | SQPRGMAVTISVKCEKISTLSCENKIISFKEMNPPDVIKDTKSDIIFFQYSVPGH |
| | | DNKMQFESSSYEGYFLACEKERDLFKLILKKEDELGDRSIMFTVQNED |
| 281 | | YFPKLSSKLSVIRNLNDQVLFIDQGNRPLFEDMTDSDCRDNAPRTIFIISMYKW |
| | | SQPRGMAVTISVKCEKISTLSCENKIISFKEMNPPDVIKDTKSDIIFFQYSVPGH |
| | | DNKMQFESSSYEGYFLACEKERDLFKLILKKEDELGDRSIMFTVQNED |
| 282 | | YFPKLGSKLSVIRNLNDQVLFIDQGNRPLFEDMTDSDCRDNAPRTIFIISMYKW |
| | | SQPRGMAVTISVKCEKISTLSCENKIISFKEMNPPDVIKDTKSDIIFFQYSVPGH |
| | | DNKVQFESSSYEGYFLACEKERDLFKLILKKEDELGDRSIMFTVQNED |
| 283 | | YFPKLGSKLSVIRNLNDQVLFIDHGNRPLFEDMTDSDCRDNAPRTIFIISMYKW |
| | | SQPRGMAVTISVKCEKISTLSCENKIISFKEMNPPDVIKDTKSDIIFFQYSVPGH |
| | | DNKMQFESSSYEGYFLACEKERDLFKLILKKEDELGDRSIMFTVQNED |
| 284 | | YFPKLGSKLSVIRNLNDQVLFIDQGNRPFFEDMTDSDCRDNAPRTIFIISMYKW |
| | | SQPRGMAVTISVKCEKISTLSCENKIISFKEMNPPDVIKDTKSDIIFFQYSVPGH |
| | | DNKMQFESSSYEGYFLACEKERDLFKLILKKEDELGDRSIMFTVQNED |
| 285 | | YFPKLASKLSVIRNLNDQVLFIDQGNRPLFEDMTDSDCRDNAPRTIFIISMYKW |
| | | SQPRGMAVTISVKCEKISTLSCENKIISFKEMNPPDVIKDTKSDIFFQYSVPGHD |
| | | NKMQFESSSYEGYFLACEKERDLFKLILKKEDELGDRSIMFTVQNED |
| 286 | | YFPKLTSKLSVIRNLNDQVLFIDQGNRPLFEDMTDSDCRDNAPRTIFIISMYKW |
| | | SQPRGMAVTISVKCEKISTLSCENKIISFKEMNPPDVIKDTKSDIIFFQYSVPGH |
| | | DNKMQFESSSYEGYFLACEKERDLFKLILKKEDELGDRSIMFTVQNED |
| 287 | | YFPKLGSKLSVIRNLNDQVLFIDQGNRPLFEDMTDSDCRDNAPRTIFIISMYKW |
| | | SQPRGMAVTISVKCEKISTLSCENKIISFKEMNPPDVIKDTKSDIIFFQVSVPGH |
| | | DNKMQFESSSYEGYFLACEKERDLFKLILKKEDELGDRSIMFTVQNED |
| 288 | | YFPKLGSKLSVIRNLNDQVLFIDQGNRPLFEDMTDSDCRDNAPRTIFIISMYKW |
| | | SQPRGIAVTISVKCEKISTLSCENKIISFKEMNPPDVIKDTKSDIIFFQYSVPGHD |
| | | NKMQFESSSYEGYFLACEKERDLFKLILKKEDELGDRSIMFTVQNED |
| 289 | | YFTKLRSKLSVIRNLNDQVLFIDQGNRPLFEDMTDSDCRDNAPRTIFIISMYKW |
| | | SQPRGMAVTISVKCEKISTLSCENKIISFKEMNPPDVIKDTKSDIIFFQYSVPGH |
| | | DNKMQFESSSYEGYFLACEKERDLFKLILKKEDELGDRSIMFTVQNED |
| 290 | | YFTKLKSKLSVIRNLNDQVLFIDQGNRPLFEDMTDSDCRDNAPRTIFIISMYK |
| | | WSQPRGMAVTISVKCEKISTLSCENKIISFKEMNPPDVIKDTKSDIIFFQYSVPG |
| | | HDNKMQFESSSYEGYFLACEKERDLFKLILKKEDELGDRSIMFTVQNED |
| 291 | | YFPKLGSKLSVIRNLNDQVLFIDQGNRPLFEDMTDSDCRDNAPRTIFIISMYKW |
| | | SQPRGMAVTISVKCEKISTLSCENKIISFKEMNPPDVIKDTKSDIIFFQFSVPGHD |
| | | NKMQFESSSYEGYFLACEKERDLFKLILKKEDELGDRSIMFTVQNED |
| 292 | | YFPKLLSKLSVIRNLNDQVLFIDQGNRPLFEDMTDSDCRDNAPRTIFIISMYKW |
| | | SQPRGMAVTISVKCEKISTLSCENKIISFKEMNPPDVIKDTKSDIIFFQYSVPGH |
| | | DNKMQFESSSYEGYFLACEKERDLFKLILKKEDELGDRSIMFTVQNED |
| 293 | | YFPKLGSKLSVIRNLNDQVLFIDQGNRPLFEDMTDSDCRDNAPRTIFIISMYKW |
| | | SQPRGMAVTISVKCEKISTLSCENKIISFKEMNPPDVIKDTKSDIIFFQYSVPGH |
| | | DNKMQFESSSYEGYFLACEKERDLFKLILKMEDELGDRSIMFTVQNED |
| 294 | | YFPKLGSKLSVIRNLNDQVLFIDQGNRPLFEDMTDSDCRDNAPRTIFIISMYKW |
| | | SQPRGMAVTISVKCEKISTLSCENKIISFKEMNPPDVIKDTKSDIIFFQYSVTGH |
| | | DNKMQFESSSYEGYFLACEKERDLFKLILKKEDELGDRSIMFTVQNED |
| 295 | | YFPKLCSKLSVIRNLNDQVLFIDQGNRPLFEDMTDSDCRDNAPRTIFIISMYKW |
| | | SQPRGMAVTISVKCEKISTLSCENKIISFKEMNPPDVIKDTKSDIIFFQYSVPGH |
| | | DNKMQFESSSYEGYFLACEKERDLFKLILKKEDELGDRSIMFTVQNED |
| 296 | | YFPKLGSKLSVIRNLNDQVLFIDQGNRPLFEDMTDSDCRDNAPRTIFIISMYKW |
| | | SQPRGMVVTISVKCEKISTLSCENKIISFKEMNPPDVIKDTKSDIIFFQYSVPGH |
| | | DNKMQFESSSYEGYFLACEKERDLFKLILKKEDELGDRSIMFTVQNED |
| 297 | | YFWKLRSKLSVIRNLNDQVLFIDQGNRPLFEDMTDSDCRDNAPRTIFIISMYK |
| | | WSQPRGMAVTISVKCEKISTLSCENKIISFKEMNPPDVIKDTKSDIIFFQYSVPG |
| | | HDNKMQFESSSYEGYFLACEKERDLFKLILKKEDELGDRSIMFTVQNED |
| 298 | | YFWKLKSKLSVIRNLNDQVLFIDQGNRPLFEDMTDSDCRDNAPRTIFIISMYK |
| | | WSQPRGMAVTISVKCEKISTLSCENKIISFKEMNPPDVIKDTKSDIIFFQYSVPG |
| | | HDNKMQFESSSYEGYFLACEKERDLFKLILKKEDELGDRSIMFTVQNED |
| 299 | | YFWKLSSKLSVIRNLNDQVLFIDQGNRPLFEDMTDSDCRDNAPRTIFIISMYK |
| | | WSQPRGMAVTISVKCEKISTLSCENKIISFKEMNPPDVIKDTKSDIIFFQYSVPG |
| | | HDNKMQFESSSYEGYFLACEKERDLFKLILKKEDELGDRSIMFTVQNED |
| 300 | mIL-18 | NFGRLHCTTAVIRNINDQVLFVDKRQPVFEDMTDIDQSASEPQTRLIIYMYKD |
| | | SEVRGLAVTLSVKDSKMSTLSCKNKIISFEEMDPPENIDDIQSDLIFFQKRVPG |
| | | HNKMEFESSLYEGHFLACQKEDDAFKLILKKKDENGDKSVMFTLTNLHQS |
| 301 | M12 | TACTTCCCGAAACTGAAAAGTAAACTGAGTGTGATTCGCAATCTGAATGA |
| | | TCAGGTTCTGTTTATTGATCAGGGTAATCGCCCGCTGTTTGAAGATATGAC |
| | | CGATAGCGATTGCCGCGATAATGCACCGCGTACCATTTTTATTATTAGTAT |
| | | GTATAAGGGTAGCGGTGCCCGTGGTATGGCAGTGACCATTAGTGTTAAAT |
| | | GCGAAAAAATTAGCACCCTGAGCTGTGAAAATAAGATTATTAGTTTCAAG |
| | | GAGATGAACCCGCCGGATACCATTAAGGATACCAAAAGTGATATTATCTT |
| | | CTTCCAGCGTAGTGTTCCGGGTCATGATAATAAGATGCAGTTTGAAAGCA |
| | | GCAGCTATGAAGGCTATTTTCTGGCATGTGAAAAAGAACGTGATCTGTTT |
| | | AAACTGATTCTGAAAAAAGAGGATGAGCTGGGCGATCGCAGCATTATGTT |
| | | TACCGTGCAGAATGAAGAT |
| 302 | M13 | TACTTCCCGAAACTGAAAAGCAAACTGAGCGTGATTCGCAATCTGAATGA |
| | | TCAGGTTCTGTTTATTGATCAGGGTAATCGTCCGCTGTTTGAAGATATGAC |
| | | CGATAGTGATTGTCGTGATAATGCACCGCGCACCATTTTTATTATTAGCAT |
| | | GTATAAGCAGAGCATCTGGCGCGGTATGGCCGTTACCATTAGCGTTAAAT |
| | | GCGAAAAAATTAGTACCCTGAGTTGTGAAAATAAGATTATTAGCTTCAAG |
| | | GAGATGAACCCGCCGGATAATATTAAGGATACCAAAAGCGATATCATCTT |
| | | TTTCCAGCGTAGTGTTCCGGGCCATGATAATAAGATGCAGTTTGAAAGTA |
| | | GCAGTTATGAAGGCTATTTTCTGGCATGTGAAAAAGAACGCGATCTGTTT |
| | | AAACTGATTCTGAAAAAAGAGGATGAGCTGGGCGATCGCAGCATTATGTT |
| | | TACCGTTCAGAATGAAGAT |
| 303 | M17 | TATTTCCCGAAACTGCGTTCTAAACTGTCCGTTATCCGTAACCTGAACGAT |
| | | CAGGTGCTGTTCATCGACCAGGGTAACCGTCCGCTGTTCGAAGATATGAC |
| | | TGACTCTGACTGTCGTGACAACGCACCGCGTACCATCTTTATCATTTCTAT |
| | | GTACAAACTGTCTACTGCGCGTGGCATGGCCGTGACCATTAGCGTGAAAT |
| | | GTGAAAAAATCTCCACTCTGTCTTGCGAAAACAAGATTATCAGCTTCAAA |
| | | GAAATGAACCCTCCTGATGGTATCAAAGATACTAAATCCGACATCATTTTC |
| | | TTCCAACGTTCTGTTCCGGGTCATGATAATAAAATGCAGTTCGAAAGCAG |
| | | CAGCTACGAAGGCTATTTCCTGGCATGTGAAAAAGAGCGTGACCTGTTCA |
| | | AACTGATCCTGAAGAAAGAAGACGAACTGGGCGACCGCTCTATCATGTTC |
| | | ACCGTACAGAACGAAGAC |
| 304 | M24 | TACTTCCCGAAACTGCGCAGCAAACTGAGTGTGATTCGCAATCTGAATGA |
| | | TCAGGTGCTGTTTATTGATCAGGGTAATCGCCCGCTGTTTGAAGATATGAC |
| | | CGATAGTGATTGTCGTGATAATGCCCCGCGTACCATTTTTATTATTAGTAT |
| | | GTATAAGTACAGCACCAGTCGTGGTATGGCCGTGACCATTAGTGTGAAAT |
| | | GTGAAAAAATTAGCACCCTGAGTIGTGAAAATAAGATTATTAGCTTCAAG |
| | | GAGATGAACCCGCCGGATAATATTAAGGATACCAAAAGTGATATCATCTT |
| | | CTTTCAGCGTAGTGTTCCGGGTCATGATAATAAGATGCAGTTTGAAAGCA |
| | | GCAGCTATGAAGGCTATTTTCTGGCATGTGAAAAAGAACGTGATCTGTTT |
| | | AAACTGATTCTGAAAAAAGAGGATGAGCTGGGCGATCGTAGTATTATGTT |
| | | TACCGTGCAGAATGAAGAT |
| 305 | MM5 | TACTTCGGCAAACTGGGCTCTAAGCTGAGCGTGATCCGTAACCTGAACGA |
| | | CCAGGTACTGTTCATTGACCAGGGTAACCGTCCGCTGTTCGAGGATATGA |
| | | CCGACAGCGACTGCCGTGATAACGCTCCGCGTACCATTTTCATTATCTCTA |
| | | TGTACAAACTGTCCACCGAGCGTGGTATGGCAGTTACGATCTCTGTCAAAT |
| | | GCGAAAAAATTTCTACTCTGTCTTGCGAAAACAAAATCATCTCCTTTAAAG |
| | | AAATGAACCCGCCAGATGGTATTAAAGATACTAAATCTGACATCATTTTCT |
| | | TCCAATCTTCTGTGCCGGGTCACGACAACAAAATGCAGTTTGAATCCAGCT |
| | | CCTATGAAGGTTACTTCCTGGCTTGTGAAAAAGAACGTGATCTGTTCAAAC |
| | | TGATCCTGAAAAAAGAGGACGAACTGGGCGACCGTAGCATCATGTTCACC |
| | | GTTCAAAATGAAGAT |
| 306 | WM6 | TACTTTGATAAACTGAAATCTAAACTGAGCGTAATCCGTAACCTGAACGA |
| | | CCAAGTACTGTTCATCGACCAAGGTAACCGTCCGCTGTTTGAAGACATGA |
| | | CCGACTCTGATTGCCGTGACAATGCACCGCGCACTATCTTTATTATCAGCA |
| | | TGTATAAAGACTCTCAGCCGCGTGGTATGGCTGTTACTATCTCCGTTAAAT |
| | | GCGAAAAAATCTCCACCCTGTCTTGCGAAAACAAGATTATCTCCTTCAAA |
| | | GAAATGAACCCGCCGGACTCCATCAAAGACACTAAATCCGACATCATCTT |
| | | CTTCCAACGCTCCGTACCGGGTCACGATAACAAAATGCAGTTTGAAAGCT |
| | | CTTCCTACGAAGGCTACTTCCTGGCGTGTGAGAAAGAGCGTGATCTGTTCA |
| | | AACTGATCCTGAAAAAGGAAGATGAACTGGGCGATCGTTCCATCATGTTC |
| | | ACCGTACAGAACGAGGAT |
| 307 | WT-SSS | YFGKLESKLSVIRNLNDQVLFIDQGNRPLFEDMTDSDSRDNAPRTIFIISMYKD |
| | | SQPRGMAVTISVKSEKISTLSSENKIISFKEMNPPDNIKDTKSDIIFFQRSVPGHD |
| | | NKMQFESSSYEGYFLACEKERDLFKLILKKEDELGDRSIMFTVQNED |
| 308 | M12-SSS | YFPKLKSKLSVIRNLNDQVLFIDQGNRPLFEDMTDSDSRDNAPRTIFIISMYKG |
| | | SGARGMAVTISVKSEKISTLSSENKIISFKEMNPPDTIKDTKSDIIFFQRSVPGHD |
| | | NKMQFESSSYEGYFLACEKERDLFKLILKKEDELGDRSIMFTVQNED |
| 309 | MM5-SSS | YFGKLGSKLSVIRNLNDQVLFIDQGNRPLFEDMTDSDSRDNAPRTIFIISMYKL |
| | | STERGMAVTISVKSEKISTLSSENKIISFKEMNPPDGIKDTKSDIIFFQSSVPGHD |
| | | NKMQFESSSYEGYFLACEKERDLFKLILKKEDELGDRSIMFTVQNED |
| 310 | M12-SSSS | YFPKLKSKLSVIRNLNDQVLFIDQGNRPLFEDMTDSDSRDNAPRTIFIISMYKG |
| | | SGARGMAVTISVKSEKISTLSSENKIISFKEMNPPDTIKDTKSDIIFFQRSVPGHD |
| | | NKMQFESSSYEGYFLASEKERDLFKLILKKEDELGDRSIMFTVQNED |
| 311 | M12-DB6 | YFPKLKSKLSVIRNLNDQVLFIDQGNRPLFEDMTDSDIRDNAPRTIFIISMYKGS |
| | | GARGMAVTISVKSEKISTLSCENKIISFKEMNPPDTIKDTKSDIIFFQRSVPGHD |
| | | NKMQFECSSYEGYFLACEKERDLFKLILKKEDELGDRSIMFTVQNED |
| 312 | M12-DB7 | YFPKLKSKLSVIRNLNDQVLFIDQGNRPLFEDMTDSDVRDNAPRTIFIISMYKG |
| | | SGARGMAVTISVKIEKISTLSCENKIISFKEMNPPDTIKDTKSDIIFFQRSVPGHD |
| | | NKMQFECSSYEGYFLAAEKERDLFKLILKKEDELGDRSIMFTVQNED |
| 313 | M12-DB8 | YFPKLKSKLSVIRNLNDQVLFIDQGNRPLFEDMTDSDSRDNAPRTIFIISMYKG |
| | | SGARGMAVTISVKIEKISTLSCENKIISFKEMNPPDTIKDTKSDIIFFQRSVPGHD |
| | | NKMQFECSSYEGYFLAIEKERDLFKLILKKEDELGDRSIMFTVQNED |
| 314 | M12-DB9 | YFPKLKSKLSVIRNLNDQVLFIDQGNRPLFEDMTDSDIRDNAPRTIFIISMYKGS |
| | | GARGMAVTISVKIEKISTLSVENKIISFKEMNPPDTIKDTKSDIIFFQRSVPGHD |
| | | NKMQFESSSYEGYFLAIEKERDLFKLILKKEDELGDRSIMFTVQNED |
| 315 | M12-DB10 | YFPKLKSKLSVIRNLNDQVLFIDQGNRPLFEDMTDSDIRDNAPRTIFIISMYKGS |
| | | GARGMAVTISVKLEKISTLSYENKIISFKEMNPPDTIKDTKSDIIFFQRSVPGHD |
| | | NKMQFESSSYEGYFLACEKERDLFKLILKKEDELGDRSIMFTVQNED |
| 316 | MM5-DB6 | YFGKLGSKLSVIRNLNDQVLFIDQGNRPLFEDMTDSDIRDNAPRTIFIISMYKL |
| | | STERGMAVTISVKSEKISTLSCENKIISFKEMNPPDGIKDTKSDIIFFQSSVPGHD |
| | | NKMQFECSSYEGYFLACEKERDLFKLILKKEDELGDRSIMFTVQNED |
| 317 | WT-IL-18- | YFGKLESKLSVIRNLNDQVLFIDQGNRPLFEDMTDSDIRDNAPRTIFIISMYKD |
| | DB6 | SQPRGMAVTISVKSEKISTLSCENKIISFKEMNPPDNIKDTKSDIIFFQRSVPGHD |
| | | NKMQFECSSYEGYFLACEKERDLFKLILKKEDELGDRSIMFTVQNED |
| 318 | WT IL-18- | YFGKLESKLSVIRNLNDQVLFIDQGNRPLFEDMTDSDCRDNAPRTIFIISMYKD |
| | DBo | SQPRGMAVTISVKCEKISTLSCENKIISFKEMNPPDNIKDTKSDIIFFQRSVPGH |
| | | DNKMQFECSSYEGYFLACEKERDLFKLILKKEDELGDRSIMFTVQNED |
| 319 | WT IL18- | YFGKLESKLSVIRNLNDQVLFIDQGNRPLFEDMTDSDSRDNAPRTIFIISMYKD |
| | SSS- | SQPRGMAVTISVKSEKISTLSSENKIISFKEMNPPDNIKDTKSDIIFFQRSVPGHD |
| | Fc_N297A | NKMQFESSSYEGYFLACEKERDLFKLILKKEDELGDRSIMFTVQNEDEPKSAD |
| | | KTHTCPPCPAPELLGGPSVFLFPPKPKDTLMISRTPEVTCVVVDVSHEDPEVKF |
| | | NWYVDGVEVHNAKTKPREEQYASTYRVVSVLTVLHQDWLNGKEYKCKVSN |
| | | KALPAPIEKTISKAKGQPREPQVYTLPPSREEMTKNQVSLTCLVKGFYPSDIAV |
| | | EWESNGQPENNYKTTPPVLDSDGSFFLYSKLTVDKSRWQQGNVFSCSVMHE |
| | | ALHNHYTQKSLSLSPGK |
| 320 | M12-SSS- | YFPKLKSKLSVIRNLNDQVLFIDQGNRPLFEDMTDSDSRDNAPRTIFIISMYKG |
| | Fc_N297A | SGARGMAVTISVKSEKISTLSSENKIISFKEMNPPDTIKDTKSDIIFFQRSVPGHD |
| | | NKMQFESSSYEGYFLACEKERDLFKLILKKEDELGDRSIMFTVQNEDEPKSAD |
| | | KTHTCPPCPAPELLGGPSVFLFPPKPKDTLMISRTPEVTCVVVDVSHEDPEVKF |
| | | NWYVDGVEVHNAKTKPREEQYASTYRVVSVLTVLHQDWLNGKEYKCKVSN |
| | | KALPAPIEKTISKAKGQPREPQVYTLPPSREEMTKNQVSLTCLVKGFYPSDIAV |
| | | EWESNGQPENNYKTTPPVLDSDGSFFLYSKLTVDKSRWQQGNVFSCSVMHE |
| | | ALHNHYTQKSLSLSPGK |
| 321 | MM5-SSS- | YFGKLGSKLSVIRNLNDQVLFIDQGNRPLFEDMTDSDSRDNAPRTIFIISMYKL |
| | Fc_N297A | STERGMAVTISVKSEKISTLSSENKIISFKEMNPPDGIKDTKSDIIFFQSSVPGHD |
| | | NKMQFESSSYEGYFLACEKERDLFKLILKKEDELGDRSIMFTVQNEDEPKSAD |
| | | KTHTCPPCPAPELLGGPSVFLFPPKPKDTLMISRTPEVTCVVVDVSHEDPEVKF |
| | | NWYVDGVEVHNAKTKPREEQYASTYRVVSVLTVLHQDWLNGKEYKCKVSN |
| | | KALPAPIEKTISKAKGQPREPQVYTLPPSREEMTKNQVSLTCLVKGFYPSDIAV |
| | | EWESNGQPENNYKTTPPVLDSDGSFFLYSKLTVDKSRWQQGNVFSCSVMHE |
| | | ALHNHYTQKSLSLSPGK |
| 322 | M12-SSSS- | YFPKLKSKLSVIRNLNDQVLFIDQGNRPLFEDMTDSDSRDNAPRTIFIISMYKG |
| | Fc_N297A | SGARGMAVTISVKSEKISTLSSENKIISFKEMNPPDTIKDTKSDIIFFQRSVPGHD |
| | | NKMQFESSSYEGYFLASEKERDLFKLILKKEDELGDRSIMFTVQNEDEPKSAD |
| | | KTHTCPPCPAPELLGGPSVFLFPPKPKDTLMISRTPEVTCVVVDVSHEDPEVKF |
| | | NWYVDGVEVHNAKTKPREEQYASTYRVVSVLTVLHQDWLNGKEYKCKVSN |
| | | KALPAPIEKTISKAKGQPREPQVYTLPPSREEMTKNQVSLTCLVKGFYPSDIAV |
| | | EWESNGQPENNYKTTPPVLDSDGSFFLYSKLTVDKSRWQQGNVFSCSVMHE |
| | | ALHNHYTQKSLSLSPGK |
| 323 | M12-DB6- | YFPKLKSKLSVIRNLNDQVLFIDQGNRPLFEDMTDSDIRDNAPRTIFIISMYKGS |
| | Fc_N297A | GARGMAVTISVKSEKISTLSCENKIISFKEMNPPDTIKDTKSDIIFFQRSVPGHD |
| | | NKMQFECSSYEGYFLACEKERDLFKLILKKEDELGDRSIMFTVQNEDEPKSAD |
| | | KTHTCPPCPAPELLGGPSVFLFPPKPKDTLMISRTPEVTCVVVDVSHEDPEVKF |
| | | NWYVDGVEVHNAKTKPREEQYASTYRVVSVLTVLHQDWLNGKEYKCKVSN |
| | | KALPAPIEKTISKAKGQPREPQVYTLPPSREEMTKNQVSLTCLVKGFYPSDIAV |
| | | EWESNGQPENNYKTTPPVLDSDGSFFLYSKLTVDKSRWQQGNVFSCSVMHE |
| | | ALHNHYTQKSLSLSPGK |
| 324 | M12-DB7- | YFPKLKSKLSVIRNLNDQVLFIDQGNRPLFEDMTDSDVRDNAPRTIFIISMYKG |
| | Fc_N297A | SGARGMAVTISVKIEKISTLSCENKIISFKEMNPPDTIKDTKSDIIFFQRSVPGHD |
| | | NKMQFECSSYEGYFLAAEKERDLFKLILKKEDELGDRSIMFTVQNEDEPKSAD |
| | | KTHTCPPCPAPELLGGPSVFLFPPKPKDTLMISRTPEVTCVVVDVSHEDPEVKF |
| | | NWYVDGVEVHNAKTKPREEQYASTYRVVSVLTVLHQDWLNGKEYKCKVSN |
| | | KALPAPIEKTISKAKGQPREPQVYTLPPSREEMTKNQVSLTCLVKGFYPSDIAV |
| | | EWESNGQPENNYKTTPPVLDSDGSFFLYSKLTVDKSRWQQGNVFSCSVMHE |
| | | ALHNHYTQKSLSLSPGK |
| 325 | M12-DB8- | YFPKLKSKLSVIRNLNDQVLFIDQGNRPLFEDMTDSDSRDNAPRTIFIISMYKG |
| | Fc_N297A | SGARGMAVTISVKIEKISTLSCENKIISFKEMNPPDTIKDTKSDIIFFQRSVPGHD |
| | | NKMQFECSSYEGYFLAIEKERDLFKLILKKEDELGDRSIMFTVQNEDEPKSAD |
| | | KTHTCPPCPAPELLGGPSVFLFPPKPKDTLMISRTPEVTCVVVDVSHEDPEVKF |
| | | NWYVDGVEVHNAKTKPREEQYASTYRVVSVLTVLHQDWLNGKEYKCKVSN |
| | | KALPAPIEKTISKAKGQPREPQVYTLPPSREEMTKNQVSLTCLVKGFYPSDIAV |
| | | EWESNGQPENNYKTTPPVLDSDGSFFLYSKLTVDKSRWQQGNVFSCSVMHE |
| | | ALHNHYTQKSLSLSPGK |
| 326 | M12-DB9- | YFPKLKSKLSVIRNLNDQVLFIDQGNRPLFEDMTDSDIRDNAPRTIFIISMYKGS |
| | Fc_N297A | GARGMAVTISVKIEKISTLSVENKIISFKEMNPPDTIKDTKSDIIFFQRSVPGHD |
| | | NKMQFESSSYEGYFLAIEKERDLFKLILKKEDELGDRSIMFTVQNEDEPKSAD |
| | | KTHTCPPCPAPELLGGPSVFLFPPKPKDTLMISRTPEVTCVVVDVSHEDPEVKF |
| | | NWYVDGVEVHNAKTKPREEQYASTYRVVSVLTVLHQDWLNGKEYKCKVSN |
| | | KALPAPIEKTISKAKGQPREPQVYTLPPSREEMTKNQVSLTCLVKGFYPSDIAV |
| | | EWESNGQPENNYKTTPPVLDSDGSFFLYSKLTVDKSRWQQGNVFSCSVMHE |
| | | ALHNHYTQKSLSLSPGK |
| 327 | M12-DB10- | YFPKLKSKLSVIRNLNDQVLFIDQGNRPLFEDMTDSDIRDNAPRTIFIISMYKGS |
| | Fc_N297A | GARGMAVTISVKLEKISTLSYENKIISFKEMNPPDTIKDTKSDIIFFQRSVPGHD |
| | | NKMQFESSSYEGYFLACEKERDLFKLILKKEDELGDRSIMFTVQNEDEPKSAD |
| | | KTHTCPPCPAPELLGGPSVFLFPPKPKDTLMISRTPEVTCVVVDVSHEDPEVKF |
| | | NWYVDGVEVHNAKTKPREEQYASTYRVVSVLTVLHQDWLNGKEYKCKVSN |
| | | KALPAPIEKTISKAKGQPREPQVYTLPPSREEMTKNQVSLTCLVKGFYPSDIAV |
| | | EWESNGQPENNYKTTPPVLDSDGSFFLYSKLTVDKSRWQQGNVFSCSVMHE |
| | | ALHNHYTQKSLSLSPGK |
| 328 | WT IL18- | YFGKLESKLSVIRNLNDQVLFIDQGNRPLFEDMTDSDIRDNAPRTIFIISMYKD |
| | DB6- | SQPRGMAVTISVKSEKISTLSCENKIISFKEMNPPDNIKDTKSDIIFFQRSVPGHD |
| | Fc_N297A | NKMQFECSSYEGYFLACEKERDLFKLILKKEDELGDRSIMFTVQNEDEPKSAD |
| | | KTHTCPPCPAPELLGGPSVFLFPPKPKDTLMISRTPEVTCVVVDVSHEDPEVKF |
| | | NWYVDGVEVHNAKTKPREEQYASTYRVVSVLTVLHQDWLNGKEYKCKVSN |
| | | KALPAPIEKTISKAKGQPREPQVYTLPPSREEMTKNQVSLTCLVKGFYPSDIAV |
| | | EWESNGQPENNYKTTPPVLDSDGSFFLYSKLTVDKSRWQQGNVFSCSVMHE |
| | | ALHNHYTQKSLSLSPGK |
| 329 | WT IL-18- | YFGKLESKLSVIRNLNDQVLFIDQGNRPLFEDMTDSDCRDNAPRTIFIISMYKD |
| | DBo- | SQPRGMAVTISVKCEKISTLSCENKIISFKEMNPPDNIKDTKSDIIFFQRSVPGH |
| | Fc_N297A | DNKMQFECSSYEGYFLACEKERDLFKLILKKEDELGDRSIMFTVQNEDEPKSA |
| | | DKTHTCPPCPAPELLGGPSVFLFPPKPKDTLMISRTPEVTCVVVDVSHEDPEVK |
| | | FNWYVDGVEVHNAKTKPREEQYASTYRVVSVLTVLHQDWLNGKEYKCKVS |
| | | NKALPAPIEKTISKAKGQPREPQVYTLPPSREEMTKNQVSLTCLVKGFYPSDIA |
| | | VEWESNGQPENNYKTTPPVLDSDGSFFLYSKLTVDKSRWQQGNVFSCSVMH |
| | | EALHNHYTQKSLSLSPGK |
| 330 | MM5-DB6- | YFGKLGSKLSVIRNLNDQVLFIDQGNRPLFEDMTDSDIRDNAPRTIFIISMYKL |
| | Fc_N297A | STERGMAVTISVKSEKISTLSCENKIISFKEMNPPDGIKDTKSDIIFFQSSVPGHD |
| | | NKMQFECSSYEGYFLACEKERDLFKLILKKEDELGDRSIMFTVQNEDEPKSAD |
| | | KTHTCPPCPAPELLGGPSVFLFPPKPKDTLMISRTPEVTCVVVDVSHEDPEVKF |
| | | NWYVDGVEVHNAKTKPREEQYASTYRVVSVLTVLHQDWLNGKEYKCKVSN |
| | | KALPAPIEKTISKAKGQPREPQVYTLPPSREEMTKNQVSLTCLVKGFYPSDIAV |
| | | EWESNGQPENNYKTTPPVLDSDGSFFLYSKLTVDKSRWQQGNVFSCSVMHE |
| | | ALHNHYTQKSLSLSPGK |
| 331 | Fc_N297A- | EPKSADKTHTCPPCPAPELLGGPSVFLFPPKPKDTLMISRTPEVTCVVVDVSHE |
| | M12-DB6 | DPEVKFNWYVDGVEVHNAKTKPREEQYASTYRVVSVLTVLHQDWLNGKEY |
| | | KCKVSNKALPAPIEKTISKAKGQPREPQVYTLPPSREEMTKNQVSLTCLVKGF |
| | | YPSDIAVEWESNGQPENNYKTTPPVLDSDGSFFLYSKLTVDKSRWQQGNVFS |
| | | CSVMHEALHNHYTQKSLSLSPGKGGGGSGGGGSGSGGGYFPKLKSKLSVIRN |
| | | LNDQVLFIDQGNRPLFEDMTDSDIRDNAPRTIFIISMYKGSGARGMAVTISVKS |
| | | EKISTLSCENKIISFKEMNPPDTIKDTKSDIIFFQRSVPGHDNKMQFECSSYEGY |
| | | FLACEKERDLFKLILKKEDELGDRSIMFTVQNED |
| 332 | Fc_N297A- | EPKSADKTHTCPPCPAPELLGGPSVFLFPPKPKDTLMISRTPEVTCVVVDVSHE |
| | MM5-DB6 | DPEVKFNWYVDGVEVHNAKTKPREEQYASTYRVVSVLTVLHQDWLNGKEY |
| | | KCKVSNKALPAPIEKTISKAKGQPREPQVYTLPPSREEMTKNQVSLTCLVKGF |
| | | YPSDIAVEWESNGQPENNYKTTPPVLDSDGSFFLYSKLTVDKSRWQQGNVFS |
| | | CSVMHEALHNHYTQKSLSLSPGKGGGGSGGGGSGSGGGYFGKLGSKLSVIRN |
| | | LNDQVLFIDQGNRPLFEDMTDSDIRDNAPRTIFIISMYKLSTERGMAVTISVKS |
| | | EKISTLSCENKIISFKEMNPPDGIKDTKSDIIFFQSSVPGHDNKMQFECSSYEGY |
| | | FLACEKERDLFKLILKKEDELGDRSIMFTVQNED |
| 333 | WT-IL-18- | TACTTCGGCAAGCTGGAGTCCAAGCTGTCCGTGATCCGGAACCTGAACGA |
| | DB6 | CCAGGTGCTGTTCATCGACCAGGGCAACAGGCCTCTCTTCGAGGATATGA |
| | | CCGACTCCGACATCCGCGACAACGCTCCTAGAACAATCTTCATCATCTCCA |
| | | TGTACAAGGACTCCCAGCCCAGGGGAATGGCTGTTACAATTTCCGTGAAG |
| | | TCCGAGAAGATCTCCACCCTGTCCTGCGAGAACAAGATCATCTCCTTCAA |
| | | GGAGATGAACCCCCCCGACAACATCAAAGACACCAAATCCGACATCATCT |
| | | TCTTCCAGCGCAGCGTGCCCGGACATGATAACAAAATGCAGTTCGAATGC |
| | | TCCTCCTACGAGGGCTACTTCCTCGCATGCGAAAAGGAAAGGGACCTGTT |
| | | CAAGCTGATCCTGAAGAAGGAGGACGAGTTGGGGGACAGGAGCATCATG |
| | | TTCACCGTCCAGAACGAGGAC |
| 334 | M12-DB6 | TACTTCCCCAAGCTGAAATCCAAACTGAGTGTGATTCGGAACCTGAATGA |
| | | CCAGGTGCTGTTCATCGACCAGGGCAACAGGCCTCTGTTTGAAGATATGA |
| | | CTGACAGTGACATCCGGGACAACGCCCCAAGAACCATCTTCATCATCTCT |
| | | ATGTACAAGGGCTCCGGGGCTAGGGGAATGGCTGTGACCATTAGCGTGAA |
| | | GTCTGAGAAGATTTCCACCCTGAGCTGCGAGAACAAGATCATTTCCTTCA |
| | | AAGAGATGAACCCCCCCGACACCATCAAGGACACCAAGTCCGACATCATC |
| | | TTCTTCCAGAGGTCCGTGCCCGGCCACGACAACAAGATGCAGTTCGAGTG |
| | | CTCCTCCTACGAGGGCTACTTCCTGGCCTGCGAGAAGGAGAGGGACCTGT |
| | | TCAAGCTGATCCTGAAGAAGGAGGACGAGCTGGGCGACAGGTCCATCATG |
| | | TTCACCGTGCAGAACGAGGAC |
| 335 | MM5-DB6 | TACTTCGGCAAGCTGGGTTCCAAGCTGTCCGTGATCCGGAACCTGAACGA |
| | | CCAGGTGCTGTTCATCGACCAGGGCAACAGGCCTCTCTTCGAGGATATGA |
| | | CCGACTCCGACATCCGCGACAACGCTCCTAGAACAATCTTCATCATCTCCA |
| | | TGTACAAGCTGTCCACCGAGAGGGGGATGGCTGTTACAATTTCCGTGAAG |
| | | TCCGAGAAAATCAGCACACTGTCCTGCGAGAACAAAATCATCTCCTTCAA |
| | | GGAGATGAACCCCCCCGACGGCATCAAGGACACCAAGTCCGACATCATCT |
| | | TCTTCCAGTCCTCCGTGCCCGGCCACGACAACAAGATGCAGTTCGAGTGCT |
| | | CCTCCTACGAGGGCTACTTCCTGGCCTGCGAGAAGGAGAGGGACCTGTTC |
| | | AAGCTGATCCTGAAGAAGGAGGACGAGCTGGGCGACAGGTCCATCATGTT |
| | | CACCGTGCAGAACGAGGAC |
| 336 | WT IL18- | YFGKLESKLSVIRNLNDQVLFIDQGNRPLFEDMTDSDSRDNAPRTIFIISMYKD |
| | SSS- | SQPRGMAVTISVKSEKISTLSSENKIISFKEMNPPDNIKDTKSDIIFFQRSVPGHD |
| | Fc_N297A; | NKMQFESSSYEGYFLACEKERDLFKLILKKEDELGDRSIMFTVQNEDEPKSAD |
| | lacking C- | KTHTCPPCPAPELLGGPSVFLFPPKPKDTLMISRTPEVTCVVVDVSHEDPEVKF |
| | terminal K | NWYVDGVEVHNAKTKPREEQYASTYRVVSVLTVLHQDWLNGKEYKCKVSN |
| | | KALPAPIEKTISKAKGQPREPQVYTLPPSREEMTKNQVSLTCLVKGFYPSDIAV |
| | | EWESNGQPENNYKTTPPVLDSDGSFFLYSKLTVDKSRWQQGNVFSCSVMHE |
| | | ALHNHYTQKSLSLSPG |
| 337 | M12-SSS- | YFPKLKSKLSVIRNLNDQVLFIDQGNRPLFEDMTDSDSRDNAPRTIFIISMYKG |
| | Fc_N297A; | SGARGMAVTISVKSEKISTLSSENKIISFKEMNPPDTIKDTKSDIIFFQRSVPGHD |
| | lacking C- | NKMQFESSSYEGYFLACEKERDLFKLILKKEDELGDRSIMFTVQNEDEPKSAD |
| | terminal K | KTHTCPPCPAPELLGGPSVFLFPPKPKDTLMISRTPEVTCVVVDVSHEDPEVKF |
| | | NWYVDGVEVHNAKTKPREEQYASTYRVVSVLTVLHQDWLNGKEYKCKVSN |
| | | KALPAPIEKTISKAKGQPREPQVYTLPPSREEMTKNQVSLTCLVKGFYPSDIAV |
| | | EWESNGQPENNYKTTPPVLDSDGSFFLYSKLTVDKSRWQQGNVFSCSVMHE |
| | | ALHNHYTQKSLSLSPG |
| 338 | MM5-SSS- | YFGKLGSKLSVIRNLNDQVLFIDQGNRPLFEDMTDSDSRDNAPRTIFIISMYKL |
| | Fc_N297A; | STERGMAVTISVKSEKISTLSSENKIISFKEMNPPDGIKDTKSDIIFFQSSVPGHD |
| | lacking C- | NKMQFESSSYEGYFLACEKERDLFKLILKKEDELGDRSIMFTVQNEDEPKSAD |
| | terminal K | KTHTCPPCPAPELLGGPSVFLFPPKPKDTLMISRTPEVTCVVVDVSHEDPEVKF |
| | | NWYVDGVEVHNAKTKPREEQYASTYRVVSVLTVLHQDWLNGKEYKCKVSN |
| | | KALPAPIEKTISKAKGQPREPQVYTLPPSREEMTKNQVSLTCLVKGFYPSDIAV |
| | | EWESNGQPENNYKTTPPVLDSDGSFFLYSKLTVDKSRWQQGNVFSCSVMHE |
| | | ALHNHYTQKSLSLSPG |
| 339 | M12-SSSS- | YFPKLKSKLSVIRNLNDQVLFIDQGNRPLFEDMTDSDSRDNAPRTIFIISMYKG |
| | Fc_N297A; | SGARGMAVTISVKSEKISTLSSENKIISFKEMNPPDTIKDTKSDIIFFQRSVPGHD |
| | lacking C- | NKMQFESSSYEGYFLASEKERDLFKLILKKEDELGDRSIMFTVQNEDEPKSAD |
| | terminal K | KTHTCPPCPAPELLGGPSVFLFPPKPKDTLMISRTPEVTCVVVDVSHEDPEVKF |
| | | NWYVDGVEVHNAKTKPREEQYASTYRVVSVLTVLHQDWLNGKEYKCKVSN |
| | | KALPAPIEKTISKAKGQPREPQVYTLPPSREEMTKNQVSLTCLVKGFYPSDIAV |
| | | EWESNGQPENNYKTTPPVLDSDGSFFLYSKLTVDKSRWQQGNVFSCSVMHE |
| | | ALHNHYTQKSLSLSPG |
| 340 | M12-DB6- | YFPKLKSKLSVIRNLNDQVLFIDQGNRPLFEDMTDSDIRDNAPRTIFIISMYKGS |
| | Fc_N297A; | GARGMAVTISVKSEKISTLSCENKIISFKEMNPPDTIKDTKSDIIFFQRSVPGHD |
| | lacking C- | NKMQFECSSYEGYFLACEKERDLFKLILKKEDELGDRSIMFTVQNEDEPKSAD |
| | terminal K | KTHTCPPCPAPELLGGPSVFLFPPKPKDTLMISRTPEVTCVVVDVSHEDPEVKF |
| | | NWYVDGVEVHNAKTKPREEQYASTYRVVSVLTVLHQDWLNGKEYKCKVSN |
| | | KALPAPIEKTISKAKGQPREPQVYTLPPSREEMTKNQVSLTCLVKGFYPSDIAV |
| | | EWESNGQPENNYKTTPPVLDSDGSFFLYSKLTVDKSRWQQGNVFSCSVMHE |
| | | ALHNHYTQKSLSLSPG |
| 341 | M12-DB7- | YFPKLKSKLSVIRNLNDQVLFIDQGNRPLFEDMTDSDVRDNAPRTIFIISMYKG |
| | Fc_N297A; | SGARGMAVTISVKIEKISTLSCENKIISFKEMNPPDTIKDTKSDIIFFQRSVPGHD |
| | lacking C- | NKMQFECSSYEGYFLAAEKERDLFKLILKKEDELGDRSIMFTVQNEDEPKSAD |
| | terminal K | KTHTCPPCPAPELLGGPSVFLFPPKPKDTLMISRTPEVTCVVVDVSHEDPEVKF |
| | | NWYVDGVEVHNAKTKPREEQYASTYRVVSVLTVLHQDWLNGKEYKCKVSN |
| | | KALPAPIEKTISKAKGQPREPQVYTLPPSREEMTKNQVSLTCLVKGFYPSDIAV |
| | | EWESNGQPENNYKTTPPVLDSDGSFFLYSKLTVDKSRWQQGNVFSCSVMHE |
| | | ALHNHYTQKSLSLSPG |
| 342 | M12-DB8- | YFPKLKSKLSVIRNLNDQVLFIDQGNRPLFEDMTDSDSRDNAPRTIFIISMYKG |
| | Fc_N297A; | SGARGMAVTISVKIEKISTLSCENKIISFKEMNPPDTIKDTKSDIIFFQRSVPGHD |
| | lacking C- | NKMQFECSSYEGYFLAIEKERDLFKLILKKEDELGDRSIMFTVQNEDEPKSAD |
| | terminal K | KTHTCPPCPAPELLGGPSVFLFPPKPKDTLMISRTPEVTCVVVDVSHEDPEVKF |
| | | NWYVDGVEVHNAKTKPREEQYASTYRVVSVLTVLHQDWLNGKEYKCKVSN |
| | | KALPAPIEKTISKAKGQPREPQVYTLPPSREEMTKNQVSLTCLVKGFYPSDIAV |
| | | EWESNGQPENNYKTTPPVLDSDGSFFLYSKLTVDKSRWQQGNVFSCSVMHE |
| | | ALHNHYTQKSLSLSPG |
| 343 | M12-DB9- | YFPKLKSKLSVIRNLNDQVLFIDQGNRPLFEDMTDSDIRDNAPRTIFIISMYKGS |
| | Fc_N297A; | GARGMAVTISVKIEKISTLSVENKIISFKEMNPPDTIKDTKSDIIFFQRSVPGHD |
| | lacking C- | NKMQFESSSYEGYFLAIEKERDLFKLILKKEDELGDRSIMFTVQNEDEPKSAD |
| | terminal K | KTHTCPPCPAPELLGGPSVFLFPPKPKDTLMISRTPEVTCVVVDVSHEDPEVKF |
| | | NWYVDGVEVHNAKTKPREEQYASTYRVVSVLTVLHQDWLNGKEYKCKVSN |
| | | KALPAPIEKTISKAKGQPREPQVYTLPPSREEMTKNQVSLTCLVKGFYPSDIAV |
| | | EWESNGQPENNYKTTPPVLDSDGSFFLYSKLTVDKSRWQQGNVFSCSVMHE |
| | | ALHNHYTQKSLSLSPG |
| 344 | M12-DB10- | YFPKLKSKLSVIRNLNDQVLFIDQGNRPLFEDMTDSDIRDNAPRTIFIISMYKGS |
| | Fc_N297A; | GARGMAVTISVKLEKISTLSYENKIISFKEMNPPDTIKDTKSDIIFFQRSVPGHD |
| | lacking C- | NKMQFESSSYEGYFLACEKERDLFKLILKKEDELGDRSIMFTVQNEDEPKSAD |
| | terminal K | KTHTCPPCPAPELLGGPSVFLFPPKPKDTLMISRTPEVTCVVVDVSHEDPEVKF |
| | | NWYVDGVEVHNAKTKPREEQYASTYRVVSVLTVLHQDWLNGKEYKCKVSN |
| | | KALPAPIEKTISKAKGQPREPQVYTLPPSREEMTKNQVSLTCLVKGFYPSDIAV |
| | | EWESNGQPENNYKTTPPVLDSDGSFFLYSKLTVDKSRWQQGNVFSCSVMHE |
| | | ALHNHYTQKSLSLSPG |
| 345 | WT IL18- | YFGKLESKLSVIRNLNDQVLFIDQGNRPLFEDMTDSDIRDNAPRTIFIISMYKD |
| | DB6- | SQPRGMAVTISVKSEKISTLSCENKIISFKEMNPPDNIKDTKSDIIFFQRSVPGHD |
| | Fc N297A; | NKMQFECSSYEGYFLACEKERDLFKLILKKEDELGDRSIMFTVQNEDEPKSAD |
| | lacking C- | KTHTCPPCPAPELLGGPSVFLFPPKPKDTLMISRTPEVTCVVVDVSHEDPEVKF |
| | terminal K | NWYVDGVEVHNAKTKPREEQYASTYRVVSVLTVLHQDWLNGKEYKCKVSN |
| | | KALPAPIEKTISKAKGQPREPQVYTLPPSREEMTKNQVSLTCLVKGFYPSDIAV |
| | | EWESNGQPENNYKTTPPVLDSDGSFFLYSKLTVDKSRWQQGNVFSCSVMHE |
| | | ALHNHYTQKSLSLSPG |
| 346 | WT IL-18- | YFGKLESKLSVIRNLNDQVLFIDQGNRPLFEDMTDSDCRDNAPRTIFIISMYKD |
| | DBo- | SQPRGMAVTISVKCEKISTLSCENKIISFKEMNPPDNIKDTKSDIIFFQRSVPGH |
| | Fc_N297A; | DNKMQFECSSYEGYFLACEKERDLFKLILKKEDELGDRSIMFTVQNEDEPKSA |
| | lacking C- | DKTHTCPPCPAPELLGGPSVFLFPPKPKDTLMISRTPEVTCVVVDVSHEDPEVK |
| | terminal K | FNWYVDGVEVHNAKTKPREEQYASTYRVVSVLTVLHQDWLNGKEYKCKVS |
| | | NKALPAPIEKTISKAKGQPREPQVYTLPPSREEMTKNQVSLTCLVKGFYPSDIA |
| | | VEWESNGQPENNYKTTPPVLDSDGSFFLYSKLTVDKSRWQQGNVFSCSVMH |
| | | EALHNHYTQKSLSLSPG |
| 347 | MM5-DB6- | YFGKLGSKLSVIRNLNDQVLFIDQGNRPLFEDMTDSDIRDNAPRTIFIISMYKL |
| | Fc_N297A; | STERGMAVTISVKSEKISTLSCENKIISFKEMNPPDGIKDTKSDIIFFQSSVPGHD |
| | lacking C- | NKMQFECSSYEGYFLACEKERDLFKLILKKEDELGDRSIMFTVQNEDEPKSAD |
| | terminal K | KTHTCPPCPAPELLGGPSVFLFPPKPKDTLMISRTPEVTCVVVDVSHEDPEVKF |
| | | NWYVDGVEVHNAKTKPREEQYASTYRVVSVLTVLHQDWLNGKEYKCKVSN |
| | | KALPAPIEKTISKAKGQPREPQVYTLPPSREEMTKNQVSLTCLVKGFYPSDIAV |
| | | EWESNGQPENNYKTTPPVLDSDGSFFLYSKLTVDKSRWQQGNVFSCSVMHE |
| | | ALHNHYTQKSLSLSPG |

1. An interleukin 18 (IL-18) variant polypeptide comprising at least one mutation at a residue selected from the group consisting of: F2, G3, E6, V11, Q24, L29, D54, Q56, P57, M60, A61, N91, K96, R104, R107, K140, N155, and I149, wherein the amino acid position is relative to a wild-type human IL-18 set forth in SEQ ID NO: 1.

2. The IL-18 variant polypeptide of claim 1, comprising at least one mutation at a residue selected from the group consisting of G3, E6, and M60, wherein the amino acid positions is relative to a wild-type human IL-18 set forth in SEQ ID NO: 1.

3. The IL-18 variant polypeptide of claim 2, comprising at least one of
a) G3P, G3S, G3E, G3N, G3D, or G3A;
b) E6K, E6T, E6R, E6A, E6M, E6L, E6G, E6H, E6S, or E6Y; or
c) M60K.

4. The IL-18 variant polypeptide of any one of claims 1-3, further comprising at least one or more mutations at a residue selected from the group consisting of V11, D54, Q56, P57, N91, K96, R104, R140, I149, and N155.

5. The IL-18 variant polypeptide of claim 4, comprising at least one of
a) D54H, D54W, D54Q, D54S, D54G, D54P, D54L, D54Y, D54F, D54R, or D54A;
b) Q56D, Q56P, Q56H, Q56G, Q56T, Q56R, Q56L, Q56I, Q56S, Q56Y, Q56E, or Q56V;
c) P57R, P57D, P57V, P57W, P57A, P57N, P57S, P57T, P57Q, P57G, P57K, P57H, P57L, P57I or P57E;
d) N91V, N91G, N91A, N91S, N91T, N91R, N91K, N91P, N91I, or N91W;
e) R104V, R104T, R104Y, R104F, R104T, R104L, R104S, R104A, R104E, R104I;
f) N155K;
g) V11I;
h) K96D or K96E;
i) I149M; or
j) K140R.

6. The IL-18 variant polypeptide of any one of claims 1-5, comprising one substitution or a set of substitution combination selected from the group consisting of:
1) G3P, E6K, D54H, Q56D, P57R, N91V, and R104V;
2) G3S, E6K, D54W, Q56P, P57D, N91G, and R104T;
3) G3P, E6K, D54W, Q56H, P57V, N91A, and R104Y;
4) G3P, E6K, D54W, Q56G, P57V, N91V, and R104F;
5) G3E, E6T, D54W, Q56P, P57W, N91V, R104T, and N155K;
6) G3P, E6R, D54W, Q56T, N91V, and R104T;
7) G3P, E6R, D54H, Q56T, P57A, and N91A;
8) G3P, E6R, D54W, Q56P, P57A, N91A, and R104L;
9) G3P, E6R, D54W, Q56R, P57A, N91S, and R104S;
10) G3P, E6R, D54Q, Q56L, P57W, and N91S;
11) G3P, E6R, D54S, Q56R, P57N, N91G, and R104T;
12) G3P, E6K, D54G, Q56G, P57A, and N91T;
13) G3P, E6K, D54Q, Q56I, and P57W;
14) G3N, E6K, D54P, Q56S, P57S, N91R, and R104A;
15) G3P, E6R, D54S, Q56Y, P57T, and N91G;
16) G3P, E6R, D54S, Q56R, P57N, N91G, and R104S;
17) G3P, E6R, D54L, Q56T, P57A, and N91G;
18) G3P, E6R, D54S, Q56R, P57R, N91G, and R104S;
19) G3P, E6K, D54H, Q56E, P57Q, and N91A;
20) G3P, E6R, D54S, Q56R, P57S, N91G, and R104S;
21) G3P, E6R, D54S, Q56S, P57T, N91G, and R104S;
22) G3P, E6R, D54Y, Q56R, P57G, N91K, and R104S;
23) G3P, E6R, D54Y, Q56T, and P57R;
24) G3P, E6R, D54Y, Q56T, and P57S;
25) G3P, E6R, D54L, Q56T, P57T, and N91R;
26) G3P, E6R, D54H, Q56D, P57K, N91V, and R104Y;
27) G3P, E6R, D54H, Q56Y, P57T, N91V, and R104Y;
28) G3P, E6A, D54W, Q56G, P57G, N91V, and R104Y;
29) G3P, E6M, D54F, Q56D, P57R, and N91P;
30) G3P, E6L, D54H, Q56T, P57V, and N91S;
31) G3P, E6R, D54H, Q56I, P57H, N91I, and R104Y;
32) G3P, E6G, D54S, Q56S, and P57R;
33) G3E, E6H, D54R, Q56T, and P57H;
34) G3P, E6R, D54H, Q56R, P57N, N91V, and R104E;
35) G3P, E6R, D54G, Q56G, P57A, and N91G;
36) G3P, E6S, D54A, Q56D, P57Q, and N91G;
37) G3P, E6G, D54Q, Q56V, and P57W;
38) G3P, E6S, D54W, Q56G, P57A, N91V, and R104I;
39) G3P, E6R, D54W, Q56P, P57G, N91V, and R104L;
40) G3D, E6K, D54P, Q56S, P57W, and N91W;
41) G3P;
42) G3P, E6K, D54G, Q56G, and P57A;
43) G3P, E6R, D54L, Q56G, P57S, and N91V;
44) G3P, E6R, V11I, D54G, Q56G, P57A, and N91G;
45) G3P, E6K, D54H, Q56Y, and P57S;
46) E6R, D54W, Q56S, and P57Q;
47) G3P, E6K, D54L, Q56T, P57Q, and N91V;
48) G3A, E6Y, D54R, Q56S, P57L, and N91G;
49) G3P, E6R, D54L, Q56T, P57I, and N91G;
50) E6G, D54L, Q56T, P57E, N91G, and R104S;
51) G3A, E6Y, D54R, Q56S, P57L, and N91A;
52) M60K, and K96D;
53) G3P, E6R, and K96E;
54) M60K;
55) G3P, and E6R;
56) G3P, and E6K;
57) G3D, E6K, and N91S;
58) G3S, and I149M;
59) G3P, E6R, and N91S;
60) E6R;
61) E6R, and N91S;
62) V11I; and
63) G3S, and K140R.

7. The IL-18 variant polypeptide of any one of claims 1-6, comprising an amino acid sequence set forth in any of SEQ ID NOs: 1-4, 8-9, 11-12, 17, 21-22, 26-28, 35, 38-39, 41, 43-44, 49-50, 54-56, 58-59, 65-66, 72-74, 81, 91, 94, 99, 104-105, 114, 116-117, 121-125, 131-134, 139-145, 147, and 149-153.

8. The IL-18 variant polypeptide of any one of claims 1-6, further comprising a S117C substitution, wherein the amino acid position is relative to a wild-type human IL-18 set forth in SEQ ID NO: 1, optionally wherein the S117C substitution promotes a C76-C117 disulfide bond.

9. The IL-18 variant polypeptide of any one of claims 1-6 and 8, further comprising a substitution at C38, wherein the amino acid position is relative to a wild-type human IL-18 set forth in SEQ ID NO: 1, optionally wherein the substitution is selected from the group consisting of C38I, C38V, C38L, and C38M, further optionally wherein the substitution is C38I or C38V.

10. The IL-18 variant polypeptide of any one of claims 1-6 and 8-9, further comprising a substitution at C68, wherein the amino acid position is relative to a wild-type human IL-18 set forth in SEQ ID NO: 1, optionally wherein the substitution is selected from the group consisting of C68I, C68V, C68S, and C68D, further optionally wherein the substitution is C68I, C68L, or C38S.

11. The IL-18 variant polypeptide of any one of claims 1-6 and 8-10, further comprising a substitution at C76 and/or C127, wherein the amino acid position is relative to a wild-type human IL-18 set forth in SEQ ID NO: 1, optionally wherein the C76 substitution is C76Y or C76V, and optionally wherein the C127 substitution is selected from the group consisting of C127A, C127I, C127Y, C127F, and C127L, further optionally wherein the C127 substitution is C127A or C127I.

12. The IL-18 variant polypeptide of any one of claims 1-6 and 8-11, wherein the variant polypeptide further comprises C38S, C68S, and C76S substitutions, optionally wherein the variant polypeptide further comprises a C127S substitution.

13. The IL-18 variant polypeptide of any one of claims 1-6 and 8-12, wherein the variant polypeptide further comprises a set of mutations selected from the group consisting of:
(a) C38I, C68S, and S117C;
(b) C38V, C68I, S117C, and C127A;
(c) C38S, C68I, S117C, and C127I;
(d) C38I, C68I, C76V, and C127I; and
(e) C38I, C68L, and C76Y.

14. An interleukin 18 (IL-18) variant polypeptide comprising at least one mutation at a residue selected from the group consisting of: C38, C68, C76, S117, and C127, wherein the amino acid position is relative to a wild-type human IL-18 set forth in SEQ ID NO: 1.

15. The IL-18 variant polypeptide of claim 14, wherein the at least one mutation comprises a substitution at C38, a substitution at C68, and an S117C substitution.

16. The IL-18 variant polypeptide of claim 14, wherein the at least one mutation is selected from the group consisting of: C38S, C68S, C76S, and C127S.

17. The IL-18 variant polypeptide of claim 16, wherein the variant polypeptide comprises C38S, C68S, and C76S mutations.

18. The IL-18 variant polypeptide of claim 14, wherein the variant polypeptide comprises a set of mutations selected from the group consisting of:
(a) C38I, C68S, and S117C;
(b) C38V, C68I, S117C, and C127A;
(c) C38S, C68I, S117C, and C127I;
(d) C38I, C68I, C76V, and C127I; and
(e) C38I, C68L, and C76Y.

19. The IL-18 variant polypeptide of any one of claims 1-18, wherein the IL-18 variant polypeptide specifically binds to IL-18 receptor α (IL-18Rα) and exhibits substantially reduced binding to IL-18 binding protein (IL-18BP) relative to the wild-type IL-18.

20. The IL-18 variant polypeptide of any one of claims 1-19, wherein the IL-18 variant polypeptide exhibits an increased binding to IL-18Rα relative to the wild-type IL-18.

21. The IL-18 variant polypeptide of any one of claims 1-20, wherein the IL-18 variant polypeptide binds to IL-18Rα with a K_{D} of less than about 5×10⁻⁵ M.

22. The IL-18 variant polypeptide of any one of claims 1-21, wherein the IL-18 variant polypeptide binds to IL-18Rα with a K_{D} of between about 5×10⁻⁵ and about 5×10⁻¹¹ M.

23. The IL-18 variant polypeptide of any one of claims 1-22, wherein the IL-18 variant polypeptide binds to IL-18BP with a K_{D} of more than 5×10⁻⁹ M.

24. The IL-18 variant polypeptide of any one of claims 1-23, wherein the IL-18 variant polypeptide exhibits no binding to IL-18BP.

25. The IL-18 variant polypeptide of any one of claims 1-24, wherein the variant polypeptide comprises a mutation at residue G3, wherein the mutation is selected from the group consisting of: G3P, G3D, G3E, G3F, G3K, G3T, G3W, G3N, and G3S, optionally wherein the mutation is G3P.

26. The IL-18 variant polypeptide of any one of claims 1-25, wherein the variant polypeptide comprises a mutation at residue E6, wherein the mutation is selected from the group consisting of: E6R, E6K, E6G, E6T, E6A, E6S, E6H, E6L, E6M, E6N, E6P, E6Q, E6V, E6W, and E6Y, optionally wherein the mutation is selected from the group consisting of: E6R, E6K, E6G, E6T, E6A, and E6S, further optionally wherein the mutation is selected from the group consisting of: E6R and E6K.

27. The IL-18 variant polypeptide of any one of claims 1-26, wherein the variant polypeptide comprises a mutation at residue D54, wherein the mutation is selected from the group consisting of: D54W, D54H, D54I, D54S, D54Q, D54L, D54M, D54Y, D54P, D54R, D54A, D54F, D54G, D54V, and D54T, optionally wherein the mutation is selected from the group consisting of: D54W, D54H, D54S, D54Q, D54L, and D54Y.

28. The IL-18 variant polypeptide of any one of claims 1-27, wherein the variant polypeptide comprises a mutation at residue N91, wherein the mutation is selected from the group consisting of: N91V, N91A, N91D, N91F, N91G, N91S, N91I, N91P, N91R, N91L, N91T, N91C, N91K, N91Y and N91W, optionally wherein the mutation is selected from the group consisting of: N91V, N91A, N91G, and N91S.

29. The IL-18 variant polypeptide of any one of claims 1-28, wherein the variant polypeptide further comprises a mutation at residue R104, wherein the mutation is selected from the group consisting of: R104S, R104Y, R104T, R104L, R104M, R104V, R104A, R104C, R104E, R104G, R104F, R104H, R104I, and R104N, optionally wherein the mutation is selected from the group consisting of: R104S, R104Y, and R104T.

30. The IL-18 variant polypeptide of any one of claims 1-28, wherein the variant polypeptide comprises no mutation at residue R104.

31. The IL-18 variant polypeptide of any one of claims 1-30, wherein the variant polypeptide comprises a mutation at residue Q56, wherein the mutation is selected from the group consisting of: Q56A, Q56T, Q56G, Q56R, Q56S, Q56D, Q56P, Q56I, Q56Y, Q56H, Q56I, Q56K, Q56W, Q56L, Q56E, Q56F, Q56N, and Q56V, optionally wherein the mutation is selected from the group consisting of: Q56T, Q56G, Q56R, Q56S, Q56D, Q56P, Q56I, and Q56Y.

32. The IL-18 variant polypeptide of any one of claims 1-30, wherein the variant polypeptide comprises no mutation at residue Q56.

33. The IL-18 variant polypeptide of any one of claims 1-32, wherein the variant polypeptide comprises a mutation at residue P57, wherein the mutation is selected from the group consisting of: P57A, P57E, P57F, P57G, P57R, P57W, P57S, P57T, P57V, P57Q, P57H, P57I, P57K, P57L, P57N, P57Y, and P57D, optionally wherein the mutation is selected from the group consisting of: P57A, P57G, P57R, P57W, P57S, P57T, and P57V.

34. The IL-18 variant polypeptide of any one of claims 1-32, wherein the variant polypeptide comprises no mutation at residue P57.

35. The IL-18 variant polypeptide of any one of claims 1-34, wherein the variant polypeptide comprises mutations at G3, E6, D54, and N91, optionally wherein the variant polypeptide comprises mutations:
(i) G3P;
(ii) E6R or E6K;
(iii) D54W, D54H, D54S, or D54Q; and
(iv) N91V, N91A, N91G, or N91S.

36. The IL-18 variant polypeptide of any one of claims 1-31, 33, and 35, wherein the variant polypeptide further comprises mutations at Q56 and P57.

37. The IL-18 variant polypeptide of any one of claims 1-6 and 8-36, wherein the variant polypeptide comprises the amino acid sequence set forth in any one of SEQ ID NOs: 2-299 and 307-318, optionally wherein the variant polypeptide comprises the amino acid sequence set forth in any one of SEQ ID NOs: 307-318, optionally wherein the variant polypeptide comprises the amino acid sequence set forth in any one of SEQ ID NOs: 307-312 and 314-317.

38. A fusion polypeptide comprising the IL-18 variant polypeptide of any one of claims 1-37 and a human IgG Fc domain or variant thereof.

39. The fusion polypeptide of claim 38, comprising a human IgG1 Fc domain variant that comprises an N297A mutation (EU numbering), optionally wherein the C-terminus of the IL-18 variant polypeptide is fused to the N-terminus of the human IgG Fc domain or variant thereof, or the N-terminus of the IL-18 variant polypeptide is fused to the C-terminus of the human IgG Fc domain or variant thereof, optionally wherein the fusion polypeptide comprises the amino acid sequence of any one of SEQ ID NOs: 319-332 and 336-347.

40. A dimer comprising two fusion polypeptides of claim 38 or claim 39.

41. A nucleic acid encoding the IL-18 variant polypeptide of any one of claims 1-37 or the fusion polypeptide of any one of claims 38-39.

42. A vector comprising the nucleic acid of claim 41.

43. A host cell comprising the nucleic acid of claim 41 or the vector of claim 42.

44. A method of producing an IL-18 variant polypeptide or fusion polypeptide, comprising:
(a) culturing the host cell of claim 43 under conditions where the IL-18 variant polypeptide or fusion polypeptide is expressed; and
(b) recovering the IL-18 variant polypeptide or fusion polypeptide produced by the host cell.

45. A pharmaceutical composition comprising the IL-18 variant polypeptide of any one of claims 1-37, the fusion polypeptide of any one of claims 38-39, the nucleic acid of claim 41, or the vector of claim 42.

46. A method of treating a disease in an individual, comprising administering to the individual an effective amount of the pharmaceutical composition of claim 45.

47. A method of activating hIL-18 receptor mediated signal in an individual, comprising administering to the individual an effective amount of the pharmaceutical composition of claim 45.

48. A method of stimulating antigen-experienced immune cells in an individual in need thereof, comprising administering to the individual an effective amount of the pharmaceutical composition of claim 45.
